(19) United States
(12) Patent Application Publication
(10) Pub. No.: US 2005/0148045 A1
(43) Pub. Date: Jul. 7, 2005

(54) Gene cluster for fostriecin biosynthesis

(75) Inventors: Ralph C. Reid, San Rafael, CA (US); Zhihao Hu, Castro Valley, CA (US); Li Tang, Foster City, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: KOSAN BIOSCIENCES, INC., A Delaware corporation, Hayward, CA (US)

(21) Appl. No.: 10/922,282

(22) Filed: Aug. 18, 2004

Related U.S. Application Data
(60) Provisional application No. 60/496,306, filed on Aug. 18, 2003.

(51) Int. Cl. C12Q 1/68; C07H 21/04; C12P 23/00; C12P 19/62; C12N 9/10; C12N 15/74; C12N 1/21

(52) U.S. Cl. 435/67; 435/76; 435/6; 435/252.35; 435/193; 536/23.2; 435/471

(57) ABSTRACT

Domains of fostriecin polyketide synthase and modification enzymes and polynucleotides encoding them are provided. Methods to prepare fostriecin in pharmaceutically useful quantities are described, as are methods to prepare fostriecin analogs and other polyketides using the polynucleotides encoding fostriecin polyketide synthase domains or modifying enzymes.

### CROSS-REFERENCE TO RELATED APPLICATIONS

This application claims benefit under 35 U.S.C. 119 to U.S. provisional application No. 60/496,306 (filed Aug. 18, 2003) the entire contents of which are incorporated herein by reference.

### FIELD OF THE INVENTION

The invention relates to materials and methods for biosynthesis of fostriecin, fostriecin derivatives and analogs, and other useful polyketides. The invention finds application in the fields of molecular biology, chemistry, recombinant DNA technology, human and veterinary medicine, and agriculture.

### BACKGROUND OF THE INVENTION

Polyketides are complex natural products that are produced by microorganisms such as fungi and mycelial bacteria. There are about 10,000 known polyketides, from which numerous pharmaceutical products in many therapeutic areas have been derived, including: adriamycin, epothilone, erythromycin, mevacor, rapamycin, tacrolimus, tetracycline, rapamycin, and many others. However, polyketides are made in very small amounts in microorganisms and are difficult to make or modify chemically. For this and other reasons, biosynthetic methods are preferred for production of therapeutically active polyketides. See PCT publication Nos. WO 93/13663; WO 95/08548; WO 96/40968; WO 97/02358; and WO 98/27203; U.S. Pat. Nos. 4,874,748; 5,063,155; 5,098,837; 5,149,639; 5,672,491; 5,712,146 and 6,410,301; Fu et al., 1994, Biochemistry 33: 9321-26; McDaniel et al., 1993, Science 262: 1546-1550; Kao et al., 1994, Science, 265: 509-12, and Rohr, 1995, Angew. Chem. Int. Ed. Engl. 34: 881-88, each of which is incorporated herein by reference.

Biosynthesis of polyketides may be accomplished by heterologous expression of Type I or modular polyketide synthase enzymes (PKSs). Type I PKSs are large multifunctional protein complexes, the protein components of which are encoded by multiple open reading frames (ORF) of PKS gene clusters. Each ORF of a Type I PKS gene cluster can encode one, two, or more modules of ketosynthase activity. Each module activates and incorporates a two-carbon (ketide) unit into the polyketide backbone. Each module also contains multiple ketide-modifying enzymatic activities, or domains. The number and order of modules, and the types of ketide-modifying domains within each module, determine the structure of the resulting product. Polyketide synthesis may also involve the activity of nonribosomal peptide synthetases (NRPSs) to catalyze incorporation of an amino acid-derived building block into the polyketide, as well as post-synthesis modification, or tailoring enzymes. The modification enzymes modify the polyketide by oxidation or reduction, addition of carbohydrate groups or methyl groups, or other modifications.

In PKS polypeptides, the regions that encode enzymatic activities (domains) are separated by linker regions. These regions collectively can be considered to define boundries of the various domains. Generally, this organization permits PKS domains of different or identical substrate specificities to be substituted (usually at the level of encoding DNA) from other PKSs by various available methodologies. Using this method, new polyketide synthases (which produce novel polyketides) can be produced.

It will be recognized from the foregoing that genetic manipulation of PKS genes and heterologous expression of PKSs can be used for the efficient production of known polyketides, and for production of novel polyketides structurally related to, but distinct from, known polyketides (see references above, and Hutchinson, 1998, Curr. Opin. Microbiol. 1: 319-29; Carreras and Santi, 1998, Curr. Opin. Biotech. 9: 403-11; and U.S. Pat. Nos. 5,712,146 and 5,672,491, each of which is incorporated herein by reference).

One valuable class of polyketides includes fosteriecin and its analogs. Fostriecin (CI-920) is a structurally novel phosphate ester produced by Streptomyces pulveraceus having potent antitumor activity. Fostriecin's antitumor activity is believed to result from selective inhibition of protein phosphatase 2A (PP2A) and protein phosphatase 4 (PP4). Both synthetic and naturally produced analogs of fostriecin with similar activities have been described. See, e.g., Lewy et al., 2002, “Fostriecin: Chemistry and Biology” Current Medicinal Chemistry 9: 2005-2032, and references cited therein, for additional information regarding fostriecin and its analogs.

The chemical structure of fostriecin and congeners PD 113,270 and PD 113, 271, as reported by Lewy et al., 2002, is shown below: 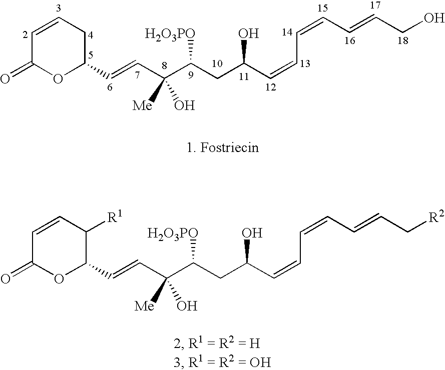
1. Fostriecin
2. PD 113, 270
3. PD 113, 271

Phase I clinical trials of fostriecin were halted due to the unpredictable chemical purity and storage instability of the compound. Accordingly, there is a need for methods for producing fostriecin and both known and novel analogs with sufficient purity and, preferably, with superior storage stability. Fostriecin is synthesized by a modular PKS and modification enzymes.

There is a need for recombinant nucleic acids, host cells, and methods of using those host cells to produce polyketides including but not limited to fostriecin and fostriecin analogs.

These and other needs are met by the materials and methods provided by the present invention.

### SUMMARY OF THE INVENTION

The present invention provides recombinant nucleic acids encoding polyketide synthases and polyketide modification enzymes. The recombinant nucleic acids of the invention are useful in the production of polyketides, including but not limited to fostriecin and fostriecin analogs and derivatives, in recombinant host cells.

In nature, the biosynthesis of fostriecin is performed by a modular PKS, the fostriecin polyketide synthase, and polyketide modification enzymes. Nucleic acids encoding the PKS, modification enzymes, and other polypeptides, have been cloned and characterized. The present invention provides polypeptide, modules, and domains of the fostriecin polyketide synthase, and corresponding nucleic acid sequences encoding them and/or parts thereof. Such compounds are useful, for example, in the production of hybrid PKS enzymes and the recombinant genes that encode them. The present invention also provides post-synthesis modification enzymes, and other proteins involved in fostriecin biosynthesis, and corresponding nucleic acid sequences encoding them and/or parts thereof.

The present invention provides these nucleic acid sequences in isolated, synthetic or recombinant form, including but not limited to isolated form sequences incorporated into a vector of the chromosomal DNA of a host cell.

The present invention also provides recombinant host cells that contain the nucleic acids of the invention. In one embodiment, the host cell provided by the invention is a Streptomyces host cell that produces a fostriecin modification enzyme and/or a domain, module, or protein of the fostriecin PKS. Methods for the genetic manipulation of Streptomyces are described in Kieser et al, “Practical Streptomyces Genetics,” The John Innes Foundation, Norwich (2000), which is incorporated herein by reference in its entirety.

Accordingly, there is provided a recombinant PKS wherein at least 10, 15, 20, or more consecutive amino acids in one or more domains of one or more modules thereof are derived from one or more domains of one or more modules of fostriecin polyketide synthase. In an embodiment at least an entire domain of a module of fostriecin polyketide synthase is included. Representative fostriecin PKS domains useful in this aspect of the invention include, for example, KR, DH, ER, AT, ACP and KS domains. In one embodiment of the invention, the PKS assembled from polypeptides encoded by DNA molecules that comprise coding sequences for PKS domains, wherein at least one encoded domain corresponds to a domain of fostriecin PKS. In such DNA molecules, the coding sequences are operably linked to control sequences so that expression therefrom in host cells is effective. In this manner, fostriecin PKS coding sequences or modules and/or domains can be made to encode PKS to biosynthesize compounds having antibiotic or other useful bioactivity other than fostriecin.

In one aspect, the invention provides a recombinant DNA molecule comprising a sequence encoding at least one domain, and optionally one or more modules, of fostriecin polyketide synthase polypeptide. In an embodiment, the recombinant DNA molecule includes a sequence encoding an open reading frame encoding a polypeptide encoded by fosA, fosB, fosC, fosD, fosE or fosf or encoding a conservative variant of such a polypeptide. In an embodiment, the recombinant DNA molecule encodes a modified fostriecin polyketide synthase polypeptide in which at least one fostriecin PKS domain is inactivated.

In one aspect, the invention provides a recombinant DNA molecule that encodes a chimeric polyketide synthase (PKS) module composed of at least a portion of fostriecin PKS and at least a portion of a second PKS for a polyketide other than fostriecin.

DNA molecules of the invention may be integrated into a host cell chromosome, or into a recombinant vector such as an expression vector in which the DNA molecule is operably linked to a promoter.

In one aspect, the invention provides a host cell comprising a recombinant DNA molecule as described above.

In one aspect, the invention provides a recombinant Streptomyces pulveraceus cell in which at least one domain-encoding region of an endogenous fostriecin polyketide synthase gene is deleted or otherwise inactivated. In an embodiment, the domain has been replaced by a different PKS domain. Also provided is a recombinant Streptomyces pulveraceus cell in which at least polypeptide-encoding ORF of the fostriecin polyketide synthase gene cluster is deleted or otherwise inactivated.

In one aspect, the invention provides an isolated, synthetic or recombinant DNA molecule having a sequence encoded by the insert of pKOS279-117.1F70; pKOS279-117.3F45; pKOS279-117.2F15; or pKos279-117.5F58. The DNA molecule may contain sequence encoding a complete fostriecin PKS module or domain.

The invention provides a method of producing a polyketide by culturing a cell under conditions under which the cell produces the polyketide, where the cell contains a recombinant polynucleotide synthase that contains at least one domain from the Streptomyces pulveraceus fostriecin polyketide synthase, and where the cell does not make the polyketide in the absence of the recombinant polynucleotide. In one embodiment, the domain is encoded by a subsequence of SEQ ID NO:1 or a sequence that hybridizes under stringent conditions to a subsequence of SEQ ID NO:1. In one embodiment the cell is not Streptomyces pulveraceus. In an embodiment, the polyketide is fostriecin, PD 113,270 or PD 113, 271.

The invention provides a method of producing a polyketide by recombinantly modifying a gene in the fostriecin PKS gene cluster of a cell comprising the gene cluster to produce a recombinant cell, or obtaining a progeny of the recombinant cell and growing the cell, or progeny, under conditions whereby a polyketide other than fostriecin is synthesized by the cell. Non-limiting examples of such modifications include (a)substitution of a fostriecin AT domain with an AT domain having a different specificity; (b) inactivation of a domain of a fostriecin polyketide synthase module, where the domain is selected from the group consisting of a KS domain, an AT domain, an ACP domain, a KR domain, a DH domain, and an ER domain; or, (c) substitution of KS domain, an ACP domain, a KR domain, a DH domain, or an ER domain with a domain having a different specificity.

The aforementioned methods can also include the step of recovering the synthesized polyketide. The recovered polyketide may be chemically modified and/or formulated for administration to a mammal.

These and other aspects of the present invention are described in more detail in the Detailed Description of the Invention, below.

### BRIEF DESCRIPTION OF THE DRAWINGS

FIGS. 1A, 1B and 1C show the organization of the fostriecin PKS biosynthetic gene cluster.

FIG. 2 shows hypothetical roles for the nine modules of the fostriecin polyketide synthase complex (modules 0-8)) by showing hypothetical PKS-bound intermediates, the product released from the PKS (in brackets) and the result of post-PKS modification enzymes (symbolized by three arrows).

FIG. 3 shows the approximate relationship of cosmids from “overlap family 1,” encoding the fostriecin PKS gene cluster as estimated during cloning.

### DETAILED DESCRIPTION OF THE INVENTION

The present invention provides recombinant materials for the production of polyketides including, but not limited to, fostriecin and its derivatives and analogs. In an aspect, the invention provides recombinant nucleic acids encoding at least one domain of a fostriecin polyketide synthase. In another aspect, the present invention provides recombinant nucleic acids encoding an enzyme involved in fostriecin biosynthesis or post synthesis modification. Methods and host cells for using these nucleic acid sequences to produce or modify a polyketide in recombinant host cells are also provided. Given the valuable properties of fostriecin and its derivatives and analogs, means to produce useful quantities of these molecules in a highly pure form is of great value. The nucleotide sequences of the fostriecin biosynthetic gene cluster encoding domains, modules and polypeptides of fostriecin polyketide synthase, and modifying enzymes, and other polypeptides can be used, for example, to make both known and novel polyketides. Further, the fostriecin modifying enzymes can be used to modify other polyketides and produce derivatives with enhanced solubility and/or bioactivity. The compounds produced using methods of the invention may be used, without limitation, as antitumor agents or for other therapeutic or research uses, as intermediates for further enzymatic or chemical modification, as agents for in vitro inhibition of protein phosphatase and/or for other therapeutic, industrial and agricultural purposes.

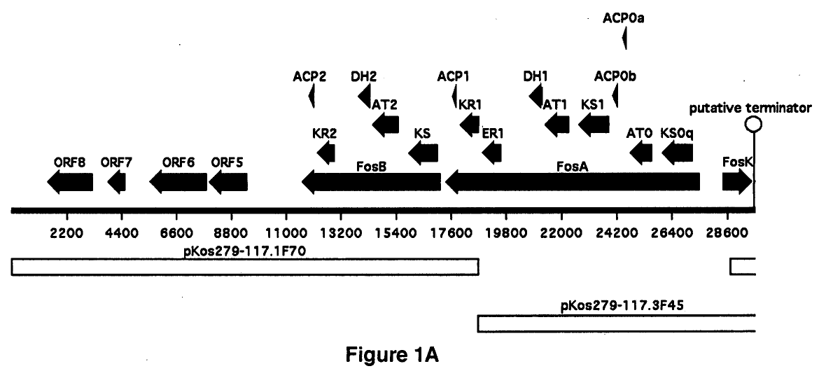

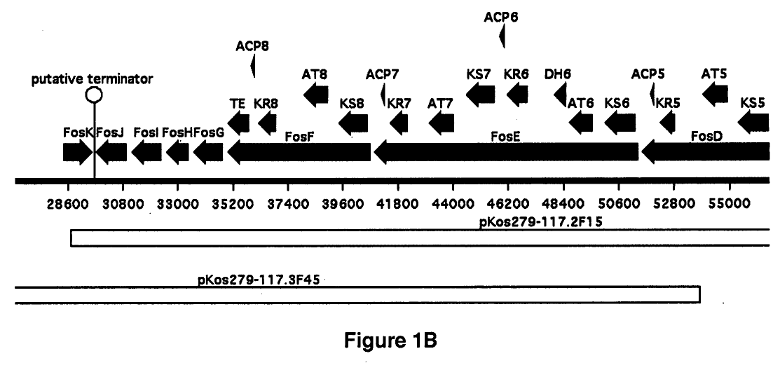

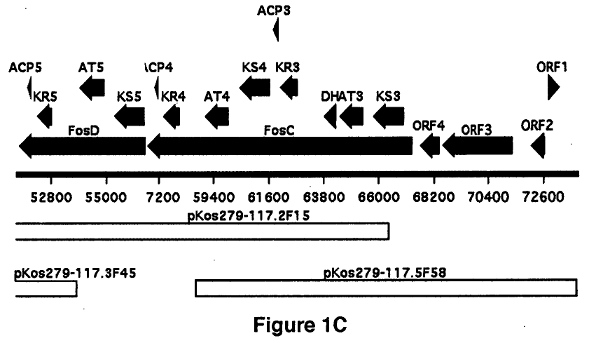

The polynucleotides encoding fostriecin PKS domains, modules and polypeptides, and encoding fostriecin modifying proteins of the present invention were isolated from Streptomyces pulveraceus as described in Example 1. Tables 1-4, and FIG. 1 describe the genes or open reading frames of the fostriecin polyketide synthase gene cluster and the encoded polypeptides, modules and domains. These tables and figure also describe the characteristics of non-coding sequences and sequences encoding other genes of the fostriecin gene cluster, including genes encoding regulatory proteins, transport proteins, and others.

It will be understood that each reference herein to a nucleic acid sequence is also intended to refer to and include the complementary sequence, unless otherwise stated or apparent from context. Provided with the nucleic acid sequences disclosed herein, it will be trivial for the reader to immediately determine the sequence of a complementary stand based on base-pairing rules (e.g., A:T, A:U, C:G). Similarly, provided with the nucleic acid sequences disclosed herein one of skill can easily, by reference to the genetic code, identify open reading frames and the amino acid sequences of encoded polypeptides.

Table 1, below, describes the positions of fostriecin polyketide synthase polypeptides, modules and domains with reference to the DNA sequence set forth in Table 3 (SEQ ID NO:1). “Complement” indicates that the polypeptide sequence is encoded by the complement of SEQ ID NO: 1. Abbreviations used in the table, and elsewhere in the specification, include: ketosynthase (“KS”) domain or activity; acyltransferase (“AT”) domain or activity; acyl carrier protein (“ACP”) domain or activity; ketoreductase (“KR”) domain or activity, a dehydratase (“DH”) domain or activity; enoylreductase (“ER”) domain or activity; thioesterase (“TE”).

TABLE 1
Fostriecin polyketide synthase ORFs, Modules and Domains

| | | | Position in SEQ ID NO: 1 |
|---|---|---|---|
| ORF | # aa | | coding strand (nucleotide pair) |
| fosC | 3542 | Modules 3-4 | complement(56750 . . . 67378) |
| | | KS3 | complement(65783 . . . 67063) |
| | | AT3 | complement(64382 . . . 65431) |
| | | DH3 | complement(63770 . . . 64357) |
| | | KR3 | complement(62039 . . . 62839) |
| | | ACP3 | complement(61757 . . . 62014) |
| | | KS4 | complement(60401 . . . 61678) |
| | | AT4 | complement(59054 . . . 60067) |
| | | KR4 | complement(57368 . . . 58105) |
| | | ACP4 | complement(57014 . . . 57271) |
| fosD | 1738 | Module 5 | complement(51497 . . . 56713) |
| | | KS5 | complement(55328 . . . 56605) |
| | | AT5 | complement(53942 . . . 54994) |
| | | KR5 | complement(52241 . . . 52918) |
| | | ACP5 | complement(51809 . . . 52066) |
| fosE | 3537 | Modules 6-7; | complement(40820 . . . 51433) |
| | | KS6 | complement(50024 . . . 51334) |
| | | AT6 | complement(48608 . . . 49648) |
| | | DH6 | complement(47993 . . . 48574) |
| | | KR6 | complement(46151 . . . 47017) |
| | | ACP6 | complement(45854 . . . 46114) |
| | | KS7 | complement(44474 . . . 45754) |
| | | AT7 | complement(43058 . . . 44098) |
| | | KR7 | complement(41429 . . . 42229) |
| | | ACP7 | complement(41093 . . . 41350) |
| fosF | 1932 | Module 8 and TE; | complement(34979 . . . 40774) |
| | | KS8 | complement(39428 . . . 40672) |
| | | AT8 | complement(38003 . . . 39079) |
| | | KR8 | complement(36212 . . . 37009) |
| | | ACP8 | complement(35912 . . . 36169) |
| | | TE | complement(34979 . . . 35911) |
| fosA | 3414 | Modules 0-1 | complement(17358 . . . 27602) |
| | | KS0q | complement(26019 . . . 27278) |
| | | AT0 | complement(24722 . . . 25640) |
| | | ACP0a | complement(24414 . . . 24671) |
| | | ACP0b | complement(24039 . . . 24296) |
| | | KS1 | complement(22701 . . . 23990) |
| | | AT1 | complement(21336 . . . 22394) |
| | | DH1 | complement(20715 . . . 21302) |
| | | ER1 | complement(18813 . . . 19685) |
| | | KR1 | complement(17952 . . . 18797) |
| | | ACP1 | complement(17631 . . . 17891) |
| fosB | 1880 | Module 2 | complement(11623 . . . 17265) |
| | | KS2 | complement(15883 . . . 17163) |
| | | AT2 | complement(14455 . . . 15576) |
| | | DH2 | complement(13855 . . . 14424) |
| | | KR2 | complement(12247 . . . 13026) |
| | | ACP2 | complement(11920 . . . 12177) |

In one aspect of the invention, purified and isolated DNA molecules are provided that comprise coding sequences for one or more domains or modules of a Streptomyces pulveraceus fostriecin polyketide synthase. Examples of such encoded domains include fostriecin polyketide synthase KR, DH, ER, AT, ACP, and KS domains. In one aspect, the invention provides DNA molecules which sequences encoding one or more polypeptides of fostriecin polyketide synthase are operably linked to expression control sequences that are effective in suitable host cells to produce fostriecin, its analogs or derivatives, or novel polyketides. In one aspect, the complete set of synthase-encoding genes is provided.

In one aspect, the invention provides an isolated or recombinant DNA molecule comprising a nucleotide sequence that encodes at least one domain, alternatively at least one module, alternatively at least one polypeptide, involved in the biosynthesis of a fostriecin.

In one aspect, the invention provides an isolated or recombinant DNA molecule encoding a polypeptide or portion thereof, including a PKS module or domain, encoded in the Streptomyces pulveraceus fostriecin polyketide synthase gene cluster sequence.

In one aspect, the invention provides an isolated or recombinant DNA molecule encoding a complete polypeptide, module or domain comprising an amino acid sequence encoded in SEQ ID NOS: 1, 23, 27 or 33, or a conservatively modified variant thereof. In one aspect, the invention provides an isolated or recombinant DNA molecule encoding a subsequence from a polypeptide, module or domain comprising an amino acid sequence encoded in SEQ ID NOS: 1, 23, 27 or 33, or a conservatively modified variant thereof. The subsequence may comprise a sequence encoding a catalytically active fragment (having an activity characteristic of the domain, e.g., AT, KR, KS, DH, ER, ACP, TE activity) of a PKS module or domain. The DNA molecule may comprise a sequence encoding a polypeptide involved in post-synthesis modification of the fostriecin precursor or encoding another polypeptide of the fostriecin gene cluster.

In one aspect, the invention provides the present invention provides an isolated or recombinant DNA molecule comprising a nucleotide sequence that encodes an open reading frame, module or domain having an amino acid sequence identical or substantially similar to an ORF, module or domain encoded by an ORF of the fostriecin polyketide synthase cluster sequence. A polypeptide, module or domain having a sequence substantially similar to a reference sequence may have substantially the same activity as the reference protein, module or domain (e.g., when integrated into an appropriate PKS framework using methods known in the art).

In an embodiment, the invention provides a nucleotide sequence that encodes a polypeptide, such as a conservatively modified variant of a polypeptide, module or domain involved in the biosynthesis of a fostriecin, and comprises at least 10, 20, 25, 30, 35, 40, 45, or 50 contiguous base pairs identical to a sequence of SEQ ID NOS: 1, 23, 27 or 33. In one aspect, the invention provides an isolated or recombinant DNA molecule comprising a nucleotide sequence that encodes at least one polypeptide, module or domain that comprises at least 10, 15, 20, 30, or 40 contiguous residues of a corresponding polypeptide, module or domain comprising a sequence of SEQ ID NOS: 1, 23, 27 or 33.

It will be understood that, due to the degeneracy of the genetic code, a large number of DNA sequences encode the amino acid sequences of the domains, modules, and proteins of the fostriecin PKS, the enzymes involved in fostriecin modification and other polypeptides encoded by the genes of the fostriecin biosynthetic gene cluster. The present invention contemplates all such DNAs. For example, it may be advantageous to optimize sequence to account for the codon preference of a host organism. The invention also contemplates naturally occurring genes encoding the fostriecin PKS and modifying (or “tailoring”) enzymes that are polymorphic or other variants.

As used herein, a conservatively modified variant of a protein or fragment (e.g., domain) has substantial sequence identity to a reference amino acid sequence or is encoded by a DNA substantial sequence identity to a reference nucleic acid sequence.

The terms “substantial identity,” “substantial sequence identity,” or “substantial similarity” in the context of nucleic acids, refers to a measure of sequence similarity between two polynucleotides. Substantial sequence identity can be determined by hybridization under stringent conditions, by direct comparison, or other means. For example, two polynucleotides can be identified as having substantial sequence identity if they are capable of specifically hybridizing to each other under stringent hybridization conditions. Other degrees of sequence identity (e.g., less than “substantial”) can be characterized by hybridization under different conditions of stringency. “Stringent hybridization conditions” refers to conditions in a range from about 5° C. to about 20° C. or 25° C. below the melting temperature (Tm) of the target sequence and a probe with exact or nearly exact complementarity to the target. As used herein, the melting temperature is the temperature at which a population of double-stranded nucleic acid molecules becomes half-dissociated into single strands. Methods for calculating the Tm of nucleic acids are well known in the art (see, e.g., Berger and Kimmel, 1987, Methods In Enzymology, Vol. 152: Guide To Molecular Cloning Techniques, San Diego: Academic Press, Inc. and Sambrook et al., 1989, Molecular Cloning: A Laboratory Manual, 2nd Ed., Vols. 1-3, Cold Spring Harbor Laboratory). Typically, stringent hybridization conditions for probes greater than 50 nucleotides are salt concentrations less than about 1.0 M sodium ion, typically about 0.01 to 1.0 M sodium ion at pH 7.0 to 8.3, and temperatures at least about 50° C., preferably at least about 60° C. As noted, stringent conditions may also be achieved with the addition of destabilizing agents such as formamide, in which case lower temperatures may be employed. Exemplary conditions include hybridization at 7% sodium dodecyl sulfate (SDS), 0.5 M NaPO₄ pH 7.0, 1 mM EDTA at 50° C.; wash with 2×SSC, 1% SDS, at 50° C.

Alternatively, substantial sequence identity can be described as a percentage identity between two nucleotide or amino acid-sequences. Two nucleic acid sequences are considered substantially identical when they are at least about 70% identical, or at least about 80% identical, or at least about 90% identical, or at least about 95% or 98% identical. Two amino acid sequences are considered substantially identical when they are at least about 60%, sequence identical, more often at least about 70%, at least about 80%, or have at least about 90% sequence identity. Percentage sequence (nucleotide or amino acid) identity is typically calculated using art known means to determine the optimal alignment between two sequences and comparing the two sequences. Optimal alignment of sequences may be conducted using the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988) Proc. Nail. Acad. Sci. U.S.A. 85: 2444, by the BLAST algorithm of Altschul (1990) J. Mol. Biol. 215: 403-410; and Shpaer (1996) Genomics 38: 179-191, or by the Needleham et al. (1970) J. Mol. Biol. 48: 443-453; and Sankoff et al., 1983, Time Warps, String Edits, and Macromolecules, The Theory and Practice of Sequence Comparison, Chapter One, Addison-Wesley, Reading, Mass.; generally by computerized implementations of these algorithms (GAP, BESTFIT, FASTA, and TFASTA in the Wisconsin Genetics Software Package, Genetics Computer Group, 575 Science Dr., Madison, Wis.; BLAST from the National Center for Biotechnology Information (http://www.ncbi.nlm.nih.gov/). In each case default parameters are used (for example the BLAST program uses as defaults a wordlength (W) of 11, the BLOSUM62 scoring matrix (see Henikoff(1992) Proc. Natl. Acad.

Sci. USA 89: 10915-10919) alignments (B) of 50, expectation (E) of 10, M=5, N=4, and a comparison of both strands).

As discussed in Example 1, the gene cluster sequences disclosed herein were determined from the inserts of cosmids pKOS279-117.1 F70, pKOS279-117.3F45, pKOS279-117.2F15, and pKos279-117.5F58. Accordingly, the invention provides an isolated or recombinant DNA molecule comprising a sequence from the insert of one or more of these cosmids. In an embodiment, the isolated or recombinant DNA molecule encodes a polypeptide or portion thereof, such as a module or domain. In an embodiment, the isolated or recombinant DNA molecule comprises at least 10, 20, 30, 40, 50 or 100 basepairs having a sequence of the cosmid insert.

The invention methods may be directed to the preparation of an individual polyketide. The polyketide may or may not be novel, but the method of preparation permits a more convenient or alternative method of preparing it. The resulting polyketides may be further modified to convert them to other useful compounds. Examples of chemical structures of that can be made using the materials and methods of the present invention include PD 113,270 and PD 113, 271, other known analogs, such as those described in Lewy et al., 2002, “Fostriecin: Chemistry and Biology” Current Medicinal Chemistry 9: 2005-2032 and the references cited therein, and novel molecules produced by modified or chimeric PKSs comprising a portion of the fosteriecin PKS sequence, molecules produced by the action of polyketide modifying enzymes from the fosteriecin PKS cluster on products of other PKSs, molecules produced by the action on products of the fosteriecin PKS of polyketide modifying enzymes from other PKSs, and the like.

As noted, in one aspect the invention provides recombinant PKS wherein at least 10, 15, 20, 30, or more consecutive amino acids in one or more domains of one or more modules thereof are derived from one or more domains of one or more modules of fostriecin polyketide synthase.

In one aspect, the invention provides a recombinant polyketide synthase derived from a naturally occurring PKS. A PKS “derived from” a naturally occurring PKS contains the scaffolding encoded by all the portion employed of the naturally occurring synthase gene, contains at least two modules that are functional, and contains mutations, deletions, or replacements of one or more of the activities of these functional modules so that the nature of the resulting polyketide is altered. This definition applies both at the protein and genetic levels.

Particular embodiments include those wherein a KS, AT, KR, DH, or ER has been inactivated (e.g., by deletion or other mutation), mutated to change its activity, and/or replaced by a version of the activity from a different PKS or from another location within the same PKS. Embodiments include derivatives where at least one noncondensation cycle enzymatic activity (KR, DH, or ER) has been inactivated (e.g., by deletion or other mutation) wherein any of these activities has been added or mutated so as to change the ultimate polyketide synthesized. There are at least five degrees of freedom for constructing a polyketide synthase in terms of the polyketide that will be produced. See, U.S. Pat. No. 6,509,455 for a discussion.

As can be appreciated by those skilled in the art, polyketide biosynthesis can be manipulated to make a product other than the product of a naturally occurring PKS biosynthetic cluster. For example, AT domains can be altered or replaced to change specificity. The variable domains within a module can be deleted and or inactivated or replaced with other variable domains found in other modules of the same PKS or from another PKS. See e.g., Katz & McDaniel, Med Res Rev 19: 543-558 (1999) and WO 98/49315. Similarly, entire modules can be deleted and/or replaced with other modules from the same PKS or another PKS. See e.g., Gokhale et al., Science 284: 482 (1999) and WO 00/47724 each of which are incorporated herein by reference. Protein subunits of different PKSs also can be mixed and matched to make compounds having the desired backbone and modifications. For example, subunits of 1 and 2 (encoding modules 1-4) of the pikromycin PKS were combined with the DEBS3 subunit to make a hybrid PKS product (see Tang et al., Science, 287: 640 (2001), WO 00/26349 and WO 99/6159).

It will be appreciated that an amino acid sequence of a protein or domain can be changed without eliminating or substantially changing the function or activity of the wild-type protein or domain, for example, by making conservative substitutions of amino acids. The present invention encompasses polypeptides that are conservatively modified variants of a polypeptide encoded in SEQ ID NO:1 and retain the activity of the wild-type polypeptide. Such polypeptides can be identified by routine screening methods. For example, a polypeptide having a substitution or combination of substitutions relative to wild-type can be prepared by mutation of DNA encoding the fostriecin cluster polypeptide or domain, and the effect (if any) of the sequence modification can be assessed by expressing the protein in a suitable host cell under conditions in which fostriecin is produced in the cell when the unmodified protein is expressed.

This assay can be carried out in Streptomyces pulveraceus by modification of endogenous genes or, alternatively, polynucleotides modified in vitro can be expressed in heterologous hosts as described elsewhere herein. Production of fostriecin at a level not less that 60% of the level produced by the wild-type sequence, preferably at least 80%, and most preferably not less than 95% of the level produced by the wild-type sequence is indicative that the modified polypeptide or domain has the same activity as the unmodified-parent. The invention includes such modified polypeptides and the nucleic acid sequences encoding them.

In other embodiments, a domain or other region of a fostriecin polyketide synthase polypeptide can be removed or otherwise inactivated or replaced with a different PKS domain.

Mutations can be introduced into PKS genes such that polypeptides with altered activity are encoded. Polypeptides with “altered activity” include those in which one or more domains are inactivated or deleted, or in which a mutation changes the substrate specificity of a domain, as well as other alterations in activity. Mutations can be made to the native sequences using conventional techniques. The substrates for mutation can be an entire cluster of genes or only one or two of them; the substrate for mutation may also be portions of one or more of these genes. Techniques for mutation include preparing synthetic oligonucleotides including the mutations and inserting the mutated sequence into the gene encoding a PKS subunit using restriction endonuclease digestion. (See, e.g., Kunkel, T. A. Proc Natl Acad Sci USA (1985) 82: 448; Geisselsoder et al. BioTechniques (1987) 5: 786.) Alternatively, the mutations can be effected using a mismatched primer (generally 10-20 nucleotides in length) that hybridizes to the native nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. (See Zoller and Smith, Methods in Enzymology (1983) 100: 468). Primer extension is effected using DNA polymerase. The product of the extension reaction is cloned, and those clones containing the mutated DNA are selected. Selection can be accomplished using the mutant primer as a hybridization probe. The technique is also applicable for generating multiple point mutations. (See, e.g., Dalbie-McFarland et al. Proc Natl Acad Sci USA (1982) 79: 6409). PCR mutagenesis can also be used for effecting the desired mutations.

Random mutagenesis of selected portions of the nucleotide sequences encoding enzymatic activities can be accomplished by several different techniques known in the art, e.g., by inserting an oligonucleotide linker randomly into a plasmid,

In addition to providing mutated forms of regions encoding enzymatic activity, regions encoding corresponding activities from different PKS synthases or from different locations in the same PKS synthase can be recovered, for example, using PCR techniques with appropriate primers. By “corresponding” activity encoding regions is meant those regions encoding the same general type of activity—e.g., a ketoreductase activity in one location of a gene cluster would “correspond” to a ketoreductase-encoding activity in another location in the gene cluster or in a different gene cluster; similarly, a complete reductase cycle could be considered corresponding—e.g., KR/DH/ER could correspond to KR alone.

If replacement of a particular target region in a host polyketide synthase is to be made, this replacement can be conducted in vitro using suitable restriction enzymes or can be effected in vivo using recombinant techniques involving homologous sequences framing the replacement gene. One such system involving plasmids of differing temperature sensitivities is described in PCT application WO 96/40968. Another useful method for modifying a PKS gene (e.g., making domain substitutions or “swaps”) is a RED/ET cloning procedure developed for constructing domain swaps or modifications in an expression plasmid without first introducing restriction sites. The method is related to ET cloning methods (see, Datansko & Wanner, 2000, Proc. Natl. Acad. Sci. U.S.A. 97, 6640-45; Muyrers et al, 2000, Genetic Engineering 22: 77-98). The RED/ET cloning procedure is used to introduce a unique restriction site in the recipient plasmid at the location of the targeted domain. This restriction site is used to subsequently linearize the recipient plasmid in a subsequent ET cloning step to introduce the modification. This linearization step is necessary in the absence of a selectable marker, which cannot be used for domain substitutions. An advantage of using this method for PKS engineering is that restriction sites do not have to be introduced in the recipient plasmid in order to construct the swap, which makes it faster and more powerful because boundary junctions can be altered more easily.

In a further aspect, the invention provides methods for expressing chimeric or hybrid PKSs and products of such PKSs. For example, the invention provides (1) encoding DNA for a chimeric PKS that is substantially patterned on a non-fostriecin producing enzyme, but which includes one or more functional domains, modules or polypeptides of fostriecin PKS; and (2) encoding DNA for a chimeric PKS that is substantially patterned on the fostriecin PKS, but which includes one or more functional domains, modules, or polypeptides of another PKS or NRPS.

With respect to item (1) above, in one embodiment, the invention provides chimeric PKS enzymes in which the genes for a non-fostriecin PKS function as accepting genes, and one or more of the above-identified coding sequences for fostriecin domains or modules are inserted as replacements for one or more domains or modules of comparable function. Construction of chimeric molecules is most effectively achieved by construction of appropriate encoding polynucleotides. In making a chimeric molecule, it is not necessary to replace an entire domain or module accepting of the PKS with an entire domain or module of fostriecin PKS: subsequences of a PKS domain or module that correspond to a peptide subsequence in an accepting domain or module, or which otherwise provide useful function, may be used as replacements. Accordingly, appropriate encoding DNAs for construction of such chimeric PKS include those that encode at least 10, 15, 20, 40 or more amino acids of a selected fostriecin domain or module.

Recombinant methods for manipulating modular PKS genes to make chimeric PKS enzymes are described in U.S. Pat. Nos. 5,672,491; 5,843,718; 5,830,750; and 5,712,146; and in PCT publication Nos. 98/49315 and 97/02358. A number of genetic engineering strategies have been used with DEBS to demonstrate that the structures of polyketides can be manipulated to produce novel natural products, primarily analogs of the erythromycins (see the patent publications referenced supra and Hutchinson, 1998, Curr Opin Microbiol. 1: 319-329, and Baltz, 1998, Trends Microbiol. 6: 76-83). In one embodiment, the components of the chimeric PKS are arranged onto polypeptides having interpolypeptide linkers that direct the assembly of the polypeptides into the functional PKS protein, such that it is not required that the PKS have the same arrangement of modules in the polypeptides as observed in natural PKSs. Suitable interpolypeptide linkers to join polypeptides and intrapolypeptide linkers to join modules within a polypeptide are described in PCT publication WO 00/47724.

A partial list of sources of PKS sequences for use in making chimeric molecules, for illustration and not limitation, includes Avermectin (U.S. Pat. No. 5,252,474; MacNeil et al., 1993, Industrial Microorganisms: Basic and Applied Molecular Genetics, Baltz, Hegeman, & Skatrud, eds. (ASM), pp. 245-256; MacNeil et al., 1992, Gene 115: 119-25); Candicidin (FRO008) (Hu et al., 1994, Mol. Microbiol. 14: 163-72); Epothilone (U.S. Pat. No. 6,303,342); Erythromycin (WO 93/13663; U.S. Pat. No. 5,824,513; Donadio et al., 1991, Science 252: 675-79; Cortes et al., 1990, Nature 348: 176-8); FK-506 (Motamedi et al., 1998, Eur. J. Biochem. 256: 528-34; Motamedi et al., 1997, Eur. J. Biochem. 244: 74-80); FK-520 (U.S. Pat. No. 6,503,737; see also Nielsen et al., 1991, Biochem. 30: 5789-96); Lovastatin (U.S. Pat. No. 5,744,350); Nemadectin (MacNeil et al., 1993, supra); Niddamycin (Kakavas et al., 1997, J. Bacteriol. 179: 7515-22); Oleandomycin (Swan et al., 1994, Mol. Gen. Genet. 242: 358-62; U.S. Pat. No. 6,388,099; Olano et al., 1998, Mol. Gen. Genet. 259: 299-308); Platenolide (EP Pat. App. 791,656); Rapamycin (Schwecke et al., 1995, Proc. Natl. Acad. Sci. USA 92: 7839-43); Aparicio et al., 1996, Gene 169: 9-16); Rifamycin (August et al., 1998, Chemistry & Biology, 5: 69-79); Soraphen (U.S. Pat. No. 5,716,849; Schupp et al., 1995, J. Bacteriology 177: 3673-79); Spiramycin (U.S. Pat. No. 5,098,837); Tylosin (EP 0 791,655; Kuhstoss et al., 1996, Gene 183: 231-36; U.S. Pat. No. 5,876,991). Additional suitable PKS coding sequences remain to be discovered and characterized, but will be available to those of skill (e.g., by reference to GenBank).

The fostriecin PKS-encoding polynucleotides of the invention may also be used in the production of libraries of PKSs (i.e., modified and chimeric PKSs comprising at least a portion of the fostriecin PKS sequence. The invention provides libraries of polyketides by generating modifications in, or using a portion of, the fostriecin PKS so that the protein complexes produced by the cluster have altered activities in one or more respects, and thus produce polyketides other than the natural fostriecin product of the PKS. Novel polyketides may thus be prepared, or polyketides in general prepared more readily, using this method. By providing a large number of different genes or gene clusters derived from a naturally occurring PKS gene cluster, each of which has been modified in a different way from the native PKS cluster, an effectively combinatorial library of polyketides can be produced as a result of the multiple variations in these activities. Expression vectors containing nucleotide sequences encoding a variety of PKS systems for the production of different polyketides can be transformed into the appropriate host cells to construct a polyketide library. In one approach, a mixture of such vectors is transformed into the selected host cells and the resulting cells plated into individual colonies and selected for successful transformants. Each individual colony has the ability to produce a particular PKS synthase and ultimately a particular polyketide. A variety of strategies can be devised to obtain a multiplicity of colonies each containing a PKS gene cluster derived from the naturally occurring host gene cluster so that each colony in the library produces a different PKS and ultimately a different polyketide. The number of different polyketides that are produced by the library is typically at least four, more typically at least ten, and preferably at least 20, more preferably at least 50, reflecting similar numbers of different altered PKS gene clusters and PKS gene products. The number of members in the library is arbitrarily chosen; however, the degrees of freedom outlined above with respect to the variation of starter, extender units, stereochemistry, oxidation state, and chain length is quite large. The polyketide producing colonies can be identified and isolated using known techniques and the produced polyketides further characterized. The polyketides produced by these colonies can be used collectively in a panel to represent a library or may be assessed individually for activity. See, for example,

Colonies in the library are induced to produce the relevant synthases and thus to produce the relevant polyketides to obtain a library of candidate polyketides. The polyketides secreted into the media can be screened for binding to desired targets, such as receptors, signaling proteins, and the like. The supernatants per se can be used for screening, or partial or complete purification of the polyketides can first be effected. Typically, such screening methods involve detecting the binding of each member of the library to receptor or other target ligand. Binding can be detected either directly or through a competition assay. Means to screen such libraries for binding are well known in the art. Alternatively, individual polyketide members of the library can be tested against a desired target. In this event, screens wherein the biological response of the target is measured can be included.

As noted above, the DNA compounds of the invention can be expressed in host cells for production of proteins and of known and novel compounds. Preferred hosts include fungal systems such as yeast and prokaryotic hosts, but single cell cultures of, for example, mammalian cells could also be used. A variety of methods for heterologous expression of PKS genes and host cells suitable for expression of these genes and production of polyketides are described, for example, in U.S. Pat. Nos. 5,843,718 and 5,830,750; WO 01/31035, WO 01/27306, and WO 02/068613; and U.S. patent application Ser. Nos. 10/087,451 (published as U.S. 2002000087451); 60/355,211; and 60/396,513 (corresponding to published application 20020045220).

Appropriate host cells for the expression of the hybrid PKS genes include those organisms capable of producing the needed precursors, such as malonyl-CoA, methylmalonyl-CoA, ethylmalonyl-CoA, and methoxymalonyl-ACP, and having phosphopantotheinylation systems capable of activating the ACP domains of modular PKSs. See, for example, U.S. Pat. No. 6,579,695. However, as disclosed in U.S. Pat. No. 6,033,883, a wide variety of hosts can be used, even though some hosts natively do not contain the appropriate post-translational mechanisms to activate the acyl carrier proteins of the synthases. Also see WO 97/13845 and WO 98/27203. The host cell may natively produce none, some, or all of the required polyketide precursors, and may be genetically engineered so as to produce the required polyketide precursors. Such hosts can be modified with the appropriate recombinant enzymes to effect these modifications. In one embodiment the host cell is a bacterium. In another embodiment the host cell is a fungus, such as a yeast cell. Suitable host cells include Streptomyces, E. coli, yeast, and other prokaryotic hosts which use control sequences compatible with Streptomyces spp. Examples of suitable hosts that either natively produce modular polyketides or have been engineered so as to produce modular polyketides include but are not limited to actinomyctes such as Streptomyces coelicolor, Streptomyces venezuelae, Streptomyces fradiae, Streptomyces ambofaciens, and Saccharopolyspora erythraea, eubacteria such as Escherichia coli, myxobacteria such as Myxococcus xanthus, and yeasts such as Saccharomyces cerevisiae.

In sone embodiments, any native modular PKS genes in the host cell have been deleted to produce a “clean host,” as described in U.S. Pat. No. 5,672,491.

Host cells can be selected, or engineered, for expression of a glycosylatation apparatus (discussed below), amide synthases, (see, for example, U.S. patent publication 20020045220 “Biosynthesis of Polyketide Synthase Substrates”). For example and not limitation, the host cell can contain the desosamine, megosamine, and/or mycarose biosynthetic genes, corresponding glycosyl transferase genes, and hydroxylase genes (e.g., picK, megK, eryK, megF, and/or eryF). Methods for glycosylating polyketides are generally known in the art and can be applied in accordance with the methods of the present invention; the glycosylation may be effected intracellularly by providing the appropriate glycosylation enzymes or may be effected in vitro using chemical synthetic means as described herein and in WO 98/493-15, incorporated herein by reference. Glycosylation with desosamine, mycarose, and/or megosamine is effected in accordance with the methods of the invention in recombinant host cells provided by the invention. Alternatively and as noted, glycosylation may be effected intracellularly using endogenous or recombinantly produced intracellular glycosylases. In addition, synthetic chemical methods may be employed.

Alternatively, the aglycone compounds can be produced in the recombinant host cell, and the desired modification (e.g., glycosylation and hydroxylation) steps carried out in vitro (e.g., using purified enzymes, isolated from native sources or recombinantly produced) or in vivo in a converting cell different from the host cell (e.g., by supplying the converting cell with the aglycone).

Modification or tailoring enzymes for modification of a product of the fostriecin PKS, a non-fostriecin PKS, or a chimeric PKS, can be those normally associated with fostriecin biosynthesis or “heterologous” tailoring enzymes. Tailoring enzymes can be expressed in the organism in which they are naturally produced, or as recombinant proteins in heterologous hosts. In some cases, the structure produced by the heterologous or hybrid PKS may be modified with different efficiencies by post-PKS tailoring enzymes from different sources. In such cases, post-PKS tailoring enzymes can be recruited from other pathways to obtain the desired compound.

In some embodiments, the host cell expresses, or is engineered to express, a polyketide “tailoring” or “modifying” enzyme. Once a PKS product is released, it is subject to post-PKS tailoring reactions. These reactions are important for biological activity and for the diversity seen among polyketides. Tailoring enzymes normally associated with polyketide biosynthesis include oxygenases, glycosyl- and methyltransferases, acyltransferases, halogenases, cyclases, aminotransferases, and hydroxylases.

In the case of fostriecin biosynthesis, tailoring enzymes include P450 hydroxylases for addition of hydroxyl groups. The PKS is expected to initially produce hydroxyls at C3, C5, C9 and C11, with the C9 hydroxyl further modified by phosphorylation, the C5 hydroxyl further reacting to help create the 6-membered lactone ring, and the C3 hydroxyl being removed by dehydration in the creation of a double bond between C2 and C3. In addition hydroxyls at C8 and C18 (and C4 in PD 113, 271) are expected to be introduced by post-PKS-acting accessory proteins. The fostriecin gene cluster encodes three cytochrome-P450-hydroxylase homologs (FosG, FosJ and FosK). Based on apparent homology between FosJ and the PlmT4 P450 hydrolase encoded in the Streptomyces phoslactomycin synthase gene cluster, apparent homology between FosK and the PlmS2 P450 hydrolase encoded in the Streptomyces phoslactomycin synthase gene cluster, evidence that PlmS2 is responsible for cyclohexyl modification at C18 but not C8 of the polyketide phoslactomycin, and the presence of hydroxyls at the tertiary C8 of fostriecin and the tertiary C8 of phoslactomycin, FosJ may produce the C8 hydroxyl of fostriecin. FosG and/or FosK are expected to modify the C4 and C8 positions, with perhaps a specific P450 for each site. The phosphorylation of the hydroxyl group at C9 is predicted to be accomplished by FosH, a distant homolog of homoserine kinases. ORF7 encodes a type II thioesterase.

The P450 hydroxylases and kinase of the fostriecin PKS gene cluster can be expressed heterologously to modify polyketides produced by non-fostriecin polyketide synthases or can be inactivated in the Fostriecin producer.

In addition to biosynthetic accessory activities, secondary metabolite clusters often code for activities such as transport and regulation. FosI appears to be a permease having a transport function. ORF1 and ORF3 are putative transcriptional regulators. ORF1 is a homolog of MarR-family transcriptional regulators, including SC07709, SC07639 and SC00447 from Streptomyces coelicolor. ORF3 is a homolog of LuxR family transcriptional regulators.

ORF2 is a homolog of a conserved family, including SC7708, SC6340 and SC5938 from Streptomyces coelicolor, and SAV1967 and SAV0886 from S. avermitilis. ORF4 is a homolog of a conserved family, including PlmT2 from the phoslactomycin biosynthetic cluster, SAV4898 from Streptomyces avermitilis and SC04633 from S. coelicolor. ORF5 is a homolog of BorL from the borrelidin biosynthetic cluster. ORF6 encodes a homolog of the product of plu4507 from Photorhabdus luminescens subsp. laumondii TTO1, and has some similarity to 3-hydroxy-3-methylglutaryl coenzyme A reductases. ORF8 encodes a homolog of chaperone protein HtpG (heat shock protein HtpG) from Streptomyces coelicolor.

Tables 2 and 4 describe the characteristics of open reading frames of the fostriecin polyketide synthase gene cluster. Table 2 shows the position of each ORF relative to SEQ ID NO: 1, as well as identifying certain homologous proteins.

TABLE 2
ORFs Encoding Additional Polypeptides Encoded in the Fostriecin polyketide synthase Cluster

| | amino | | Position in SEQ ID NO: 1 | | |
|---|---|---|---|---|---|
| ORF | acids | putative function | coding strand(nucleotide pair) | homology | % identity |
| orf1 | 165 | MarR-family | SEQ ID NO 1(72775 . . . 73272) | SC07709 (142 aa; 43% | 43%/137aa |
| | | transcriptional | | identity/137 aa), | |
| | | regulator | | SC07639 and SC00447 | |
| | | | | from Streptomyces | |
| | | | | coelicolor | |
| orf2 | 213 | | complement(72055 . . . 72696) | SC7708 (216 aa; 48% | 48%/216aa |
| | | | | identity/192 aa), | |
| | | | | SC6340 and SC5938 from | |
| | | | | Streptomyces coelicolor | |
| orf3 | 967 | LuxR-family | complement(68498 . . . 71401) | PikD (Streptomyces | 29%/977aa |
| | | transcriptional | | venezuelae) | |
| | | regulator | | | |
| orf4 | 295 | unknown | complement(67600 . . . 68487) | PlmT2 from the | 41%/212aa |
| | | | | phoslactomycin | |
| | | | | biosynthetic cluster | |
| fosG | 409 | P450; possible C8 or | complement(33643 . . . 34872) | ORF4 from the mitomycin | 48%/395aa |
| | | C4-hydroxylase | | C biosynthetic cluster | |
| | | | | in Streptomyces | |
| | | | | lavendulae | |
| fosH | 316 | polyketide kinase | complement(32552 . . . 33502) | PlmT5 from the | 43%/259aa |
| | | | | phoslactomycin | |
| | | | | biosynthetic cluster | |
| fosI | 444 | polyketide export | complement(31111 . . . 32445) | PlmS4 from the | 50%/431aa |
| | | | | phoslactomycin | |
| | | | | biosynthetic cluster | |
| | | | | [COG0477: Permeases of | |
| | | | | the major facilitator | |
| | | | | superfamily] | |
| fosJ | 420 | P450; possible C4- or | complement(29742 . . . 31004) | PlmT4 from the | 54%/397aa |
| | | C8-hydroxylase | | phoslactomycin | |
| | | | | biosynthetic cluster | |
| fosK | 398 | P450; possible C18- | SEQ ID NO 1(28443 . . . 29639) | PlmS2 from the | 57%/404aa |
| | | hydroxylase | | phoslactomycin | |
| | | | | biosynthetic cluster | |
| orf5 | 538 | | complement(7892 . . . 9508) | BorL from the | 30%/536aa |
| | | | | borrelidin biosynthetic | |
| | | | | cluster | |
| orf6 | 781 | homology to 3-hydroxy- | complement(5550 . . . 7895) | plu4507 from | 39%/774aa |
| | | 3-methylglutaryl | | Photorhabdus | |
| | | coenzyme A reductases | | luminescens subsp. | |
| | | | | laumondii TTO1 | |
| orf7 | 258 | thioesterase (TEII | complement(3840 . . . 4616) | AveG (Streptomyces | 52%/238aa |
| | | family) | | avermitilis) | |
| orf8 | 633 | chaperone protein htpG | complement(1424 . . . 3325) | HtpG (Streptomyces | 79%/638aa |
| | | (heat shock protein | | coelicolor) | |
| | | htpG) | | | |

*fosG, H, I, J and K were previously called ORFs 1, 2, 3, 4 and 5

It will be apparent to the reader that a variety of recombinant vectors can be utilized in the practice of aspects of the invention. As used herein, “vector” refers to polynucleotide elements that are used to introduce recombinant nucleic acid into cells for either expression or replication. Selection and use of such vehicles is routine in the art. An “expression vector” includes vectors capable of expressing DNAs that are operatively linked with regulatory sequences, such as promoter regions. Thus, an expression vector refers to a recombinant DNA or RNA construct, such as a plasmid, a phage, recombinant virus or other vector that, upon introduction into an appropriate host cell, results in expression of the cloned DNA. Appropriate expression vectors are well known to those of skill in the art and include those that are replicable in eukaryotic cells and/or prokaryotic cells and those that remain episomal or those which integrate into the host cell genome.

The vectors used to perform the various operations to replace the enzymatic activity in the host PKS genes or to support mutations in these regions of the host PKS genes may be chosen to contain control sequences operably linked to the resulting coding sequences in a manner that expression of the coding sequences may be effected in an appropriate host. Suitable control sequences include those which function in eukaryotic and prokaryotic host cells. If the cloning vectors employed to obtain PKS genes encoding derived PKS lack control sequences for expression operably linked to the encoding nucleotide sequences, the nucleotide sequences are inserted into appropriate expression vectors. This can be done individually, or using a pool of isolated encoding nucleotide sequences, which can be inserted into host vectors, the resulting vectors transformed or transfected into host cells, and the resulting cells plated out into individual colonies.

Suitable control sequences for single cell cultures of various types of organisms are well known in the art. Control systems for expression in yeast are widely available and are routinely used. Control elements include promoters, optionally containing operator sequences, and other elements depending on the nature of the host, such as ribosome binding sites.

Particularly useful promoters for prokaryotic hosts include those from PKS gene clusters which result in the production of polyketides as secondary metabolites, including those from Type I or aromatic (Type II) PKS gene clusters. Examples are act promoters, tcm promoters, spiramycin promoters, and the like. However, other bacterial promoters, such as those derived from sugar metabolizing enzymes, such as galactose, lactose (lac) and maltose, are also useful. Additional examples include promoters derived from biosynthetic enzymes such as for tryptophan (trp), the β-lactamase (bla), bacteriophage lambda PL, and T5. In addition, synthetic promoters, such as the tac promoter (U.S. Pat. No. 4,551,433), can be used.

As noted, particularly useful control sequences are those which themselves, or with suitable regulatory systems, activate expression during transition from growth to stationary phase in the vegetative mycelium. The system contained in the plasmid identified as pCK7, i.e., the actI/actIII promoter pair and the actII-ORF4 (an activator gene), is particularly preferred. Particularly preferred hosts are those which lack their own means for producing polyketides so that a cleaner result is obtained. Illustrative control sequences, vectors, and host cells of these types include the modified S. coelicolor CH999 and vectors described in PCT publication WO 96/40968 and similar strains of S. lividans. See U.S. Pat. Nos. 5,672,491; 5,830,750, 5,843,718; and 6,177,262, each of which is incorporated herein by reference.

Other regulatory sequences may also be desirable which allow for regulation of expression of the PKS sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

Selectable markers can also be included in the recombinant expression vectors. A variety of markers are known which are useful in selecting for transformed cell lines and generally comprise a gene whose expression confers a selectable phenotype on transformed cells when the cells are grown in an appropriate selective medium. Such markers include, for example, genes which confer antibiotic resistance or sensitivity to the plasmid. Alternatively, several polyketides are naturally colored, and this characteristic provides a built-in marker for screening cells successfully transformed by the present constructs.

The various PKS nucleotide sequences, or a mixture of such sequences, can be cloned into one or more recombinant vectors as individual cassettes, with separate control elements or under the control of a single promoter. The PKS subunits or components can include flanking restriction sites to allow for the easy deletion and insertion of other PKS subunits so that hybrid or chimeric PKSs can be generated. The design of such restriction sites is known to those of skill in the art and can be accomplished using the techniques described above, such as site-directed mutagenesis and PCR. Methods for introducing the recombinant vectors of the present invention into suitable hosts are known to those of skill in the art and typically include the use of CaCl₂ other agents, such as divalent cations, lipofection, DMSO, protoplast transformation, conjugation, and electroporation.

Thus, the present invention provides recombinant DNA molecules and vectors comprising those recombinant DNA molecules that encode all or a portion of the fostriecin PKS and/or fostriecin modification enzymes and that, when transformed into a host cell and the host cell is cultured under conditions that lead to the expression of said fostriecin PKS and/or modification enzymes, results in the production of polyketides including but not limited to fostriecin and/or analogs or derivatives thereof in useful quantities. The present invention also provides recombinant host cells comprising those recombinant vectors.

Suitable culture conditions for production of polyketides using the cells of the invention will vary according to the host cell and the nature of the polyketide being produced, but will be know to those of skill in the art. See, for example, the examples below and WO 98/27203 “Production of Polyketides in Bacteria and Yeast” and WO 01/83803 “Overproduction Hosts for Biosynthesis of Polyketides.”

The polyketide product produced by host cells of the invention can be recovered (i.e., separated from the producing cells and at least partially purified) using routine techniques (e.g., extraction from broth followed by chromatography).

The compositions, cells and methods of the invention may be directed to the preparation of an individual polyketide or a number of polyketides. The polyketide may or may not be novel, but the method of preparation permits a more convenient or alternative method of preparing it. It will be understood that the resulting polyketides may be further modified to convert them to other useful compounds. For example, an ester linkage may be added to produce a “pharmaceutically acceptable ester” (i.e., an ester that hydrolyzes under physiologically relevant conditions to produce a compound or a salt thereof). Illustrative examples of suitable ester groups include but are not limited to formates, acetates, propionates, butyrates, succinates, and ethylsuccinates.

The polyketide product can be modified by addition of a protecting group, for example to produce prodrug forms. A variety of protecting groups are disclosed, for example, in T. H. Greene and P. G. M. Wuts, Protective Groups in Organic Synthesis, Third Edition, John Wiley & Sons, New York (1999). Prodrugs are in general functional derivatives of the compounds that are readily convertible in vivo into the required compound. Conventional procedures for the selection and preparation of suitable prodrug derivatives are described, for example, in “Design of Prodrugs,” H. Bundgaard ed., Elsevier, 1985.

Similarly, improvements in water solubility of a polyketide compound can be achieved by addition of groups containing solubilizing functionalities to the compound or by removal of hydrophobic groups from the compound, so as to decrease the lipophilicity of the compound. Typical groups containing solubilizing functionalities include, but are not limited to: 2-(dimethylaminoethyl)amino, piperidinyl, N-alkylpiperidinyl, hexahydropyranyl, furfuryl, tetrahydrofurfuryl, pyrrolidinyl, N-alkylpyrrolidinyl, piperazinylamino, N-alkylpiperazinyl, morpholinyl, N-alkylaziridinylmethyl, (1-azabicyclo[1.3.0]hex-1-yl)ethyl, 2-(N-methylpyrrolidin-2-yl)ethyl, 2-(4-imidazolyl)ethyl, 2-(1-methyl-4-imidazolyl)ethyl, 2-(1-methyl-5-imidazolyl)ethyl, 2-(4-pyridyl)ethyl, and 3-(4-morpholino)-1-propyl. Solubilizing groups can be added by reaction with amines. Typical amines containing solubilizing functionalities include 2-(dimethylamino)-ethylamine, 4-aminopiperidine, 4-amino-1-methylpiperidine, 4-aminohexahydropyran, furfurylamine, tetrahydrofurfurylamine, 3-(aminomethyl)-tetrahydrofuran, 2-(amino-methyl)pyrrolidine, 2-(aminomethyl)-1-methylpyrrolidine, 1-methylpiperazine, morpholine, 1-methyl-2(aminomethyl)aziridine, 1-(2-aminoethyl)-1-azabicyclo-[1.3.0]hexane, 1-(2-aminoethyl)piperazine, 4-(2-aminoethyl)morpholine, 1-(2-amino-ethyl)pyrrolidine, 2-(2-aminoethyl)pyridine, 2-fluoroethylamine, 2,2-difluoroethylamine, and the like.

In addition to post synthesis chemical or biosynthetic modifications, various polyketide forms or compositions can be produced, including but not limited to mixtures of polyketides, enantiomers, diastereomers, geometrical isomers, polymorphic crystalline forms and solvates, and combinations and mixtures thereof can be produced.

Many other modifications of polyketides produced according to the invention will be apparent to those of skill, and can be accomplished using techniques of pharmaceutical chemistry.

Prior to use the PKS product (whether modified or not) can be formulated for storage, stability or administration. For example, the polyketide products can be formulated as a “pharmaceutically acceptable salt.” Suitable pharmaceutically acceptable salts of compounds include acid addition salts which may, for example, be formed by mixing a solution of the compound with a solution of a pharmaceutically acceptable acid such as hydrochloric acid, hydrobromic acid, sulfuric acid, fumaric acid, maleic acid, succinic acid, benzoic acid, acetic acid, citric acid, tartaric acid, phosphoric acid, carbonic acid, or the like. Where the compounds carry one or more acidic moieties, pharmaceutically acceptable salts may be formed by treatment of a solution of the compound with a solution of a pharmaceutically acceptable base, such as lithium hydroxide, sodium hydroxide, potassium hydroxide, tetraalkylammonium hydroxide, lithium carbonate, sodium carbonate, potassium carbonate, ammonia, alkylamines, or the like.

Prior to administration to a mammal the PKS product will be formulated as a pharmaceutical composition according to methods well known in the art, e.g., combination with a pharmaceutically acceptable carrier. The term “pharmaceutically acceptable carrier” refers to a medium that is used to prepare a desired dosage form of a compound. A pharmaceutically acceptable carrier can include one or more solvents, diluents, or other liquid vehicles; dispersion or suspension aids; surface active agents; isotonic agents; thickening or emulsifying agents; preservatives; solid binders; lubricants; and the like. Remington's Pharmaceutical Sciences, Fifteenth Edition, E. W. Martin (Mack Publishing Co., Easton, Pa., 1975) and Handbook of Pharmaceutical Excipients, Third Edition, A. H. Kibbe ed. (American Pharmaceutical Assoc. 2000), disclose various carriers used in formulating pharmaceutical compositions and known techniques for the preparation thereof.

The composition may be administered in any suitable form such as solid, semisolid, or liquid form. See Pharmaceutical Dosage Forms and Drug Delivery Systems, 5^{th} edition, Lippicott Williams & Wilkins (1991). In an embodiment, for illustration and not limitation, the polyketide is combined in admixture with an organic or inorganic carrier or excipient suitable for external, enteral, or parenteral application. The active ingredient may be compounded, for example, with the usual non-toxic, pharmaceutically acceptable carriers for tablets, pellets, capsules, suppositories, pessaries, solutions, emulsions, suspensions, and any other form suitable for use. The carriers that can be used include water, glucose, lactose, gum acacia, gelatin, mannitol, starch paste, magnesium trisilicate, talc, corn starch, keratin, colloidal silica, potato starch, urea, and other carriers suitable for use in manufacturing preparations, in solid, semi-solid, or liquified form. In addition, auxiliary stabilizing, thickening, and coloring agents and perfumes may be used.

It will be appreciated by those of skill that recombinant polynucleotides and polypeptides of the invention have a variety of uses, including, but not limited to, those described above and including use as probes and primers (e.g., for gene amplification or targeting) or as enzymes, or components of enzymes, useful for the synthesis or modification of polyketides. Recombinant polypeptides encoded by the fostriecin PKS gene cluster are also useful as antigens for production of antibodies. Such antibodies find use for purification of bacterial (e.g., Streptomyces pulveraceus) proteins, detection and typing of bacteria, and particularly, as tools for strain improvement (e.g., to assay PKS protein levels to identify “up-regulated” strains in which levels of polyketide producing or modifying proteins are elevated) or assessment of efficiency of expression of recombinant proteins. Polyclonal and monoclonal antibodies can be made by well known and routine methods (see, e.g., Harlow and Lane, 1988, ANTIBODIES: A LABORATORY MANUAL, COLD SPRING HARBOR LABORATORY, New York; Koehler and Milstein 1075, Nature 256: 495). In selecting-polypeptide sequences for antibody induction, it is not to retain biological activity; however, the protein fragment must be immunogenic, and preferably antigenic (as can be determined by routine methods). Generally the protein fragment is produced by recombinant expression of a DNA comprising at least about 60, more often at least about 200, or even at least about 500 or more base pairs of protein coding sequence, such as a polypeptide, module or domain derived from a fostriecin polyketide synthase (PKS) gene cluster. Methods for expression of recombinant proteins are well known. (See, e.g., Ausubel et al., 2002, Current Protocols In Molecular Biology, Greene Publishing and Wiley-Interscience, New York.)

### EXAMPLES

The following examples are provided to illustrate, but are not intended to limit, the present invention.

### Example 1

### Cloning and Sequencing of Gene Cluster for Fostriecin Biosynthesis

Growth of Organism and Extraction of Genomic DNA.

For genomic DNA extraction, a spore stock of Streptomyces pulveraceus subsp. fostreus ATCCC 31906 was used to inoculate 35 ml of Tryptitone Soy Broth (TSB) liquid media. After two days growth in 30° C., a 10 ml portion of the cell suspension was centrifuged (10,000×g). The pellet was suspended into 3.5 ml of buffer 1(Tris, 50 mM, pH7.5; 20 mM EDTA, 150 μg/ml RNase (Sigma-Aldrich) and 1 mg/ml of lysozyme (Sigma)). After incubation of the mixture at 37° C. for 30 min, the salt concentration was adjusted by adding 850 μl 5 M NaCl solution, then the mixture was extracted two times with phenol:chloroform:isoamylaclohol (25: 24: 1, vol/vol) with gentle agitation followed by centrifugation for 10 min at 3500×g. After precipitation with 1 vol of isopropanol, the genomic DNA knot was spooled on a glass rod and redissolved in 500 μl of water.

Genomic Library Preparation

Approximately 10 μg of genomic DNA was partially digested with Sau3A1 (1 hr incubation using dilutions of the enzyme) and the digested DNA was run on an agarose gel with DNA standards. One of the conditions used was found to have generated fragments of size 30-45 kb. The DNA from this digestion was ligated with pSuperCos-1 (Stratagene), pre-linearized with BamHI and XbaI and the ligation mixture was packaged using a Gigapack XIII (Stragene) in vitro packaging Kit and the mixture was subsequently used for infection of Escherichia coli DH5α employing protocols supplied by the manufacturer.

Identification of Fostriecin Biosynthetic Gene Cluster

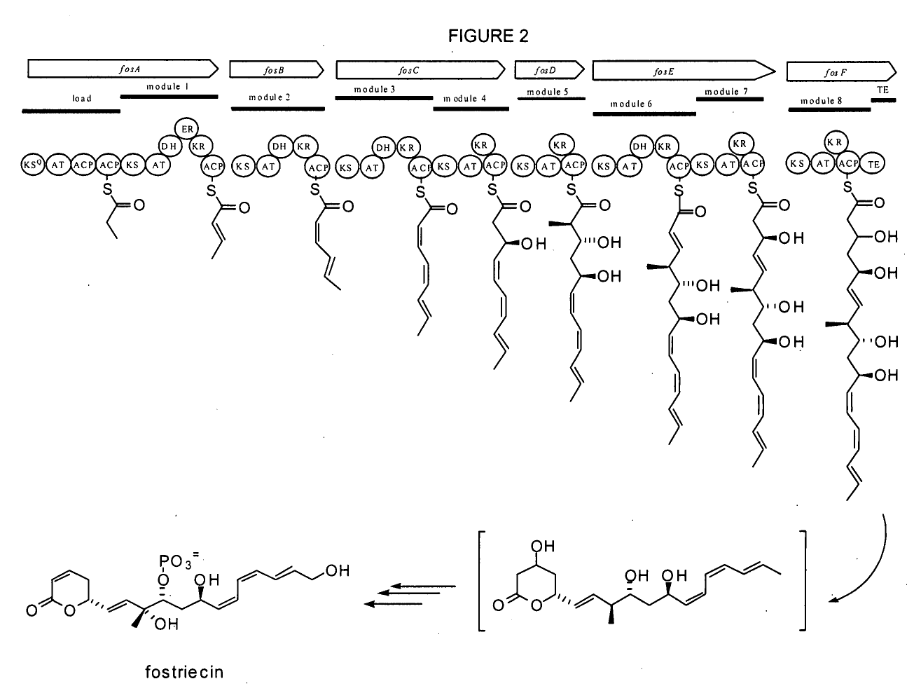

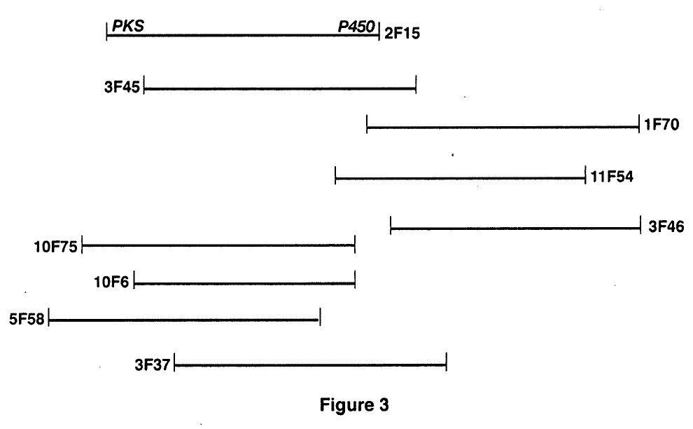

To find the gene cluster for fostriecin biosynthesis, cosmids from 1 IX 95 E. coli transductants resulted from the above ligation mixture were sequenced with using convergent primersT7cos (5′-CATAATACGACTCACTATAGGG) [SEQ ID NO: 21] and T3cos-1 (5′-TTCCCCGAAAAGTGCCAC) [SEQ ID NO: 22]. After BLAST analysis, the sequences revealed 28 cosmids carrying DNA fragment encoded type I or type II PKS (polyketide synthase) genes at either of ends or both ends. Based on sequence and restriction enzyme maps of the 21 cosmids most likely related to modular PKS, most could be assigned to two major groups (“overlap family 1” and “overlap family 3”). Since overlap family 3 carries genes (homologous to gdmI and K) for methoxymalonyl-ACP, which is not needed for the biosynthesis of fostriecin, we focused on overlap family 1 (See FIG. 2). Based on the relation of among these cosmids, we chose to sequence pKOS279-117.3F70, pKOS279-117.3F45, pKOS279-117.2F15, and pKos279-117.5F58 from the overlap family 1. Other cosmids in this family were pKOS279-127.11 F54; pKOS279-127.10F6; pKOS279-127.10F75; pKOS279-127.3F46; pKOS279-127.5F58.

DNA Sequencing

In initial sequencing efforts the sequence of inserts of three cosmids (pKOS279-117.1F70, pKOS279-117.3F45 and pKOS279-117.2F15) was determined. The results of this sequencing effort are provided in the appended sequence listings (which are part of and incorporated into this specification) as SEQ ID NOs: 23, 27 and 33. Small gaps in the sequence indicated as “x” or “n.” Complete or partial open reading frames (ORFs) encoded by these sequences can be determined by reference to the genetic code and are also provided in SEQ ID NOs: 24-26,28-32 and 34-36. Complete sequencing was carried out using (pKOS279-117.1F70, pKOS279-117.3F45, pKOS279-117.2F15, and pKos279-117.5F58).

TABLE 3

FOSTRIECIN SYNTHASE GENE CLUSTER
from Streptomyces pulveraceus subsp. fostreus ATCC31906
source 1 . . . 18774 from pKos279-117.1F70
source 18651 . . . 29679 from pKos279-117.3F45
source 28694 . . . 29679 from pKos279-117.2F15
source 29683 . . . 53913 from pKos279-117.3F45
source 29683 . . . 66484 from pKos279-117.2F15
source 58636 . . . >73984 from pKos279-117.5F58
29680 . . . 29682 is an unsequenced
fragment of putative hairpin terminator″

| | |
|---|---|
| 1 | AGGCCGACGG GCAGTGCCCG CGGCGTATCG GTGGCCGCCC ACGCGCGGGC GGGGAACGCA |
| 61 | CGGACGGCCC CCGCGGCGAG CGCGGCCACG GGCAGCGCCG ATCCTGCGCC GAGGAAGCCG |
| 121 | GGCCGGCTGA AGGCGGGATG GGGGCGGGGG GTGGGCGAGT CGGGGCCGTG AGGGGTGGGC |
| 181 | GAGTCGGGGC CGTGGGGGGT GGTCTGACTG GCCAACGGAG TACTCCTTCA CACACGTCGG |
| 241 | GCGAAGCAGG ACGGCTCAGC TGTACGGCGG ACAGGGACGG GAGCGACCGG TCCCGAAGCG |
| 301 | GGATTGAAAC GTTTCAATAA GGTGGTGCGG TCGTACGGTA CGAGAGTGAC CAAGGGGGGT |
| 361 | CAAGGGGTTC GGCACACCGT TTGGGACGGC CCCACGCCCG CCTCGCGCGC ACCTCGGCGC |
| 421 | ACTCGCCACG CACACTCGGC CTCGAACACC CGGCGAGGAA GCCCGTGGGA TCGGCAGGAT |
| 481 | CCGGCGACAG CGGCTGCGGA GCGCGGTGAC GCACACTGGG AAAGGCACCC CATCGAGGTG |
| 541 | CTCCGAGATC GTCGAGGGAA CCGTAGGCGG CCGGGATCCT GCGGCACGGA CCGGGAGGGC |
| 601 | CATGACACCA GGTACGACAC CAGGTATACC CGGACGACGG TCGGGCGCAC TGCGTCGGCA |
| 661 | TGCCGTGCGC CTCCTGGCGA AGCCGAGAGG CCGCCCCGCA CCGGCCCCTC CCCCGCGCTC |
| 721 | CGCCCAGCTG CGGGCGCTCA CCGCCCTGCT GGACGAGGCG GTGGCGGCGC AGGCACCGGC |
| 781 | GGACCGGAGG GTGGCGGCCT GCGGCGAACC GGGCCCCCTC GCCGGGCAGA CCGCCCGGGA |
| 841 | AGCCGGACGG CAGTACAGGG TCCTGCACGG GCTGCACGCG CGCGTGCGCG ATCTGCCGCT |
| 901 | GACGGAGGCC GATCTCGTCC GGGCCCAGGA GTACGCGGGA CGCCTGCTCT CCTACGGCCA |
| 961 | GTGGATGATG CGCGAGGCCA TGGACCTCGC CTTCCCCTCG AACCCGCGTC CGAGCGTCGA |
| 1021 | GGCGGCCGGG CTCCACCTCA ACGGCCTGGG AAGGCCCGCC GACGACCTGC GCAGGCTCCG |
| 1081 | CGACGCCCTC CGCTCCGAGT GCGGCGGCGG ACGGGCGGGT CGAGGGCACT GAGCCGGAGG |
| 1141 | GCCGTCGCGG ACGGTGGTCC GCGTCGTACG GGAGCGGGCC GCACCGGTCA AGGGCAGGAC |
| 1201 | GCCGCAGAGC TCTGCGATCC GGCCGTCGCG GGCGGTGCGG TGCGGTTCGG CGCGGTCGCA |
| 1261 | CGCGTTCGGT GCGGTTCGGT GCCGTGACGC GGTCGGCCGA CCGTCATGGG CGGCGGCGCC |
| 1321 | GACGCGTACC CGGGCCCCTT CCGGCCGAGA GCCGGATACG CGTCGAGGCC GCCGGTCCGG |
| 1381 | TGGACGCCGC CGTCCGGCTC GGCGGCCGGC GGCGCTGTCC GGCTCAGCGA AGCGTGCGCT |
| 1441 | CCAGTCGCTC CGCCACCAGC TTCACGAAAC GCCCCGGGTG CGTCGGCCGC CCGCGCTCCG |
| 1501 | CGAGCACCGC GAGTCCGTAC AGCAGGTCGG CGGTCTCGGC GAGCCCGGAG CGGTCCTCAC |
| 1561 | CCTCCTGGTA GGCCTGGTTC AGGCCCTGGA CCAGGGGGTG GGCGGGGTTG AGTTCGAGGA |
| 1621 | TCCGCCGGGC GGACGGAATC TCCTGGCCCA TGGCCCGGTA CATGCTCTCC AGCGCCGGGG |
| 1681 | TGAGGTCATG GGCGTCGGAG ACGACACAGG CCGGGGAGAC GGTCAGCCGG GTCGACAGGC |
| 1741 | GGACGTCCTT CATCTCCTCC CCGAGGTGTT CCTTCATCCA GCCCAGCAGA GCGGCGTACG |
| 1801 | TCTCGGCCTG CTTCTCCCGC CCGCCGTCGG CCTGTTCGCC GCCCTGGACG TCGAGATCGA |
| 1861 | TCTCGGCCTT GGCGACGGAC CTCAGCCGCT TGCCCTCGAA CTCGGCCACG ACGTCGACCC |
| 1921 | ACACCTCGTC GACGGGGTCG GTGAGCAGGA GGACCTCAAG ACCCCGGTCC CGGAACGCCT |
| 1981 | CCATGTGCGG GGAGTTCTCG ATGGTCTGCC GGGACGCGCC GGTCATGTAG TAGATGTCGT |
| 2041 | CGTGGGCTTC CTTCATCCGC TCCAGGTACT GCTGGAGCGT GGTCGGCGTC TCCTCGGCGT |
| 2101 | GGGTGCTGGC GAAGGACGGC ACGGCCAGCA GGGCGTCGCG GTCGTCGGTG TCGCCGAGGA |
| 2161 | AGCCCTCCTT CAGTACGGCG CCGAACTCCC GCCAGAACGC GGCGTACTTG TCGGCGTCGT |
| 2221 | TCGCCTTCAT CTCCTTGACC GAGGACAGGA CCTTCTTGGT CAGCCGGCGC TGGATCATCC |
| 2281 | GGATGTGCCG GTCCTGCTGG AGGATGTCCC GGGAGACGTT GAGCGAGAGG TCCTGCGCGT |
| 2341 | CGACGACACC CTTGACGAAG CGGAGGTGGG GCGGCAGCAG CGCCTCGCAG TCGTCCATGA |
| 2401 | TCAGTACGCG CTTGACGTAC AGCTGCAGAC CGCGGCGGAA GTCCCGGGTG AACAGGTCGT |
| 2461 | GGGGCGCGTG AGCGGGAAGG AACAGCAGCG CCTGGTACTC GAAGGTGCCC TCGGCCTGGA |
| 2521 | GGCGGATCGT CTCGAGGGGG TGGGGCCAGT CGTGGCTGAC GTGCTTGTAC AGCTCGTGGT |
| 2581 | ACTCGTCGTC GGAGACCTCG TCGGGCGAGC GTGCCCACAG CGCGTTCATC GAGTTCAGCG |
| 2641 | TCTCGGGTTC GGGCGTTTCC TCGCCGTCGG TCGCCTGCGG GAGGAGCCGG ACCGGCCAGG |
| 2701 | TGATGAAGTC GGAGTACCGC TTGACGATCT CCTTGATGTT CCAGGCGGAG GTGTAGTGGT |
| 2761 | GCAGTTGGTC GTCGGGGTCG GCCGGCTTGA GGTGGAGCGT GACGGCACTG CCCTGCGGCA |
| 2821 | GGTCGTCGAC CGTCTCCAGG GTGTAGGTGG CGTCACCGCG CGACGACCAC CGCGTGCCGC |
| 2881 | TGCGCTCCCC GGCACGCCGG GTCACCAGGG TCATCTCGTC GGCCACCATG AAGCCGGAGT |
| 2941 | AGAAAGCGAC GGCGAACTGT CCGATGAGGC CGTCGGCCCC GGCCGCGTCC TGCGCCTCCT |
| 3001 | TCAGCTCCTG GAGGAAGGCG GCCGTGCCCG AATTGGCGAT GGTGGCGATG AGCTTGGCGA |
| 3061 | CCTCGTCGTA CGACATCCCG ATGCCGTTGT CCCGCACGGT GAGCGTACGG GCCTTCTGGT |
| 3121 | GGAGCTCGAT CTCGATGTGC GGGTCGGACG TGTCGGCGTC GAGCCCGTCG TCCCGCAACG |
| 3181 | CGGCGAGACG CAGCTTGTCG AGCGCGTCGG AGGCGTTGGA GACGAGCTCG CGCAGGAAGA |
| 3241 | CGTCCTTGTT CGAGTAGACC GAGTGGATCA TCAGCTGGAG CAGCTGGCGT GGTTCTACCT |
| 3301 | GGAACTCGAA CGTTTCGGTC GCCATGCTTC GTATTCCTCA CAGGTTCCTG GGTGGCCGAA |
| 3361 | TCGGGCGAGA GCCACTGTAA GACACCAAGT CGGCGCATTG TCACCGCCGT TCGCCGCGCG |
| 3421 | GCGTCCGCAT CTGCGTCTGC GTCTGCGTCA GACCTCGCCG TGGGCGCGCC TGCCCCGGCC |
| 3481 | GTCCCGCCAG GACGTGGGGC AGGTGCCCCG CGCGGTCGCG GCCCCGGCGT CGGCGAACAC |
| 3541 | GGGCGCTCCA GCCGCCTTCG GAAGGCATCC CGGATGCGGA GCGGAGACCT TCGAACACGC |
| 3601 | CGGTCCTGAC CCGGTCGCAC GCCCCTGCTC CGGCTCGCTC CCGGGGATCC GGCACAATCG |
| 3661 | GACCGCGGAC CGACGGCCGC ATGCGCCTTC CTGGTGTGGT GCCGGACGGA GCCGTGGGAT |
| 3721 | CTGCGCTCCT ACGGCCACGT CGTCAAGCTG GAGCAGGAAC GTCTCGCCTA CCGGGCCCGC |
| 3781 | CGCACTCCCG CTTCGGCCGC TCCTGTCGCC GCAAGGCCCC GGCGACGCCC ACCGCCCCAT |
| 3841 | CACACGTACG GAGGGGCGAC CAGCAAGGTG TTGCGGATCG CCTCGACGAT CTCAGGCTGG |
| 3901 | TGCTGCACCA GGTAGAAGTG GCCGCCCGGC AGCAGTGTCA GGTCGAACGA ACCGGCGGTG |
| 3961 | TGGTCGGCCC ACAGGTTCAT CTGGCCCTCG TCGACCTTCG GGTCCTGCGC GCCGAGGAAG |
| 4021 | CCGCGGATGG GACAGGCCAC AGGCGGGCCG GGCACGTAGC GGTAGGTCTC GATGAGGGGG |
| 4081 | TACTCGTTGC GCAGCGGGGG CATGATCATC TCGATGATCT CGGGATCGTC GAACATCCGG |
| 4141 | GTGTCCGTGC CCTCCAGGCC GCGCAGTTCG GCCAGCACGT CTTCCTGCGA CATGGCGTGC |
| 4201 | ACCCGCTCGG CGCGGTTGAC CGACGGGGCG CCGCGGCCAG ACGCGAAGAG CGCCGTCACC |
| 4261 | GGCGTCGTGG AGTCCTCGAG GAGGCGGATG ACCTCGAAGG CCACCGCCGC CCGCATGCTG |
| 4321 | TGCCCGAAGA ACGCCGTCGG TACGGGCGGT TCTCCGCACA GCGCCTCGGC GACGTGGGCG |
| 4381 | GGGAGGTCTT GCAGCGTGGC GGGGAACGGG TCGGCCCTGC GGTCCTGCGG CCCCGGGTAC |
| 4441 | TGGACGGCGA CGATGTCGAT CTCCGGGGCC AGGGCACGCG CGAGCGGCAT GTAGAAGCTG |
| 4501 | GCGGAACCGC CCGCGTGCGG CAGGCAGACG AGCCGGTGGC GGGCGTCCGG GGCGTTCGTG |
| 4561 | TAACGGCGCA GCCACGCCTG CCGGTCCGTG GACGGTTTCG GGGTCGGGGC GTACATCAAG |
| 4621 | GTTTCTCCAG AGTGCGGAAG GCGAAGCGCC GAGGCGGAGG TCGCCCGCGG CGGTGGGTGC |
| 4681 | GTCAGTGGGC GACCGCGGCC CCCGGTTCGG TGACCGCGGT GTGCTGCTCC ATGACCCGGC |
| 4741 | TCGGACGCGC CGGCCCTGTC ACCGGCGCCC GAGCCTGTGT GTCCAGTCCC AGCAGGCCGG |
| 4801 | CCACCTCGTC GGCGACGGAC GTCGCGTCGC GGCCGACGCA GTCGACCCGG TGCACGGTGA |
| 4861 | CGCGGCCGGA GAGCGCGTCG AGCACGTCGT CGTAGGCGTG GGCGAGCCCG GTGAGCAGCC |
| 4921 | GGGCGTCCTC GTGCAGTTCG GTGCCGTTCC GGCGGCTGCG GGTGCGCCGC AGTGCCTCGT |
| 4981 | CGACCGGCAG TCGGAGCCGT ACCACGGCGT CCGGCGGGGC GGTCGAGAAC ATGTCGGCCA |
| 5041 | GGCGCTGCGC CAGTTCCTCG CGGGGCGCGG CGGCGAGCCG CAGCAGGTCC AGGCCGAGCG |
| 5101 | CCCACGGATC CGTACCGCCC GAGCAGGCCT CCAGCCAATC CCGCACCTCG CGCGCGGTCT |
| 5161 | CCGGACCCTG CTCCGCCCAC CAGCGCCGGA CGGCGTCGCC GGGATCGGGA TCGCCCGCGA |
| 5221 | TCCGCGCGAA CATCGGCAGA TAGACGAGGG GGTCGACCAG CGGGTGCCTG TCCGCCAGGA |
| 5281 | CGATCGCGCC ACGCTGCGTG GCGCGGCGCT CGGCCGGGGC GTACTGGGAC AGTTGCAGAT |
| 5341 | AGAGCACCGC GACCTTCAGC GGCGAACTGC CGATCAGGTC CGCGGCGGCG GACGCCCGCG |
| 5401 | CCAGGTGCAG GGAGCGCGTC GCCTCGGGGC TGTCCGGGTC CTCGTGCGCG CGGATCGCGT |
| 5461 | GGACGACGGA AACGCCGGGC ACGGTGCTCA GCAGCCGCGC CACCGTCGTC TTGCCGGTGG |
| 5521 | CGTCGATGCC CACCAGGGCG GCCCGCATCT CACTCACCGT CCCGGGCGGC TTCCAGGTTC |
| 5581 | CACAGGTGGA ACGTGGTGTC CAGCACGGCC GGGAGGAACG GCAGTTCCCG GTGGCTGTGC |
| 5641 | GCGGCGGTGT ACAGCTGGAG GCGGCTCAGG AGCATCTCAC GCAGCGCGAG CCCGTAGCGG |
| 5701 | CGCCGCCACT GTTCCGGCTG CGGCACGGTC GCGTCCGGTC CGGCCGCGGC GGCGACCGCG |
| 5761 | GCGCGGTGCA CCTCCAGATG GTGGTCGACC TCGTCGGTGG TGCTGTGCGG GGTGAGGGTG |
| 5821 | AAGGCCAGCA GTTCGGCCAG GTCGCGCTGG GGCACTGCGA CGGTGGCCAG CTCCCAGTCG |
| 5881 | TAGGCGGTGA CGCGTTCGCT CTGTCGGCTG ATGTTGCGGG GGTTGAAGTC GTTGTGGACG |
| 5941 | AGTGTGCGGG GCATCGCGTC CATCTCCTGC ACCCAGAACT GCGCCTCGGC CGCCGCCGCG |
| 6001 | AGCGCCGTCC TGGTGCGCTG CGGCGTCATC AGTTCGGGCA GTTCCGCGGC GTTGTGGCGG |
| 6061 | ACCAGCGCGT CCCACAGTTC GCGTGCGTTG ACGAGGTGGG CGGTCGTGCC GTCGCGGTAG |
| 6121 | AGCCAGCGCT CGGCCAGGAT GTGCTGGTCG CGTCCGAGCC AGTGCCCGTG CACGGGGGCG |
| 6181 | ATGGCGCGCA GCGCGCGGTC CAGGTCGGTG CGGCTCCACG GCCCGGTCGT GATGTCGAGG |
| 6241 | CGTTCCATGA GGATCACGTA CGCTTGGCGC GCCTCGTCCT GGATGATGCC GTAGCAGACC |
| 6301 | GGGAGCAGGC TGGTCAGTAC GCCTTCGGGC CGCCGGAAGA CGGCCAGTTC GCGCCGGTGG |
| 6361 | GCGGCAGGGA AGTGGCTGCC GCCGCCCCAG GTCTCGCAGG CCGAGGACAC CTCGGGGCCG |
| 6421 | CACAGCGAGG CGATCCGGCC GATGCCGGCG GCGATGTCCT CGCCCCGCGG TTTGGCCTTC |
| 6481 | GCCACCAGTT CGGCGGTGGT CTGCGGGCGG TCGTCCTCGG TCCAGCTCAC GGTGATGGGG |
| 6541 | ATGACGCCGG TCAGCTTGCG CCGTTCGCCG AGGGCGCGGA GTTCGGTGGA GATGCCGTCG |
| 6601 | CCGACCATCG CCGGCGGCCG TACGACGTCG GTGACGCGCA GCCGGGGGCT GTGGAGCCGC |
| 6661 | TCGGCCATGG CCGGCTGGAG GAGGCCGGGG CGGAGGTCGT CGGCGCGCAG CCAGTCCACC |
| 6721 | CGCCGGGCCC GGCCCAGCCG CCGGTGCGCA TCGGCGAACT GGCCGCTGAC CAGGGCCGAC |
| 6781 | GCGGTCGACA CGTCGAGCGC CAGGGCGAAG CCGGCGATGA TCTCGGCCAG CGGGGCCGTG |
| 6841 | CCGCCTTCGC CGCGGCATCC GAGGACACCG AGCCAGTCGC GCTGGTCGGG CAGGCCGGTG |
| 6901 | CCGCCGCCGA CGGTGCCGAT CACGAGGTTC GGCAGCAGGA GCGTCGCGAT CAGGTCGTCG |
| 6961 | CCGTCGGAGT CGAAGGAGAG CACGGACACA GCGGACTCGT GCACGCAGGC GATGTCGTGT |
| 7021 | CCGGTGGCCA CGAACAGCGC GGGGATCACG TTGGCGGCGT TGATGCCGTA CCCGGTGATG |
| 7081 | CCGGCCTGCT GGGCTCCGAT GACGGGGACC CGGTGTCCGC GGGCGATCGC CGCCGGGGTG |
| 7141 | GTCTTCAGGA CCGAGGCGAC CACGTCGCCG GGGATGACGC ACTCGGCGGT GACCCGGGTG |
| 7201 | CCGCGGCGGG CGAGCAGCGA GACCGAGCTG ACCTTCTTGT CACTGCTCAG GTTGCCTTCG |
| 7261 | AGCAGCGTGT TCCGCGGTCG CAGCCCGGGC TCGTCGGCGA GCACCTGGTT CAGCCAGGTG |
| 7321 | CAGATCTGCC AGGTGGCCGC GGTGGTCATG TTCTGTCCGG GCGCGTCCGC GGTCTCGAAG |
| 7381 | ACGAACGGCA CGTGCAGGTA GCGGCCGATC TGGTACGGGT CGACCGCGAC GAGCCGGGCG |
| 7441 | TGCTGCGAGA CCAGCCGGAC CTGGTCCTCC AGCTGCGGGC GGCGGGTGCC GAGCCACCGG |
| 7501 | CTGAAGCGGG CGGCGCCGGC CAGGTCGTCG AACTCGAAGG CGGGCGCGCG GCTCATCCGC |
| 7561 | TGGGAGAGCA CCCGGGTGGA CACTCCGCCG GCCAGGCTCA GGGCGCGTGC GCCGCGGGAG |
| 7621 | GCGGAGGCTA CGAGCGCGCC CTCGGTGGTG GCCATCGGGG CGACGACGGC TTCCCGGACG |
| 7681 | CCCTGGCCGC GGAACTGCAG CGGTCCGGCG AGCCCCACCG GGACCTCGAC CGATCCGGCG |
| 7741 | AAGTTCTCCA GGTTCCCGGT CAGCGACGCG GCCTCGATCG CCGTGTGTGC GGCGGAATCG |
| 7801 | AGGGTCGCGC CGGTCCGGGC GAGCAGCCAT GCCAGCCGCG CTGCGCGGGC CTGTTCGGTG |
| 7861 | TACCGGCCAC GGCCCGGTAT CGCGTCGTCG GTCACGCGTG TGCCCTTTCG TCGGCGGTGA |
| 7921 | TCCGCGCCCA GCTGTCGGGC GCGGGCTGGA AGATCCTGTC CTTGACGCGG GTGCGGCGCT |
| 7981 | GGCCGGAGCG GATCGCGTGC TCCCACTCGC GCTGGAAGGC GTCCTGGTGC ACCTGGAGCA |
| 8041 | CCTCGACGGG GGCCAGGGCT CCGGCGTCGC GCGCCGCGCG GTATCCGTCG GCGGTCTCGC |
| 8101 | CCAGGTGCTT GTCCAGCAGC GTCGCGAAAC CGGCGGGCAG CGGTCCCGGC AGTGCGGAGG |
| 8161 | CGATCGCCGC GCGGAACCGG TCCGTTCCGG TCTCGACCGT CGCGTTGCGC AGCTCCGCCG |
| 8221 | CGGTGTCGGC CAGGGTGCCG GCCAGGGCGC GGACCACCGC GCGTTCCGTC AGGTCCACGC |
| 8281 | CGGCGGCCGT GCGCAGCACG TCACGTCCGG TGTAGGCGAT GCGCGGCGTA CGCCCGACGT |
| 8341 | GGTCGACGAC GTGCACGACG TCGTTGACGG GGCACCGGTA CAGACCGCCG ATGTGGCTGA |
| 8401 | GTACGAGGTG GTAGTCGCGG CCGGGTTCGA GTTCGGCGGC GGTCACGGTC GGGCTGTCCT |
| 8461 | CGCGGATCGG GTCGGCGGCG TCGGCGAACT CGAAGTAGCA GCCGGGCAGA TAGAGCGGGG |
| 8521 | CGGGGTTGGG ATGGTCGTCG ACGGGGACGG CGACCGGGCC CTCCGAGGAT CCGATCGGCG |
| 8581 | CGGCGAACAG GCGTACGCCG GGACCGTAGC GTTCGCGCAC GCGTGGCAGG TAGAGCGAGG |
| 8641 | CGAGGGCGCT GTTCCACGCG ACGGCGGCCC GGAGGTTGGG GCACAGGTGG TACGGATCCA |
| 8701 | GGACGCCGTA CTCGTCGGCG CGGCGCGCGA TCTGCTCGGC GCGCCGCGGA TCGGGCGTGG |
| 8761 | TGTGCGGCAC TCCGCCGACC GTGCCGCGGG CGATCTCCTC GACGATCCGG GGCCACTGGG |
| 8821 | CGGCCAGTTG GTGGGGCAGC CGGGCGATCA GGGCCGGGTT GACGCCGATC AGGACCTTGA |
| 8881 | TGTGGCGTTC GGCGGCGAGC CGCAGTTGAA GGTATGCCCG CTCCCACGGG TCGGCGTCGG |
| 8941 | AGAGCTGCTC GGGGATCGTG GCCCAGGCGG CGCCGTCCTC CGGCCGGGCG CCCTCGCCGA |
| 9001 | AGAGGCGGTG GTCGATCTGG CTGGGGCCCA GATGCGGCCG GCCGTCGGCG GTGCGCGCGT |
| 9061 | GCGGCGAGGT CGGATCGCGC CACAGGTTCA GCACGCCGCC GGGGTCCGCG GCGAGGTCGG |
| 9121 | GGAACGCGCC GAGCAGGACG GCGAAACTGG CGTGGTAGAA GGGCAGGAAG CAGCGTTTCA |
| 9181 | TGTAGGTGGG CGTGACCGGG ATGCGCTTCT CCTGGCGGGT GCTGGCGCTG GAGGAGAAAA |
| 9241 | ACGCGACCGG CCGCTCGGCG GTCAGGACCC CGTCCTCGCC GGCAATCGCC CGCTCGATCC |
| 9301 | AGGGACCGAA CGCGTTCTGC GTGCGGATCG GCAGGGCCTT GCGGAACTCC TCGGCGCCGC |
| 9361 | TTCGCTCGTT CAGGCCGTGC TCGCGCAGGT AGCTCGTCGC ACCGTTCGCC GCGAGGAGTT |
| 9421 | CCGCGAGGAC CGTCTGCTGG GTCTGGTCGG GGTGGTCGAG CGTGGCGAGG AACCTGTGGT |
| 9481 | GTTCGGTCAG AATGGGTTCG GTGTGCAAGC TTTCCCTCCA GCGCGGTCGG AGAAGAGGGC |
| 9541 | TCCGTACAGG CGGAGCCGCT CGGGGTCGGC GGTGTCGTGC GTGATCCGGC TGTCGACGGG |
| 9601 | GTGGGGACGG GCCGGGTCGA GCGCGGCGCG CAGCCGCGGT GGGTCGAGCG CCGGGCGGTG |
| 9661 | CGTCACGTGC ACGGTGTCGC CGGGACGGAC CAGACCGCGC AGGGCGGCCT CGGGGAAGAG |
| 9721 | GTGATGGGTG TGGACGAGGT AGTCCAGGAC GGTGACGTCC GCGGTCGCCA TCACGGCCTC |
| 9781 | GGCCCGGAAT CCGGTGTCGG GGCCGGGCAG CCGCACGGTG AAGGGCTCCG GCGCGGGGGC |
| 9841 | CGGTGCCGGA GCCGTGAAGG CGCGGAACAG CCGTGCCAGG TTCTTCAGTT GCGGCGTCGG |
| 9901 | CACCGGGCCG TACCAGCTCA GCGCGTTGTC GGGGGTCGCG TACCCGTCCG GGAACCGGGC |
| 9961 | CGCCACCGTG GCGGGCCGGC CCGCCAGGAA GATGGTGCGG GTGAACGTCC GCAGGCATGC |
| 10021 | GTCGGCCGGC TGCTCGGGGA GCGAGAGCGC GAAGTCGAGG AGGCCGCGGA CGAAGGACTG |
| 10081 | GGGGGTGAGG GrGTCCACCA GGACCGCCAC GGTGTGGAAG TGGTGTCCCG GTTGGTCGTC |
| 10141 | GAGGGGGGCC AGGCGGGCCG CCGCGGAGGC TTGCAGCAAC GTGTGGAGCG AACCATGCGG |
| 10201 | GTCTTCGACG TCCGGGAGGT CGATGTCCGG GAGCGCGGGG TCGCTCACGC GGGGTGGGGT |
| 10261 | CTGTCCGGTC ATACGAGCAC CTCGCGCAAC AGGGCGGGCA GCGGCGCCGG GCGCGGTCCG |
| 10321 | AGCAGCGAGC AGGCCAGGCG GTGCGGGTCG ACATAGCCGT GCGGGCTGAG CGGCAGTACC |
| 10381 | GGGTCCTCCC GTTCGTCCGA CGGCCCCAGG GCCAGGATCC GGGCCGCGCG CCCGACGCAG |
| 10441 | CGGCGCAGCA GGTCGATCCG GAAGGACTGG CTGCACTGCC GGTCGAGCAG ACCGACGAAG |
| 10501 | TAGAGGCGCA TGCCGAGCCG GTAGGCGTCG GCGTCCGCGG AGGCGGAGAT GGCCGACAGG |
| 10561 | TCCGAGAGCG TCGAGCCGGT CCACCCGCCG TACTTGCTGG AGACGACCTT GCCGCCGATC |
| 10621 | GGCACCCGGG TGAGCGGGAG CCGGGTGGTG TGGGCGCCGA ACTCGGCCAG GATCCGGTCA |
| 10681 | AGGAGCAGGT AGTCGACGCC GAGCCCTTCG TCGAAGAGCA GGAGGAAGAG CCTGCCGGGC |
| 10741 | GCGACGGCGG GCAGCAGCGA GCGCAGGATC GGGAGCAGGT AGTTCGCGTG GCCGTCCGTC |
| 10801 | GAGACGATCT GCCGGATCGG CACGCCCCAG CGGGCGCCGT CCAGGTAGAC GGGCCCGCCG |
| 10861 | TGCGGCCTGT GGTCGATGAG CAGCCCGCGG TCCGCGAGGG CCGCCAGGGC GCGCTGCTCC |
| 10921 | GAGCAGGTCA GCGGGCGGGT GTCGGTCAGG CCGGGGTCGC GCACGTGCAG CAGGTCGAGT |
| 10981 | TCGGTGCGCC ACAGCTCCAG CAGGCGGTGG GAGGCCGGAT GGATCCAGCC GTCCCGGTGG |
| 11041 | ATCCGGGCGA AGTAGGGGTC GAGTGCGCGG GGGTGGGTCG TCGGACGGCC GCGGTGGAAC |
| 11101 | TGGAGGTAGC GCCGGCCGAT CGCGGTCTCG TCCTCGCCGG AGCAGTCCGT GTCCGGTTCC |
| 11161 | GTGGGGTCCA GGTGGTTCCA GAACGCCGTG GTCTGGGTCG TGAGCGTGGA CATCCGGGGA |
| 11221 | TTCCAGACCA GGGTGGTGGG GCCGAGGGTG GCCGTCGCCT TGAACAGGGC GTCGGCCCAC |
| 11281 | AGCAGGCCTT TGACATGGGT GGGGGTCAGC GGGTTGGTGG GGGTGATCGT CACCGGGGCG |
| 11341 | ATCACGAACT CCCTCGCGGC GCGGCGGGCG CCGGGATGCG CGCGGTGTGC GCGGCTCGCG |
| 11401 | GGTGTGGGCG GGCCGGTCGT CGCGGTTCGT ACGGTGCTCG GGGTGCGCAC GGTGCTCGAG |
| 11461 | GTGCGCAGGG TGGTGCTCAT GGATGGCTCC TGTCGATGTC TGCCGCGACC GGGCGGACGA |
| 11521 | GGTCGTGCGC GGCGCGCGAC ATGCCGGTCG CGGCGCAGGA CGTGCCGGTT GCGGCGCGCG |
| 11581 | CCGCGTGGGC CGTGACGCGC GCCGCGTTCG AATCGACAGC GGTCACGACG TGGTCGGCCG |
| 11641 | GGACGGGTCG GACGCCGTCC CGTCGGAGGC CCCGTATTCG GCGTGGAGGA ACTGCATGAG |
| 11701 | GTCGGTCGCG CTCGCCGTCT CCAGGTCATC GGTGCCGGAG TTCCTGCCGG CTCCCCCGGC |
| 11761 | TCCGGTGGCC GTGCCGGAGA GCCGGTCCAG GAGCGCGGAG AGACGGGTGG CGAGCGCGGC |
| 11821 | CCGGGTGTCT GCCGGTGTGA ACGGCGAGAG CACCTTGGCG TCAAGGCGGT CGAAATCCTC |
| 11881 | GAGGACCGGA CCGTCGTCGG CGGGGGCTTC CTCGAGGTGC AGTTCCGTGA GCAGCCGCTC |
| 11941 | GGCGAGCGAC TCGGGCGTGG GGTGGTCGAA CGCCAGCGTG GCCGGCAGCC GGGCGCCGAC |
| 12001 | GAGTTCGCCG AGCCTGTTCC GCAGCTCCAC GGACATCATC GAGTCGAAGC CGAGTTCCCG |
| 12061 | CAGCGCCCGG TCCGCCTGCA CCTGTTGCGG GTCGCCCCGC CCGATGAGCT GCGCCACGTG |
| 12121 | GGTGCGCACG ACGTGGAGCA GTACGGGCAG CCGCTCGTCG TCGGGCAGCG CGCCCACCCG |
| 12181 | GTCCTTCAGC GCGCCCGCGG TGGTGCCCGC GCCGGGGAGG GCGGGGGACG CTGCGTCCCG |
| 12241 | GCCGCCGCCC GCCGTGTCCG CGGGCGCGAA TCCGCTGAGC AGCGGGCTGG GGCGAAGGAC |
| 12301 | GGTGAACGCG GGGAGGAAGC GCGACCAGGC CACGTCCACC ACGACCGGGT CGGTGTCGCC |
| 12361 | CGGCACGGCG CGCTCGAGCG CTTCGACGGG GCTGGGCACG GCGAGCGGGA GCAGCCCGGG |
| 12421 | CTTGCGCAGT TCCCGGTCCT CGTACTCGGT GACCATGCCG CCGCCCGACC AGGGCCCCCA |
| 12481 | GGCGATGGAG ACGGCGGACG CGCCCCGCTC GCGGCGTCGG CGGGCGAGCG CGTCGAGGCA |
| 12541 | GGCGTTGCCC GCGGCGTAGG CGGCCTGGTC CGCGGCGCCC CAGATCCCGG CGATGGAGGA |
| 12601 | GAACATGACG AACTCGTCGG CGTCGGGCAG CAGTTCGTCG AGCAACAGCG CGCCGTTCAC |
| 12661 | CTTCGCCGCC ATGTCGGTCT CGAAAGCCTC GGGGTCGACG TCCGCGATGC GCGCGTACCG |
| 12721 | GATCACGCCG GCGGCGTGGA CGAGGGTACG CACCGGGTCG TCCTCGGCAT CAAGACGGGA |
| 12781 | GAGCAGTTGC GTGACGGCGG TGCGGTCGGT GATGTCGATC GCCGCGGCTT CGGCCCGGAC |
| 12841 | GCCCGATTCG CCGAGTTCTG CCAGGAGTTC GGCGGCACGG GGTGACTCGG CGCCGCGGCG |
| 12901 | GGAGAGCAGG ACCAGGCGGT CGGCCGTGCC GAGAGCGGCG AGGGGGCGGG GGACGTGCGC |
| 12961 | GCCCAGGCCG CCGGTTCCGC CGGTGATCAG GACGGTGCGC TTGGGCTGCC AGGTTCCCGC |
| 13021 | GTCGGCCGCG GGTGCGGTGC CGAGCCGGCG GGCCCACAGG CGCCCGTGGC GCACCGGGAG |
| 13081 | CTGGTCCTCG GGGCCATGGC CGGTGAGCAC GCCGGCGAGC AGACGTGCGG TGGTCCGCCC |
| 13141 | GGCGTTGTCG TCAGCCGTCC CGAGGTCGGC CGTCTCCGGA ACGTCGACGA GTCCGGCCCA |
| 13201 | GCGCGAAGGG AGTTCGAGGG CGGCGACCCG GCCGAGCCCC CAGGCGCCCG CCGCGGCCAC |
| 13261 | GTGCGTCGCG GGGTCGTGGA CACCGACGCC GACCGCGCCG CTGGTCACGC ACCAGACCGG |
| 13321 | CGCGTCGAGC CGCTCGGTCT CCCGCAGCGC GGGGAGCAGT TCGGAGGCGT CGGCGGGGCA |
| 13381 | GACGAGCACG CCTTCGAGCG GTGTCGAGGC CGATGCATCC CATTCGGTGG ACGTGAGGAC |
| 13441 | GTGCTCGAAC AGCTCGGCGA GTCCGGGGCG TGCGGTGCCG AAGAGCAGCC AGGTGCCGGG |
| 13501 | CACGCGCTCG GCGATCCGCG CGTGGCTCAG CGGCTCCCAC CGCACGGCGA AGGTCGCGGC |
| 13561 | CTGTCCGACG GCCCCTTCGG CCGCCTGGAG CTGTGTGCGC TCCACGGCGC GCAGGATCAG |
| 13621 | CGATTGCATG GCGAGGACCG GGGTTCCGGC GGCGTCCGTG ATCCACACCG AGGTCGCGTC |
| 13681 | CGGCCGCGGG CGCAGCCGTA GGCGGACGCG CCGCACGTCC GTGGCGAAGA GGGTGACGCC |
| 13741 | GCCGAAGGAG AAGGGCAGGC GCACCTCGTC GTCGGTCTCG TAGAAGCTGC GGGTGATGGG |
| 13801 | CAGGGCGTGC AACGAGGCGT CCAGCAGAGC GGGGTGGGCG CCGAAGCCGT AGGGCTGGTC |
| 13861 | GTGGGGCAGG ACGACCTGCG CGAAGAGATC GTCGCCGCGC CGCCACAGCG CCTTGAGCGA |
| 13921 | CCGGAAGGCG GGGCCGTACT CGTAGCCGCG CTCGGCGAGG TCCGGGTAGA ACGTGTCACC |
| 13981 | GGGGATCTGT TCCGCGCCCG CGGGCGGCCA CACCGCGCCG GTCCAGTCGG GCGTGAAGCC |
| 14041 | GTCGGTGTCC ACGCGGGAGG CGGTCACGAC GCCGGTGGCG TGCAGCGTCC AGTCCTCGCC |
| 14101 | CGGCGTACGC GTGCGGATCA GCAGTTCGCG CTCGCGGCCC TGGTCGGGTG CGACCCAGAC |
| 14161 | CTGGAGGTCG CGGGCCCGGC CGCCCGGGAA CACCATCGGG GCGCGCAGGA CGAGTTCTTC |
| 14221 | GACGCGGCCC GCGCCGACCG CGCGCGCGGC CTCCAGCGCG AGTTCCACGA ATCCGGTGCC |
| 14281 | GGGCAGCAGC AGGGTGCCCA TGACGGCGTG GTGGGGCAGC CACGGGTCCG TGCCGGGCGC |
| 14341 | GAGGCGGCCG GAGAACAGGA CGCCGCCCCC GCCGGGGAGG TGCGTGCGCT GGGACAGCAT |
| 14401 | CGGGTGCGGC AGCGCGTCGG CCCCGGCGCC GGGGCCGGTA CCCGAGGACG GGGCCGTCAG |
| 14461 | CCAGTAGTTC TCGTGCTGGA AGGGGTAGGT GGGCAGGTCG AGGTCCGGGA CCGTGGCGCT |
| 14521 | GCCGCGCTCG TGTCCGCGGC CGTCGGCGGT ATCGGCGTTG TCGGCGTCCG CCTCGGCATG |
| 14581 | GTCGGCGAAC CAGGTGACCG GCGTCCCGGT CACGTGCAGG GTCGCAAGGG CCCTGAGGAA |
| 14641 | CGTGTCGGGC TCGGGCTGCC CGGCACGCAG CGTCGGCACG AGCGCCGCGG GAGCGGCGGA |
| 14701 | CGCGTCCTCG AGTGTCTCGG CGGCGAGCGG CGGGAGTGTG GGGTGCGCGG TGAGTTCGAG |
| 14761 | GTAGCGGGTG GTGCCGAGTC CGTGCAGGGT GGTGACGGCG TCGGGGTGGC GGACGGTGTG |
| 14821 | GCGCAGCTGT TCGGTCCAGT GGTCGGCGGT CGTGATCCGG TCCTGCTCGG CGAGCAGTCC |
| 14881 | GGTGAGCGTC GAGACGATGG GAATGCGCGG CGCGCGGTAC GTGAGCCCCG CCGCGATGCG |
| 14941 | GCGGAACTCG TCCAGGATCT GGTCCTGGTG CGGACTGTGG AAGGCGTGGC TGACGGTCAG |
| 15001 | CCGCCTGCTC CTGATCCCCC GCTCCGCCAG CTGTGCGGCG ATGTCCGCGA GGACCTCGGG |
| 15061 | ATCACCGGAC AGGACGGTCG ACTCCGGCGC GTTGACCGCC GCGAGCGAGA CGACGTCCTC |
| 15121 | ACGCCCCGCC ACGAGACCCC GCGCGGTGGC CTCGCCGGCC TGGAGGGCGA GGATGGTGCC |
| 15181 | GGGGGTGGTG ATCTGCTGCA TCAGACGGGC CCGGTGGAAG ACCAGGGTGG CCGCATCAGC |
| 15241 | CAAAGAGAGC ATCCCGGCGG CATGCGCCGC GGACAGCTCA CCGATCGAAT GCCCCACCAG |
| 15301 | ATGGTCCGGC CGCACACCGA ACGACTCCAG CAGCCGGAAC AACGCGGTGT GCAGAACGAA |
| 15361 | CAACGCCGGC TGCGTGAACG CGGTCCGGTT CAGCAGTTCC GCCCCCTCCG ACCCCGGCCC |
| 15421 | CGCGAACATG ACCTCACGCA GGGAGCGGCC CAGCAGGGGA TCGAAGACCG CACAGGCCTC |
| 15481 | ATCGACGGCA GCGGCGAACA CGGGATACGA CGCATACAAC TCCCGCCCCG CACCCGGGCG |
| 15541 | TTGACTGCCC TGACCGGAGA ACAGGAACGC AGTCCTGCCC GTGGTGACCT GCCCGCGGAC |
| 15601 | GAGGCTCGGG TGCCCGGAGC CCGACGCGAG CGCCGACAAC GCCTCGGTCA GTCCGGCACG |
| 15661 | GTCGGCGCCG ATGAGGGCGG CGCGCTGCTG GAACTGCGAC CGGGTCGTCG CGAGCGCGCG |
| 15721 | GGCGAGGTGA CCGGTGCCGG TCTGCGGGCG GGCGGCGAGG AACTCCGTCA GGCGGTCGGC |
| 15781 | CTGCGCGCGC AGCGGGTCGG GGCTTTTCGC GGAGACCAGC CATACGGCGG GGTCTGCGGA |
| 15841 | GTCGGCTTCG GCCTCGTACG CGGTCCGCTC CTTGAGCGGA GGCTCTTCGA GGATCAGGTG |
| 15901 | CGCGTTGGTA CCACTGATCC CGAACGACGA CACGGCGGCA CGACGCGGCC GCTCCCCCGC |
| 15961 | CTCCCAGACG ACCGGCCCGG TCAACAACCT GACCTCACCC GCCTCGGAGT CCACATGGGG |
| 16021 | CGAAGGCTCG TCGACATGCA AGGTCCTGGG AAGCACCCCG CCCCGCATCG CCATGACCAT |
| 16081 | CTTGATCACA CCACCCACAC CCGGCGCGGC CTGCGTGTGC CCGATGTTCG ACTTCAGCGA |
| 16141 | ACCGAGCCAC AACGGACGAC CCTCGAACCG GCCCTGTCCA TAGGTGGCCA GCAACGCCTG |
| 16201 | CGGGTCGATC GGGTCACCGA GCGTGGTGCC GGTGCCGTGC GCCTCGACGG GGTCGATGTC |
| 16261 | GGCGGCCTCG AGCCCGGCGT CGGCCAGGGC CTGACGGATC ACCCGCTGCT GCGAGGGGCC |
| 16321 | GTTCGGCGGG GTCAGACCAT TGCTCGCACC GTCCTGATTG ACCGCCGAGC CGCGAATGAC |
| 16381 | CGCGAGCACA CGGTGCCCGT TGGGCCGCGC GTCACCGAGA CGTTCGAGCA CCAGCATGCC |
| 16441 | CACACGCTCG CCCCACGGCG TACCGTCCGC CGGCGCCGCG AACGACTTGC ACCGTCGGTC |
| 16501 | CGGCGCCAGG CCCCGCTGCC GGCTGAACTC CACGAAGGTG CCGGGGTTCG CCATCACCGT |
| 16561 | CACGCGGCCG GCCAGCGCCA CGTCGCACTC GCCCGAGCGC AGCGCCTGCG TCGCCAGATG |
| 16621 | GATGGCGACC AGCGAGGAGG AGCACGCGGT GTCGAGCGTG ACCGCCGGGC CCTCCAGGCC |
| 16681 | CAGCGTGTAG GCGATCCGGC CGGACAGGAC GCTGGTCGTG GTGCCGGTCA GCAGGTGACC |
| 16741 | GCTGACTCCC TGCCCCGTCG CCTCGTACAG GCGCGGCGCG TACTCCTGCG GCATCACGCC |
| 16801 | GACGAACACG CCGGCCCGGC TCCCGGCGAG GCTCAGCGGA TCGATGCCGG CCCGCTGCAC |
| 16861 | CGCCTCCCAC GAGGTCTCCA GCACAAGGCG CTGCTGGGGG TCGATGGGCA AGGCCTCACG |
| 16921 | GCGGCTGATG CCGAAGAACG GCGCGTCGAA CTGTCCGGCG TCGTGCAGGA AGCGTCCCTC |
| 16981 | CCGGACGTAC ACCTTGCCCT CGGCGTGCGG GTCGGGGTCG TAGAGCCGCT CCAGGTCCCA |
| 17041 | GCCACGTCGC GAGGGGAATC CGCTCACCGC GTCGATTCCC CCGGCGGCCA GCTCCCACAA |
| 17101 | CTGCTCCGGG TCCGTCACGC CGCCCGGATA ACGGCATGCC ATGCCGAGGA TCGCGATCGG |
| 17161 | TTCCCGCTTG CGTGCCTCGA TGTCCCTGAT GCGCAGGCGC GTCTGGTGCA GGTCGGCGGT |
| 17221 | GACCCGCTTG AGGTAGTCGC GGAGCTTGTC GTCGTTCGCT GGCATGGAGG AACCTGCGCC |
| 17281 | TTGTGCGTGT TCTGCATCGC GCGGCCGGTC CGGTGGGACC GACCGCGCGG CGTGGCGGAG |
| 17341 | TCGGACTGGG GGGACCGTCA GGTCAGTCCG AGTTCGTTGT CGATGAAGTC GAAGATCTCG |
| 17401 | TCGTCGCTGG CCGACTCGAA TCGGTCGGTG GCCCCGGCGG CGTCCTCGGC GTCCGGCCGG |
| 17461 | CCCGCTTCCT GCCGGCCGTG CAGCCACACG AGCAGGTCCT CGAGGCGGGC GGTGAACGGG |
| 17521 | GTGAGTGTCG CCTCGTCCGC GTCGGTGGCG ATCGCCGTGT CCACCAGCCG GTCCAGTTCG |
| 17581 | GCGAGGACCG CCAGGCTGTC GGACGTGGCA GTCCGCGGGG TCTGCGAGGC CAGTCGCGTG |
| 17641 | TAGAGGAAGT CGGCCACGGC CAAGGGCGTC GGGTGGTCGA AGATCAGTGT GCCGGGGAGC |
| 17701 | CGCAGACCGC TGACGGCACC GAGCCGGTTG CGCAGCTCCA ACGCGGTGAG CGAGTCGAAG |
| 17761 | CTCAGCTCCT TGAATCTCCG CTCCGGGTCC ACCGTCGCCG CGGTCGCGTG TCCCAGGACC |
| 17821 | GCCGCGGCGC TGTCCTGAAG CAGCTCCAGC AGCATCCGTC GTCGCGCGGC CTCGGTCGGC |
| 17881 | AGTTCGGCCA CGCGCCGGGC CAGGTCCGGT CCCGCGTCCA CCGCGGTCTG GCGCGGTGCG |
| 17941 | GGGAGGCGGG CGCCGTCGGC TGTTGCGGCG GCCTGTGGCA GACGCCGTAC GGGCCGGGCC |
| 18001 | GGTGCGACGT GCCGGAACAG CAGCGGCACC TCGGCGACGT CCCCCGCTCT CCCGGCACCA |
| 18061 | CGGGCGCTTT CGGCCAGGGC CGCGAGGTCG AGCCGTACCG GCACCAGGGC CGGTACCGCC |
| 18121 | GCGTCGCGCG TGGCGTCGAA GAGGGCGAGG CCCTCGTCGG TGGACAGGGG CAGTACGCCG |
| 18181 | GTGCGTGCCA TCCGGTTCAG ATCGGTCGCG CTCAACGCCT GCGTCATACC GCCGGCGGCG |
| 18241 | TGCTCCCAGG GGCCCCACGC CGCGGCGACC GCGGGCAGCC CGCACCGGTG TCGGGGGTAT |
| 18301 | GCGGCCAGAC CGTCCAGGAA GGCGTTCGCG GCGGCGTAGT TGCCCTGTCC GGCGTGGCCG |
| 18361 | ATCACTCCGG CGACGGACGA GAACAGTACG AAAGCCGACA GGTCGGCGTG GGCGGTGAGT |
| 18421 | TCGTGCAGAT TCCACGCCGC GTCCGCCTTG GGCCGCAGCA CGGTGTCGAG CGGCTCGGGC |
| 18481 | GTCAGTGCTT CGACCGGGGC GTCGTCGAGC ACGCCCGCGG CGTGCACCAG GGCGGTCAGT |
| 18541 | GGATGCGCCT CCGGGACCGC AGCGACGACC CGCGCCAGCG CCGTGCGGTC CGGCGCGTCG |
| 18601 | CAGGCCTCGA CGGTCACCTC GACGGCGCCC GCCGCGCGCA GTTCCGCGAC GATCTCGGTC |
| 18661 | ACGCCCTCGG CGGCCGGGCC GCGCCGCCCG GTGAGCAGCA GTCGCCTGAC GCCGTGCCGG |
| 18721 | GTGACGAGGT GGCGGGCCAC GTTGCCGCCC AGCGCGCCCG TGCCGCCGGT GATCAGGACG |
| 18781 | GTCCCCTCCG GGTCCAGCGG CGCCGGGACG GTGAGGACGA CCTTGCCGAC GTGGCGGGGC |
| 18841 | CGGCTCAGGT GCCGGAACGC GTCCTGCGCG CGCCGCACGT CCCACGCGGC GACCGGGAGC |
| 18901 | GGACGCAGCG CGCCGCGCTC GAACAGCTCC GTCAGCTCGC CCAGCATGGC GCCGACGTGC |
| 18961 | GGGGGTTCCA GCTTCGTCAG ATCGAACGAG CGGTGGTCGA CGCCCGGGTG CCGGGCGGCG |
| 19021 | ACCGCCTCGG AGTCGGGGGT GTCGGTGCTG CCCGTGTCGA CGAACCGGCC GCCGGACGAC |
| 19081 | AGCAGCCGCA GCGAGGCGTC CACGGCGTCT CCGGCCGGGC AGTCGAGTAC CACGTCCACG |
| 19141 | GCGGCGCCGC CGGTCGCGGC CCGGAACCGG GCCGCGAACT CCTGGGTGTG CGACGGCGCG |
| 19201 | AGGTGCGTGT CGTCGAGCCC ATGTGCACCC AGGTCCGCCC ATTCGCCGGG GCCCGCTGTG |
| 19261 | CCGAACACGT CGGCGCCCCG GTGCCGGGCG AGCTGCGCCG CCGCCAGGCC GAGGCCGTCG |
| 19321 | GCCACCGAGT GGATCAGCAG CCGCTCGCCC GGTACGACGG CGGCCAGTTC GACGAGTGCG |
| 19381 | TGATAGGCCG TCAGGAACGC GACGGGCACC GACGCGGCCT GCGTGAACGT CCAGCCCGCC |
| 19441 | GGGATACGAG CGAGGGTGTG CGGCTCCGCC ACGGCCTGGG GGCGGAAGTT GCCGGTGAGC |
| 19501 | AGGCCGAACA CCCGGTCGCC GACGGCGAGA TCGGACACGC CGGGGGCGGT CTCGGTGACC |
| 19561 | ACACCCGCGC CTTCGAGCCC GAGGATGTCG TGGTCCGGCG GTGTGTGGCG GGCGAGGACC |
| 19621 | GGGGCGCGGA GATCGAGTCC GGCGGCGCGC ACCGCGATCC TCACCTGCCC GGTGGCGAGC |
| 19681 | GGGGCGCGCT GGGGCGCCCG GACCTGCTCG GCGACGTCGG GCGGTGCGGG CGACGGGACG |
| 19741 | TCGGCGTCCT GCGCGGCGCG AGGGAGTCGC GGTACGAGCA CCTCGCCGTC GCGCACCGCG |
| 19801 | AGTTGCGGTT CGCCGGTGCG CAGGGCGGCG GGCAGGGCGG CGTATCCGTC GGACGGGTCG |
| 19861 | CCGTCGATGT CGACGAGGGT GAAGCAGCCC GGGTTCTCGG TCTGGGCGGT GCGCACCAGG |
| 19921 | CCCCAGGCGG TGGCCTGCGG CAGGCCGAGT GCGTCCGGCG CCGTCGCCGG GGCCGTCGCA |
| 19981 | TCGTGCGTGA CGAGGAGCAG GCGGCCGACC GTGAGGGCGG GCGGGTCGAG CCAGGACGTG |
| 20041 | AGGAGGGCGA GCGTGCGGCG GGCGACGGCG TGCGCGCCGG CCGCACCTCC TTCGTGCTCT |
| 20101 | CGCGTCCGCT CTCCTTCGTG GGGCGGAAGT TCGGCTCCGT GCGCAGCGAG ATCGGCGAGC |
| 20161 | ACGACGGACG GGTGCGGGTC ACCGTTCTCG ATGCCGTGGA CCAGGGCGTC GAGGTTCGGG |
| 20221 | TACGTGCGGA TGTCGACCGC GTCGGCCGCC AGCGTGGAGA GCGGATCGGG CACGGGGCTC |
| 20281 | CGACCGATGA GCGCCGACTG CTCCGGCGCG TCCGGAGCCG CGGCGCCGAC CGGTGCCGCT |
| 20341 | GCCGGTGTGG CGGACGAGTC GACGTGGTAG AGCGACCGGG GTCCGGCCGG TTGCGTGCCG |
| 20401 | GAGCCGTCGC TCAACCGGTC CAACACCACG GGACGCAGGG ACAGTTCCCC TGCGGTCAGT |
| 20461 | ATGAGGGGGC CGGACGGATC GCTCGCGACG ACGCGGACGG TGGTCGGCCC GGTCCGGGTG |
| 20521 | AACCGGACCC GCAGCGCGGT GGCGCCCAGC GCGTGCAGGG CGACATCGCC CCAGGAGAAG |
| 20581 | GGCAGCAGCG TGCCCGAGGC ATCGGTTCCG GACACCCCGT CCGCGAGCAG TCCGCTGCGC |
| 20641 | AGCAGGGCGT GCAGCGAGGC GTCGAGCAGC GCCGGGTGCA CCGAGAACCG GTCCACGTCG |
| 20701 | TCCGAGGCGG CGGCATCGTC ACCCAGCTCG ACCTGTGCGA ACACGTCATC GTCCGTGCGC |
| 20761 | CACGCGGCGG TCAGCAACCG GAAGTCGGGG CCGTACTCGT AGCCGGTCAG GGCCAGAGCG |
| 20821 | GGGTAGAGGT CCGTCAGGTC GACCGGCGCC GCGTCGGCGG GCGGCCACTG CGGTGCACGG |
| 20881 | TCCGCGGCGT CGGGCGCCTC GGTGGGCCCC AGGGCTCCGC TCGCGTGCCG GGTGCAGGAG |
| 20941 | GACGAGCCGG AGGCGTCGTC GCCGGCGGGC GCCGGGCGCG AATGGACGGC GAAGGCGCGC |
| 21001 | AGGCCGGACT CGTCCGCCTC CTGGACGGTC ACCGGGATGT CGACGCCGCG CTCGCGGGGC |
| 21061 | AGCACGAGCG GTGCCTGAAG CGCGAGTTGC GCGACAGCCG GGTGCTCCCC CGCGCCGTCC |
| 21121 | GACGCGGCAT GCAGCACCAG ATCGAGCAGG GCGGTGCCGG GCAGCAGCGT CGTGCCGTGG |
| 21181 | ATCGCATGAT CGGCGAGCCA GGGGTGCGTG AGCGTGCCGA TACGCCCGGT GTGGACGAAG |
| 21241 | CCGCCGCCCT CGGGCAGTTC GACGGCCGCC GCGAGCAGCG GATGCGGCGT GCGCGTCAGC |
| 21301 | CCGGCCTGCG TGACATCGGC GCGCGGCGCG GGCGGCGTGA GCCAGTAACG CTCGCGCTGG |
| 21361 | AAGGCGTACG TGGGCAGTTC GGGCAGAGCA GCGGAGCGAG GGCCGGGGAG TGCCGGCCAG |
| 21421 | GGGACGTCGG CACCGCTCGT GTGCAGGGTC GCGAGGGCGC GCAGCAGGGC GTCGTGCTCC |
| 21481 | GGCCGTCCGT GGCGCAGCAC CGGGACCAGA GCCGCGGGGC TCTCCTCCAG GGTCTCGGCG |
| 21541 | ACCAGCGTGG CCAGCGTCGG AGTGGGAGTG AGTTCGAGGT AGCGGGTGGT GCCGAGCCCG |
| 21601 | TGCAGGGTGG TGACGGCATC GGCGTGGCGG ACGGTGCGGC GCAGCTGTTC GGTCCAGTAG |
| 21661 | TCGGCGGTCG TGATCCGGTC CTGCTCGGCG AGCAGTCGGG TGAGCGTCGA GACGATGGGA |
| 21721 | ATGCGCGGGG CCCGGTACGT GAGCCGCGCC GCGATCCGGC GGAACTCGTC CAGGATCTGG |
| 21781 | TCCTGGTGCG GACTGTGGAA GGCGTGACTG ACGGTCAGCC GCCTGGTCCT GATCCCCCGC |
| 21841 | TCCGCCAGCT GTGCGGCGAT GTCCGCGAGG ACCTCGGGAT CACCGGACAG GACGGTCGAC |
| 21901 | TCCGGCGCGT TGACCGCCGC GAGCGAGACC ACGTCCTCAC GCCCCGCCAC GAGACCCCGC |
| 21961 | GCGGTGGCCT CGCCGGCCTG GAGGGCGAGC ATGGTGCCGG GCGTGGTGAT CTGCTGCATC |
| 22021 | AGACGGGCCC GGTGGAAGAC CAGCGTGGCC GCATCCGCCA AAGAGAGCAT CCCGGCGGCA |
| 22081 | TGCGCCGCGG ACAGCTCACG GATCGAATGC CCCACCAGAT GGTCCGGCCG CACACCGAAC |
| 22141 | GACTCCAGGA GCCGGAACAA CGCGGTGTGC AGAACGAACA ACACCGGCTG CGTGAACGCG |
| 22201 | GTCCGGTTCA GCAGTTCCGC CCCCTCCGAC CCCGGCCCCG CGAACATGAC CTCACGCAGC |
| 22261 | GAGCGGCCCA GCAGCGGATC GAAGAGCGCA CACGCCTCAT CGACGGCAGC GGCGAACACG |
| 22321 | GGATACGACG CATACAACTC CCGCCCCGCA CCCGGGCGCT GACTGCCCTG ACCGGAGAAC |
| 22381 | AGGAACGCAG TCCTGCCCAC CGTGGCCCGA CCACGTACCA CCATGGGATG CCCGGCACCC |
| 22441 | GAGGCAAGCG CGGACAGCGC CTCGGCGAGT GCGTCCCGGT CCTGGGCGAC GACCGCCGCC |
| 22501 | CGGTGGTCGA AGTGCGTACG GCCGGTGGCC AGGGCCCGAG CGGCCCGGCG GATGCCGACC |
| 22561 | TCCGTCCGGG TCCTGGCGAA CTCGGCCAGC CGGCCGGCCT GTTCCCCGAG CGCGTCGGCT |
| 22621 | TTCTTCGCGG AGACCAGCCA TACGGCGGGG TCTGCGGAGT CGGCTTCGGC CTCGTACGCG |
| 22681 | GTCCGCTCCT TGACCGGAGG CTCTTCGAGG ATCAGGTGCG CGTTGGTACC ACTGATCCCG |
| 22741 | AACGACGACA CGGCGGCACG ACGCGGCCGC TCCCCCGCCT CCCAGACGAC CGGCCCGGTC |
| 22801 | AACAGCCTGA CCTCACCCGC CTCCCAGTCC ACATGCGGCG AAGGCTCGTC CACATGCAAG |
| 22861 | GTCCTGGGAA GCAGCCCGCC CCGCATCGCC ATGACCATCT TGATCACACC ACCCACACCC |
| 22921 | GCCGGGGCCT GCGTGTGCCC GATGTTGGAC TTCAGCGAAC CGAGCCACAA CGGACGACCC |
| 22981 | TCCGACCGAC CGTGTCCATA GGTGGCCAGC AACGCCCGTG CCTCGATCGG GTCAGCGAGC |
| 23041 | GCCGTTCCCG TCGCATGAGC CTGGACGGCG TGGACATCGG GGGCCTCCAG CCCCGCGTCG |
| 23101 | GCCAGGGCCT GAGGGATCAC CCGCTGCTGG GACGGGCCGT TCGGCGCGGT CAGACCATTG |
| 23161 | CTCGCACCGT CCTGATTGAC CGCCGAGCCG CGAATGACCG CGAGGACACG GTGCCCGTTG |
| 23221 | CGCCGCGCGT CACCGAGACG TTCGAGCACC AGCATGGCCA CACGCTCGGC CCATGAGGTG |
| 23281 | CCGTGGGCGG CGGCGGAGAA CGACTTGCAC CGTCCGTCGC CCGCGAGGCG GCGCTGCCTC |
| 23341 | GCGAATTCGA GGAACATGCC GGGGCTCGCC ATGACGGCGG CGCCGCCTGC GAGCGCCAGT |
| 23401 | TCGCATTCGC CGTTACGCAG CGACGGGGGG GCGAGGTGCG CGGCGACGAG CGACGACGAG |
| 23461 | CAGGGGGTGT CCACCGTCAT CGCGGGGCCG TCGAACCCGA AGGTGTAGGC GATGCGTCCG |
| 23521 | GAGGCCACGG TGACGGTGCT GCCGGTCAGC AGATAGCCGC CGACGCTTCC CGCCGTCTCG |
| 23581 | GGGAGCGTCT CGTGCAGCCG GGGGCCGTAT TCCATGGCCG TCGGGCCGAC GAACACGCCG |
| 23641 | GTGCGGCTTC CGGCCAGGCC GGTCGGGTCG ATGCCGGCCC GCTCCACGGC CTCCCAGGAG |
| 23701 | GTCTCCAGCA GGAGACGCTG CTGCGGGTCG ACGGCCAGGG GCTCGCGGGG CGAGATGCCG |
| 23761 | AAGAACTGCG CGTCGAACCG GTCGGCGTCG TAGAGGAAAC CGCCCTCGCG CGCGTAGGTC |
| 23821 | CGGCCCGGTG CGTCGGGGTC CGGGTCGTAG AGGCCCTCCA GGTCCCAGCC ACGGTTCTCG |
| 23881 | GGGAACACGT GGATCGCGTC GGCGGCCTCG GCGACAAGCT GCCACAGGGC TTCGGGGGAA |
| 23941 | TCGGCGGCGC CGGGGTAACG GCAGGCCATG CCGACGATCG CGATCGGCTC GTCGGAGACC |
| 24001 | GAGGGCGGCG ACGTGGCGTC GCTCTGCGTG GTACGGGCCA GCTCCGCTCC CAGCACTCGT |
| 24061 | GCCAGGGCTC GTGGCGTCGG AGTGTCGTAC AACAGGGTTG CCGGCAGGCT CAGTTGGAGC |
| 24121 | ACAGCGGCCA GCCGGTCGCA CAGGTCCTCC GCGGACTGCG ACTCCAGGCC GAGATCGTTG |
| 24181 | AAGGAGCGGG CCAGGTCCAC TTCGCGCGGA TCGGAGTGAC CGAGCAGCGC CGCCGCCTCG |
| 24241 | TCACGGATGA GGTCCAGCAA CTGCTCGTCA CGCCCGGCAG GAGCGGCCTC GACCAGCCCT |
| 24301 | CGCAGCCAGT CGGAGTCCCG CACGGACGCG GGTGCGGAGA CAGCGGACGT CTTCTCCGGG |
| 24361 | CGGGCCGTGA CGGCCGATGC GGCCGACGCG GTGGCACGCC GCGCGGCACC CTTCAACTCC |
| 24421 | AGACCCAACA CCTGCGCGAG GACCTTCGGC GTCGGGCTCT CGTACAACAG CGTTGCGGGC |
| 24481 | AGACGCAGTT GCAGCACGGA ACCCAACCGC TCGACCAACT CCACACCCGA CGCCGACTCC |
| 24541 | AGGCCGAGGT CCTTGAACGA GCGGGCGAGA TGCACTTCGC GCGGATCGGA GTGACCGAGC |
| 24601 | ACCGCCGCCG CCTCGTCACG GATGAGGTCC AGCAACTGCT CGTCACGCCC GGCAGGAGCG |
| 24661 | GGCTCGACCA GCCCTCGCAG CCAGTCGGAG TCCCGCACGG ACGCAGACGC GGACACAGGG |
| 24721 | GGCCCGGAGG CGGGCACAGC GGCGCCAGCG GGAGCAGCAG GGTTCGGCGT CGGAACGGCG |
| 24781 | GCAGCGCCCT GGCGTGCCAC GGGCGCGGAC GTCGGCGTGG GCTCGGGCCA ATACCGGCGC |
| 24841 | CGGTCGAAGG CGTAGCCGGG CAGTTCGACG CGGCGCGCCG CCGGAAGGCC GTAGAGCGCC |
| 24901 | GGCCAGTCGA CGGCAGCGCC CCGCACGTGC GCGGCGGCGA GCGAGGACAG CAGCCGCGGC |
| 24961 | CGGCCGCCGT CGCCGCGGCC CAGGGCGGGA ATGCCGACCG CGCCCGCGGC GTCGAGGAGT |
| 25021 | TCCAGGATCT CGGGCGGCAG CACGGCGTGC GGGCCGACCT CGATGAAGAC GGTGTGCCCG |
| 25081 | TCGTCCATCA GTTCCTCGAC GGGCGGATGG AAGGGCGCCG GCTGCCGGAA GTTGCGGTAG |
| 25141 | CAGTGGTCCG CGTCCAGGGC GGGGGTGTCC ACCGGACCGC CGAGGGTCGT CGACTGGAAG |
| 25201 | CGCGTCCGGC TCGGCGTGGG CTCGATGCCG CTCAGCTCGT CGAGGAGCGC GTCGCGCACC |
| 25261 | GCCTCGGCCT GCTCGCCGTG CGCACGGCCG CGTGCGACGA CGAGAGCCGC GGCGTCCTGG |
| 25321 | AGGGTGAGCG CGCCGATGCT GTACGCGGCG GCGATTTCGC CCGCGGCGTG GCCGAGCACG |
| 25381 | GCATGGGGCT GGACGCCGAG CGTGCGCCAG GTGTGCGCGA GGGCGGTCGT GACGGCGAAC |
| 25441 | AGCACGGGCT GGACGTGGTC GGGGGTGTCC GGCAGGGTCT CCGGACCGGT GAGGTGGTCG |
| 25501 | AGGAGGGACC AGCCGGTGAG CGGGTCGAGG GCGGCGGCGG CGGCCTCCAC GTGCTCGCGG |
| 25561 | AAGACGGGCA GGGTCGCCAT CAGGTCGCGG ACCGTGCCGG CCCACTGCAC GCCCTGGCCG |
| 25621 | GGAAACACGA ACACGGTCTT GGGCCCGGTG CCGGGCGTGG TGCCGGTCCC GGGGTCGGGC |
| 25681 | GGGGTGCCGC GCAGCAGGCC GTCCGAGGGG CGGCCCTGCG CGAGGGTGCG CAGCTGGGAG |
| 25741 | AGCAGGGCGG ACCGGTCGCC GCCGAACGCG GCGGCGGGGT GCTCGTGATG CGTGCGAATG |
| 25801 | GTGGCCAGGC CGCGCGCCAC GGTGGCGGCG TCCAGCTCGG GGTGCTGCTC CAGGTGCGCG |
| 25861 | GCGAGGGCCG CGGGCTGACC GCGCAGCGCC GCCTCGCTGC GCGCGGAGAT CAGCCACGGC |
| 25921 | GACGCGACGT GCTGCGGGAC GGCAACGGGA ACAGGAAGGG TCGCGGGCGG GGCCGCCGAT |
| 25981 | GACGTCGCGG GTGACGGCGC CGACGACTCG GCCGGGGCGT CGCAGAGCAC GACGTGGCAG |
| 26041 | TTGGTGCCGC CCATGCCGAA CGAGCTGACG CGTGCGACGA TCCGACCGTC CGGGCGCGGC |
| 26101 | CACGGTGTCA GCCCGACCTG GACGCGCAGG CTGAGCTCGT CGAAGGCGAT CTTCGGGTTC |
| 26161 | GGCGTGACGA AGTTCAGGCT CGGCGGCAGC TTCCGGTGCC GGATGGCCAG AGCCGTCTTG |
| 26221 | ACCAGGCCCA CCACGCCCGA AGCACCCTCG AGATGCCCGA TGTTGGTGTT CGCGGAGCCC |
| 26281 | ACCAGCAGCG CGTTGTCGGC CACCCGGCCC ACGCGCGCGC CGAAGGCGGT GCCGAGCGCC |
| 26341 | GCCGCCTCTA TCGGGTCGCC CACGGCGGTG CCCGTGCCGT GCAGTTCCAC GTACTGCACG |
| 26401 | TCGCGGGGGG CCACGTCCGC CTGCCGGCAG GCGGCGCGCA GCAGCTCCGT CTGCGCGGGC |
| 26461 | GCGCTGGGCA CCGTCAGTCC GTCGGTGGCG CGGTCGTTGT TGACGGCGCT GCCCCGGATG |
| 26521 | ACGCAGTACA CGAAGTCGCC GTCGGCACGG GCCTGTTCCA GCGGCTTGAG TACGACCAGT |
| 26581 | GCGCGGCCCT CGCCGCGAAC GTACCCGTTG GCCCGCGCGT CGAAGGTGTG GCAGCGGGCG |
| 26641 | TCGGGCGAGA GGGCGCCGAA GCTCATCGAC GCGGCCATGC CGTCCGGCGC GGCGATCAGG |
| 26701 | TTCACGCCGC CCGCCAGCGC CACCCGCGAG TCGCCGCGGC GCAGGCTCTC GCAGGCGAGG |
| 26761 | TGCACGGCGA CCAGCGACGA GGCCTGCGCG GCGTCCAGGG TCATGCTGGG GCCGCGCAGG |
| 26821 | CCCAGCGTGT ACGACACACG GTTCGGGATG AGCCCACGGC CCATGCCGGT CATCGTGTGC |
| 26881 | TGGTTGAAGG AGGCGGTTCC GGCCCGGGCG ACGACGCTGC GGTAGTCGTC CCAGATCGCT |
| 26941 | CCGACGAACA CTCCGGTGCC GCTGCCGCCG AGCGAGGCGG GGACGATCGC GGCGTCCTCC |
| 27001 | AGCGGCTGCC AGCTCAGTTC CAGCATCAGC CGCTGCTGGG GGTCCATCGC CCGTGCCTCG |
| 27061 | TGCGGCGAGA TACCGAAGAA CCCGGGGTCG AAGGTGTCGA TCCGGTCCAG GTAGGCGCGG |
| 27121 | TACCGGGCCG CACCGGTGGG CGTGGCGGCC GGGTCGGGCC AGCGGTCGGC GGGCGTCTCG |
| 27181 | CCCACCGCGT CCACGCCCTC GCTCAGCAGC CGCCAGAAAG TCGCAGGGTC CGGAGCCGGC |
| 27241 | GGCAGCCGAC AGGCCATACC GACGACGGCG ATGGGCATGA ACCCTTCAGA GATGAAGCGC |
| 27301 | ACCCTCGACG GATATGGAGG AGTCGCCGCG GTGGGCGGCG GGCCGGCCGA GCGTCTCCAA |
| 27361 | CGACGGAAGG AAGTCGAGCT CTTCCAGCGG ACGATCGGTG AAGAAGCGCG CGATCGTCTC |
| 27421 | GGCCCAGTCG GCGGCAGGCG CGAGGAACAC GAGATGGTCG GCCTCCGGGA TGACGGCGAA |
| 27481 | CAGCGCACCC TCGATCTCGG CGGGCAGGGC GCGCGCGCCC TCCATGGTGG CGAAGGTGTC |
| 27541 | GTGCTCCCCG ACCAGGCACA GGCTGGGTAC GCCGCTGATG CCGCCGGGCA GGACGGCATC |
| 27601 | ATCCTGGAGG AGGAGGTCCG AGACGTTGAG GTAGCCGGGA AGATCGGGCT CCTCGATCGT |
| 27661 | GCTGAAGCGA CCGTTGAGGG CCGTGCGGAC GATTTCACGG TTGCGCACCG TGACCGCGGG |
| 27721 | GTGCAGGCAC ATGAGCAGGT CCAGCAGACC TTCGGCGAAC TCGGCGAAGC GGCCCGCCGT |
| 27781 | GAGGATCGGA TACATCTGCG TCATCCGCTC CCGGTTGCGC GGCGGGCAGT CGGCGGCGCC |
| 27841 | GGCCAGGACG AGACGGGAGA CCCGCGACGG GCTCTGCTGG GCGTAGCGGT AGGCCGGCGG |
| 27901 | GAATCCGTTG CTGATCCCCA GCAGGTTCAC ACGCGGCAGC CCCAGCTCGT CGATGAGGTG |
| 27961 | GGCGAGGGCT TCGGTCTGGA CGTCATAGCG GCCTTCGGCG GGCACGGGGT CCGCCGTGCG |
| 28021 | CGAGCCCGGC AGATCCACAC AGACGATGGT GGCCGTGTCC TGCCAGTACT TGTCGAACCG |
| 28081 | CCGGTAGCTG AACTTGTCCT GGTACGCACC GGACAGCACA ACAAAAGGCT CGGTGACCGG |
| 28141 | TGCTTCGCAT TCGACCATCC GGTAGCTGAA CGCAAGACCT TTGTAATGAA GCTCTTCGAG |
| 28201 | CTGCTCGCGC GGATTTCCGG CGCCCACGGA TCAGCTCCTC GAATTTCGGG CGGATGTGCA |
| 28261 | CGGACGGACA ACGGATACAC GTCGGTGCAT GAGCCCGATC TTTGTCGCCG GCCAGGGCAC |
| 28321 | CGACAACCCC TATTTCCCCC CTTAGCCGAA CCGGCTTGCC GGATCGGAGG TGGTCGGAGC |
| 28381 | TGCGAGATGA GTCCCGATAC GAATCCTCTC CAGATTCACC CCCTGGCACA CGACCCATCG |
| 28441 | ACATGTATTC TCGGCGTATG CCCATCATCG AACTTGGCGA ATACGGGCCA GACTTTCTCG |
| 28501 | CAGATCGTTA CCCGTATTAC GCGAAACTCC GCGAGGAGGG ACGCGTGCAC GAGGTACGGG |
| 28561 | CCCCGGACGG CTATCGATTC TGGCTGATCG TCGGATATGC CGAGGGGCGC GCCGCCCTGA |
| 28621 | CCGATTCGCG GCTGGTCAAG GCACGCGACA CGATGGCGAC GTCCGAGGCG TCGCCACTGG |
| 28681 | GCAAGCATGT GCTGATCGCC GACCCGCCGG ACCACACCCG GCTGCGCAAG CTGATCTCCC |
| 28741 | GGGAGTTCAC CGTGCGGCGG GTGGACAACC TGCGCCCGCG CATCCAGGAA CTCACCGACG |
| 28801 | ACTTGGTGGA CGTCATGCTG CCGGCGGGGC GGGCCGACCT GGTGGAGGCG CTGGCCCGGC |
| 28861 | CGCTGCCGAT GGCCGTGCTG TGCGAACTGC TCGGAGTGCC GAACGCCGAC CGGGACGAGT |
| 28921 | TCCACTCCTG GGCCAAGGGC ATCCTCGCGC CGCAGAACGG GAGCGAGACG CACACGGCCG |
| 28981 | TCAAGGCCTT GATGAGTTAT CTCGACGACC TGATCGAGGA CAAGCGGCAC GGAGAGCCCA |
| 29041 | CCGGTGACCT GCTGTCGGGT GTCATACGCA CCACCATGGA GAAGGGCGAC CGCCTCTCCT |
| 29101 | CGGAGGAAGT GCGCTCCACG GCCTTCGTCC TGATGATCGC CGGACACGAG ACGACGGCGA |
| 29161 | ACGTCATCTC CAACGGAACG CGGGCGCTGC TCACGCACCG GGACCAACTG GACCTGCTGC |
| 29221 | GCTCCGACAT GGACCTCCTC GACGGCGCCG TCGAGGAGAT GCTCCGCTAC GACGGCTCGC |
| 29281 | TGGAGAGCAC GACCAAGCGG TTCACCGGTG TGCCGGTCCA GATCGGCGAC ACGGTCATCC |
| 29341 | CGCCGGGCGA GACGGTGCTG GTCAGCCTCG CGTCGGCGGA CCGCGACCCG GCGAACTTCG |
| 29401 | ACGACCCCGA CCGCTTCGAC ATCCGTCGCG GCACCCCGGC CGGCGTCGGC CACCTCGCGT |
| 29461 | TCGGGCACGG GATCCACTAC TGCCTGGGAG CCTCACTCGC CCGCGCAGAG GGCCGGATCG |
| 29521 | CGTTCCGCGC GCTGCTGGAG CGCTGCCCCG ACCTCGAACT CGACCCCGAG GCACCGCCGT |
| 29581 | TCGAGTGGAT GCCGGGGGTT CTGGTCCGGG GCGTGCAGCG GTTGTCGCTG CGCTGGTAGG |
| 29641 | CCGAAGAGAG GCACGTATAC GGATGCAACG GCGAAGCGGN NNCCGCTTCG CGATGAGGGT |
| 29701 | GTGACCGCCG ACGGTGTGGC TGTCGGCGGC ATGACCACTC GTCACCAGGT GACGGGCAGG |
| 29761 | CTCTTCGCTC CCTGGACGTC AAGTCCCGTG GAGAACGGGA TCTCGTTCGG CGCGACGGCC |
| 29821 | AGGCGCAGCT GCGGGATGCG GCTGAACAGC CGTCCGTAGA CCACCTCCAG CTCCATGCGT |
| 29881 | GCCACGCTGT GCGCGACGGA CAGGTGGATG GCGGAGGCGA AGGCGAGGTG GTCGGGGGCC |
| 29941 | GACCGCTGGA TGTCCAGGTC GTCGGGGCGC TCGTACACCG ATTCGTCCCG GTCGGCTGAG |
| 30001 | CTGATCAGGC AGATGATTCC GTCGCCGGGC CGGATGGTCT GCCCGCCGAT CTCGATCTCC |
| 30061 | TCCACCGCCA CGCGCTTGGG CGCGAACTCG GCGACGGTGA GGTAGCGCAG CAGTTCCTCG |
| 30121 | GTGGCCGCCG CGGCCCGTTC GGGCTCCTTG GGCAGCAGGG CGGCCTGCTC GGGGTGGCCG |
| 30181 | AGCAGCGCCA GCACAGCGGT GGTGATGATC TTGACGGTGG TGTCGTATCC GGCGCCGAGC |
| 30241 | AGCAGCCCGA TCTGCATGAG CAGGTTCGTC TCCGTGAGCC TGCCTTCGTC GGCGAAGCCG |
| 30301 | ACCAGGCGGC TGACGATGTT GCGGTCCGGT TCCGCACGGC GCAGTTCGAC CAGTTCCTTG |
| 30361 | AGCTGCTCGA ACAGTTCACG GCGGGCGGCG ACGGCGGTTT CGGCGGAGCC GTCCTCGTCG |
| 30421 | AACAGCCGGG CGGTGGTCTC GCCGAACCGG GCCATCGCCT CGGCAGGCAC CGCGAGCAGC |
| 30481 | CAGCCGACGA CCGTCGACGG GACAGGAGGG GCCAGCGCCG GCACGAGGTC GGCGGGGGTC |
| 30541 | GGGCCCTCCA GCATCCGGTC GATGAGCCCG TCGACGAGCT GCTCGGTCGC CGGACGCAGC |
| 30601 | GCCGGCACCC GCTTGATCAG GAAGTGCGGC GCGAGCATCC GCCGGAGTTC GGTGTGGTCG |
| 30661 | GGCGGGTCCA TCCGGCCCAG CGGCCGTACG CCGCCCTCCG CGGTGTTCAT GGCCTTCGCG |
| 30721 | ACCTCACTCA GCCATGGGAA ACGGGGGTTG AGCGCGTCCA CGCTGGCCCG GGAGTCGGAG |
| 30781 | AGGATCTTGC GCACGTCCTC GTGCCGGCTG ATCAGCCAGG CCTCCCGCCC GTTGTAGAGC |
| 30841 | CGCGCCTTGG ACACCGGCTG CTCGGCGCGC AACGTGCGGT AGACGGGCGG GGGATCCATC |
| 30901 | GGGCAGCCGG GCGCCTTCGG CATCGGATAT GCCGGCGGCG TCTCGGAGGA GCTGTCCGTC |
| 30961 | GTCAGGGGCG GTGTACCGGT GGGCACGGCG TCGGTCGAGG TCATCGTGGC GACCCTCCTG |
| 31021 | GGAAGACTTC TGGTGGGGTG GTTCTGTCGC GGCGGGCGCG TGTCACGTTT CGACAGGCCT |
| 31081 | GGGCGCGCGT CACATCTCGG TGGGTGGGTG TCATGCCCTG GCCGACACAA GCCCGTGGTT |
| 31141 | CCGTTCGGCC CGCTTCGAGC CGATGTACGA GAGGACCGTC GCGCACAGCG CGAGGGCGGC |
| 31201 | GCCGACCGCG ACCGACGTGG CAGGACCCGA GGCCTCCAGC AGCACGCCGG TCACCGGCAT |
| 31261 | GCTGAGCGCG GGGCCGCCGG TGCCGGCGGT GGCCAGCCAG CCGAAGGCTT CCGCCCGGCG |
| 31321 | CTGCTGCGGC ACCGCTCCGG ACACCTGCGG GAAGTTGCCG GCCATGCTCG GGGCGATGGC |
| 31381 | GGTGCCACCG AGGAAGAGCA CGACCAGCAC GAGCCAGACC GGAGCGGTTT CGGTGACCGG |
| 31441 | CGGCAGCAGC AGCGCGAGCA GCGCCATGCC GACTGCCATC GCCGCGAACC GGGCGCTGTG |
| 31501 | CGGCACGTCC TTGCGCAGGG CGCCCATGCC GAAGCCGCCG ACCACGGATC CGATCGTCCA |
| 31561 | GCATGCGATG AGCACGCCGG ACAGTCCGGA CTCGTGGCTG TCGCGGGCCC AGGCGACCAG |
| 31621 | GGAGAGGTTC ACCGAGAAGA GGGCGGCCAT CATCACCAGG GTCACGACGA TGGCGAGGGT |
| 31681 | GAACTTCGGC AGGGGGAAGA GGGTACGTCG CTCCTCGGGC TCCGGTGCGG ATGCTCTCGT |
| 31741 | GACCGCGGAC GCCGACGTGG CCGTGTCTCC TTCCGTGCCG GCGTGCGCTC CCGTTTCCGC |
| 31801 | TGCTGTCCGC GTCTCTGGTT GTGGTTTCGC GGCGGTCCGC TCCGCCCCGC CGCGGATCCC |
| 31861 | GGCGCTGCCC AGCGCGGCGG CGAAGGCGAG CGCGCCGAGG AACGCCACGA CGCCGCACGC |
| 31921 | GATGACGGCG TATCCGGGGT CGAGGGCGGT CACGAGCAGT GAGGTGAGCA GCGGCCCGGT |
| 31981 | GGTCTGCACC ACCTCCGAGC CGGTGGCCTC CAGTGTGAAC AGCGTGCGGG CGAGTTCGCC |
| 32041 | GGGCACGATC TTGGGCCACA CCGGGCGGCT GACCTGCGAG ATGGGCACGG TGCTCATTCC |
| 32101 | GGTGAGCAGG GCGACCAGCA CCGCGATCGG CCAGCCGCCG CCCGGCACGG TGCGGGTCAT |
| 32161 | CGTCACCAGT GCACCGAGGC CGACGAGGTA GCCGATGCCG GTCAGGACCA GCAGCTTGCG |
| 32221 | GACCGCGCCC CGGTCCGCGG CGCGGCCCCG GGCGGGTCCG ACGAGCGCCT GCCCCGCGGT |
| 32281 | GAGCGCCCCG CCGAGCACGC CGGCGGCGAC GTAGGAATCG CTCGAGCCGA CCAGCACCAG |
| 32341 | CGTGAGGCCG ATCGGCAGCA TGGAGACGTT GAGGCGGGGC AGCATGGACC AGAGGAACAG |
| 32401 | GCGCGGCAGG TGCGGAATGC CTGCCAATGC CTTGTAGGAC TTCACGGTTC ACACCCTGTT |
| 32461 | TCGCAAGTCA TGACGCATAC GCGCCCGCCG TGCGGTTCGG CGCCGGCGGA CGCTGCGTCT |
| 32521 | TGATCAGGTC TCTTCGGACG TTCGGGGCCG CTCAGAAAAC CTGGAAACGG GCGGCGATGC |
| 32581 | GGTCGGCGTT GTCGGGGTTC TGCCGGGCCC GCTCGATCAG CTTCTCGTTG CTCCAGGTGC |
| 32641 | TGGCGGTGGT GGCTGTCTCG TCGCCCGCGT TCTGGCGTGC GGGGTAGATC CAGCCCTCGG |
| 32701 | CGGCGCAGCG GAACCGGAGG ATGTCGGCGA GGAAACGGAG TTCGGCCCGG TCGGCGGGCG |
| 32761 | CCTCCTCGAA GTAGCCGCGC ACGAGCGGCT CGCCGTCCGT GTCGTCCTCC AAGTAGCTGA |
| 32821 | GTGTGGTGGC CAGTTCCAGC ATGCAGGGTG CGTACATGGC CTCCGACCAG TCGAGCAGAG |
| 32881 | CCGCGGTGTC GCCGAGGACC CGGAACTCCT TGGCCGCGGC GTCGGAGTTG ATCAGGCCGA |
| 32941 | TGGTCAGGTC GTCGGGCGAC AGCGCGCCGC CCGCCTGCTG CAGCGTGCGC CGGATCCAGT |
| 33001 | GGTGTGGCTT GAGGAAGTCC TGTTCGAGCA GGAAGAGTTC GAGCACCTCG TTCCAGCGCG |
| 33061 | GCACGCCCTC GGGGACGGGG GCGTGCAGCA GGACGGAGTC GATCCGTCCG AGGGTCCGGC |
| 33121 | CGACGGCGCG CAGGTGGGCC GGGTGGGTCT CGTCGACACG CTCACCGTCG AGGTAGGTCA |
| 33181 | GCAGGCTGTA GCAGAAGTCG CCCTGATAGG CGGTGACTTC TCCGCCGGTG GTCGGCAGCG |
| 33241 | GGCCGCCGGC GGCGATGCCG TGCCGCTCCA CCTCCTGGGC CAGCAGGAGT CGTGCGGTGA |
| 33301 | GCTTCGGCCC CATGTCCTTG CGGAGGGCCT TGACGACGTG GCGTACGCCG TGGTGCCGGA |
| 33361 | GGAGGTAGGT GTGGGAGCTG TACCCCTCGT CTCCGGGCAG CCGCTCCCAC GGCTCGACGG |
| 33421 | TCCAGTCCTG CCAGCCCCAG AGGGTCAGCG GCAGGTCGTG CGCTTCGGGT ACCGCGGCTG |
| 33481 | CGGCGTCGGT CGATTCCGGC ATTGAAGGGG TTTTCGTTGT CTGGTCGTGA TGTTCCGAGT |
| 33541 | TCCATGTCCG ACGGCCGCGC GGTCGCGGGG TCGCGCGGTC GCGCGGTCGC GCGGTCGCGC |
| 33601 | GGTGGCGCGG TCGCGGCATC ATGTGGTCAT GTGGTCATGC GGTCACGGGG CAGGGTCATG |
| 33661 | GTCCGCGGGC GCGTCCCAGG CGACGGGGAG CGAATTGACG CCGTACACCA CGGAGTTCGC |
| 33721 | GCGGAACTCG ATGTCCTCGA AGGGCACGGC GAGGCGGAGC GTGGGGAAGC GGTCGAACAG |
| 33781 | GCGCAGGTAG GTCACCTTCA TCTCGACGCT GGCGAGGTGG TGGCCGATGC ACAGGTGGAT |
| 33841 | GCGGTGGCCG AACGCCAGGT TGCGCTCCTC CGCCCGGTCG AGGCGCAGCC GGTGCGGGTC |
| 33901 | GGTGTAGAGC TCCGGGTCGT GGTTGGCCGC GGCCATCGAA CCGACCACGG TCTCGCGCGC |
| 33961 | CTTGATGAGG TGCGCGCCGA TCTCCACGTC CTCGGTCGCC AGGCGGGCGA AGTTGAACTG |
| 34021 | CACGATGGAC AGGTAGCGCA GCAGTTCCTC GACGGCGGTG TCGATGAGCG ACGGATCGGA |
| 34081 | GGGCAGGAGG GCGAGCTGGT GCGGGTTGCG CAGCGGGGCC AGGGTGGCGA GGCTGAGGAT |
| 34141 | GTTCGAGGTC GACTCGTGCC CGGCCATCAG CATCAGGGCG CACATTCGGG CGATCTCGAA |
| 34201 | CTCGGATATC CGGTCCTTCT CCTCCGCTGG ATCGAGCAGA TCGCTGATCA GGTCGTCCGA |
| 34261 | CGGGTTCTTC TTCTTGTCCT CGATCAGGTC CAGCATGAAC TTGGTGCCTT CGAGGACGGT |
| 34321 | GCGCTGCTGC TCCTCGTCGG ACAGTTGGGT GTCGAGGATG CTCAGGGACC AGCGCTGGAA |
| 34381 | GTCGTCGCGC GCGTCGTAGG GCACGCCGAG CAGGTCGCAG ATGATGAGCG ACGGGATGGG |
| 34441 | CAGGGCGTAG GCGGTGACGA GGTCGACCGG GCCGCCCGCC GTCTCCTCCA TCCGGTCCAG |
| 34501 | GTGCTCACGG GTGTACTGCT CGATCTTCGG CTCAAGAGCC TTGATCTTCC GGACCGCGAA |
| 34561 | ACGGCTCGCG GCCAGTCGCC GGTAACGGGT GTGGTCCGGC GGGTCCATGG TGAAGAAGGC |
| 34621 | GCCGGGCAGC GGCGTGGGGC GGCCCTCGGT ACGACGAGTG CGGGCGGCCT TCTCCCGGAC |
| 34681 | CGAGCTGAAG GACGGGTTCG CCAGCATCGC CTTGACGTCC GCATGGCGGG TGAGCAGCCA |
| 34741 | GCCCTGGTTG CCGTCCGGGA AGGCGATGGG ACTCACGGGC GCCTCGGCGC GCACGTTCGC |
| 34801 | GTAGCCGTCC GGGGGGCTGA AGGGGTCGCT GCGGTGCAGG GGCAGGCCCT GCAGGAGAGA |
| 34861 | AAGAGTGTTC ATGGTCGTCC TTCCTCAGAG GGCGTCGGTG TTTCGAGTCG GTGCGTACTG |
| 34921 | AGGGGTGCGG AGTGGTACGG GGCGGGCGCG GACGCGCGGC GGCCCCGTAC CTGCTCGATC |
| 34981 | AGGGCCGGGT GAGGCCCGGC GTACGGCTCA GCCACTCGTC CACCGCCGCG GCCGTGGTCT |
| 35041 | CGGAGAATTC GCCGATCATG GTGCAGTGGT CGCCCGGCAC CTGCGTCTCC TCGTGGTCGA |
| 35101 | GGGGCCACGC CGCCTGCGAG TCGGGGCCGG CCATGGGTTC CTCGGGGGAA CCCGGGATGC |
| 35161 | AGCTGTCCGG GCGGACGAAG AGAGTCGGCA CGGCGAGTTG CCGGGGCTGC CAGCCGCGGA |
| 35221 | ACATACCGCG GTACGTACCG AGGGCGGTGA GGCCGTCGTA GTGCATCGAC GTGAACCGCA |
| 35281 | TCCGGCGCTC GACGACCTCG TAGGTCATGG CCTTGCGCAT CTCCAGCGTC ATGCTGTCGG |
| 35341 | GCGGGTAGGT GTCCAGCAGG ACGACGCCGA CCGGGCCTGT TCCGCGCTCC TCCAGCCAAG |
| 35401 | TGGCGGCGGC CTGGGCGAGC CAGCCGCTGG ACGAGTAGCC GAGCAGCGCG TACGGCCGGC |
| 35461 | CGTCCGCCGC ACGCAGCACC GCCTCCGCCA GTGTCTCGAT CAGCAGCTCC AGGGAAGCGG |
| 35521 | CGAGAGGCTC GCCGGCCATG AAGCCCGGGA CGGTGACCAC GGAGACCCGG CGGCGGCCAC |
| 35581 | GGAAGTGGTT GGCGAGGCGG GCGAACTGGA GCGAGCCGTC CAGGGGCGCG AACGGCGGGA |
| 35641 | AGGAGAGGAG CTGCGGCTCC GCCTCGCCGT GGCCGAGGGT GGTGACGTGC GCGCCGCGGC |
| 35701 | CCAGGTCCTC GGCGCCGTGG AATCTGGTGC GCAGGGCGGA GGCGCTGCTG AGGAAGGCCT |
| 35761 | CGACCTCCTG CATCCGGGCT TGCAACGAGA GCTTGCGGTA GATGCCGACG ACGGAGTCGT |
| 35821 | TGGAGTCCTG CGGCGGCGCG GCGGCCAGGG AGGCGGACGG CGCCGCGGAC ACGCCGGGCG |
| 35881 | CCGGTGACGC GCCGGCGTGG CCGCCCGGCG CGGCATTGTC CAGCAGATGG GTCACCAGGG |
| 35941 | CGCCGAGCGT CGGATGGTCG AACACCAGGG AGCTGGGCAG CGCCAGGCCG GTCAACACGG |
| 36001 | TCAGCCGGCC GCGCAGTTCG ACCGCGGCCA GGGAGTCGAA GCCGAGCGCC AGGAACTCCT |
| 36061 | GGTCCTCGGT GATCGTGCCG GCGTCCGCGT GCCCGAGGAC GCGCGCGGCG TGGCGACGGA |
| 36121 | CGATATCGAG GACGTAAGGG CGCTGATCGG CGAGCGGAAG GTCCGGCAGG CGCTGCCATT |
| 36181 | CGCCGGGCTG CGCGGCACCG GTGGGTCCGG CATCGGGGTG TCCGGCACCG GACTCCGTAT |
| 36241 | CGTCGAAGTC GGCGAACAGG TGGTTCGGCC GGCCCGCGGT GAAGACGCCG ATGAACCGTG |
| 36301 | GCCAGTCCAG GTCCGCGACG ACGATGGCGG TGTCCTCCTG CCTGACCGCC CGGTCGAGGG |
| 36361 | CGGTGGTGGC CAAGCGCGGG GTGAGCGGCC GAAGGCCCCG GCGCTGCATC TCCTGCGGGA |
| 36421 | ACTGTTCTCC GGCCATGCCG CCGCCGCTCC AGGGCCCCCA GGCCAGCGTG GTGGCGGCGG |
| 36481 | CGCGGCGGGG GCGGCGGCGC TCGACCAGCG CGTCCAGGAA GGCGTTCCCG GCCGCGTACG |
| 36541 | CACCGCCGCG GGTGGTGGCC GAGGTCCCCG CGATGGACGA GTAGACGACG AACGCGGCGA |
| 36601 | GTCGGTCGCC CAGCACCTCG TCCAGGATGA GGGCACCGGT GACCTTCGCG TCGACGACGG |
| 36661 | CGGCGAATTC GGTCGCGTCG AGATCAGCCA GCGGATGTTC GGCCGCCACG CCCGCCGTGT |
| 36721 | GCACGACCGC ACCGACGGGG GCACCGCGGC CGGCCAGGTC GGGGGCGAGC GCCGGGAGCT |
| 36781 | CGTCGCGGCT GGTCACGTCG GAGGACACCA GGTCCACCGT GGCACCGTGG GCGGCCAGTT |
| 36841 | CGGCCCGCAG GTCCGCGGCG CCGGGGGCGT CGGGCCCCTG GCGGCTGGCG AGGACGAGGT |
| 36901 | GCGGGGCACC CTGCTCGGCG AGCCGACGCG CCGTGTGCGC GCCGAGGGCG CCGGTGCCTC |
| 36961 | CCGTGATGAG GACCGAGCCG TGCGACCACC AGGGTTCGCG CGCGGCGGGC GGCTGCGGAT |
| 37021 | CACCGGTCCG CCCGGTGGCG TCGGCCCCCT CCGGGGCGAC GAGGGATTCC GGGGCGACGG |
| 37081 | GGCGTACGGG CTCGGGTGCG CCGTCCGGGC CTGGGGGCCG CAGGCGGCGC ACCCGTGCTC |
| 37141 | CGTCGGCACG CAGCGCGACC TGGTCCTCAC CACTGGATCC GGCCAGCAGC GCCGCCAGGC |
| 37201 | CGAAGGAGGC CTCGGCCGTC GCGGCGAGGG CGTGCCCGTC TGCCGCGGAC AGGTCGGGCG |
| 37261 | CGGGCAGGTC GACCAGGCCG CCCCACAGGG TGGGGTGTTC GAGGGCCGCG ACCCGTCCGA |
| 37321 | GGCCCCAGAC CTGGGCCTGC CACGGGTCGG GAGCGTCGTC GGACGCCGTC GCGCGGACCG |
| 37381 | CTCCGCGGGT GAGCGTCCAC AACCGGGTCG CGCTCCAGCC CGTGTCGAGC AGCGCTTGGA |
| 37441 | GAAGGCACAC GGAGGCCCAG GCGCCGGAGC CGACGCCGCG CGGCCCGGTG TGCTCGCGGC |
| 37501 | CGGACAGGGC GAGCAGCGAG ACCACTCCGG CGGGAGTGTC GTCGAGCCCG TTCAGCAGCT |
| 37561 | TGGCGATGGT CTGGCGGTCG ATGTCCTCGG GCGCGAGGGA CAGCGACTTC ACCTCGGCGC |
| 37621 | CGGCATCGGT CAGCACCCGA CGCACCTCGC CGTGCAGCCC GTCGTGCAGC CCGTCGTTGT |
| 37681 | CGAGCAGGTG GCCCGCGCGC AGGTCGCCTT CGGGTACGAC GATCAGCGAG GTGCCGTGCA |
| 37741 | GGGTGGCGGG CCCCTCGGGG GCGTGCTGCG CGGTCGGCCG CTCCCAGGCG ACGCGATAGC |
| 37801 | GCCATCCGTC CGTCTCGGAG GCCTCGATGT GCGTCTGGTG CCAGTCGCCG AGCGCGGGCA |
| 37861 | GGACGGTGTG CAGCGGAGCG TCGGGGTCCA CGCCGAGGTC GCTCGCCAGC CGCTGGAGGT |
| 37921 | CCTGTTCCTG GACGACCTTC CAGAACGCGC CGTCGCTCCC GGCCGTCCGG GATGCCGCCG |
| 37981 | ATCCGGGCCG GACGGAGGCG CCCTTGAGCC AGTGGTGTTC GTGCTGGAAG GCGTAGGTGG |
| 38041 | GGAGTTCACG GGCGAGGTCG TCGGCCCGGC CGAGAGCGGT CCAATCGACC TGGTGACCAC |
| 38101 | GCGCGTGCAC CCGGGCCAGC ATGCCGAGGA ACGCCCGGGT GTCCGTGGAA CGCCGGCTCA |
| 38161 | GCGTGGGCAC GAACGCCACG TCCCGCGCCG CCGAGTCCCG CTCGGCGGAC GCGGCGCGCA |
| 38221 | CGCGCTCGCC CAGCGCCGTC AGGACGGGGT CGGGCCCGAG TTCGACGACG GTCGCGACGC |
| 38281 | CCTGGGCCAG GACCGCGCCG ACTCCGTCCC CGAACCGCAC CGCTTCGCGC ACGTGCCGCA |
| 38341 | CCCAGTACTC CGGCGAGCAC AGCTCCTCGG CGTCCGCGAT CGTGCCGGTC ACGTTGGACA |
| 38401 | CGACCGGAAT CGACGGGGCA CGGAACTCCA CCTGCGCCAG CACGTCCGCG AACTCGGCGA |
| 38461 | GCATCGGCTC CATCAACGGC GAATGGAACG CGTGGCTGAC CGCCAACGCC CGTGTGCGCC |
| 38521 | GCCCCCGTCC GGCAAAGATG TCCGCGATCT GGTCCACCGC GGCGTCCTGA CCGGACACGA |
| 38581 | CCACAGCCCC TGGAGCGTTC ACGGCTGCCA GCGACACCAT GCCCCCGGCA GCCGCCACAT |
| 38641 | CCGCGACGAG CGGCGCGACC TCCTCCTCGG TGGCCTCCAC CGCCACCATC CGCCCACCCG |
| 38701 | ACGGCAACGA ACCCATCAAC CGGGCCCGGG CCACCACCAC CCGCACCGCA TCCGCCAACG |
| 38761 | ACCACACACC CGCCACATAC GCGGCGGACA ACTCCCCCAG CGAATGCCCG ATCAACACAT |
| 38821 | CCGCACGCAC ACCGAAAGAC TCCGCCAGCC GATACAACGC CACCTCGACC GCGAACAACG |
| 38881 | CAGGCTGAGC AACCCCCGTA TCCTCCAAAA CCCCCGCATC ATCACCGAAG ACCACCCCAA |
| 38941 | GCAGCTCTGC TCCCGTCTGC GCCTCGACCT CCGCACACAC CTCGTCCAAC GCAGCCGCGA |
| 39001 | AGACCGGGAA CCGCCCATAC AACTCACGCC CCATCCCCGG ACGCTGCGAG CCCTGACCCG |
| 39061 | AGAACGCCAC ACCCACACCA CCAGCGACAC GACGCTCAAA CACCACACCA CCGGCAGCGG |
| 39121 | AACCGTCACC CCGCGCAACC CCACCCACAC CGGCCAACAA CTCGTCCAAC GACCCACCAC |
| 39181 | TGACCACAGC ACTGTGATCG AACACCGAAC GCGACGACAC CAACGCCAGA CCCACACCCC |
| 39241 | CCACATCCAG CGCACCACCC CCGCGTCCCG CCACGAACGC CGCAAGCCGC GCCGCCTGAG |
| 39301 | CCCGCACCGC ACCCTCAGTA CGACCCGACA CAACCCACGG CAACTCCCCA GCAACCACCA |
| 39361 | GCGCTTGAGT GGACTCCACC GGAACCTGAG CGGACCCCAC CGGAGCTTCA GTGGATTCCA |
| 39421 | CGGGCTCGTG CTCCAGGATC ACGTGCGCGT TCGTCCCGCT GATACCGAAC GACGACACAC |
| 39481 | CCGCCCGCCG CGCACGACCC GTCTGCGGCC ACTGCCGAGC CCGCGTCAAC AACTCCACCG |
| 39541 | CACCCGCAGA CCAATCCACA TGCGGCGACG GCTGCGACAC ATGCAACGTC CGCGGCAACA |
| 39601 | CCCCGTGCCG CATCGCCATC ACCATCTTGA TCACGCCACC GACACCGGCA GCCGCCTGCG |
| 39661 | TATGACCGAT GTTCGACTTC AACGACCCCA GCCACAACGG ACGGCCCTCC GCCCGGCCCT |
| 39721 | GCCCGTACGT CGCGATCAAC GCCTGCGCCT CGATCGGATC ACCCAGCCTC GTCCCCGTCC |
| 39781 | CGTGCGCCTC CATCACATCC ACGTCCGACG TCGACAACCC CGCACCCGCC AACGCCCGCA |
| 39841 | CGATCACCCG CTGCTGCGAG GGACCGTTCG GCGCCGTCAA CCCGTTCGAC GCACCGTCCT |
| 39901 | GGTTCACCGC ACTGCCCCGC ACCACCGCCA ACACCTCGTG CCCGTTGCGC CGCGCGTCCG |
| 39961 | ACAAACGCTC CAGCACCACC ACACGCACAG CCTCGGACCA GCCCGTCCCC TCCGCATCCG |
| 40021 | CGGAAAAAGA ACGACACCGG CCGTCCGCCG ACAGAGGACC CTGACGACCG AACTCCACGA |
| 40081 | AGGCGTACGG CGTCGCCATC ACCGTCACAC CACCCGCGAG CGCCAACGAA CACTCCCCCG |
| 40141 | CACGCAACGA CTGCACCGCC AGATGCAACG CCACGAACGA CGACGAACAC GCCGTGTCCA |
| 40201 | CCGTCACCGC AGGAGCCTCG AACCCGAACG AATACGAGAC CCGGCCCGAG ATGACGGAGC |
| 40261 | TGGCCGATCG CGTTCCGCCG AGGCCTTCGG GCGCGTCGAC GATCTCCGTG CCGACCAGGC |
| 40321 | CGTAGCCCTG GACGGCGCCG CGCATGAAGA CGCCAACCGG CTTGCCGCGC AACGAGTCGG |
| 40381 | CGCTGATGCC GGACCGCTCC ACCGCCTCCG AGCAGGTGTC CAGGGCGATG CGCTGCTGGG |
| 40441 | GGTCCATGGC GGCGGCATCG CGCGGCGAGA TGCCGAAGAA GCCCGCGTCG AACTGCGCGG |
| 40501 | CGTCGTGCAG GAACCGGCCG CCCGCGGCAG GCAGTCGTCC GAGGTCCGAG CCGCGGTGGG |
| 40561 | CCGGGAACGG CGAGATCGCG TCCCGCCCCT CGGCGACCAG TCGCCACAGG TCCTCGGGCG |
| 40621 | AGGCAACCCG GCCCGGATAC TTGCAGGCCA TGCCGACGAG CGCGATCGGC TCGTTCCCGG |
| 40681 | CTGCCTCGAG TTCGCGCAGC CGGCCGCGGG TAGGCAGCAG ATCGCCGGTC AGTTCCTTGA |
| 40741 | CGTAGTGGGG AAGCTTGTCT TCGTTGGTGG ACACGGTGCG CCAGCTCCTT GTTGGTGCTG |
| 40801 | AGGTTTGCGA ACGCCGGCGT CAGGAGATGC GGAATTCCTT CTCGATCAGA TCGAAGAGCT |
| 40861 | GATCGTCGGT TGCCGAGTCG AGTTGCTGGG GAGCTGTTTC CGCCGCGCCG GAGGCGGCGG |
| 40921 | TGGGCTCGTC GTCGGCGTTC TGGAACCTGG TCAACAGGTT GGAAAGCCGC AGGGTGATAC |
| 40981 | GGCCGCGGGC GGCCGGGTCG GACCCGGCGT CGAGCGCGGC GAGGGCCGCT TCGAGCCGGT |
| 41041 | CCAGCTCACC GAGGACCGCC GACGCGCCCG ACGCGGCGCC GAGCCGCTCC GCGAGGGCGG |
| 41101 | TCGCGACGAC CTGTGCCAGG GCCGCGGGCG TGGGGTGGTC GAAGACGAGC GTGGCGGGCA |
| 41161 | GCTTGAGGCC GGTGAGCTTT TCGAGCCGCT GCCGCAGACG GACTGCAGCC AGCGAGTCGA |
| 41221 | ACCCGAGTTC CTGGAAAGGG CGTTCCGGCT CGATCGTGCC GCCCGAGGCG TGCCCGAGTT |
| 41281 | CCGCGGCGGC CTGCGTGCAG ACGGTCTCCA GCAGCACGCG TCGGGGTTCC CCGCCGGACA |
| 41341 | GCGCGGTCCA GCGGGCGAGG AAGGGGGTGG CCCCTGCGGT ACCGGCGTCG CTGTCGCCCG |
| 41401 | GGCCGGAGAC GTCGTCCGCA CCGGCCGTGC CGGCCGCCGT CCCGTCCGCG CCGACGCCGC |
| 41461 | CGCGTTCCGT TTGCACGGTG CGGAGCGGGT CGAACAGGGG GCTCGGACGG TTGACGGTGA |
| 41521 | AGATGTCGGC CAGGCGTGAC CAGTCGATGT CGGCGAGGAC GACGGTGCCG TAGTCCGCCC |
| 41581 | TCACGACGCG GCCGAACGCG GCGACGGCCT CCTCCGGGTC GAGCGCGCTG ACGCCGCGGG |
| 41641 | CCTGCATCTC CCGTGTGAGG CGTTCGTCGG CCATGCCGCC CCCGCCCCAG GGGCCCCAGG |
| 41701 | CGAGGGCGGT GCCGGGACGT CCCTGGGCGC GCGGCGGCTC GATCAGCGCG TCGAGATGCG |
| 41761 | CGTTTCCTGC GGCGTAGGCA CCGGCACGAG CGCTGCCCCA GACCCCGGCG ATGGAGGAGT |
| 41821 | AGACGACGAA CGCGGCGAGT CCGTCGCCCA AGACCTCATC GAGGACCTGC GCGCCGACGA |
| 41881 | CCTTCGCGCG TACGACGGCG GCGTAGCCGT CCTCGTCGAG CTCCGCGAGC GGCAGTTCCG |
| 41941 | AGGCGACGCC CGCGGTGTGG ACCACGGTGC TGAGCGGCGT TCCCGCGTCG GCAAGCCTGT |
| 42001 | CCCGGAGGGC GGCGAGGGCC ACGGCGTCGG TGACGTCGCA GGACTCGACG ACGACGTCGG |
| 42061 | CACCCCGCTC CTCGAGTTCC GTGCGCAGCG CGGCCACCGC GGGTGCGGCA GGGCCCTGAC |
| 42121 | GGCTGGTGAG GACGAGGGTC CGCGCGCCGT TCCGGGCGAG CCAGCGCGCC GTCTGCGCGC |
| 42181 | CGAGGGCGCC GGTGCCGCCG GTGATCAGGA CGGAGCCGTC GGTCGAGGGC GCCGTGGAGC |
| 42241 | CGGTCTCCGG CTCCGGCACG CTCGTGGGTA CCGTGCCGCG GATCAGCCGA CGACCGAGCA |
| 42301 | ACAGCTGGCC TCGCAGGGCG ATTTGGTCGT CACCGGTGGT GTTGGCAAGG GCAGCGGCAA |
| 42361 | GGCCGGTGAG GTGCGCTGCG GGACTCGTAC CGGAGACGTC GATCAGACCG CCCCAGAGGG |
| 42421 | TGGGGTGTTC GAGGGCCGCG ACCCGTCCGA GGCCGCAGAC CTGGGCCTGC CACGGGTCCG |
| 42481 | GCGCCGCGTC GTCGGCCTGC GCACACACCG CGTCGCACGT GAGGGCCCAT ACGCGGGTGT |
| 42541 | CGACTCCCGC GTCCTGCACG GTGTGCAGGA GATCGAGCAC GGCCAGGGCG CCGGTCGCGA |
| 42601 | TGCGGCGCTC CCGGTCGCCG TCGCGCTGGG CGCCGACCGC GGGCAGGCAG AGCACGCCGC |
| 42661 | GGGGCGGCAC TGCGGCCAGC CGTGCGCGCA GCTCGGCCGT CTGGCACCGC TCCACGCGCG |
| 42721 | CCCCCGGGTC GACGAGCGCG TTTTCGACCG CAATTACGAG GTCCGGTTGG ACGGGCTCGC |
| 42781 | CGGGCACCGC GACCAGCCAC GTGCCGTCGA GCGGCACCGC GTCCGTCGGC GACGGAAGCT |
| 42841 | CCGTCCAGGA GACGCGGTAG CGCAGGGCGT CGGCCGCTGG GAGCCGGGCC TGTTCCCTGG |
| 42901 | ACCAGGTCTG GAGCGCGGGC AGCACCGCGG TGAGCGGCGC GTCCGGCGCG AGGCCGAGGG |
| 42961 | TATGGGCGAG GCGGTCGACG TCCTGCTGCG CGACCGCGTT CAGGAGTACG GCCTGCTCCG |
| 43021 | GGACGTGCTC GACGACGCCG GGCTCGGGCG CGGGGGCGTC GAGCCAGTAA CGCTGATGCT |
| 43081 | GGAAGGGGTA GGTGGGGAGT TCACGGGCGA GGTCGTTCGG CCGGCCGAGA GCGGTCCAGT |
| 43141 | CGAGCTGGTG ACGACGCGCG TGCACCCGGG CCAGAGCCGT CAGGAAACCG TTCACATCAC |
| 43201 | CCGTCCGCCG CCCCAGGGTG GGCAGGAACA CGGCACCGTT GTCGACGACT CCCGGATGCG |
| 43261 | AGGGACCCAT CGCCGTCAAC ACCGGCTCGG GCCCCAGCTC GACAACGGTC GCGACGCCCT |
| 43321 | GCGCCAGGAC CGCACGGACC CCGTCCCCGA ACCGCACCGC TTCCCGCACG TGCCGCACCC |
| 43381 | AGTACTCCGG CGAGCACAAC TCCGCAGCGG ATGCGACCTC ACCCGTCACG TTCGACACGA |
| 43441 | CCGGAATCGA CGGGGCACGG AACTCCACGC GCGCCAGCAC GTGCGGGAAC CCGGGGAGCA |
| 43501 | TCGGCTCCAT CAACGGCGAA TGGAACGCGT GCGAGACACG AAGACGAGTC GCACGACGCC |
| 43561 | CTCCGCCGCG CGCCCGGTCC ACCACCGCCT GAACGGCACC CTCCAGACCC GAAACAACCA |
| 43621 | CCGCCGCCGG CCCGTTGACA GCAGCGATCA GCGCACCGTC CACCAGCCAG CGGGACACCT |
| 43681 | CCTCCTCGGT GGCCTCCACC GCCACCATCC GGCCACCCGA CGGCAACGAA CGCATCAACC |
| 43741 | GGCCCCGGGC CACCACCACC CGCACCGCAT CCGCCAACGA CCACACAGCC GCCACATACG |
| 43801 | CGGCGGACAA CTCCCCCAGC GAATGCCCGA TCAACACATC CGCACGCACA CCGAAAGACT |
| 43861 | CCGCCAGCCG ATACAACGCC ACCTCGACCG GGAACAACGC AGGCTGAGCA AGCCCCGTAT |
| 43921 | CGTCCAAAAC CCGCGCATCA TCACCGAAGA CCACCGAAAG CAGCTCTGCT CCCGTCTGCG |
| 43981 | CCTCGACCTC CGCACAGACC TCGTCCAACG CAGCCGCGAA GACCGGGAAC CGCCCATACA |
| 44041 | ACTCACGCCC CATCCGCGGA CGCTGCGAGC CCTGACCCGA GAACGCCACA CCCACACCAC |
| 44101 | CCGCGACACG ACGCTCAAGC ACCACACCAC CGGCAGCGGA ACCGTCACCC CGCGCAACCC |
| 44161 | CACCCACACC GGCCAACAAC TCGTCCAACG ACCCACCACT GACCACAGCA CTGTGATCGA |
| 44221 | ACACCGACCG CGACGACACC AGCGCCAGAC CCACACCCCC CACATCCAGC GCCCCCGCAC |
| 44281 | CGCCCCCGCG TCCCGCCACG AACGCCGCAA GCCGCGCCGC CTGAGCCCGC ACCGCACCCT |
| 44341 | CAGTACGACC CGACACAACC CACGGCAACT CCCCAGCAAC CAACGGAGCT TCAGTGGACT |
| 44401 | CCACCCGAGC CTGCACAGAC CCCACCGGAA CCTGAGCGGA CCCCACCGGA GCTTCAGTGG |
| 44461 | ATTCCACGGG CTCGTGCTCC AGGATCACGT GCGCGTTCGT CCCGCTGATA CCGAACGACG |
| 44521 | ACACCCCCGC CCGCCGCGCA CGACCCGTCT CCGGCCACTG CCGAGCCCGC GTCAACAACT |
| 44581 | CCACCGCACC CGCAGACCAA TCCACATGCG GCGACGGCTG CGACACATGC AACGTCCGCG |
| 44641 | GCAACACCCC GTGCCGCATC GCCATCACCA TCTTGATCAC ACCACCCACA CCGGCAGCCG |
| 44701 | CCTGCGTATG ACCGATGTTC GACTTCAACG ATCCCAGCCA CAACGGACGG CCCTCCGCCC |
| 44761 | GGCCCTGCCC GTACGTCGCG ATCAACGCCT GCGCCTCGAT CGGATCACCC AGCCTCGTCC |
| 44821 | CCGTCCCGTG CGCCTCGACC GCGTCCACAT CCGCCACGGA AAGTCCCGCG CCCGCCAGTG |
| 44881 | CCTGGCGAAT CACGCGCTGC TGGGACGGGC CGTTCGGCGC CGTGAGCCCG TTCGACGCAC |
| 44941 | CGTCCTGGTT CACCGCACTA CCCCGCACCA CCGCCAACAC CTCGTGCCCG TTGCGCCGCG |
| 45001 | CGTCCGACAA ACGCTCCAGC ACCACCACAC CCACACCCTC GGACCAACCG GTGCCCGATG |
| 45061 | CGTCGGCGGA GAACGAACGG CACCGGCCGT CGACGGCCAG TCCGCCGTGG CGTCCGAACT |
| 45121 | CCACGAACGC GTACGGCGTC GCCATCACCG TCACGCCACC GGCGAGCGCC ATCGAGCACT |
| 45181 | CCCCCGCACG CAACGACTGT GCCGCCAGAT GCATCGCGAC CAGCGACGAC GAACACGCCG |
| 45241 | TGTCCACCGT CACCGCAGGA CCCTCGAACC CGAACGAGTA CGAGACCCGT CCCGACGCGA |
| 45301 | TGCTGCCGGA GCTTCCGTTG CTGATGTAGC CCTCGTACCC CTGCGGCGAG CGGTTCAGGT |
| 45361 | GGCGGGCGCC GTAGTCGTTG TACATGACGC CCATGAACAC GCCGGTGCGG CTGCCGGTGA |
| 45421 | GCGTCTCCGG CCGGGTGCCG GCCGACTCCA GGGCCTCCCA GGAGGTCTCC AGGAGCAGTC |
| 45481 | GCTGCTGCGG GTCGGTCGCG GTCGCCTCGC GTGGCGAGAT GCCGAAGAAC TCCGCGTCGA |
| 45541 | ACTGGGCCGC GTCGTGCAGG AACCCGCCCT CACGGGTGTA CGTCTTGCCG GGCTGCTGCG |
| 45601 | GGTCCGGGTC GTAGATGCCG TCGAGGTCCC AGCCGCGGTC GGCCGGGAAC GGCGAGATGG |
| 45661 | CGTCCCGCCC CTCGGCCACC AGCCGCCACA GGTCCTCGGG CGAGGCAACC CCGCCCGGAT |
| 45721 | ACTTGCAGGC CATGCCGACG ATGACGATCG GGTCGTCGCC CGCATCCTGC GGATGGCTCG |
| 45781 | CCGACGCCCG CGCCGCGGGC TCGGCGACCG CGAGCGCGGT GGACCCGGCC GGCTCCCCGA |
| 45841 | CGACTCCGCG AGCGAGTTCG TCGTACAGGA ATTCCGCGAC CGCGAGCGGA GTCGGGTGGT |
| 45901 | CGAAGACCAG CGTCGCCGGA AGGCGCACGC CGGTGGCGGC GCCGAGCTGG TTGCGCAGTT |
| 45961 | CGACGGCGGT GAGGGAGTCG AGGCCGAGCC GGTTGAACGG CTGGGCGCGG TCGACGGCCT |
| 46021 | CGCGGTCGGC GTGTCCGAGC ACGTACGCGA CCTTCTCCGC GACGAGGCCG CCGAGGATCT |
| 46081 | GAAGGCGCTC CTCGCGGTCG GCCACGCGAA GCTCGGCGAG CAGCGGCGCG CTCGCGCTGC |
| 46141 | CGGCGGTCGC CGCGGTGCCG CCGGCCACAC GGGAAGAGGG TCGGGGCCTG GTGCTGACGA |
| 46201 | CCGCTTGGAA GACGGCGGGC AGCGATCCGG CCGCGGCCTG CTCGTCGAGG ACGGGGGCGT |
| 46261 | TCAGCCGGGC GGGCACGAGC AGGCCGTCGG CGTGCGTTCC GACGGTCTCC GGGCCGGCCT |
| 46321 | CCGAAGCGCC GAAGGCTGCG CCGGCTGCTG CGAGAGCGGC GTCGAACAGC GTGACGCCCT |
| 46381 | GTTCGCGGCT GATCTCCAGG AGGCCGGTGC GCTTCAGCCG GGCCACGTTG GCCCGGTCGA |
| 46441 | GCTCCGCGGT CATCCCGCCC TCGGTGCTCC ACAGGCCCCA CGCGAGCGAG ACGCCCGGCA |
| 46501 | GCCCGAGCGC CCGGCGCCGA CGCGCCAGTG CGTCGAGGAA GGCGTTGGCT GCGGCGTAGT |
| 46561 | TGGCCTGTCC GGCCCCGCCG AAGACACCGG CGACCGAGGA GAACAGGACG AACGCGGAGA |
| 46621 | GGGGGGCCGG CGACGTCAGG TCGTGGAGGT GCAGCGCGGC GTCGGCCTTC GCGCGCAGCA |
| 46681 | GCTTCGTCAG CTGCGCGGGA GTGAGGGATT CGAGGAGCCC GTCGTCGAGT ACGCCCGCGG |
| 46741 | TGTGGACCAC GCCGGTGAGC GGATGGTCGC CCGGTACGCC GGCGAGCAGT TCGGCGACGG |
| 46801 | CGGAGCGGTC GGACATGTCG CAGGCGGCGA GCGTCACGTC GGGCCCGAGT GGTTCCAGTT |
| 46861 | CCGCGATGAG CTCGGCGGCG CCCGGGGCGT CCGGACCACG GGGGCTGGTC AGCAGCAGGT |
| 46921 | GCCGCACTCC GTGGACGGTG ACGAGGTGGC GGGCGAAGAG CGAGCCGAGG TCGCCGGTGC |
| 46981 | CACCGGTGAT CAGGACCGTT CCCTCGCCGG AGAAGGCGGG GGCGTGGGCG GAGCCATCGG |
| 47041 | CGTCGGTCGT CTCGGGGGCC GCGATCGGGC GGAGTCGCGG GACGGAGGGG ACCCCGGCAC |
| 47101 | GGAGCGCGAT CTGCGGGTGC GCGACGACGC CGGTGCCCGT GCCGATGTCT GTGCCGTTGC |
| 47161 | CGGTGCCGAG CAGCGTCGGC AGGGCGCGCA GCGAGGCCTC CTCCCCGTCG GTGTCGATCA |
| 47221 | GGCGGAACCG GCCCGGGTGC TCAAGCTGGG CGGTGCGTAT CAGGCCGCCC ACGGCGGGGG |
| 47281 | ACGCCAGGTC GACGGCTGCG GCCTCGGCCG CGTCGACCGC GAGCGCACCG CGGGTGAGCA |
| 47341 | GGGTCGCGGT GACGGATGCG AAGCGCGGCT GCTTCAGCCA GTCCTGGAGG AGATGGAGGA |
| 47401 | GCGTCTGCGT CCGCTGATGG GTGGCCGCGG CGATGTCGCC GTCCAGTCCG CCCAGGGCGG |
| 47461 | GCAGCGCTAT CAGGACGTCA GGAGGCGCCT CGGCCGGATC GGGGTCGATG GCGGCGGCGA |
| 47521 | GGGCTGGGAG GTCGGCGTGG CGGCGCGCCG GAACGGATTC GACGGACCAG TCGGCGCCGA |
| 47581 | GGTCGAGGAG CGCCAATCCG GTCCCGGAGG CGGACACGGC CGCAGACACA GCGGAACCGG |
| 47641 | CGCTTTGCAG CGGCGACGAC GCGATGCCGT ACAGCGATTC GACGGTCGAC ACGCGGGCCG |
| 47701 | AGCGAAGCTG CTCCAGCGTC ACGGGACGGA GCGCCAGCGA CCGCACGGTG GCGAGTGCCG |
| 47761 | CCCCCGCCTC GTCCAGCAGC TCCACGGAGG TCGCGCCCTC ACCGAGGCGG CGTATGCGGA |
| 47821 | CCGGGGCCGA ACTCACGGCG GTGGCGTGCA GGGTGACGCC GCTCCAGGAG AACGGCAGAT |
| 47881 | GGCACTCGTC CTGCCCGGAC AGCAGGTGGG GCAGCGCGAG CGAGTGCAGG GCGGTGTGCA |
| 47941 | GGAGCGCCGG ATGGATGCTG AAACGCCCGG CGTCGTCCGT CTGCCGGGTG GGCAGACGGA |
| 48001 | CTTCGGCGTA GACGGTGTCG CCGTGCCGCC ACGCCGTGTG CAGCCCGCGG AACGCGGGAG |
| 48061 | CGTAGTCGAA TCGGAGGCCG ATCAACCGCT CGTAGACGGC ATCGAGGTCG ACCGGTGTGG |
| 48121 | CCCCGCGCGG AGGCCATGCC TGCACCTGCG ACGGCTCCGG GGCGAGCACC GGGGCCGGCT |
| 48181 | CCGCCGCGTC GGAACGGAGG GTGCCGGTGG CGTGCCGGGT CCAGGGGGCG TCCTCGGCGG |
| 48241 | CGTGGACAGG GCGGGAGTGC ACGGTGATCG CACGGCCGCC GCCGGCCGCC TCGGCCTCGC |
| 48301 | CGACCAGGAC CTGCACGACG TGGGCCGACC GGCCGTCGAG GATGAGCGGC GCCTCCAGCG |
| 48361 | TGAGTTCCTC GACGGCCGCG CAGCCCACGC GGGCCGCGGC GGTGAAGGCG AGTTCGAGGA |
| 48421 | ACGCGGTCCC GGGCAGCAGC GTCGAGCCGA GCACGACGTG GTCGCCGAGC CACGGGTGGG |
| 48481 | TTCCCGGGGA GATCGTGCCG GTCAGCACTG TGCCACCGGT GCCGGGCAAG GTGACCGCGG |
| 48541 | CGCCGAGGAA CGGGTGGTCG ACGGCGCGCA TACCGAGGTG CGCGGCGTCG ACGGAGGCGG |
| 48601 | CATTGCCGGC AAGCCAGTGG TGTTCGTGCT GGAAGGCGTA GGTGGGGAGT TCACGGGCGA |
| 48661 | GGTCGTTCGC CCGGCCGAGC GCGGTCCAGT CGACCTGGTG ACCACGCGCG TGCACCCGGG |
| 48721 | CCAGAGCCGT CAGGAAACCG TTCACATCAC CCGTCCGCGG CCCCAGGGTG GGGAGGAACA |
| 48781 | CGGCACCGTT CTGGACGACT CCCGGATGCG AGGCACCCAT CGCCGTCAAC ACCGCCTCGG |
| 48841 | GCCCCAGCTC GACAACGGTC GCGACGCCCT GCGCCAGGAC CGCACCGACC CCGTCCCCGA |
| 48901 | ACCGCACCGC TTCCCGCACG TGCCGCACCC AGTACTCCGG CGAGCACAAC TCCGCAGCGG |
| 48961 | ATGGGACCTC ACCCGTCACG TTCGACACGA CCGGAATCGA CGGGGCACGG AACTCCACCC |
| 49021 | GCGCGAGCAC CTGCGCGAAC CCGGGGAGCA TCGGCTCCAT CAACGGCGAA TGGAACGCGT |
| 49081 | GGGAGACACG AAGACGAGTC GCACGACGCC GTCCCCCGCG CGCCCGCTCC ACCACCGCCT |
| 49141 | CAACGGCACC CTCCACACCC GAAACAACCA CCGCCGCCGG CCCGTTGACA GCAGCGATCA |
| 49201 | CCGCACCGTC CAGCAGCCAG CCCGACACCT CCTCCTCGGT GGCCTCCACC GCCACCATGC |
| 49261 | GCCCACCGGA CGGCAACGAA CCCATCAACC GGCCCCGGGC CACCACCACC CGCAGCGCAT |
| 49321 | CCGCCAACGA CCACACACCC GCCACATACG CGGCGGACAA CTCCCCCAGC GAATGCCCGA |
| 49381 | TCAACACATC CGCACGCACA CCGAAAGACT CCGCCAGCCG ATACAACGCC ACCTCGACCG |
| 49441 | CGAACAACGC AGGGTGAGCA ACCCCCGTAT CCTCCAAAAC CCCCGCATCA TCACCGAAGA |
| 49501 | CCACCGAAAG CAGCTGTGCT CCCGTCTGCG CCTCGACCTC CGCACACACC TCGTCCAACG |
| 49561 | CAGCCGCGAA GAGGGGGAAC CGCCCATACA ACTGACGCCC CATGCCCGGA CGCTGCGAGC |
| 49621 | CCTGACCCGA GAACGCCACA CCGACACCAC CCGCGACACG AGGCTCAAGC ACCACACCAC |
| 49681 | CGGCAGCGGA ACCGTCACCC CGCGCAACCC CACCCACACC GGCCAACAAC TCGTCCAACG |
| 49741 | ACCCACCACT GACCACAGGA CTGTGATCGA ACACCGACCG CGACGACACC AGCGCCAGAC |
| 49801 | CCACACCCCC CACATCCAGC GCCCCCGCAC CGCCCCCGCG TCCCGCCACG AACGCCGCAA |
| 49861 | GCCGCGCCGC CTGAGCCCGC ACCGCACCCT CAGTACGACC CGACACAACC CACGGCAACT |
| 49921 | CCCCAGCAAC CAACGGAGCT TCAGTGGACT CCACCCGAGC CTGCACAGAC CCCACCGGAA |
| 49981 | CCTGAGCGGA CCCGACCGGA GCTTCAGTGG ATTCCACGGG GTCGTGGTCC AGGATCACGT |
| 50041 | GCGCGTTCGT CCCGCTGATA CCGAACGACG ACACACCCGC CCGCCGCGCA CGACCCGTGT |
| 50101 | CCGGCCACTG CCGAGCCCGC GTCAACAACT CCACCGCACG CGCAGAGCAA TCCACATGCG |
| 50161 | GCGACGGCTG CGACACATGC AACGTCCGCG GGAAGACCCC GTGCCGCATC GCCATCACCA |
| 50221 | TCTTGATCAC ACCACCCACA CGGGCAGCCG CCTGCGTATG ACCGATGTTC GACTTCAACG |
| 50281 | ACCCCAGCCA CAAGGGAGGG CGCTCCGCCC GGCCCTGGCC ATACGTCGCG ATCAACGCCT |
| 50341 | GCGCCTCGAT CGGATCACCC AGCCTCGTCC CCGTCGCGTG CGCCTGCATC ACATCGACGT |
| 50401 | CGGACGTCGA CAACGCCGCA CCCGCCAACG CCCGCAGGAT CACGGGCTGC TGCGACGGAC |
| 50461 | CGTTCGGCGC CGTCAACCCG TTCGACGCAC CGTCCTGGTT CACCGCACTG CCCCGCACCA |
| 50521 | CCGCCAACAC GTCGTGCCCG TTGCGCCGCG CGTCCGAGAA ACGCTCCAGC ACCACCACAC |
| 50581 | CCACACCCTC CGACCAGCCC GTCCCCTCCG CATCCGCGGA AAAAGAACGA CACCGGCCGT |
| 50641 | CCGCCGACAG ACCACCCTGA CGACCGAACT CCACGAACGC GTACGGCGTC GCCATCACCG |
| 50701 | TCACACCACC CGCGAGCGCC AACGAACACT CCCCCGCACG CAACGACTGC ACCGCCAGAT |
| 50761 | GCAACGCCAC CAACGACGAC GAACACGCCG TGTCCACCGT CACCGCAGGA CCCTCGAACC |
| 50821 | CGAACGAATA CGAAAGGCGT CCGGAGAGCA CGGAGCTGGC GTTGCCGGTG ATGAGGAATC |
| 50881 | CGGAGGCCTC GGTCGCACGG GACTCGCGCA GGTGTCCGAG GTAGTCCTGC ATGCCGGCGC |
| 50941 | CGATGAACAC GCCGGTGTCG CCGCCGCGCA GCGACTCGGG GACGATGCCG GTGCGCTCGA |
| 51001 | TCGCCTCCCA CGAGGTCTCC AGCAGCAGCC GCTGCTGGGG GTCCATGGCG GCGGCCTCGC |
| 51061 | GCGGCGAGAT GCCGAAGAAG CCCGCGTCGA ACTCCGCGGC GTCGTGCAGG AACCCACCGC |
| 51121 | CCGCGGTGAG CAGCCCGTCG GGAGCGGACG TCGAGCCGTC ACCGGCCACC CCGCCACTGG |
| 51181 | CCGGCCCGGC GCCGACGCCG TCGAGGTCCC AGCCGCGGTC GGCCGGGAAC GGCGAGATCG |
| 51241 | CGTCCCGCCC CTCGGCCACC AACTCCCACA GGTCCTCGGG CGAGGCCACC CCGCCCCGAT |
| 51301 | ACTTGCAGGC CATGCCGACG ACGGCGATGG GTTCCCGGGC CGCGTCTTCG ACGTCCTGGA |
| 51361 | GCCCACCACC CGTCTCGTGC AGCTCGGCGC TGACGCGCTT CAGGTAATCC AACAGCTGGT |
| 51421 | TCTCGGATGC CATTTCCCGC TCTCCCCATC AATTCCCGGA GGGTTCTCCA CTTGCCGCCG |
| 51481 | ACGACTCAGG ACTCGTCTAT CCCGGGCCCT CCAGCGGGGA GATGCCGAGC TGCCGGGTGA |
| 51541 | CGAAGTCGAG GACTTCGTCC TCGGTGGCCG ACTCCAGCAG CGCCCCCGTG TCCGGTGTGC |
| 51601 | CCCTCTCGGA CGAGGACCCG GAGGGCGAGG AGAACATCAC CGTCTCGGAA GCGGACCGTT |
| 51661 | TGCCTTCCCA GCCGTCCGCG AGCGCCCTGA GCCGGGACGC CGCGGCCTTC CGCAGGGCGG |
| 51721 | CGTCCAGCCG GAGTTCCTCG AGGCTCTGCG CCACTCGGTA CAGCTCGGCG AGGGCGGATT |
| 51781 | CCGCGGTGAC GGCCCGCTCC TCCGGCTGGA TCAGGCCGTG CAGTTGCGCG GCGAGTGCCT |
| 51841 | TGGGCGTCGG GTGGTCGAAG ACCATCGTGG CGGCCAGGTC CACGCCCGCG GCCCGCTGCA |
| 51901 | GTCTGTTCCG CAGCGCCATC GCGGTCATCG AGTCGATCCC CAGCTCCTTG AAGGGCTGGT |
| 51961 | CGACGGCGAG GGCGGCGGGC TCGGCGTAGC CGAGCACCGC CGCCGCGTGA TCACGGACGG |
| 52021 | TGTCCAGGAG CAGGCGGTCC GCGTCGGTCC GCGCCATGCC GGGCAGTCGC CGGGCGAGCG |
| 52081 | TGGGACCACC CGCGCCGCCC GCGCCGTCGG AACGCCCGGC GTCGGAGCCG TCGTCCGCGG |
| 52141 | CTTCCCCGGA TCGTGCGAAT GCGGCGAGCA GCGGGCTGGG CCGGGCCGCG GTGAAGCCGT |
| 52201 | CGGCGAACAG CGGCCATTCG ATCTCGGCCA GCACCTGGCT CGCGGGGCCG TCCTGGGCCA |
| 52261 | GCGCGAGGTC GAGGGCCCGC ACCCCGAGTT CGGGCTCGAT CGGCGGCAGT CCGTTGCGCC |
| 52321 | GCATCCGCTG TTCCGTGGCC GCGTCGACCA GACCGCCGCC GCCCCAGGGC CCCCAGGCGA |
| 52381 | TCGAGAGGGC CGGGAGGCCC GCGGCGCGGC GGTGCTCGGC GACGGCGTCG AGCACCGCGT |
| 52441 | TGGCCGCGGC GTAGTTGCCC TGGCCGATGC CGCCGACGGT ACCGACGAAG CCGGAGTAGA |
| 52501 | GCACGAACGA CGACAGGTTC AGGCCGGCGG TCAGTTCGTG CAGGTGCCAG GCGCCGAGCG |
| 52561 | CCTTGGGGCC GAGAACCGCG TCGAGCCGGT CGGCGTCCAG GTTCTCCAGT GCGGCGTCGT |
| 52621 | CGAGGACCGC CGCCGCGTGC ACCACGGAGA CGAGCGGCCC GTCGGCCGGT ACCCACTCCA |
| 52681 | GCAGGGCGCG TACGGCCTCG CCGTCGGCGA GATCGCAGGC GGCGATCGTC ACGCGCGCGC |
| 52741 | CCATGCTCTC GAGGTCGGCG CCAAGTCCGT CCGCGTCCAG CGCCTCAGGA CCGCGGCGGC |
| 52801 | TGGTGAGCAG CAGATGTTGC CCGCCCTGCG CCGCCAGCCG GCGGGCCAGG CGGCTGCCGA |
| 52861 | GGGCGCCGGT GCCGCCGGTG ATCAGGACCG TGCCCTCGCG CGGCCACACC GCATCCGCGC |
| 52921 | CCACGTTCCC GTCCGTGTCC GTGTCCGTGT CCGTGTCCGA CAGGGAAACC TGACCGCCGT |
| 52981 | CGCGTCCGCC GGTCGTGGGG TCCGGCAGCC GGAACGGGGT GCGGACGAGG CGTCGGGCGA |
| 53041 | GCACGCCCGT CGGGCGCAGC GCGAGTTGGT CCTCGTCGCC CGGGTCGGCC AGCACCGCGC |
| 53101 | AGAGCTGAGC GGCGGTGGCG GCGTCCGGCG CGGTCCGTCC GTCCTGCCCG TCGGCCTCAC |
| 53161 | GGGGCGCACG GGGCAGGTCG ATCAGACCGC CCCACAGCGT GGGGTGTTCG AGGGCGGCGA |
| 53221 | CGCGGCCGAG GCCCCAGACC GCGGCGGCGT GCGGTGCCGC CGGAGGGTCG GTCGGGTCCG |
| 53281 | TTCCCACGGC GCCGTGCGTG AGGCACCACA GCCGGCCCGG CAGGTTTGTC TGCGCGAGCG |
| 53341 | CCCGCACCAG GAGCGTCCTC CCGGCCAGGC CGCGCCCGAC CGCGGGGCGT CCAGGTAGCG |
| 53401 | GGCGGTGGTC GAGCGCGAGC AGGGAGAGGA TCCCGGGCGG CCGGGCATCG GCCGCCGCGG |
| 53461 | CGTGCAGCGA CGGGGCCCAT GCGGCCGGGT CGGTGTCCTC GCGCGGGTCG AGTTCCACGA |
| 53521 | GCAGCGCCCG GCCGCCCGCG GCCTCGACGG CGTCGAGCAC CGACGCGACG AGCCGGTGGT |
| 53581 | CGGGGGCGAA TCGGTGCTCG GCGCCGGGTA TGGCGAGCAG CCAGGTGCCG GCGAGGGGGG |
| 53641 | GGACGGCGCC CGGGGCGTGC AGGACCGGCT CCCAGCGGAC GCCGTAGCGC CAACTGCCCA |
| 53701 | GCCGTGCGTG CTCCAGGTGG TCGCGCCGCC ATGCCGCGAG GGCGGGCAGC AGGGCGTCCG |
| 53761 | CCGACTCGCC GTCCTTCAGC CCGAG6GCGT CCAGGAGCGC GGCGGGGTCG GCCTCCAGCG |
| 53821 | CCTCCCACAG ACCCGAGTCG GCCGCCTGCG CGGCATGCGG CGCGGACGCT CCGGCCAGGG |
| 53881 | CCGAGGGGCG CCCGCCCCGG CCGGCCTCGG ATCCGGCCCC GGAGCCATTC GTGGTGCGCG |
| 53941 | GCGTCGGCGA GAAGCGGCTG CGCTGGAAGG GATAGGTGGG CAGGTGGGTC ACCCGGCCGT |
| 54001 | CGCTGTCGCG CAGCAGGGCG GCCCAGTCCA CGTCGAGACC GTGCACCCAG CCCTCGGGAG |
| 54061 | CCGAGGTGAG GAAGCGGTCG GCTCCGCCCG GGTCGCGGGG CAGCGTCGGC ACGGTCGCCG |
| 54121 | GTCGGACGCC CGCCTCCTCC GCGGTGCGTT CCATCGCCGC GGTGAGCAGC GGGTGCGGGC |
| 54181 | TGATTTCGAC GAATACGGCG TCCCCGGACT CGGCGAGGCG GCGGACCGCG GCCTCGAACT |
| 54241 | CCACCGGGGC CCGGAGGTTG CGCACCCAGT ACTCCGCGGT CAGTTCGCCG TCGCGGACCG |
| 54301 | GCTCGCGGGT GAGGGTGGAC AGCATCTCGA CCTCGGGGCG GCCCGGTGCG ACGTCCGCGA |
| 54361 | GCTCCGTCAG GATCCGCGGG CCGATCCGGT GGACGTGCGG CGAGTGCGAC GCGTAGTCGA |
| 54421 | CCGCGATCCG CCGGAGGCGC ACGTTGTCGG CCTCGCAGAC GGTGAAGAAC TCGTCCAGCG |
| 54481 | CGTCGGCGTC GCGGGACACC ACGGTGGCCT CGGGGCCGTT GGCGGCGGCG AGGAAGACGC |
| 54541 | GGCCTTGGAA GGGCGCGAGG CGGGGGGCGG TCTCGGCTCG GCCGAGCGCG ACGGACAGCA |
| 54601 | TGCCGCCGGC GCCCGCGATC CCGGTCAGCG TGGGGCTGCG CAAGGCCAGG ATCTTCGCGG |
| 54661 | CGTGTTCGAG GCTGAGGGCG CCCGCGACGC AGGCCGCCGC GACCTCGCCC TGGGAGTGGC |
| 54721 | CGACCACCGT GTGGGGGCGG ACGCCGACGG AGCGGCACAG CTCGGCCAGG GACACCATCA |
| 54781 | TCGCGAACAG CACGGGCTGG AGGAGGTCGA CCCGGTCCAG GGACGGTTCG GCGGGGTCGC |
| 54841 | CGTTCAGCAC GTCGAGGAGC GACCAGTCGA CGTGCGGGGC GAGCGCGTCG GCGCAGTCGC |
| 54901 | GCACCCGCGC GGCGAACACC GGCGAGCTGT GCAGCAGTTC GCGAGCCATG GCGGCCCACT |
| 54961 | GCGCGCCCTG TCCGGGGAAG ACGAACACCA CCCGGCGGCG AGGGGCTGCG GCGCCGTCAC |
| 55021 | CGTCCACCCG GTTCGCGCTC GGCACGCCCG GGGCCAGCGA CTCGAGTTCG GCGACGACCG |
| 55081 | TCGGACGGTC GCCGCCGAGC ACGACGGCCC GCTGCTCGAA GGCCGTCCTG GTCGTCGCGA |
| 55141 | GCGACAGCGC CACATCGGCC AGGGGCGACT CGTCGGCGGT CAGGCGCCTG GCGATCAGCG |
| 55201 | GGGCCTGGGC CGACAGGGCG TCGGGGGTGC GTGCGGAGAC CAGGAAGGGC AGAACTCCCG |
| 55261 | ATCCCTGAGC CGGAGTTCCG GTGCCCTTGG CCGTCGGCGC GGGTACGTCC GGTTCCGGGT |
| 55321 | CGGGGGCCTG TTCGAGGATC ACGTGTGCGT TGGTGGCGCT GATGCCGAAG GACGAGATGC |
| 55381 | CGGCGCGGCG GGGCCGGTCG GTCCGCGGCC ACGGCTGTGC CTCGGTGAGC AGGCGGACGG |
| 55441 | CGCCCGCCGA CCAGTCGACG TGCGTGGCCG GCCGGTCGAC GTGCAGCGTG CGCGGCAGCA |
| 55501 | CGCCGTCGCG CATGGCCATC ACCATCTTGA TGATGCCGCC GACCCCGGCC GCGCCCTGCG |
| 55561 | TGTGACCCAG GTTCGACTTC AACGAACCAA GCCAGAGCGG GCGTTCGGCC GGGTGCTCGC |
| 55621 | GCCCGTAGGT GGCGAGCAGC GCCCGTGCCT CGATCGGGTC GCCGAGCGTC GTGCCGGTGC |
| 55681 | CGTGCGCCTC CACGACGTCG ACGTCTGCGG CCTGGACCTG CGCGTCCTGG AGGGCGCGCC |
| 55741 | AGATCACGCG CTCCTGCGCG GGGGCGCTCG GCGCGGCCAG ACCGTTGGAC GCGCCGTCCT |
| 55801 | GGTTGACCGC CGAACCGCGC ACGACGGCCA GCACGCGGTG CCCGTTGCGC TGCGCGTCCG |
| 55861 | AGAGGCGTTC GAGGACAAGC ATGCCGGCGC CCTCACCCCA GGAGGTGCCG TCGGCGGCGT |
| 55921 | CCGCGAAGGA CTTGCTGCGG CCGTCCGCGG CGAGGGCACG CTGCCGGGAG AACTCGGTGA |
| 55981 | AGGTGATGGG CGTCGGCATG ACTGTGACGC CGCCTGCGAG CGCCAGCGAG CATTCGCGGC |
| 56041 | GGCGCAGCGA CTGGGCGGCC AGATGCAGCG CCACCAGCGA CGACGAGCAG GCGGTGTCGA |
| 56101 | TCGTGACCGG GGGCCCGGAC AGCCCGAGGG CGTAGGAGAC ACGGCCCGAG ACGACGGGGC |
| 56161 | TGTTGTTGCC CCTCACGAGG TAGGCCTCGT ACTCCTCGGA CGAGGCCTGC AAGGGCACGT |
| 56221 | CGTAGAACGA GTAGTAGGCG CCGGTGAACA CGCCGGTCTC GCTCTCGGGC AGCGTGTCCG |
| 56281 | GGGCGATGCC GGCGCGCTCC AGGGACTCCC AGGCCACTTC GAGCATGAGG CGCTGCTGCG |
| 56341 | GGTCCATGGC GACGGCCTCG CGGGGCGAGA GGGCGAAGAA ACGTGCGTCG AACCAGGCGG |
| 56401 | CGCCGTGCAG GAACCCGCCC TCGCGGACGT ACGAGGTGCC CTCGTCGGTG CCGTCGCCGG |
| 56461 | CGAAGAGCGC GTGGAGGTCC CAGTCCCGGT CCTTGGGGAA GGCGCCGATC GCGTCGCGGC |
| 56521 | CCGTGCGCAC CAGGTCCCAC AGCGCCTCGG GGGAGTCGGA GCCGCCGGGG AAGCGGCAGC |
| 56581 | CCATGCCCAC GATCGGGATG GGCTCGGTGG CGCGGCGCTC GGTGTCCTTC AGGCGCCGGC |
| 56641 | GGGAGTCCTG GAGGTCGCCG GTGACCTTCC CCAGCGCCTG AAGAAGCTGT TCCTGGCTTA |
| 56701 | CGTCGTCAGA CATCCACGCG AGCGTTTTCC ACTGTCGGGC AAAGCACTGT CACTCGAAGC |
| 56761 | CCTTCTCGAT CAGCGCCAGC AGGTCGGAGG CGCTGGGGGT CGCGATGGCC GCGCCGCCGT |
| 56821 | CCGTGTCGTC GTCCGCGGCT TGTGCGGCCG GTTCGGTGTC GCGGGGTGTC TCGCGCAGCC |
| 56881 | GGGCGGCCAG CGCGCCGAGC CGGGTGGCGA GCCGGTCCCG GTCGGCGGCG GAGCCTTCGA |
| 56941 | GGTCCGCGAG CGACCGCTCC AGCGAGGCGA GGTCGGCCAG GGCGCGGGCG GCCTGTCCGC |
| 57001 | CGCCGTCCGG GACGGCTTGG CGGCAGAGGA ACGCGGCGAC GTCGGTCGGC GTCGGATGCT |
| 57061 | CGTAGACGAG CGTGGCGGGC AGGTCGAGAC CGGTGGACTC GCGCAGCCGG GTGCGGAGTT |
| 57121 | CGACGGCCAT GAGGGAGTCG AAGCCGAGGT CCTTGAAGGC GTGGTGCGGC GCGACGTCCC |
| 57181 | GGGCCGCGGC GTGACCGAGC ACGGCGGCGA TCTGGGCGCG CACTAGGTCG AGCGCGACGC |
| 57241 | GCTGCTGTTC GTCGGGACTG TGGCCGGCCA GCACTTTGGC GAAGTCCGGC GTGGTGCCGC |
| 57301 | GGGTCTCGTC GGTGCCCGGA CCCGCGCCGG TGAGGCCGGC GAGGTCGGAG GCGGCACCGG |
| 57361 | GCACCGGAAC GAGTCGGGCG AGCAGCGGGC TGGGGCGGGC GGCGGTGAAG ACGGCGGTGA |
| 57421 | AGCGTCCCCA GTCGACGTCC GCGACGGTGA CCGTGGTGTC GCCGTGCCGC AGCGCCCGGT |
| 57481 | CCAGGGCGGT GACGGCGAGT TCCGGCGCCA TCGGGGCGAT GCCGAGTCGG CGCAGGTCGT |
| 57541 | CACCGAAGCT CCCGGCAGGC ATGCCGCCGC CGTCCCAGGG GCCCCAGGCG ATGGCCTTGC |
| 57601 | CTGCGGCGCC CCGGGCGCGG CGCCGCTCCA TCAGCGCGTC GACATGCGCG TTCGAGGGGG |
| 57661 | CGTAGGCGCC GCGGGACGCG TTGCCCCAGA CGGCTGCGAC GGAGGTGAAG GCGACGAACG |
| 57721 | CCGACAGGCC GTCGCCGAAG AGCTCGTCGA GGTTGCGGGC GCCGGTCACC TTCGGCGCCA |
| 57781 | TGACGGTCGG GAAGGTGTCG GCGTCGAGCG CGGCGACGGG GTTCTGGTCC CCGGTGCCCG |
| 57841 | CGGCGTGCAC CACCGCGGCG AGGGGCGTGT CCGCGGCGGC GAACCGGGCG GCCAGTTCCT |
| 57901 | CGACGTCGGC CCGGCGGGAG ACGTCGCAGT CCGCGAGGAC GAGTTCGGTG CCGTGCCGCG |
| 57961 | CCAGTTCGGC GCGGAGCCGG TGCGCGTCGG CGAAGCCACT GGCGCGGGGG CTGGCGAGCA |
| 58021 | CGACGATGGG CGCGCCGGAC TCGGCGAGCC GGCGGGCGGT GTGGACGGCG AGGGCGCCGG |
| 58081 | TGCCGGTGAT CAGCACCGGT CCCGAGGACC ACAGGTCCGC GTCCTCGGTG TCCTCGGCGG |
| 58141 | GCCGTTCGTG GGGGCTCACG GTCGGCTCGG CGCGGGTCAG GCGCGGGGCG TGGACGCTGT |
| 58201 | CGTCCCGCAG CGCGAGCTGG TCGTCGCCGC CGGATCGCCC GGCGAGGACG GCGGCCAGCC |
| 58261 | GGGCGAGCCC GCCGCAGCCG AGGGGATCGT CGTCCGTGCT CGCCGCGAGG TCGATCAACG |
| 58321 | CGCCCCAGAG TCCCGGGTGT TCGAGTCCGA CGACCCGGCC GAGGCCCCAG ACCTGGGCCT |
| 58381 | GCCACGGGTC CGGCGCCGCG TCACTCTCCA GCGCCCGCAC CGGGCCACGG GTCAGGGTCC |
| 58441 | ACAGCGGCGG GTCCGTGCGG CCCGTGTCGA GCAGAGCCTG TACGAGCAGC AGGGTGGCGT |
| 58501 | CCGCGGCGGC CGACACGCCG TCGCGGCGCG CGGTCTCACC GCTCGGCCAG GGCAGGGCGA |
| 58561 | GCACGCCCGC GAACCGCTCG TCCGACGCCG GGGTCCGGTC CAGTTGCCGG GCGAGTTCCT |
| 58621 | TCGCCAGGGT CGCGCGATCG GCGGTCGCCA CGTCGACCGT CAGGAGGCGG GTCGTCGCGC |
| 58681 | CCGCGTGGGC.CAGTGCGGCC GTGACGGCGT CGGCGAGCGC GGCCGACGCG CGGTCTTCGG |
| 58741 | CGGTGTCGGC GTCGGATTCG GGGGCGACGA TGAGCCAGGT GCCGTCAAGT GCCGCCGCCG |
| 58801 | GGGGCGCGGC GTCCGGAAGA CGTTCCCAGG TGACGTCGTA GCGCCACCTG TCGGTTCTGG |
| 58861 | CGATCATGTC CTGTCCGCGC CGCCAGTTGC CGAGCGCGGG GAGCACGGCG CTCACCGGCG |
| 58921 | CGTCGGCGCC GAGGCCGAGC GTGGGGGCCA GGCGGTCGAG GTTCTGCTCC TCGACGACCT |
| 58981 | GCCAGAACGC CGTGTCCTCC GCGCTGTGCG TGCCGTCCGG CGCGGCCGGA ACGTCCGCCG |
| 59041 | TGTCCGGTGC GGCGTCCAGC CAGTAGCGCC GGTGCCGGAA GGCGTACGTC GGCAGTGCGA |
| 59101 | CACGCCGGGC GCGGGGCGTG AGCGCGGTCC AGTCGACCGG CACGCCGTTG ACGTAGGTCC |
| 59161 | GGCTCAGCGC GGTCAGCACG GTCGCCGCCT CCGGCCGGCC GCGCCGGGAG GAGGCCGCGA |
| 59221 | CGGGCCGAGT CCCCTCGAAC AGCGGCGTCA GGGCGGCGTC CGGGCCGATC TCGATCAGCA |
| 59281 | CGTCCGCGTC CGGCAGGCCG CGCACCGCGT CGGCGAAGCG CACCGCGCGC CGCGCGTGCT |
| 59341 | CGATCCAGTA CTCGGGGGTG TCGAAGGCGT GCGTGGTGTC CGCGGCCGGG CTGACCTGGA |
| 59401 | TCTCGGCCGG GTGGAAGGTG AGGCCGTCGA CCACGGCGGC GAACTGCGCA AGCATCGGTT |
| 59461 | CCATCAGCGG GGAGTGGAAC GCATGGCTCA CCGGCAGCCG GGTCGTCCTG CGGCCCTCGC |
| 59521 | CGCGCCAGTG CTGCGCGATG CGCGTGACCG CGTCCTCGGC TCCCGAGACG ACGACCGCGG |
| 59581 | AAGGCCCGTT CACCGCCGCG ATCTCGGCTT CGGAAGTGTC CAGGAGGTAG GCGAGGCTCG |
| 59641 | CGGCGACCTC GTCGTCGGTC GCGTGGATCG CGACCATCGC GCCGCCCTCG GGCAGCGCCT |
| 59701 | GCATCAGGCG TCCGCGGGCG ACCACGAGCC GGGCGGCGTC CTCGACGGAC AGCACGCCGG |
| 59761 | CGACCACGGC CGCCGCGATC TCCCCGACGG AGTGCCCGGC GAGGGCCGAG AACTCGACGC |
| 59821 | CCCACGACAT CCACAGTCGC GCCATGGCGA GTTCGAAGGC GAACAGGGCG GGCTGCGCGA |
| 59881 | ACTCGGTTCG CTCCAGCACC TCCGCGGGCT CCTGCCACAG TACGTCGCGC AGCGGACGTC |
| 59941 | CGAGCAGGGG GTGGATCACG TCGCAGATGT CGTCCAGGGC GGCGGCGAAC ACCGGGAAGT |
| 60001 | GCTCGGCGAG TTGACGCCCC ATCGCGGGCC GCTGTGCTCC CTGGCCGGAA AACAGCGCCG |
| 60061 | CGGCCCGTGT GCCGCCCGTG GTGCCGGCCT CGGGACCGGT GACCGCCTCG GCCGGGTGCA |
| 60121 | CCGCGTCCGG GGTACCGAGG GCCGCGAGCG CACCGAGCAG GCCGTCGCGG TCCTCCGCGA |
| 60181 | CGACCACCGC GCGGTGGTGG AAGTCGCTGG GCGTGGCGGC CAGCGAATAC GCCACGTCCC |
| 60241 | GGAGACCGAG TCCGGGGTTG GCGAGCAGGT GGCCGTGCAG GGCTCGGGCC TGTGCGTGCA |
| 60301 | GACCGTCCCG CGTACGGGCG TTGACGGGGA TCGCGACGGC GACGGGGGCG TCGTCGTCCG |
| 60361 | TAGCGGCCTC GGGGCTCTGC GGCACGTGCC GCGCGTCGCC CTCTTCCAGG ACCACGTGCG |
| 60421 | CGTTCGTGCG GCTGATGCCG AACGACGACA CCGCCGCCAG CCGGGGACGC TCCGTGCGCG |
| 60481 | GCCAGGGCCG TTCCTCCGTC AGCAGCTCCA GCGCGCCCGC CGACCAGTCC ACGTCGCTGG |
| 60541 | AGGGCTTCTC GGCGTGCAGG GTCCTGGGCA GCAGTTCGTG GCGCAGCGCC TGCACCATCT |
| 60601 | TGATCACGCC GCCGACACGC GCCGCCGCCT GGGCATGGCC GATGTTCGAC TTGAGGGAGC |
| 60661 | CGAGGTACAC CGGGCGGCCT GCCGGAGGCC GCGCGCCGTA CTCGGCGAGC ACGGCCTGCG |
| 60721 | CCTCGATCGG GTCGCCGAGC CGGGTGGCGG TGCCGTGCGC CTCGACCGCA TCGACGTCGG |
| 60781 | CGGCGGGGAC GCGCGCCTGC TCCAGCGCGG CCTGGATGAC GCGGTGCTGG GCCGGCCGGT |
| 60841 | TCGGGGCGGT CAGACCGTTG GACGGGCCGT CCTGGTTCAC CGCGGAGCCG CGCACCACGG |
| 60901 | CCAGGACGTC GTGCCCGAGG CGCGGCGCGT GGGAGAGCCG TTCCACCGCG AGCACGCGCA |
| 60961 | CGCCCTCGGC CCAGCCGGTG CCGTGCGCGT CGTCGGAGAA CGACTTGCAG CGGCCGTCCG |
| 61021 | CGGCCAGGCC GCCCTGGCGG GCGAACTCGA CGAAGAGGTG CGGCGAGGCC ATGACGGTCG |
| 61081 | CCCCGCCCGC GAGGGGGAGA TCGCATTCGC CCGCGCGCAG TGCCTGGCAG GCCAGGTGCA |
| 61141 | GGGCGACGAG GGAGGACGAG CAGGCGGTGT CCAGGGTGAC CGCGGGGCCT TCGAGACCGA |
| 61201 | GCAGATAGGA GACCCGGCCG GAGATCACGC TGTTGCTGTT GGCGATCCGG ATGATGCCTT |
| 61261 | CGAGGGTCTC CGGGGTGCCG TTCAGCCTGC TGAAGTAGTC GTTGTACATG ACGCCCGCGA |
| 61321 | ACACAGCGGT GCGGCTGCCG CGCAGCGTCT TGGGGTCGAT CCCGGCGTGC TCGACGCTCT |
| 61381 | CCCAGGGTGC CTCCAGCAGG AGCCGCTGCT GCGGGTCCAT CGGCAGCGCC TCGCGGGGAG |
| 61441 | GGATGCCGAA GAAGGCGGCG TCGAACCCGG CGGGGTCGTC GAGGAATCCG CCGCCACGGG |
| 61501 | CGTACGTGGT GCCGGTCGCG GCGGGGTCGG GGCTGTAGAT GGGCGTCAGG TCCCAGCCGC |
| 61561 | GGTCGGCGGG GAAGGGCGCG ATGGCGTCAC GGCCCTCGGC CAGCAGCCGC CAGAGGTCGT |
| 61621 | CGGGCGGGCT GACGCCGCCG GGGTAGCGGC AGGCCATGGC GACGATCGCG ATGGGCTCGT |
| 61681 | CCCGCTCCGT CCTGCCGGTG GTGAAGTCGG GCTTCCTGTC GGGCATTACG GTGTCGCCGG |
| 61741 | TCCCCGTGGG CGGTGCGAGC CGTATTTCCA GGTGGCGGGC GAGCGCGGCC GACGTCGGGT |
| 61801 | GGTCGAAGAC GAGCGTCGCG GGGAGGTGGA CACCGGTCTC GGCGGAGAGC CGGTTGCGCA |
| 61861 | GTTGCACCGA CATGAGCGAG TCGAAGCCCA GCTCGCCGAG CGGGCGATCG GCGTCGATCC |
| 61921 | GGCCCGCGTC GGCGTGGCCG ACCGCCGCGG CGATCTCGGC GCGCACAAGG TCGAGGAGAT |
| 61981 | CCGTGCCCCG GTAGGCGGAC CGCGCGGGGC GCGCCCCTGC GGCGGCGCGG GGCGCGGCGC |
| 62041 | CGGACGATGC CGCGCCGCGC GCCCGTACGA GGTCGGTGAA CAGCGGGGCC GGGCGGACGG |
| 62101 | CGGTGAACAG CGGCAGGAAC CGCGACCAGG CGACGTCCGC GAGGAGGCAG TCGGTGCCGA |
| 62161 | GCGCGAGCGA GCGGGCCAGC GCGGTCACGG CGTCGGCCGC GGGCAGCGGG ATCAGGCCGC |
| 62221 | TGCGACGGAA CCGCTGTTCC CGGGTCGCGT CCAGCATGCC GCCGCCCGCC CACGGTCCGA |
| 62281 | AGGCGAGGGA CGTCGCGGCG CGGCCCTGGG CACGGCGGCG CGCGGCGAGC GCGTCCAGTG |
| 62341 | CGGCGTTGCC GGCGCCGTAC CGGGCCTGCC CGGGGGCGCC CCAGATTCCG GATACGGAGG |
| 62401 | AGAAGAGGAG GAAGGCATCG AGGCCGGCGT CGGCGAGCAC GTCGTCGAGG ACGAGGGCGC |
| 62461 | CGCGCGTCTT CGCGTCGAGC ACGGCGCGGA AGTCGTCCGC ATCCGTCTCC AGCAGCGGGG |
| 62521 | CCTCGTGCGC GATGCCGGCG GCGTGGAAGA CGGCGCGGAT CGGCGCGCCC GACGCCGCGG |
| 62581 | CCTCCTGGAC GACAGCGGCC ATCGCGGCGC GGTCGGCGGT GTCGGCGGCG ACGACGCGCA |
| 62641 | CCGGGACGCC GGTGGGGGAC AGCTCCTCCA GCAGCTCCGC GGCGCCGGGC GGTTCGGGGC |
| 62701 | CGCGGCGGGA GACCAGCAGC AACGAGCATC CGTCCGCGAC CTGTTGCGTT GCGGACCCGG |
| 62761 | CCGCCAGTCC CGCGACCCAG CGGGCGACGT GCCCGCCGAG CGCCCCCGTG CCGCCGGTGA |
| 62821 | TCAGCACCGT GCCGGAGGGC TGCCAGTGCG TGAGGTCCTG GTTCGGCGCC GGGTCGTTGT |
| 62881 | CCGGTGCGGA CCTGACGACG GGTGGTACCT CGAGCCCGTC GCCCGCCACG CGTACCTGGT |
| 62941 | CCTCCCGGGT GTCCCCGGCG AGGAGGGCGG CCAACTGTCC GAGGGGCGGT TCGGCGCCGT |
| 63001 | CGGCAGGCAG GTCGATCAAG CCGCCCCAGG TATCGGGCAG TTCGAGGGCG GCGGCGCGGC |
| 63061 | CGGCTCCCCA GACCGCCGCG GCGTCCGGGT CGGTGCCGCT GTCCCGGGTC AGGCACCACA |
| 63121 | CCTTGGCGGT CACGCGGGCG TCGGTGAGCG CCTGGAGCGC GGTCAGCAGG GTCTCGGGGC |
| 63181 | GGTCCGCGAA GCACAGTAGG CCCGCGAAGC CGTCGGCGAT CGCGTCGGCC GGCTGAGCGG |
| 63241 | AGAGGGGGCA GAGGGCGGAC GCGAGCGTGG CGCGGTCCGC GGCCTCGTGC GGCGGGACGG |
| 63301 | TCACGGTGTG CTCGACGAGT TCGCCCACCC ACGCGGGGAT GTCCGACGCG CCGTCCGCCG |
| 63361 | CCGCGGGCGG GACGACCAGC CAGGTGCCCG GCACGGGGAC GGTACGCGGC GCGAGGTCGG |
| 63421 | CGGGCTGCCA TGTCAGCTCG TACCGCAGCC GGTCGAGGGC GGCGTCGGCC TGCGCCGACA |
| 63481 | ACCGGGCCGG GTCCGCCTCG CGGAGCACCA GCTCGCGGAC GGTGACGACC GGCTCTCCGT |
| 63541 | CGCTCGTCAG CGCGTCCAGC CGCGCGCTGT GCCCCGCAGT GCGCAGCCGG AGCCGTATCG |
| 63601 | GGGCGCCGTC CGCGGTGGCC GAGCCCCGCG ACGGGCACAG GGTGGCACCG TTGAAGGCGA |
| 63661 | ACGGCAGCCG GATCCGCCGG CTCTCGTCCC CGGTCAGGGC GAGCGGGTGC AGCGCGGCGT |
| 63721 | CGAGCAGCGC CGGGTGGATG CCGAAGCGCC CGTCGTACCG GCCGTCGGGC AGCGCGACCT |
| 63781 | CGGAGAACAG GTCGTCGCCC GCCCGCCATA CGGCACGCAG GCCGCGGAAG GCGGGTGCGT |
| 63841 | AGCCGTAGCC GCGGTCGGCC AGATCCGCGT ACAGGTGGTG GACCGGTACG GGTTGCGCCC |
| 63901 | CGGCGGGCGG CCACGCGCGC ACGGCCCAGT CGACGTCGCT GTGTTCGGGC TCGTGAGGTT |
| 63961 | CATGTCGTTC CGGCAGGGGC TCCGTGAGTG TCCCGGTGAC GTGCACCGTC CACTGGCCTG |
| 64021 | GCACGTGGCC CGTCGCCTGC CGCGCGCGAA TCGTCAGGTC AGGAGCGGCC GTCCCTTCCG |
| 64081 | ACGGGCCGAC GCAGACCTGA ACGTCCGGGC CGCCGTCGCC CGGCACCAGC GGGGCCTGGA |
| 64141 | TGACGAGTTC GGAGATCTCA CGGCAGCCCG CCTGCCGGGG GGGCTGTGCG GCCAGTTCGA |
| 64201 | CGAAGCGCGC GCCGGGGAAC AGGACGCTGC CGGGTATCGG GTGGTCCGCG AGCCACGGGT |
| 64261 | CGGCGGACGG GGAGATGTGG CCGGTGAGCA CGAGGCCGCC GGGGCCGGGC AGTTCCACCG |
| 64321 | CGCCGGACAG CAGCGGGTGC GCGAGCGGGT CGGTCCCGGC ACCGGCGGTC GCCCGGGGCG |
| 64381 | GCGCGAGCCA GTAGGTGTGG CGTTGGAACG CGTACGGAGG CAGGTCGACG AGGGGCGCGG |
| 64441 | CGGGCAGCAG CGGCGCCCAG TCGACGCGCA GGCCGTGTGC GTAGGCCTGG GCGAGGGTGG |
| 64501 | TGAGGACGGT GTGCGTCTCG GACCGGTCGC GCCGGGTGCT GGCGAGGACG GTACGCCCGT |
| 64561 | CGTCCCCATG GGCCTGCGCA GCACCCACCA TCGGTGCGAG GAGGGCGTCC GGGCCGATCT |
| 64621 | CCACGAGGAC GTCGGCGGCG GCGAGGCCGC GGACCGCGTC GTGGAAACGG ACCGCGTTGC |
| 64681 | GGGCGTGGTC GATCCAGTAG TCCGCGCTGT GGAAGGGGTG GTGGCTCGCC GCCGAGGCCG |
| 64741 | CGATAGGAGT CTTCGGAATG CTGAAGGTCA GGCCCCTGAC GACCTCGGCG AAGTCGTCGA |
| 64801 | GCATGGGCTC CATCAGAGCG GAGTGGAAGG CGTGGCTGAC ACGCAGGCGC GTCGTACGGC |
| 64861 | GTGCGGGACC GCGCCAGATC TCGCCCACGC GCTCCACGGC GTCGAGCGCA CCCGAGACGA |
| 64921 | GGACGGCATC GGGCCCGTTG ACGGCGGCGA TGTCCACGGC GCGGTCGGCC GCGTGCGGAC |
| 64981 | CGTCCGTGAC CGTGGCCAGC GTTTCGGCCG CCTCCGTCTC GCTCGCCGCG ATCGCGAGCA |
| 65041 | TCGCCCCGCC CTGGGGCAGC GCCTGCATCA ACCGGCCTCG GGCCACGACG AGTCGGGCGG |
| 65101 | CGTCCTGAAG GGTGAGCACC TCGGCGACGA CCGCGGCGGC GATCTCCCCG ACGGAGTGGC |
| 65161 | CGGCGAGGGC GGTGAAGGTC ACGCCCCAGG ACTGCCACAG GCGTGCCAGA GCGAATTCGA |
| 65221 | AGGCGAACAG GGCGGGTTGC GCGAACTCGG TGCGGGCGAC GGTCTGTGCG TCGGACTCCC |
| 65281 | ACATCACCTC TCGCAGGGGG CGCCCGAGGA GCGGGTCGAC GCAGGCGCAC ACCTGGTCGA |
| 65341 | GCGGGGCGGC GAAGGCCGGG AAGTGCGCGT CGAGTTCGCG GCCCATGCCG GGCCGCTGCG |
| 65401 | CACCCTGGCC GGTGAACAGC GCGGCGACCC GTCCGGCGCC CGCCTCGACC GGGGCGGTGT |
| 65461 | CGGCGAGCCC GGCGAGCAGG CCCGCGCGGT CCCCGGCGAG CGCGACGCGG TGGGCGAACA |
| 65521 | GCGACCTGTG GTCCACGAGG TTGCGGGCGG CGTCCACGAT CGGCAGCCCG GGGTGGAGCG |
| 65581 | CCAAGTGGTC AGGCAGCGCG GTCGCCTGGG CCCGGGCCGC TTCGGGCGTC TTGCCGGAGA |
| 65641 | CGACCACCGG GACGGCGACG CGCGGGCAGT CCTGGCCGTT CGCGGCGGAG TCCTGATCCA |
| 65701 | CGGACCGCCG GGTGTTCGCG GCGGAACCCT GGCCCGCGAG CCGCTGCGCG TCCGCGGTCG |
| 65761 | CACCCGCGTC CTCGGACACG AACTCCTCGA GGATGACGTG CGCGTTGGTG CCGCTCATGC |
| 65821 | GGAACGACGA GATGCCCGCC CGGCGCGGGC CGGCCGGGGC GTCCCATTCC CGGGCCTGCG |
| 65881 | CGAGCAGCCG CACCGTTGGG GCCGACCAGT CCACCTCGGT CGTGGGGTTC TCCGCGTACA |
| 65941 | GGCTGGTGGG CATGACGCCG TGGCGCATCG CCTGCACCAT CTTGATGACG CCCGCGACAC |
| 66001 | CGGCCGCCGC CTGTGTGTGC GCGAGGTTCG ACTTGAGCGA GCCGAGGTAG AGCGGGCGGC |
| 66061 | CGGCCCGGCG GCTGCGCCCG TAGGTGGCCA TCAGCGCCTG TGCGTCGATG GGGTCGCCGA |
| 66121 | GCCGGGTGCC GGTGCCGTGC GCCTCGACGA CGTCGACCTC GTCGGCCGAG AGCCCTGCGC |
| 66181 | TGCTCAGGGC CGTCTCGATG AGGCGCTGCT GAGCCGGGCC GCTCGGTGCG GTCAGGCCGT |
| 66241 | TGGACGCGCC GTCCTGGTTC ACCGCGGACC CACGCACCAC GGCCAGCACA CGGTGGCCCA |
| 66301 | GCCGCCGGGC GTCCGACAGC CGCTCCAGAG CCAGGACGCC CACGCCCTCC GACCATCCGG |
| 66361 | TGCGGTCGCC GTCGTCGGCG AAGGAGCGGC AGCGCCCGTC GGTGGAGACG ACCCGCTGGC |
| 66421 | GGCTGTACTC GATGAAGAAC AGGGGGCTGG ACATCACCGC GACGCCGCCG GCCAGCGCGA |
| 66481 | GATCGCACTC GCCGCTGCGC AGCGCCTGCG CGGCCAGGTG CAGCGAGACC AGCGAGGAGG |
| 66541 | AGCAGGCGGT GTCGACCGTG ACCGCGGGGC CTTCGAACCC GTACAGGTAC GAGACCCGGC |
| 66601 | CGACTGCCAT GCTGCCCGCG CTGCCCGAGT GGATATAGCC GTCGTATCCG TCGGGCGCGG |
| 66661 | CCGTGGCGAA CCGGCCGCCG TAGTCGTGGT GCATGACGCC GGTGAACACG CCCGTCCGGC |
| 66721 | TGCCGCGCAG GGTCGCCGGG GGAATGCCCG CGTCCTCCAA GGCCCGCCAG GTCGTCTCCA |
| 66781 | GCAGCAGCCG CTGTTGGGGG TCCATGGAGA AGGCCTCGCG GGGGCTGAGC CCGAAGAAGT |
| 66841 | CGGCGTCGAA CAGGTCGATG TCGCGGAGGA ATCCGCGCTC GCGCGTATAC GAACGGGCGG |
| 66901 | TCGCGTCGGG GTCGGGGTCG TAGAGGGCTG CGACGTCGCA GCCCCGGTCC GTCGGGAAGC |
| 66961 | CGGTGATCGC GTCGCGTCCC TCGGCCACCA GGTCCCACAG CTCCTCGGGC GAGGTCACCC |
| 67021 | CGCCCGGGTA GCGGCAGCTC ATGCCGACGA TCGCGATCGG CTCGCGGGCC GGGGCCTCCG |
| 67081 | CATCGCGCAG CCGGGATCGC GTACGTTGCA GGTCGGGGGT CACCTTCCGC AGGTAGTCGA |
| 67141 | CGAGCTGCTG TTGGTCAGTC ATGTTCCTCG CCCATCGGCG TACGGCGGGT TCGCTGGCTT |
| 67201 | CGCGAACCCG GCATCGAATG AACTGCACGA GCCCGCCGAC CGGATCGAAT CCGGCGGTCT |
| 67261 | TCGTCTCGGC TCTCAATGCG GGGCGGACTG CGGCGGCCGT GCCGAATCGG ATTTCTTCGA |
| 67321 | TCCAAGCACG GAAACAGCGG CGCCCCCTAC TCAGGCACCC CCCTAAAACA CCCGGCATGG |
| 67381 | GCTTCGGTTG GGGTTGGACC AGGGGTGATG CGGCAGCGCC GGATGGGCGG GCACGGCATG |
| 67441 | CAAGAGGCGG GCGGGGGCAG CGGGGATCGC GGCCGGCGCC CGGCGCGGTT CAGTCGTCCC |
| 67501 | CAGGGAACCG TGGATCAATG GCTCCACGAA CCACGGATCT ACGGATCTGG AGGGAACTGG |
| 67561 | AGGGGATACG GATCCGGAGG GGACACCAGA ATTCAGGATT CAGGAAGCCG GTGAGTCGGC |
| 67621 | ACGGTTCTCG GCGGCTCCCT CGGCACGCTC TTCGGCACAC CACCATGGCG GCTGTACGGT |
| 67681 | GGGCTGGGGG ACCGAGCCGA GAGCGGCCCG GATGGGCTGG GCGCCCGCCG AGACGGGCAG |
| 67741 | CACGCCGCGC ACCGGCTCCG GCGCGAACAG CGCCTCGCAG ATCGTGTCGG CGAGATCCGT |
| 67801 | CCTGGAGACC GTGCCCTCGG GCTGCGGGTC GGCGCGGCCG CGTACCGTGA CCAGGCCGGT |
| 67861 | GGCCGGGGAG TTGTCGAGCA TCCCGGGACG CAGCACGCAC CAGTCCAGGT CGTGTCCGGC |
| 67921 | GAGCTTCCGC TCCACGTCCC GGTTCTCCGC GAGGTACGAC TCCAGCTCGT CACCGAGCCT |
| 67981 | GGTGTGCAGC TCGTCGTCGG GCAGATATGC GCTGACCAGG ACGAAGCGCC GGATGCCGAC |
| 68041 | CAACTGCGCG ACCCCCATCA ACTCCCCTAC GAGCGAGGTG GACGAGGTGT CCGTGGCGTC |
| 68101 | CGGGCCCCAG GCGGTCCCGG TGGCCACGGC GATCCCGCCG CAGTCGCCCA TCGCCCGTAG |
| 68161 | CGCCGCGGGG CTGGTCCTGT GCGCCTCGGA CGCGATCACC AGCGGGTCTG TCCCTGCGGC |
| 68221 | CCGCAACGAC TCGCTGTGCC CGGCCTCCCC GATGAGCCCC ACCGGGGTCA GGCCGCGGGC |
| 68281 | GAGGATCGAC TCGGCGAGCC GCCGGCCCAG GGCGCTCGTG ACGGCGAGGA CGACGACGTT |
| 68341 | GTTCTTCCCC GCCATCGGCC CCGCCTGCGC CGTCCGTGCG GCTTCCGCCC TCAGCGCGGC |
| 68401 | ATCGGCTGTC CGCGGGGCGT CCGCCGTCTC ATCGGCTTTC GATATCGGGA TGTTCGAGAT |
| 68461 | GTCTTCTTTC ATCGGCTCGG TCGCCATATC AGTCCGCTCA CGCCACGTCC TGGATTTCCG |
| 68521 | CGGGCGTGTG GTCCGGAGCA CCGCGCGATT CGACGATGGC GCCGATCTCG GCGCGGCGGG |
| 68581 | TGATATCAAA TTTTCGGTAA ATACGGGTCA AGTGCTGTTC AATGGTGCTC GCGGTGACGT |
| 68641 | AGAGCGACTC CGCGATTTCG CGGTTCGTGT ATCCCTGAGC CGCCATTCTG GCGACATTCC |
| 68701 | ACTCGGCGGG GGTGAGGCTG CGCCCGTCGC GTCGTGCCGG GGGCGGCGGG ACCTTGCGCC |
| 68761 | TCGCCTGCGA TCCGCCGGAC ATCTCCGCAA GCGCCCACTG CGCCCCGCAG CCGCGCGCCG |
| 68821 | TCTTCTCGGC GAGCCGCATC GTCTTACGAC CGCCGGCCAG GTCTCCGGTG TGCTTGTAGA |
| 68881 | TCTGCGCGAG CTGGCCGAGC GCGCGGGCGT ATTCGAGTTC GTCGCCGCAG GACTGGAGAA |
| 68941 | CGGCGATGGA TTTCCGGCAC GCCAGCGCCC GTTCCCCCGG CGGGACCGTC GCCACCCGCA |
| 69001 | GGCGCAGCCC GATGCCGCGC GCCCGGGTGT TCGAGTCCGG CGAGAACGCC AGCTGCTCGT |
| 69061 | CGATGAGCTC CGCCGCCTCG GCGTACTCGT CCAGCTTCAG CAGCGCCTCC GCGAGGTCCA |
| 69121 | GGCGCCACGG CACCAGGCCC GGCATCTCCA TGCCCCAGGC CGTGAGGATG GCGCCGCAGC |
| 69181 | TGCGGAAATC CGCGACGGCG GCGCGCAGGG CGCCGGTCGC CAGCCGGAAA GGCCCGCGGG |
| 69241 | CGCGCAGGTA CACCAGGCGG TACGGGCTGG CCAGGTCCGC CTCGGTCATC GGGCGGGCGA |
| 69301 | GTTGCGCGGC TGCCTCCTCG AAGCGTCCTT GCTCGGTCAG CATCAGCGGG GACAGGCCGC |
| 69361 | GCGGCAGACC GGCGAGCACG CCCCACCGGT CCGCGTCCCA GCGCTCGAAG CCGCGGCGCA |
| 69421 | CGGCCGTCTG GGCCGCGACG AGGTCACCCT TCCAGCAGGC AGCGACCGCC TCGGCCACGC |
| 69481 | TGAGCATGGA GGCCAGCGGC AGCGGCATAC GTTCGTTGTC GGCGAGGGCG GCGTTGTGGG |
| 69541 | CGAGGGCGGC GTACCAGGGG GCGCGCGGGT CGAGTCGTCC CGTCGCCATC AGGCAGAACA |
| 69601 | GCGCCACGAG CATCGGCTCC AGGCTCGAAT AGTCGAAGTT GGCGGACTGG AGGATCTCCT |
| 69661 | CCGCGCAGGT CACCGCGGCT TCCGCGTCTT TCACGGCCTT CGCGGAACCT GCGGAATCCG |
| 69721 | CGGAACCCGT GGGCCGTGGA CCGGAGTTCG GCGCATCGGA GCACTGGACG CCCGGGGCGA |
| 69781 | GCAGCCACGA GAGCCGTTGC GCGCTGGACA GCCACGAGAA TCCCTCGGCG ATCAGCTGGG |
| 69841 | GGTGCGGCTG GTGGTCGGGC GCGGGCGCGT CCGGGCAGGC GTCGGGGAAC AGATGACGCA |
| 69901 | ACCACAGCCC GCTGAGCGTT CTCTGGACCT CGGCACGGAC ATCCGCGGCG CCGAGGGCGT |
| 69961 | CGCCGGGACT GACAGGACCG ACAGCGTTGC CGCGTCCAGG CGCGGGTTCG GCCAGGCGCA |
| 70021 | GCATGTCGGA CGCCTGGGCC ATCTCGCCGT GTCTGGCCAG ATGCCGGGAG AGACGGGCTA |
| 70081 | ACGAACCCGG CTTGAAAGTG CCGTCGGACG CGGCAGCCGC CAGGCGCCGC AGCCGCGGTG |
| 70141 | CGGTGAGCGC CGGGTCCATC CACCACACCA TGTCGGTGAT CCGGACGCCG GCCTCCTCCC |
| 70201 | GCAGGTCGGG CCCGGAGCCC CAGAACGAGG CGGACTCCAG GAGTTGGACA GCCAGCCGGT |
| 70261 | GCCGGCCGAG ACGCGAGGCG TGCTCGGCGG CCTCATAGAG GACCCCACCC GCCCAGGGCT |
| 70321 | GCGGCGCGAT CCCGGCCTCG TGCAGATACG GAGCGATCTC CCAGGCGGGC ACGCCCTGTT |
| 70381 | CGTGCAGGAC CTGGGCGGCA CGCAGCCGTA AGGACCGCAG TGGCGCGGGG TGCGTGGACC |
| 70441 | AGAGGACGGC GTCGCGCAGA TACGGATGGA GGGTCATGTC GGGGCGCACG ATCCCGCTCG |
| 70501 | CGATGGCCTG GCCCGCCACG TGGGAGACGT ACTCGGCATC CTTCTCCAGC ACGTTGCACA |
| 70561 | GCCGCTGGGG TGTGCACTGC GCGTCGAGGA CCGCGACGGC CTCCACCAGG TTCATCACCT |
| 70621 | CACGGGAGGT GTGCTCGCGG AGCCGCCGGG CCAGGCCGGC CGACGGAGGT CCGTCCGGCG |
| 70681 | CGTCCGGATC CCGGGCGATC GCGGCCGCGG CCTCGTGCAG CATCGCGAGG GTCAGGGCCG |
| 70741 | GGTTGCGGCC GGTGATCGGA TGGATCGCCT CTGCCAGAGG GTCGGCGCGG GGGGCGGGGA |
| 70801 | CGCGGGCGCG CGCCAGTTCA GCGACCGCGG CCCGGCCCAG CGGCCGTACG CGAACGCGCT |
| 70861 | CCGCCCAACC CGCGGCGAGC TCACGCAGTT CATCGACGAT CGAGGGCACC GTAGCGGCGC |
| 70921 | TGACGCCGGT CAGCAGGACC CTGATCCTTC CGGTGTGCGT GAGGCGGACC ACCTCGCGGA |
| 70981 | CGAACACCTG GGATTCGGGG GCCAGCAGGT CCGTCTCGTC CACCGCCAGC AGCAGCGAGC |
| 71041 | GGGTGGCGGC GGCCACGAGG CGCCGCGCGT GGTCGCCGGG CTCTCGCGCG CCGAACTCGC |
| 71101 | CCAGTTCCTC GGCGTATCCG ACGCCGCACA GACTGCCGCG CACGGTCAGG ACGCCGATCC |
| 71161 | CCATCCGTCG CGCTTCGGCG CACGCCGCTT CCAGCAAGGC CGTCTTGCCG CACAGGGCAG |
| 71221 | GGGCCTCGAT CACGCAGAGC CCGGCGCTGT CGGCCGGACG GCGTCGCAGC CATCGTCCTA |
| 71281 | GTTCGGCGAG CTCTCCATCG CGGTCGACGA GAGACACCGC ACAATTCGAC TCGGACCCGG |
| 71341 | AACTCGGCTT GGGCCGGCAA CTCGACCTGA ACACCGGGTT ACCACCGCCG GGTCGAGTCA |
| 71401 | TGGTTCACAC ATCGACGTCC CCGCGCGGCC GCCGCATATA ACACGCGGTG GACGTCTCGC |
| 71461 | GGGCCCTGAC AACGCGACAG GTGGTCAGCC CGCCCGTTCA GGGTGTAAGC GACAGCTTTC |
| 71521 | ATGGCTTCGT CTTTCGAAAG AGTGGATCGG CGGGCACGAA GGTGACCGCT GCTTTTGCCG |
| 71581 | TGGGCGACAG AGCGAGTGCA CAGCAAAGCG GTCCCCCAGT GCCTGGGCCC ATCGCCGGCG |
| 71641 | AATGAAAGGG TGGGCCGGCG CGATGGTCAA GTCATGCCGG CGGGGCGACA GACGGAGTGC |
| 71701 | GGGCCGCGCT GAGCAGGTCC GGTCCGGTCC GGTTCATCGT CCCCGTGACG CGACGACGCA |
| 71761 | GAGGCGGGCG TCCGGTGGGA CGCTTCGGAG CGAATTGCAT GATGAGACGT TCCCCCGTTG |
| 71821 | CGTAGTGCGG CCCACCGACC CCGGTACCGG CTGCCCTTTT CTGAATTCTT GCCCAACAGA |
| 71881 | CATGTGCGAT GGGGGATCAG GTTGGTCAAC AATGAGTTAA CCCTATGTGA GGTGAGGACA |
| 71941 | GCATGCTGCA AGGGCGTGGT CGAGTTGGCG ACATCCGGAC GCCGACCGGC CTCGGCCGCG |
| 72001 | GTGAACGTCA GCATCCCGCG GCCTACCGGC GCAGGGCAGC ACAGCCGACC GGGCTCACAC |
| 72061 | GGTGATCGCC TCCCCCTCCG GGACGATGGT CAGAGGCACC TCGGTCAGCG CCGCCGGGCT |
| 72121 | GAGGAACCGG GCCATCGACT CCTGACCGGT CCGGCTGAGC ATGATCTCGT GGATCTGGAC |
| 72181 | GAGACGGTTG GGGGGGACCT CGCGTACGTA GTCGGCCGCC CGGCCGAGCT GGGTCCACGG |
| 72241 | CCCGCTGGTG GGCAGCAGCA GCGTGTCGAG GGGCGCGGGC GGTACGTGGT ACGCGTCGCC |
| 72301 | GGGGTGGTGG ACCCGGCCGT CGACGAGGTA GCCGACGTTG GTGACGCGGG GGATGTCGCG |
| 72361 | GTGGATCGCG GCATGCAGGT CGCCGGACAC CGCCACGTCA AAACCGGCGA CGTCGAGGCG |
| 72421 | GTCGCCGTCC GCGACCGCGA CGACCTGGCC GCGCCGGGCC GCGCAGCGTC CGACCACACG |
| 72481 | GACCGGTCCG TAGACCCGCA GGCCGGGGCG GGCGTCCAGG GCCCGGGCGA TCAGGTCCTC |
| 72541 | GTCGAAGTGG TCGAAGTGGT CGTGCGTGAT CAGGAGGGCG TCGGCGGCGG CGACCACCTC |
| 72601 | GTTCGCGTCA GGGGTGAACG TGCCTGGGTC GATGGCGATC CGCCCACCGT CGTTGACCAG |
| 72661 | CGACACACAT GCGTGGGCGT GCTTGATCAG CTGCACTGCC GGCTCCTATC GGTGTGATGT |
| 72721 | ACAGGGTGCA CTGTAATACA GTCGCCCCTG TACTCTTTTG CTAGACTGGG GGGTGTGAAC |
| 72781 | GACGCGCGTT CGAACGAACC GGCCCCCCTC CCCGACGAGT TGGCGGTCCG CCTCCGGGCG |
| 72841 | GTTGTCGGCA CCCTGGTCCG TAGCGCCCGT ACCGTCGATC GGCTCGCATC CGTTCCGGCG |
| 72901 | GCGGTGCTCG GCCTCCTCGA CACGCGGGGG CCGATGACCA CGGCCGACCT GGCGGCGACC |
| 72961 | CGCGGAGTGC GCCACCAGAC GATGGCCGCG ACCGTCAGGG AACTGACCGA GGCCGGGTTC |
| 73021 | CTGGCCTCAC GCACCGATCC GGGCGATGCC CGGAGGAAGG TCCTCGCTCT GACGAAAGCG |
| 73081 | GGGAAGAAGG CGCTCGACAC GGACCGCCGT CAGCGCGTCG GCGTGCTTGC CGACGCGCTG |
| 73141 | GAGGAAACGC TGGACGATGA GGAGCGGGGC GCCTTGGCGC ACGCCCTTGA CCTCATCGAT |
| 73201 | CGGATCAGCG GCAGCATGCG GGGGGGCGAC TCCTTCTCCG GCGAGCGCGA GTTCAACACC |
| 73261 | GGAGCATGGT GACGAGGCCG GAGTAGCCTG CTCCGGCTGG TTTAGGCGCG TTTTCCACGC |
| 73321 | GCGCGCGAAC GCGTGGGGGT TGTCGAACAT GACGTTGTGG CCGGTGTCCG GGACGATGCG |
| 73381 | GAGCTTCACC CCCGCGCTCT CCGCAGCCCG TCCCTGCCGG GCAGTTCGCC GCGCGGGTCG |
| 73441 | CCCTGAAGGC GGATGCGTTC CATGCGTGCC CGCTCGGGCA TCACCCCCAT CGCCGGGCCG |
| 73501 | GTGCCGCGAT AGAGGTCAAA TGGCTGAGAA GTCCCTGGTG CGAGCGGATC CGTCGCCGCT |
| 73561 | ACCCGCGCGA GGGCGGCATG GACCAGCTCC ACCTCGAGCT CCCCGGACGT GCCTCCGCGC |
| 73621 | CGGCCAACAC CACCAAGACG ACGTCTCGTT GACCGGCTGC ACCACAAGAA AGGACATAAT |
| 73681 | CCCGATCTCG CTGGTTCGTC GGCGGCCCGG GGTGCCCGTT GAACGGTCGT GAACCGGGCT |
| 73741 | GAACGAGACG GAAACCGGGA CGGCCCGTAC GGGGCGTTGT CAGTGGCCTT ACCTCGTGCC |
| 73801 | GCATCAGACA GCGTTCACCC TGTAGCACCC CGGGAAAAGT CGTCGAAACA GTCTCCCTGC |
| 73861 | AGGAACCGAG GATGGCCCGC CTCCGGCCCT TGACGGCTGG CTGGGCGGAC GCGGACTCGG |
| 73921 | GGGCTAGAGT GGGTTGCTTG CCGGTGGCCT CCGGGGCTCG CAACGGCCCG TCCCAGCCGT |
| 73981 | CGCC |

TABLE 4
FOSTRIECIN SYNTHASE GENE CLUSTER

| | |
|---|---|
| ORF 8 | |
| MATETFEFQVEARQLLQLMIHSVYSNKDVFLRELVS | SEQ ID NO.: 19 |
| NASDALDKLRLAALRDDGLDADTSDPHIEIELDQKA | |
| RTLTVRDNGIGMSYDEVGKLIGTIANSGTAAFLQEL | |
| KEAQDAAGAEGLIGQFGVGFYSGFMVADEMTLVTRR | |
| AGERSGTRWSSRGEGTYTLETVDDVPQGSAVTLHLK | |
| PADADDQLHDYTSAWKIKEIVKRYSDFITWPVRLLP | |
| QATDGEETPEPETLNSMKALWARSRDEVSDDEYHEL | |
| YKHVSHDWRDPLETIRLQAEGTFEYQALLFLPAHAP | |
| HDLFTRDFRRGLQLYVKRVLIMDDCEALLPPHLRFV | |
| KGVVDAQDLSLNVSREILQQDRHIRMIQRRLTKKVL | |
| SSVKEMKANDADKYAAFWREFGAVLKEGLLGDTDDR | |
| DALLAVASFASTHAEETPTTLQQYVERMKEGQDDIY | |
| YMTGASRQTIENSPHMEAFRDRGLEVLLLTDPVDEV | |
| WVDVVGEFEGKRLRSVAKGEIDLDVQGGEQADGGRE | |
| KQAETYAALLGWMKEHLGEEMKDVRLSTRLTVSPAC | |
| VVSDAHDLTPALESMYRAMGQEIPSARRILELNPAH | |
| PLVQGLNQAYQEGEDRSGLAETADLLYGLAVLAEGG | |
| RPTHPGRFVKLVAERLERTLR*″ | |
| ORF7 | |
| MYAPTPKPSTDRQAWLRRYTNAPDARHRLVCLPHAG | SEQ ID NO.: 18 |
| GSASFYMPLARALAPEIDIVAVQYPGRQDRRADPFP | |
| ATLQDLAAHVAEALCGEPAVPTAFFGHSMGAAVAFE | |
| VIRLLEDSTTPVTALFASGRGAPSVNRGERVHAMSQ | |
| EDVLAELRGLEGTDSRMFDDPEIIEMIMPPLRNDYR | |
| LIETYRYVPGPPVACPIRGFLGAQDPKVDEGEMKLW | |
| ADHTAGSFDLTLLPGGHFYLVQHQPEIVEAIRNTLL | |
| VAPPYV*″ | |
| ORF6 | |
| MTDDAIPGRGRYTEQARAARLAWLLARTGATLDSAA | SEQ ID NO.: 17 |
| HTAIEAASLTGNLENFAGSVEVPVGLAGPLQFRGQG | |
| VREAVVAPMATTEGALVASASRGARALSLAGGVSTR | |
| VLSQRMSRAPAFEFDDLAGAARFSRWLGTRRPQLED | |
| QVRLVSQHARLVAVDPYQIGRYLHVRFVFETADAAG | |
| QNMTTAATWQICTWLNEVLADEPGLRPRNTLLEGNL | |
| SSDKKVSSVSLLAGRGTRVTAECVIPGDVVASVLKT | |
| TPAAIARGHRVAVIGGQQAGMTGYGINAANVIAALF | |
| VATGQDIACVHESAVSVLSFDSDGDDLIATLLLPNL | |
| VIGTVGGGTGLPDQRDWLGVLGCRGEGGTARLAEII | |
| AGFALALDVSTASALVSGQFADAHRRLGRARRVDWL | |
| RADDLGPGLLQPAMAERLDSPRLRVTDVVRAPAMVG | |
| DGISTELGALGERRKLTGVIPMTVSWTEDDGPQTTA | |
| ELVAKAKPRGEEIAAGIGRIASLCGPEVSSAWETWG | |
| GGSDFPAAHRRELAVFRRPEGVLTSLLPVCYGIIED | |
| EAREAYVILMERLDITTGPWSRTDVDRALRAIAPVH | |
| GHWLGRDQQILAEGWLYRDGTTAHLVKARELWEALV | |
| RHNAAELPELMTPQRTRTALAAAAEAEFWVQEMDAM | |
| PRTLVHNDFNPRNISRQSERVTAYDWELATVAVPQR | |
| DLAELLAFTLTPHSTTDEVDHHLEVHRAAVAAAAGP | |
| DATVPQPEQWRRGYGLALREMLLSRLQLYTAAHSHR | |
| ELPFLPAVLDTTFHLWNLEAARDGE*″ | |
| ORF5 | |
| MHTERILTEHHRFLATLDHPEQTQQTVLAELLAANG | SEQ ID NO.: 16 |
| ATSYLREHGLNERSGAEEFRKALPIRTQNAFGPWIE | |
| RAIAGEDGVLTAERPVAFFSSSGSTGQEKRIPVTPT | |
| YMKRCFLPFYHASFAVLLGAFPDLAADPGGVLNLWR | |
| DPTSPHARTADGRPHLGPSQIDHRLFGEGGGPEDGA | |
| AWATIPEQLSDADPWERAYLQLRLAAERDIKVLIGV | |
| NPALIAGLPHQLAAQWPRIVEEIARGTVGGVPHTTP | |
| DPRRAEQIARRADEYGVLDPYHLWPNLRAAVAWNSA | |
| LASLYLPRVRERYGPGVRLFAAPIGSSEGPVAVPVD | |
| DHPNAAPLYLPGCYFEFADAAEPIREDSPTVTAAEL | |
| EPGRDYHLVLSHIGGLYRCAVNDVVHVVDHVGRTPR | |
| IAYTGRDVLRTAGGVDLTERAVVRALAGTLADTGAE | |
| LRNATVETGTDRFRAAIASALPGPLPAGFATLLDKH | |
| LGETADGYRAARDAGALAPVEVLQVHQDAFQREWEH | |
| AIRSGQRRTRVKDRIFQPAPDSWARITADERAHA*″ | |
| FosB (module 2)″ | |
| MPANDDKLRDYLKRVTADLHQTRLRIRDIEARKREP | SEQ ID NO.: 7 |
| IAIVGMACRYPGGVTDPEQLWELAAGGIDAVSGFPS | |
| GRGWDLEGLYDPDPDAEGKVYVREGGFLHDAGQFDA | |
| PFFGISRREALAIDPQQRLVLETSWEAVERAGIDPL | |
| SLAGSRAGVFVGVMPQEYGPRLYEATGQGVSGHLLT | |
| GTTTSVLSGRIAYTLGLEGPAVTLDTACSSSLVAMH | |
| LATQALRSGECEVALAGGVTVMANPGTFVEFSRQRG | |
| LAPDGRCKSFAAAADGTAWGEGVGMLVLERLGDARR | |
| NGHRVLAVIRGSAVNQDGASNGLTAPNGPSQQRVIR | |
| QALADAGLEAADIDAVEAHGTGTTLGDPIEAQALLA | |
| TYGQGRFEGRPLWLGSLKSNIGHTQAAAGVGGVIKM | |
| VMAMRGGVLPRTLHVDEPSPHVDWEAGEVRLLTGPV | |
| VWEAGERPRRAAVSSFGISGTNAHLILEEPPVKERT | |
| AYEAEADSADPAVWLVSAKSPDALRAQADRLTEFLA | |
| ARPQTGTGHLARALATTRSQFEQRAALIGADRAGLT | |
| EALSALASGSGHPSLVRGQVTTGRTAFLFSGQGSQR | |
| PGAGRELYASYPVFAAAVDEACAVFDPLLGRSLREV | |
| MFAGPGSEGAELLNRTAFTQPALFVLHTALFRLLES | |
| FGVRPDHLVGHSIGELSAAHAAGMLSLADAATLVFH | |
| RARLMQQITTPGTMLALQAGEATARGLVAGREDVVS | |
| LAAVNAPESTVLSGDPEVLADIAAQLAERGIRSRRL | |
| TVSHAFHSPHQDQILDEFRRIAAGLTYRAPRIPIVS | |
| TLTGLLAEQDRITTADHWTEQLRHTVRHADAVTTLH | |
| GLGTTRYLELTAHPTLAPLAAETLEDASAAPAALVP | |
| TLRAGQPEPDTFLRALATLHVTGTPVTWFADHAEAD | |
| ADNADTADGRGHERGRATVPHLDLPTYPFQHENYWL | |
| TAPSSGTGPGAGADALPHPMLSQRTDLPGGGGVLFS | |
| GRLAPGTDPWLPDHAVMGTLLLPGTGFVELALEAAR | |
| AVGAGRVEELVLRAPMVFPGGRARDLQVWVAPDQGG | |
| ERELLIRTRTPGEDWTLHATGVVTASRVDTDGFTPD | |
| WTGAVWPPAGAEQIPGDTFYPDLAERGYEYGPAFRS | |
| VKALWRRGDDLFAEVVLPEDQPYGFGAHPALLDASL | |
| HALPITRSFYETDDEVRLPFSFGGVSLFATDVRRVR | |
| VRLRPRPEATSVWITDAAGTPVLAMESLILRAVERT | |
| QLQAAEGAVGQAATFAVRWEPLSEARIAERVPGTWL | |
| LFGTARPGLAELFEHVLTSTEWDASASTPVEGVLVC | |
| PADASELLAALRETERLDAPVWCVTSGAVGVGVDDP | |
| ATDVAAAGAWGLGRVAALELPSRWAGLVDVPETADL | |
| GTADDNAGRTTARLLAGVLTGDGAEDQLAVRDGRLW | |
| ARRLGTAPAADAGTWQPKGTVLITGGTGGLGAHVAR | |
| RLAALGTADRLVLLSRRGAESPGAAELLAELGESGV | |
| RAEAAAIDITDRTAVTQLLSRLDAEDDPVRTVVHAA | |
| GVIRYARIADVDPEAFETDMAAKVNGALLLDELLPD | |
| ADEFVMFSSIAGIWGAADQAAYAAGNACLDALARRR | |
| RERGASAVSIAWGPWSGGGMVTEYEDRELRKRGLLP | |
| LAVPSAVEALERAVPGDTDPVVVDVAWSRFLPAFTV | |
| LRPSPLLSGFAPADTAGGGRDAASAALPGAGTTAGA | |
| LKDRVGALPEDERLPVLLDVVRTHVAQLIGRGDPQQ | |
| VQADRALRELGFDSMMSVELRNRLGELVGARLPATL | |
| AFDHPTPESLAERLLTELDLDEAPADDGPVLEDFDR | |
| LEAKVLSPFTPADTRAALATRLSALLDRLSGTGTGA | |
| GGAGRNSGTDDLETASASDLMQFLDAEYGASDGTAS | |
| DPSRPTTS*″ | |
| FosA (modules 0-1) | |
| MMPSCPAASAAYPACAWSGSTTPSPPWRARAPWPPR | SEQ ID NO.: 6 |
| SRVRCSPSSRRPTISCSSGVPPSGPRRSRASSPIVR | |
| WKSSTSLRRWRRSAGPPRTAATPPYPSRVRFMSEGF | |
| MPIAVVGMACRLPAAPDPATFWRLLSEGVDAVGETP | |
| ADRWPDAAGTPTGAARYGAYLDRIDTFDPGFFGISP | |
| HEARAMDPQQRLMLELSWEALEDAAIVPASLGGSGT | |
| GVFVGAIWDDYRSVVARAGTASFNQHTMTGMGRGLI | |
| ANRVSYTLGLRGPSMTVDAAQASSLVAVHLACESLR | |
| RGESRVALAGGVNLIAAPEGMAASMSFGALSPDGRC | |
| HTFDARANGYVRGEGGALVVLKPLEQARADGDFVYC | |
| VIRGSAVNNDGATDGLTVPSAPAQTELLRAACRQAD | |
| VAPGDVQYVELHGTGTAVGDPIEAAALGTAFGAGVG | |
| RVADNALLVGSAKTNIGHLEGASGVVGLVKTALAIR | |
| HRKLPPSLNFVTPNPKIAFDELSLRVQVGLTPWPRP | |
| DGPIVAGVSSFGMGGTNCHVVLCDAPAESSAPSPAT | |
| SSAAPPATVPVPVAVPQDVASPWLISARSEAALRGQ | |
| AAALAAHLEQHPELDAATVARGLATIRTHHEHRAAA | |
| FGGDRSALLSELRTLAQGRPSDGLLRGTAPDPGTGT | |
| TPGTGPKTVFVFPGQGVQWAGTVRDLMATLPVFREH | |
| VEAAAAALDPLTGWSLVDHLTGPETLPDTPDHVQPV | |
| LFAVTTALAHTWRTLGVQPHAVLGHAAGEIAAAYSI | |
| GALTLQDAAALVVARGRAHGEQAEAVRDALLDELSG | |
| IEPRPSGTRFQSTTLGGPVDTAALDADHWYRNFRQP | |
| APFHPAVEELMDDGHTVFIEVGPHAVLPPEILELLD | |
| AAGAVGIPALGRGDGGRPRLLSSLAAAHVRGAAVDW | |
| PALYGLPAARRVELPGYAFDRRRYWPEPTPTSAPVA | |
| RQGAAAVPTPNPAAPAGAAVPASGPPVSASASVRDS | |
| DWLRGLVEAAPAGRDEQLLDLIRDEAAAVLGHSDPR | |
| EVDLARSFKDLGLESASGVELVERLGSVLQLRLPAT | |
| LLYESPTPKVLAQVLGLELKGAARRATASAASAVTA | |
| RPEKTSAVSAPASVRDSDWLRGLVEAAPAGRDEQLL | |
| DLIRDEAAAVLGHSDPREVDLARSFNDLGLESQSAE | |
| DLCERLAAVLQLSVPATLLYDSPTPRALARVLGAEL | |
| AGTTQSDATSAASVSDEPIAIVGMACRYPGAADSPE | |
| ALWQLVAEGADAIDVFPENRGWDLEGLYDPDPDAPG | |
| RTYAREGGFLYEADRFDAQFFGISPREALAVDPQQR | |
| LLLETSWEAVERAGIDPTGLAGSRTGVFVGATAMEY | |
| GPRLHETVPETAGSVGGYLLTGSTVSVASGRIAYTF | |
| GFEGPAMTVDTACSSSLVAAHLAARSLRNGECELAL | |
| AGGAAVMASPGMFVEFARQRGLAGDGRCKSFSAAAD | |
| GTSWAEGVGMLVLERLGDARRNGHRVLAVIRGSAVN | |
| QDGASNGLTAPNGPSQQRVIRQALADAGLEAADVDA | |
| VEAHGTGTALGDPIEARALLATYGQGRSEGRPLWLG | |
| SLKSNIGHTQAAAGVGGVIKMVMAMRGGVLPRTLHV | |
| DEPSPHVDWEAGEVRLLTGPVVWEAGERPRRAAVSS | |
| FGISGTNAHLILEEPPVKERTAYEAEADSADPAVWL | |
| VSAKKADALGEQAGRLAEFARTRTEVGIRRAARALA | |
| TGRTHFDHRAAVVAQDRDALAEALSALASGAGHPMV | |
| VRGRATVGRTAFLFSGQGSQRPGAGRELYASYPVFA | |
| AAVDEACAVFDPLLGRSLREVMFAGPGSEGAELLNR | |
| TAFTQPVLFVLHTALFRLLESFGVRPDHLVGHSIGE | |
| LSAAHAAGMLSLADAATLVFHRARLMQQITTPGTML | |
| ALQAGEATARGLVAGREDVVSLAAVNAPESTVLSGD | |
| PEVLADIAAQLAERGIRSRRLTVSHAFHSPHQDQIL | |
| DEFRRIAAGLTYRAPRIPIVSTLTGLLAEQDRITTA | |
| DYWTEQLRRTVRHADAVTTLHGLGTTRYLELTPTPT | |
| LATLVAETLEESPAALVPVLRHGRPEHDALLRALAT | |
| LHTSGADVAWPALPGPRSAALPELPTYAFQRERYWL | |
| TPPAPRADVTQAGLTGTPHPLLAAAVELPEGGGFVH | |
| TGRIGTLTHPWLADHAIHGTTLLPGTALLDLVLHAA | |
| SDGAGEHPAVAELALQAPLVLPGERGVDIRVTVQEA | |
| DESGLRAFAVHSRPAPAGDDASGSSSWTRHASGALG | |
| PTEAPDAADRAPQWPPADAAPVDLTDLYPALALTGY | |
| EYGPDFRLLTAAWRTDDDVFAQVELGDDAAASDDVD | |
| RFSVHPALLDASLHALLRSGLLADGVSGTDASGTLL | |
| PFSWGDVALHALGATALRVRFTRTGPTTVRVVASDP | |
| SGALILTAGELSLRPVVLDRLSDGSGTEAGGPRSLY | |
| HVEWSATPAAAPVGAAAPDAPEQWALIGRSPVPDPV | |
| STLAAEAVDIRTYPNLDALVHGTENGDPHPSVVLAD | |
| LAAHGAELPAHEGERTGEHEGGAAGAHAVARRTLAL | |
| LTSWLDAPALTVGRLVLVTHDATAAATAPDALGLPQ | |
| ATAWGLVRSAQTENPGCFTLVDIDGDPSDGYAALPA | |
| ALRTGEPQLAVRDGEVLVPRLARAAQDADVPWPAPA | |
| DVAEQVGAAQRAPLGTGQVRIAVRAAGVDLRAPVLA | |
| RDTPPDHDILGLEGAGVVTETGPGVSDLAVGDRVFG | |
| LLTGNFGPQAVAERDTLARIPAGWTFTQAASVPVAF | |
| LTAYHALVELAAVVPGERLLIHSVADGVGLAAAQLA | |
| RHRGAEVFGTAGPGEWADLRAHGLDDTHLAPSHTQE | |
| FAARFRAATGGAGVDVVLDCPAGDAVDASLRLLSSG | |
| GRFVETGRTDTPDSEAVAARHPGVDHRSFDLTKLEP | |
| AHVGAMLGELTELFERGALRPLPVAAWDVRRAQDAF | |
| RHLSRPRHVGKVVLTVPAPLDPEGTVLITGGTGALG | |
| GNVARHLVTRHGVRRLVLTGRRGPAAEGVTEIVAEL | |
| PAAGAVEVTVEACDAADRTALARVVAAVPEAHPLTA | |
| VVHAAGVLDDAPVEALTPERLDTVLRPKADAAWNLH | |
| ELTAHADLSAFVLFSSVAGVIGHAGQGNYAAANAFL | |
| DGLAAYRRHRCGLPAVAAAWGPWEHAAGGMTQALSA | |
| TDLNRMARTGVLPLSTDEGLALFDATRDAAVPAVVP | |
| VRLDLAALAESAGGAGRAGDVAEVPLLFRHVAPARP | |
| VRRLPQAAATADGARLPAPRQTAVDAGPDLARRVAE | |
| LPTEAARRGMLLELVQDSAAAVLGHATAATVDPERR | |
| FKELSFDSLTALELRNRLGAVSGLRLPGTLIFDHPT | |
| PLAVADFLYTRLASQTPRTATSDSLAVLAELDRLVD | |
| TAIATDADEATLTRFTARLEDLLVWLHGRQEAGRPD | |
| AEDAAGATDRFESASDDEIFDFIDNELGLT*″ | |
| FosK | |
| MYSRRMPIIELAEYGPDFLADPYPYYAKLREEGPVH | SEQ ID NO.: 15 |
| EVRAPDGYRFWLIVGYAEGRAALTDSRLVKARDTMA | |
| TSEASPLGKHVLIADPPDHTRLRKLISREFTVRRVD | |
| NLRPRIQELTDDLLDVMLPAGRADLVEALARPLPIA | |
| VLCELLGVPNADRDEFHSWAKGILAPQNPTETHTAV | |
| KALMSYLDDLIEDKRHGEPTGDLLSGLIRTSIENGD | |
| RLSSEEVRSTAFLLMIAGHETTANLISNGTRALLTH | |
| RDQLDLLRSDMDLLDGAVEEMLRYDGSLESTTKRFT | |
| GVPVQIGDTVIPPGETVLVSLASADRDPANFDDPDR | |
| FDIRRGTPAGVGHLAFGHGIHYCLGASLARAEGRIA | |
| FRALLERCPDLELDPEAPPFEWMPGVLVRGVQRLSL | |
| RW*″ | |
| Fos J | |
| MTSTDAVPTGTPPLTTDSSSETPPAYPMPKAPGCPM | SEQ ID NO.: 14 |
| DPPPVYRTLRAEQPVSKARLYNGREAWLISRHEDVR | |
| KILSDSRASVDALNPGFPWLSEVAKAMNTAEGGVRP | |
| LGRMDPPDHTELRRMLAPHFLIKRVRALRPATEELV | |
| DGLIDRMLEGPSPADLVPALARPVPSTVVGWLLGVP | |
| AEAMAREGETTARLEDEDGSAETAVAARGELEEQLK | |
| ELVELRRAEPDGNIVSRLVGFADEGRLTETNLLMQI | |
| GLLLGAGYDTTVKMITTGVLALLGHPEQAALLAKEP | |
| ERAAGATEELLRYLTVAEFAPKRVAVEEIEIGGQTI | |
| RPGDGIICLISSADRDESVYERPDELDIQRSARDHL | |
| AFGSGIHLCVGHSVARMELEVVYGRLFSRIPQLRLA | |
| VAPNEIPFSRGLDVQGAKSLPVTW*″ | |
| FosI | |
| MKSYKALAGIPHLPRLFLWSMLARLNVSMLPIGLTL | SEQ ID NO.: 13 |
| VLVGWSDSYVAAGVLGGALTAGQALVGPARGRAADR | |
| GAVRKLLVLTGIGYLVGLGALVTMTRTVPGGGWPIA | |
| VLVALLTGMSTVPISQVSRAVWPKIVPGELGRTLFT | |
| LEATGSEVVQTTGPLLTSLLVTALDPGYAVIACGVV | |
| AFVGALAFAAALGSAGIRGGAERTAAKPQAETGTAA | |
| ETGADAGTEGDTATSASAVTRASAPEPEERRTLFAL | |
| PKFTLAIVVTLVMMAALFSVNLSLVAWARDSHESGL | |
| SGVLIACWTIGSVVGGFGMGALRKDVPHSARFAANA | |
| VGMALLAVLLPPVTETAPVWLVLVVLFLGGTAIAPS | |
| MAGNFAQVSGAVPQERRAEAFGWLATAGTGGAALSM | |
| PVTGVLLEASGPATSVAVGAALALCATVLSYIGSKR | |
| AERNDGLVSARA*″ | |
| FosH | |
| MPESTDAAAAVPEAHDLPLTLWGWQDWTVEPWERLP | SEQ ID NO.: 12 |
| GDEGYSSHTYLVRHDGVRHVVKAVRKDMGPKLTAGL | |
| LVAQEVERHGIAAGGPLPTTGGEVTAYQGDFCYSLL | |
| TYLDGERVDETDPAHLRAVGRTLGRIDSVLLHAPVP | |
| EGVPRWNEVLELFLLEQDFLKGHDWIRRTLEQAGGA | |
| LSPDDLTIGLINCDAAAKEFRVLGDTAGLLDWSEAM | |
| YAPCMLELATTLSYLEDETDGEPLVRGYFEEGPADR | |
| AELGFLADILRFRCAAEGWIYAARQNAGDETGTTAS | |
| TWSNEKLIERARQNAENADRIAARFQVF*″ | |
| FosG″ | |
| MNTLSLLQGLPLHRSDPFSPPDGYAKVRAEAPVSPI | SEQ ID NO.: 11 |
| AFPDGNQGWLLTRHADVKAMLANPSFSSVREKAART | |
| RRTEGRPTPLPGAFFTMDPPDHTRYRRLAASRFAVR | |
| KIKALEPKIEQYTREHLDRMEETGGGPVDLVTAYAL | |
| PIPSLIICDLLGVPYDARDDFQRWSLSILDTELSEE | |
| EQQRTVLEGTKFMLDLIEDKKKNPSDDLISDLLDPA | |
| EEKDRISEFEIAGMCGLMLMAGHESTSNMLSLGTLA | |
| ALRNPDQLALLRSDPSLIDTAVEELLRYLSIVQFNF | |
| ARLATEDVEIGGQLIKACETVVGSMAAANHDPEVYT | |
| DPHRLRLDRAEERNLAFGHGIHLCIGHQLARVEMKV | |
| TYLRLFERFPTLRLAVPFEDIEFRANSVVYGVNSLP | |
| VAWDAPADHDPAP*″ | |
| FosF(module 8 and thioesterase) | |
| MSTNEDKLRHYVKELTGDLLRTRGRLRELEAAGNEP | SEQ ID NO.: 5 |
| IALVGMACKYPGGVASPEDLWRLVAEGRDAISPFPA | |
| DRGWDLGRLPAAGGGFLHDAAEFDAGFFGISPRDAA | |
| AMDPQQRIALETCWEAVERSGISADSLRGKPVGVFM | |
| GGAVQGYGLVGTEIVDAPEGVGGTGSASSVISGRVS | |
| YSFGFEGPAVTVDTACSSSLVALHLAVQSLRAGECS | |
| LALAGGVTVMATPYAFVEFGRQGGLSADGRCRSFSA | |
| DAEGTGWSEGVGVVVLERLSDARRNGHEVLAVVRGS | |
| AVNQDGASNGLTAPNGPSQQRVIVRALAGAGLSTSD | |
| VDVMEAHGTGTRLGDPIEAQALIATYGQGRAEGRPL | |
| WLGSLKSNIGHTQAAAGVGGVIKMVMAMRHGVLPRT | |
| LHVSQPSPHVDWSAGAVELLTRARQWPQTGRARRAG | |
| VSSFGISGTNAHVILEHEPVESTEAPVGSAQVPVES | |
| TQALVVAGELPWVVSGRTEGAVRAQAARLAAFVAGR | |
| GGGALDVGGVGLALVSSRSVFDHSAVVSGGSLDELL | |
| AGVGGVARGDGSAAGGVVFERRVAGGVGVAFSGQGS | |
| QRPGMGRELYGRFPVFAAALDEVCAEVEAQTGAELL | |
| GVVFGDDAGVLEDTGVAQPALFAVEVALYRLAESFG | |
| VRADVLIGHSLGELSAAYVAGVWSLADAVRVVVARA | |
| RLMGSLPSGGRMVAVEATEEEVAPLVADVAAAGGMV | |
| SLAAVNAPGAVVVSGQDAAVDQIADIFAGRGRRTRA | |
| LAVSHAFHSPLMEPMLAEFADVLAQVEFRAPSIPVV | |
| SNVTGTIADAEELCSPEYWVRHVREAVRFGDGVGAV | |
| LAQGVATVVELGPDPVLTALGERVRAASAERDSAAR | |
| DVAFVPTLSRRSTDTRAFLGMLARVHARGHQVDWTA | |
| LGRADDLARELPTYAFQHEHHWLKGASVRPGSAASR | |
| TAGSDGAFWKVVQEQDLQRLASDLGVDPDAPLHTVL | |
| PALGDWHQTHIEASETDGWRYRVAWERPTAQHAPEG | |
| PATLHGTWLIVVPEGDLRAGHLLDNDGLHDGLHGEV | |
| RRVLTDAGAEVKSLSLAPEDIDRQTIAKLLNGLDDT | |
| PAGVVSLLALSGREHTGPRGVGSGAWASVCLLQALL | |
| DTGWSATRLWTLTRGAVRATASDDAPDPWQAQVWGL | |
| GRVAALEHPTLWGGLVDLPAPDLSAADGHALAATAE | |
| ASFGLAALLAGSSGEDQVALRADCARVRRLRPAGPD | |
| GAPEPVRPVAPESLVAPEGADATGRTGDPQPPAARE | |
| PWWSHGSVLITGGTGALGAHTARRLAEQGAPHLVLA | |
| SRQGPDAPGAADLRAELAAHGATVDLVSCDVTSRDE | |
| VAALAADLAGRGAPVGAVVHTAGVAAEHPLADLDAT | |
| EFAAVVDAKVTGAVILDEVLGDGLAAFVVYSSIAGT | |
| WGSTRGGAYAAGNAFLDALVERRRARRAAATTLAWG | |
| PWSGGGMAGEEFRQEMQRRGLRPLTPRLATTALDRA | |
| VRQEDTAIVVADLDWPRFIGVFTAGRPNHLFADFDD | |
| TESGAGHPDAGRTGAAQPGEWQRLPDLPLADQRPYV | |
| LDIVRREAARVLGHADAGTITEDQEFLALGFDSLAA | |
| VELRGRLTVLTGLALPSSLVFDHPTLGALVTHLLDN | |
| AAPGGDAGASPAPGVSAAPSASVAAAPPQDSNDSVV | |
| GIYRKLSLQGRMQEVEAFLSSASALRTRFHGAEDLG | |
| RGAHVTTLGHGEAEPQLVCFPPFAPVDGSLQFARLA | |
| NHFRGRRRVSVVTVPGFMAGEPLAASLEVLIETLAE | |
| AVLRAADGRPYALLGYSSSGWLAQAAATWLEERGTG | |
| PVGVVLLDTYPPDSMTLEMRKANTYEVVERRMRFTS | |
| MHYDGLTALGTYRGMFRGWQPRQLAVPTLFVRPDSC | |
| IPGSPEEPMAGPDWQAAWPLDHEETQVPGDHCTMIG | |
| EFSETTAAAVDEWLSRTPGLTRP*″ | |
| EasE (modules 6-7) | |
| MASENQLLDYLKRVSAELHETRGRLQDVEDAAREPI | SEQ ID NO.: 4 |
| AVVGMACKYPGGVASPEDLWELVAEGRDAISPEPAD | |
| RGWDLDGVGAGPASGGVAGDGSTSAPDGLLTAGGGF | |
| LHDAAEFDAGFFGISPREAAAMDPQQRLLLETSWEA | |
| IERTGIVPESLRGGDTGVFIGAGMQDYLGHLRESRA | |
| TEASGFLITGNASSVLSGRLSYSFGFEGPAVTVDTA | |
| CSSSLVALHLAVQSLRAGECSLALAGGVTVMATPYA | |
| EVEFGRQGGLSADGRCRSFSADAEGTGWSEGVGVVV | |
| LERLSDARRNGHEVLAVVRGSAVNQDGASNGLTAPN | |
| GPSQQRVIVRALAGAGLSTSDVDVMEAHGTGTRLGD | |
| PIEAQALIATYGQGRAEGRPLWLGSLKSNIGHTQAA | |
| AGVGGVIKMVMAMRHGVLPRTLHVSQPSPHVDWSAG | |
| AVELLTRARQWPETGRARRAGVSSEGISGTNAHVIL | |
| EHEPVESTEAPVGSAQVPVGSVQARVESTEAPLVAG | |
| ELPWVVSGRTEGAVRAQAARLAAEVAGRGGGAGALD | |
| VGGVGLALVSSRSVFDHSAVVSGGSLDELLAGVGGV | |
| ARGDGSAAGGVVLERRVAGGVGVAFSGQGSQRPGMG | |
| RELYGRFPVFAAALDEVCAEVEAQTGAELLSVVFGD | |
| DAGVLEDTGVAQPALFAVEVALYRLAESFGVRADVL | |
| IGHSLGELSAAYVAGVWSLADAVRVVVARGRLMGSL | |
| PSGGRMVAVEATEEEVSGWLVDGAVIAAVNGPAAVV | |
| VSGVEGAVEAVVERARGGGRRATRLRVSHAFHSPLM | |
| EPMLAGFAQVLARVEFRAPSIPVVSNVTGEVASAAE | |
| LCSPEYWVRHVREAVRFGDGVGAVLAQGVATVVELG | |
| PEAVLTAMGASHPGVVENGAVFLPTLGRRTGDVNGF | |
| LTALARVHARGHQVDWTALGRANDLARELPTYAFQH | |
| EHHWLAGNAASVDAAHLGMRAVDHPFLGAAVTLPGT | |
| GGTVLTGTISPGTHPWLGDHVVLGSTLLPGTAFLEL | |
| AFTAAARVGCAGVEELTLEAPLILDGGSAHVVQVLV | |
| GEAEAAGGGRAITVHSRPVHAAEDAPWTRHATGTLR | |
| SDAAEPAPVLAPEPSQVQAWPPRGATPVDLDAVYER | |
| LIGLGFDYGPAFRGLHTAWRDGDTVYAEVRLPTRQT | |
| DDAGRFSIHPALLDTALHSLALPDLLSGQDECHLPF | |
| SWSGVTLHATAVSSARVRIRRLGEGATSVELLDEAG | |
| AALATVRSLALRPVTLEQLRSARVSSVESLYGIAWS | |
| PLESAGSAVSAAVSASGTGLALLDLGADWSVESVPA | |
| RRHADLAALAAAIDADPAEAPRDVLIALPALGGVDG | |
| DIAAATHQRTQTVLHLLQDWLKQPRFASVTATLLTR | |
| GALAVDAAEAAAVDLASAAVGGLIRSAQLEHPGRFR | |
| LIDTDGEEASLRALPTLLGTGNGTDIGTGTGVVAEP | |
| QIALRAGVPSVPRLRAIAAPETTDADGSADAPAFSG | |
| EGTVLITGGTGDLGSLFARHLVTVHGVRHLLLTSRR | |
| GPDAPGAAELIAELEALGADVTLAACDMSDRSAVAE | |
| LLAGVPGDHPLTGVVHTAGVLDDGLLESLTPAQLTK | |
| VLRAKADAALHLHELTSRAPLSAFVLFSSVAGVFGG | |
| AGQANYAAANAFLDALARRRRALGLPGVSLAWGLWS | |
| TEGGMTAELDRANVARLKRTGLLEISREQGVTLFDA | |
| ALAAAGAAFGASEAGPETVGTHADGLLVPARLNAPV | |
| LDEQAAAGSLPAVFQAVVSTRPRPSSRVAGGTAATA | |
| GSASAPLLAELRVADREERLQILGGLVAEKVAYVLG | |
| HADREAVDRAQPFNRLGLDSLTAVELRNQLGAATGV | |
| RLPATLVFDHPTPLAVAEELYDELARGVLGEPAGST | |
| ALAVAEPAARASASHPQDAGDDPIVIVGMACKYPGG | |
| VASPEDLWRLVAEGRDAISPEPADRGWDLDGIYDPD | |
| PQQPGKTYTREGGFLHDAAQFDAEFFGISPREATAT | |
| DPQQRLLLETSWEALESAGTRPETLTGSRTGVEMGV | |
| MYNDYGARHLNRSPQGYEGYISNGSSGSIASGRVSY | |
| SFGFEGPAVTVDTACSSSLVANHLAAQSLRAGECSM | |
| ALAGGVTVMATPYAFVEFGRHGGLAVDGRCRSFSAD | |
| ASGTGWSEGVGVVVLERLSDARRNGHEVLAVVRGSA | |
| VNQDGASNGLTAPNGPSQQRVIRQALAGAGLSVADV | |
| DAVEAHGTGTRLGDPIEAQALIATYGQGRAEGRPLW | |
| LGSLKSNIGHTQAAAGVGGVIKMVMAMRHGVLPRTL | |
| HVSQPSPHVDWSAGAVELLTRARQWPETGRARRAGV | |
| SSFGISGTNAHVILEHEPVESTEAPVGSAQVPVGSV | |
| QARVESTEAPLVAGELPWVVSGRTEGAVRAQAARLA | |
| AFVAGRGGGAGALDVGGVGLALVSSRSVFDHSAVVS | |
| GGSLDELLAGVGGVARGDGSAAGGVVLERRVAGGVG | |
| VAFSGQGSQRPGMGRELYGRPPVFAAALDEVCAEVE | |
| AQTGAELLSVVPGDDAGVLEDTGVAQPALFAVEVAL | |
| YRLAESFGVRADVLIGHSLGELSAAYVAGVWSLADA | |
| VRVVVARGRLMGSLPSGGRMVAVEATEEEVSGWLVD | |
| GAVIAAVNGPAAVVVSGVEGAVEAVVERARGGGRRA | |
| TRLRVSHAFHSPLMEPNLAGFAQVLARVEFRAPSIP | |
| VVSNVTGEVASAAELCSPEYWVRHVREAVRFGDGVG | |
| AVLAQGVATVVELGPEAVLTAMGASHPGVVENGAVF | |
| LPTLGRRTGDVNGFLTALARVHARGHQVDWTALGRA | |
| NDLARELPTYAFQHQRYWLDAPAPEPGVVEHVPEQA | |
| VLLNAVAQQDVDGLAHTLGLAPDAPLTAVLPALQTW | |
| SREQARLAAADALRYRVSWTELPSPTDAVPLDGTWL | |
| VAVPGEPVEPDLVIAVEKALVDAGARVERCETAELR | |
| ARLAAVAPRGVLCLPAVGAQRDRDRERGIATGALAV | |
| LDLLHTVQDAGVDTRVWALTCDAVCAQADDAAPDPW | |
| QAQVWGLGRVAALEHPTLWGGLTDVSGTSPAAQLTG | |
| LAAALANTTGDDQIALRGELLLGRRLIRGTVPTSVP | |
| EPETGSTAPWTDGSVLITGGTGALGAQTARWLARNG | |
| ARTLVLTSRQGPAAPAVAALRTELEERGADVVVESC | |
| DVTDAVALAALRDRLADAGTPVSTVVHTAGVASELP | |
| LAELDEDGYAAVVRAKVVGAQVLDEVLGDGLAAFVV | |
| YSSIAGVWGSARAGAYAAGNAHLDALIERRRAQGRP | |
| GTALAWGPWGGGGMADERLTREMQARGVSALDPEEA | |
| VAAFGRVVRADYGTVVLADTDWSRLADIFTVNRPSP | |
| LFDPLRTVETERGGVGADGTAAGTAGADDVSGPGDS | |
| DAGTAGATPFVARWTALSGGERRRVLVETVCTQAAA | |
| ELGHASGGTIEPERPFQELCFDSLAAVGLRQRLEKL | |
| TGLKLPATLVFDHPTPAALAQVVASALAERVGGASG | |
| ASAVLGELDRLEAALAALDAGSDPAARGRITLRLSN | |
| LLTRFQNADDEPTAASGAAETAAEQLDSATDDQLFD | |
| LIEKEFGIS*″ | |
| FosD″ (module 5) | |
| MSDDVSQEQLLQALRKVTGDLQDSRRRLKDTERRAT | SEQ ID NO.: 3 |
| EPIAIVGMGCRFPGGSDSPEALWDLVRTGRDAIGAF | |
| PKDRDWDLDALFAGDGTEEGTSYVREGGFLHGAAWF | |
| DAGFTGVSPREAVAMDPQQRLMLEVAWESLERAGIA | |
| PDTLRESETGVFTGAYYSFYDVPLQASSEEYEGYLV | |
| TGNNSAVVSGRVSYALGLSGPAVTIDTACSSSLVAL | |
| HLAAQSLRRGECSLALAGGVTVMPTPITFTEFSRQR | |
| GLAADGRSKSFADAADGTSWGEGAGMLVLERLSDAQ | |
| RNGHRVLAVVRGSAVNQDGASNGLAAPSGPAQERVI | |
| WRALQDAQVQAADVDVVEAHGTGTTLGDPIEARALL | |
| ATYGREHPAERPLWLGSLKSNLGHTQGAAGVGGIIK | |
| MVMAMRDGVLPRTLHVDRPATHVDWSAGAVRLLTEA | |
| QPWPRTDRPRRAGISSFGISGTNAHVILEQAPDPEP | |
| DVPAPTAKGTGTPAQGSGVLPFLVSARTPDALSAQA | |
| RLICRRLTADESPLADVALSLATTRTAFEQRAVVLG | |
| GDRATVVAELESLAAGVPSANRVDGDGAAAPRRRVV | |
| EVFPGQGAQWAGMARELLHSSPVFAARVRECADALA | |
| PHVDWSLLDVLNGEPGEPSLDRVDVVQPVLFAMMVS | |
| LAELWRSVGVRPHTVVGHSQGEVAAACVAGALSLED | |
| AAKIVALRSRTLTGIAGAGGMLSVALGRAETAARLA | |
| PFEGRVFLAAANGPEATVVSGDADALDEFFTVCEAD | |
| NVRVRRIAVDYASHSPHVDRTGPRILTELADVAPGR | |
| PEVEMLSTVTGEPVRDGELTAEYWVRNLRAPVEFEA | |
| AVRRLAESGDAVFVEISPHPLLTGAMERTAEEAGVR | |
| PATVPTLRRDAGGADRFLTSAAEGWVHGLDVDWAAL | |
| LRDSDGRVTDLPTYPFQRSRFWPTPRTTNGSGAGSE | |
| ACRGGRPSALAGASAPHAAQAADSGLWEALEADPAA | |
| LLDALGLKDGESADALLPALAAWRRDHLEQARLGSW | |
| RYGVRWEPVLHAPGAVPALAGTWLLAIPGAEHGFGP | |
| DHRLVGWVLDAVEAAGGRALLVELDPREDTDPAAWA | |
| ASLHAAAADARPAGILSLLALDDRPLPGRPAVGRGL | |
| AGTTLLVRALAQTNLPGRLWCLTHGAVGTDPTDPPA | |
| APHAAAVWGLGRVAALEHPTLWGGLIDLPRAPREAD | |
| GQDGRTAPDAATAAQLCAVLADPGDEDQLALRPTGV | |
| LARRLVRTPFRLPDPTTGGRDGGQVSLSDTDTDTDT | |
| DGNVGADAVWPREGTVLITGGTGALGSRLARRLAAQ | |
| GAQHLLLTSRRGPEALDADGLGADLESMGARVTIAA | |
| CDLADREAVRALLESVPADAPLVSVVHAAAVLDDAA | |
| LENLDADRLDAVLGPKALGAWHLHELTAGLNLSSFV | |
| LYSGFVGTVGGIGQGNYAAANAVLDALAEHRRAAGL | |
| PALSIAWGPWGGGGLVDAATEQRMRRNCLPPIEPEL | |
| GVRALDLALAQDGPASQVLAEIEWPLFADGFTAARP | |
| SPLLAAFARSGEAADDGSDAGRSDGAGGAGGPTLAR | |
| RLPGMARTDADRLLLDTVRDHAAAVLCYADPAALAV | |
| DQPFKELGIDSMTAMALRNRLQRAAGVDLPATMVFD | |
| HPTPKALAAQLHGLIQPEERAVTAESALAELYRVAQ | |
| SLEELRLDAALRKAAASRLRALADGWEGKRSASETV | |
| MFSSPSGSSSERGTPDTGALLESATEDEVLDFVTRQ | |
| LGISPLEGPG*″ | |
| FosC (modules 3-4) | |
| MPGVLGGCLSRGRRCFRAWIEEIRFGTAAAVRAALR | SEQ ID NO.: 2 |
| AETKTAGFDPVGGLVQFIRCRVREASEPAVRRWARN | |
| MTDEQQLVEYLRKVTADLQRTRSRLRDAEAAAREPI | |
| AIVGMSCRYPGGVTSPEELWELVAEGRDAITGFPTD | |
| RGWDVAGLYDPDPDATGRSYTREGGFLGDIDLFDAD | |
| FFGLSPREAFSMDPQQRLLLETTWRALEDAGIPPAT | |
| LRGSRTGVFTGVMHHDYGGRFATAAPDGYEGYIHSG | |
| SAGSMAVGRVSYLYGFEGPAVTVDTACSSSLVSLHL | |
| AAQALRSGECDLALAGGVAVMSSPLFFIEYSRQRVV | |
| STDGRCRSFADDGDGTGWSEGVGVLALERLSDARRL | |
| GHRVLAVVRGSAVNQDGASNGLTAPSGPAQQRLIET | |
| ALSSAGLSADEVDVVEAHGTGTRLGDPIEAQALMAT | |
| YGRSRRAGRPLYLGSLKSNLGHTQAAAGVAGVIKMV | |
| QANRHGVMPSSLYAENPTTEVDWSAGTVRLLAQARE | |
| WDAPAGPRRAGISSFGMSGTNAHVILEEFVSEDAGA | |
| TADAQRLAGQGSAANTRRSVDQDSAANGQDWARVAV | |
| PVVVSGKTPEAARAQATALRDHLAVHPGLPIVDAAR | |
| NLVDHRSLFAHRVAVAGDRAGLLAGLADTAPVEAGA | |
| GRVAALFTGQGAQRPGMGRELDAHFPAFAAALDEVC | |
| ACVDPLLGRGLREVMWESDAQTVARTEFAQPALFAF | |
| EFALARLWQSWGVTFTALAGHSVGEIAAAVVAEVLT | |
| LQDAARLVVARGRLMQALPEGGAMLAIAASETEAAE | |
| TLATVTDGPHAADGAVDIAAVNGPDAVVVSGALDAV | |
| ERVGEIWRGRGRRTTRLRVSHAFHSALMEPMLDDFA | |
| EVVRGLTFSIPKTAIAASAASDHPFHSADYWIDHAR | |
| NAVRFHDAVRGLAAADVLVEIGPDAVLAPMVGAAQA | |
| HGDDGRTVLASTRRDRSETHTVLTTLAQAYAHGVRV | |
| DWAALLPAAPLVDLPPYAFQRQSYWLAPPRATAGAG | |
| TDALAHPLLSGAVELPGPGGLVLTGHISPSADPWLA | |
| DHAIAGSVLFPGAGFVELAAQAARQAGCREISELVI | |
| QAPLVPGDGGADVQVWVGPSEGTAGRELTIRARQAT | |
| GQVPGQWTVHVTGTLTEPLPERHEPHEPERSDVDWA | |
| VGAWPPAGAEPVPVQDLYADLADRGYGYGPAFRGLR | |
| AVWRAGDDLFSEVALPDGRYDGRFGIHPALLDAALH | |
| PLALTGDESRRIRLPFAFNGATVWPSRGSATADGAP | |
| IRVRLRTAGDSARLDAVTSDGEPVVTVRELVLREAD | |
| PARLSAQADAALDRLRYEVTWQPADLAPRTVPVPGT | |
| WLVVPPAAADGASDIPAWVGELVEHTVTVPPHEAAD | |
| RATLASALCALSAQPADAIADGFAGVLCFADRPETL | |
| LTALQALTDARVTAKVWCLTRDSGTDPDAAAVWGAG | |
| RAAALELPDTWGGLIDLPADGAEPPLGQLAALLAGD | |
| TREDQVRVAGDGLEVRRVVRSAPDNDPAPNQDLTHW | |
| QPSGTVLITGGTGALGGHVARWVAGLAAGSATQQVA | |
| DGCSLLLVSRRGPEAPGAAELLEELSATGVPVRVVA | |
| ADTADRAAMAAVVQEAAASGAPIRAVFHAAGIAHEA | |
| PLLETDADDFRAVLDGKTRGALVLDDVLADAGLDAF | |
| VLFSSVSGIWGAAGQAGYGAGNAALDALAARRRAQG | |
| RAATSLAFGPWACGGMVDATREQRFRRSGLIPLPAA | |
| DAVTALARSLALGTDCVLADVAWSRFLPLFTAVRPA | |
| PLFTDLVRARGAASSGAAPRAAAGAGPARSAYRGTD | |
| LLDLVRAEIAAAVCHADAGRIDADRPLGELGFDSLM | |
| SVQLRNRLSAETGVQLPATLVFDHPTSAALARHLEI | |
| RLAPPTGTGDTVMPDRKPDFTTGRTERDEPIAIVGM | |
| ACRYPGGVSAPDDLWRLLAEGRDAIAPFPADRGWDL | |
| TRIYSPDPAATGTTYARGGGELDDPAGFDAAFFGIP | |
| PREALAMDPQQRLLLEAAWESVEHAGIDPKTLRGSR | |
| TGVFAGVMYNDYFSRLNGTPESLEGITGIANSNSVM | |
| SGRVSYLLGLEGPAVTLDTACSSSLVALHLACQALR | |
| AGECDLALAGGATVMASPHLFVEFARQGGLAADGRC | |
| KSFSDDADGTGWAEGVGVLAVERLSDARRLGHDVLA | |
| VVRGSAVNQDGASNGLTAPNGPAQQRVIQAALEQAR | |
| VAAADVDAVEAHGTGTRLGDPIEAQAVLAEYGARRP | |
| AGRPVYLGSLKSNIGHAQAAAGVGGVIKMVQALRHE | |
| LLPRTLHAEKPSSDVDWSAGALELLTEERPWPRTER | |
| PRLAAVSSFGISGTNAHVVLEEGDARHVPQSPEAGT | |
| DDDAPVAVAIPVNARTRDGLHAQARALHGHLVANPG | |
| LGLRDVAYSLAATRSDFDHRAVVVAEDRDGLLGALA | |
| ALGTPDAVHPAEAVTGPEAGTTGGTRAAALFSGQGA | |
| QRPGMGRELAEHFPVFAAALDETCDVIDPLLGRPLR | |
| DVLWQEPAEVLERTEFAQPALFAFELAMARLWMSWG | |
| VEFSALAGHSVGEIAAAVVAGVLSVEDAARLVVARG | |
| RLMQALPEGGAMVAIQATDDEVAASLAYLVDTSEAE | |
| IAAVNGPSAVVVSGAEDAVTRIAEHWRGEGRRTTRL | |
| RVSHAFHSPLMEPMLAQFAAVVDGLTFHPAEIQVSP | |
| AADTTHAFDTPEYWTEHARRAVRFADAVRGLPDADV | |
| LIEIGPDAALTPLFEGTRPVAASSRRGRPEAATVLT | |
| ALSRTYVNGVPVDWTALTPGARRVALPTYAFRHRRY | |
| WLDAAPDTADVPAAPDGTHSAEDTAFWQVVEEQNLD | |
| GLAPTLGLGADAPLSAVLPALGNWRRGQDMTARTDR | |
| WRYHVTWERLPDAAPPAAALDGTWLIVAPESDADTA | |
| EDGASAALADAVTAALADAGATTRLLTVDVATADRA | |
| TLAKELARELDRTPASDERFAGVLALPWPSGETARR | |
| DGVSAAADATLLLVQALLDTGRTDPPLWTLTRGAVR | |
| ALESDAAPDPWQAQVWGLGRVVGLEHPGLWGGLIDL | |
| AASTDDDPLGCGGLARLAAVLACRSGGDDQLALRDD | |
| SVHARRLTRAEPTVSPHERPAEDTEDADLWSSGPVL | |
| ITGTGALAVHTARRLAESGAPIVVLASRRASGFADA | |
| DRLRAELARHGTELVLADCDVSRRADVEELAARFAA | |
| ADTPVGAVVHAAGTGDQNPVAALDADTFATVMAAKV | |
| TGARNLDEVFGDGLSAFVAFTSVAGVWGNASGGAYA | |
| ASNAHVDALMERRRARGAAGKAIAWGPWDGGGMAAG | |
| SFGDDLRRLGIAPMAPELAVTALDRALRHGDTTVTV | |
| ADVDWGRFTAVFTAARPSPLLAGLVPVPGAASDLAG | |
| VTGAGPGTDETGGTTPDFAKVLAGHSPDEQQRVALD | |
| LVRAQIAAVLGHAAARDVAPHHAFKDLGFDSLMAVE | |
| LRTRLRESTGLDLPATLVYEHPTPTDVAAFLCRQAV | |
| PDGGGQAARALADLASLERSLADLEGSAADRDRLAT | |
| RLGALAARLRETPRDTEPAAQAADDDTDGGAAIATA | |
| SASDLLALIEKGFE*″ | |
| ORF4 | |
| MATEPMKEDISNMPISKADETADAPRTADAALRAEA | SEQ ID NO.: 10 |
| ARTAQAGPMAGKKNVVVLGVTSALGRRLAESILARG | |
| LTPVGLIGEAGHSESLRAAGTEPLVIASEADRTSPA | |
| ALRAMGECGGIAVATGTGWGPDATDTSSTSLVGELM | |
| GVAQLVGIRRFVLVSAYLPDDELHTRLGDELESYLA | |
| EKRDVERKLAGHDLDWCVVRPGMLDNSPATGLVTVR | |
| GGADPQPEGTVSRTDLAETICEALFAPEPVRGVLAV | |
| SAGAQPIRAALGSVPQATVQPAWWCAEERAEGAAEK | |
| RADSPAS*″ | |
| ORF3 | |
| MTRPGGGNPVFRSSCGPKPSSGSESNCAVSLVDRDG | SEQ ID NO.: 9 |
| ELAELGRWLRRRPADSAGLCVIEGPALCGKTALLEA | |
| ACAEARRMGIGVLTVRGSVCGVGYAEELGEFGAREP | |
| GEQARRLVAAATRSLLLAVDETDLLAPESQVFVREV | |
| VRLTHTGRIRVLLTGVSAATVPSIVDELRELAAGWA | |
| ERVRVRPLGRAAVAELARARVPGPRADALAEAIHPI | |
| TGGNPGLTLAMLDEAAAAIGRDPDAPDGPPSAGLAR | |
| RLREHTSREVMNLVEAVAVLDAQCTPERLWNVLEKD | |
| AEYVSHVAGQAIASGIVRPDMTLHPYLRDAVLWSTH | |
| PAGLRSLRLRAAQVLHEQGVPAWEIAPYLHEAGIAP | |
| QPWAGGVLYEAAEHASRLGRHRLAVQLLESASFWGS | |
| GPDLREEAGVRITDMVWWMDPALTAPRLRRLAAAAS | |
| DGTFKPGSLARLSRHLARHGEMAQASDMLRLAEPAP | |
| GRGNAVGAVSPGDALGAADVRAEVQRTLSGLWLRHL | |
| FPDACPDAPAPDDQPHPQLIAEGFSWLSSAQRLSWL | |
| LAPGVQCSDAPNSGPRPTGSADSAGSAKAVKDAEAA | |
| VTCAEEILQSANFDYSSLEPMLVALFCLMATGRLDP | |
| RAPWYAALADNAALADNERMPLPLASMLSVAEAVAA | |
| WWKGDLVAAQTAVRRGFERWDADRWGVLAGLPRGLS | |
| ALMLTEQGRFEEAAAQLARPMTEADLASPYGLVYLR | |
| ARGRFRLATGALRAAVADERSCGAILTAWGMEMPGL | |
| VPWRLDLAEALLKLEEYAEAAELIDEQLAFSPDSNT | |
| RARGIGLRLRVATVPPGERALACRKSIAVLQSCGDE | |
| LEYARALGQLAQIYKHTGDLAGGRKTMRLAEKTARG | |
| CGAQWALAEMSGGSQARRKVPPPAARRDGRSLTPAE | |
| WNVARMAAQGYTNREIAESLYVTASTIEQHLTRIYR | |
| KFDIRRRAEIGAIVESRCAPDHTPAEIQDVA*″ | |
| ORF2 | |
| MQLIKHAHACVSLVKDGGRIAIDPGTFTPDAKEVVA | SEQ ID NO.: 8 |
| AADAVLITHDHFDHFDEDLIARALDARPGLRVYGPV | |
| RVVGRWAARRGQVVAVADGDRLDVAGFDVAVSGDLH | |
| AAIHRDIPRVTNVGYLVDGRVHHPGDAYHVPPAPVD | |
| TLLLPTSGPWTQLGRAADYVREVAPNRLVQIHEIML | |
| SRTGQESMARFLSPAALTEVPLTIVPEGEAITV″ | |
| ORF1 | |
| MNDAPSNEPAPLPDELAVRLRAVVGTLVRSARTVDR | SEQ ID NO.: 20 |
| LASVPAAVLGLLDTRGPMTTADLAATRGVRHQTMAA | |
| TVRELTEAGFLASRTDPGDARRKVLALTKAGKKALD | |
| TDRRQRVGVLADALEETLDDEDRRALAHALDLIDRI | |
| SGSIRGGHSFSGEREFNTGAW*″ | |

All publications and patent documents cited herein are incorporated herein by reference as if each such publication or document was specifically and individually indicated to be incorporated herein by reference.

Although the present invention has been described in detail with reference to specific embodiments, those of skill in the art will recognize that modifications and improvements are within the scope and spirit of the invention. Citation of publications and patent documents is not intended as an admission that any such document is pertinent prior art, nor does it constitute any admission as to the contents or date of the same. The invention having now been described by way of written description, those of skill in the art will recognize that the invention can be practiced in a variety of embodiments and that the foregoing description are for purposes of illustration and not limitation of the following claims.

1. A method of producing a polyketide, comprising culturing a cell under conditions under which the cell produces the polyketide, wherein said cell comprises a recombinant polynucleotide synthase that comprises at least one domain from the Streptomyces pulveraceus fostriecin polyketide synthase, and wherein said cell does not make the polyketide in the absence of said recombinant polynucleotide.

2. The method of claim 1 wherein the domain is encoded by a subsequence of SEQ ID NO:1 or a sequence that hybridizes under stringent conditions to a subsequence of SEQ ID NO:1.

3. The method of claim 1 wherein the cell is not Streptomyces pulveraceus.

4. The method of claim 3 wherein the polyketide is fostriecin, PD 113,270 or PD 113, 271.

5. The method of claim 4 further comprising recovering said polyketide from the cell.

6. A recombinant DNA molecule comprising a sequence encoding at least one domain of fostriecin polyketide synthase polypeptide.

7. The recombinant DNA molecule of claim 6 that encodes one or more modules of fostriecin polyketide synthase.

8. The recombinant DNA molecule of claim 6 that encodes a chimeric polyketide synthase (PKS) module composed of at least a portion of fostriecin PKS and at least a portion of a second PKS for a polyketide other than fostriecin.

9. The recombinant DNA molecule of claim 6 comprising a sequence encoding an open reading frame encoding a polypeptide encoded by fosA, fosB, fosC, fosD, fosE or fosF or encoding a conservative variant of such a polypeptide.

10. The recombinant DNA molecule of claim 6 that encodes a modified fostriecin polyketide synthase polypeptide that differs from the fostriecin polyketide synthase polypeptide encoded in SEQ ID NO:1 by inactivation of at least one fostriecin PKS domain.

11. A recombinant DNA expression vector comprising the DNA molecule of claim 6 operably linked to a promoter.

12. A host cell comprising the DNA molecule of claim 6.

13. A recombinant Streptomyces pulveraceus cell in which at least one domain-encoding region of an endogenous fostriecin polyketide synthase gene is deleted or otherwise inactivated.

14. The cell of claim 13 wherein said domain has been replaced by a different PKS domain.

15. A recombinant Streptomyces pulveraceus cell in which at least polypeptide-encoding ORF of the fostriecin polyketide synthase gene cluster is deleted or otherwise inactivated.

16. A method of producing a polyketide, which method comprises growing the recombinant host cell of claim 12 under conditions whereby a polyketide synthesized by a PKS comprising a protein encoded by said recombinant DNA molecule is produced in the cell and recovering the synthesized polyketide.

17. The method of claim 16 further comprising chemically modifying said recovered polyketide.

18. The method of claim 17 further comprising formulating said polyketide for administration to a mammal.

19. A method of producing a polyketide, which method comprises growing the recombinant host cell of claim 14 under conditions whereby a polyketide is produced in the cell and recovering the synthesized polyketide.

20. A method of producing a polyketide comprising
(a) recombinantly modifying a gene in the fostriecin PKS gene cluster of a cell comprising said gene cluster to produce a recombinant cell, or obtaining a progeny of said recombinant cell;
(b) growing said recombinant cell comprising a DNA encoding a modified or progeny under conditions whereby a polyketide other than fostriecin is synthesized by the cell and,
(c) recovering the synthesized polyketide.

21. The method of claim 20 wherein said modifying comprises:
(a) substitution of a fostriecin AT domain with an AT domain having a different specificity;
(b) inactivation of a domain of a fostriecin polyketide synthase module, wherein said domain is selected from the group consisting of a KS domain, an AT domain, an ACP domain, a KR domain, a DH domain, and an ER domain; or,
(c) substitution of KS domain, an ACP domain, a KR domain, a DH domain, or an ER domain with a domain having a different specificity.


SEQUENCE LISTING (36 sequences)

SEQ ID NO: 1 - DNA, 73984 bp, Streptomyces pulveraceus
  misc_feature (29680)..(29682): n is a, c, g, or t
aggccgacgggcagtgcccgcggcgtatcggtggccgcccacgcgcgggcggggaacgcaccgacggcccccgcggcgagcgcgcccacggccagcgccgatcctgcgccgaggaagccgcgccggctgaaggcgggatgggcgcggggggtgggcgagtcggggccgtgaggggtgggcgagtcggggccgtggggggtggtctgactggccaacggagtactccttcacacacgtcgggcgaagcaggacggctcagctgtacggcggacacggacgggagcgaccggtcccgaagcgggattgaaacgtttcaataagctggtgcggtcgtaccgtacgagagtcaccaaggggggtcaaggggttcggcacaccgtttgggacggccccacgcccgcctcgcgcgcacctcggcgcactcgccacgcacactcggcctcgaacacccggcgaggaagcccgtgcgatcggcaggatccggcgacaccggctgcggagcgcggtgacgcacactgggaaaggcaccccatcgaggtgctccgagatcgtcgagggaaccgtaggcggccgggatcctgcggcacggaccgggagggccatgacaccaggtacgacaccaggtatacccggacgacggtcgggcgcactgcgtcggcatgccgtgcgcctcctggcgaagccgagacgccgccccgcaccggcccctcccccgcgctccgcccagctgcgggcgctcaccgccctgctggacgaggcggtggcggcgcaggcaccggcggaccggacggtggcggcctgcggcgaaccgggccccctcgccgggcagaccgcccgggaagccggacggcagtacagggtcctgcaccggctgcacgcgcgcgtgcgcgatctgccgctgacggaggccgatctcgtccgggcccaggagtacgcgggacgcctgctctcctacggccagtggatgatgcgcgaggccatggacctcgccttcccctcgaacccgcgtccgagcgtcgaggcggcccggctccacctcaacggcctgggaaggcccgccgacgacctgcgcaggctccgcgacgccctccgctccgagtgcggcggcggacccgcgggtcgagggcactgagccggacggccgtcgcggacggtggtccgcgtcgtacgggagcgggccgcaccggtcaaggccaggacgccgcagagctctgcgatccggccgtcgcgggcggtgcggtgccgttcggcgcggtcccacgcgttccgtgcggttcggtgccgtgacgcggtcggccgaccgtcatgggcggcggcgccgacgcgtacccgggccccttccggccgagagccggatacgcgtcgacgccgccggtccggtggacgccgccgtccggctcggcggccggcggcgctgtccggctcagcgaagcgtgcgctccagtcgctccgccaccagcttcacgaaacgccccgggtgcgtcggccgcccgccctccgcgagcaccgcgagtccgtacagcaggtcggcggtctcggcgagcccggagcggtcctcaccctcctggtaggcctggttcaggccctggaccagggggtgggcggggttgagttcgaggatccgccgggcggacggaatctcctggcccatggcccggtacatgctctccagcgccggggtgaggtcatgggcgtcggagacgacacaggccggggagacggtcagccgggtcgacaggcggacgtccttcatctcctccccgaggtgttccttcatccagcccagcagagcggcgtacgtctcggcctgcttctcccgcccgccgtcggcctgttcgccgccctggacgtcgagatcgatctcgcccttggcgacggacctcagccgcttgccctcgaactcgcccacgacgtcgacccacacctcgtcgacggggtcggtgagcagcaggacctcaagaccccggtcccggaacgcctccatgtgcggggagttctcgatggtctgccgggacgcgccggtcatgtagtagatgtcgtcctggccttccttcatccgctccacgtactgctggagcgtggtcggcgtctcctcggcgtgggtgctggcgaaggacgccacggccagcagggcgtcgcggtcgtcggtgtcgccgagcaagccctccttcagtacggcgccgaactcccgccagaacgcggcgtacttgtcggcgtcgttcgccttcatctccttgaccgaggacaggaccttcttggtcagccggcgctggatcatccggatgtgccggtcctgctggaggatctcccgggagacgttgagcgagaggtcctgcgcgtcgacgacacccttgacgaagcggaggtggggcggcagcagcgcctcgcagtcgtccatgatcagtacgcgcttgacgtacagctgcagaccgcggcggaagtcccgggtgaacaggtcgtggggcgcgtgagccggaaggaacagcagcgcctggtactcgaaggtgccctcggcctggaggcggatcgtctccagggggtcgcgccagtcgtggctgacgtgcttgtacagctcgtggtactcgtcgtcggagacctcgtcgcgcgagcgtgcccacagcgccttcatcgagttcagcgtctcgggttcgggcgtttcctcgccgtcggtcgcctgcgggaggagccggaccggccaggtgatgaagtcggagtaccgcttgacgatctccttgatcttccaggcggaggtgtagtcgtgcagttggtcgtcggcgtcggccggcttgaggtggagcgtgacggcactgccctgcggcacgtcgtcgaccgtctccagggtgtacgtgccctcaccgcgcgacgaccaccgcgtgccgctgcgctccccggcacgccgggtcaccagggtcatctcgtcggccaccatgaagccggagtagaaaccgacgccgaactgtccgatgagcccctcggccccggccgcgtcctgcgcctccttcagctcctggaggaaggcggccgtgcccgaattggcgatggtgccgatgagcttgccgacctcgtcgtacgacatcccgatgccgttgtcccgcacggtgagcgtacgggccttctggtcgagctcgatctcgatgtgcgggtcggacgtgtcggcgtcgagcccgtcgtcccgcaacgcggcgagacgcagcttgtcgagcgcgtcggaggcgttggagacgagctcgcgcaggaagacgtccttgttcgagtagaccgagtggatcatcagctggagcagctggcgtgcttctacctggaactcgaacgtttcggtcgccatgcttcgtattcctcacaggttcctgggtggccgaatcgggcgacagccactgtaagacaccaagtcggcgcattgtcaccgccgttcgccgcgcggcgtccgcatctgcgtctgcgtctgcgtcagacctcgccgtgggcgcgcctgccccggccgtcccgccaggacgtggggcaggtgccccgcgcggtcgcggccccggcgtcggcgaacacgggcgctccagccgccttcggaaggcatcccggatgcggagcggagaccttcgaacacgccggtcctgacccggtcgcacgcccctgctccggctcgctcccggggatccggcacaatcgcaccgcggaccgacggccgcatccgccttcctgctgtggtgccggacggagccgtgggatctgcgctcctacggccacgtcgtcaagctggagcaggaacgtctcgcctaccgggcccgccgcactcccgcttcggccgctcctgtcgccgcaaggccccgccgacgcccaccgccccatcacacgtacggaggggcgaccagcaaggtgttgcggatcgcctcgacgatctcaggctggtgctgcaccaggtagaagtggccgcccggcagcagtgtcaggtcgaacgaaccggcggtgtggtcggcccacagcttcatctcgccctcgtcgaccttcgggtcctgcgcgccgaggaacccgcggatcggacaggccacaggcgggccgggcacgtagcggtaggtctcgatgaggcggtagtcgttgcgcagcgggggcatgatcatctcgatgatctcgggatcgtcgaacatccggctgtccgtgccctccaggccgcgcagttcggccagcacgtcttcctgcgacatggcgtgcacccgctcgccgcggttgaccgacggggcgccgcggccagacgcgaagagcgccgtcaccggcgtcgtggagtcctcgaggaggcggatgacctcgaaggccaccgccgcccccatgctgtgcccgaagaacgccgtcggtacggccggttctccgcacagcgcctcggcgacgtgggcggcgaggtcttgcagcgtggcggggaacgggtcggccctgcggtcctgccgccccgggtactggacggcgacgatgtcgatctccggggccagggcacgcgcgagcggcatgtagaagctggcggaaccgcccgcgtgcggcaggcagacgagccggtggcgggcgtccggggcgttcgtgtaacggcgcagccacgcctgccggtccgtggacggtttcggggtcggggcgtacatcaaggtttctccagagtgcggaaggcgaagcgccgaggcggaggtcgcccgcggcggtgggtgcgtcagtgggcgaccgcggcccccggttcggtgaccgcggtgtgctgctccatgacccggctcggacgcgccggccctgtcaccggcgcccgagcctgtgtgtccactcccagcaggccggccacctcgtcggcgacggacgtcgcgtcgcggccgacgcagtcgacccggtgcaccgtgacgcggccggagagcccgtcgagcacgtcgtcgtaggcgtgggcgagcccggtgagcagccgggcgtcctcgtgcagttcggtgccgttccggcggctgcgggtgcgccgcagtgcctcgtcgaccggcagtcggagccgtaccacggcgtccggcggggcggtcgagaacatgtcggccaggcgctgcgccagttcctcgcggggcgcggcggcgagccgcagcaggtccaggccgagcgcccacggatccgtaccgcccgagcaggcctccagccaatcccgcacctcgcgcgcggtctccggaccctgctccgcccaccagcgccggacggcgtcgccgggatcgggatcgcccgcgatccgcgcgaacatcggcagatagacgagggggtcgaccagcgggtgcctgtccgccaggacgatcgcgccacgctgcgtggcgcggcgctcggccggggcgtactggcacagttgcagatagagcaccgcgaccttcagcggcgaactgccgatcaggtccgcggcggcggacgcccgcgccaggtgcagggagcgcgtcgcctcggggctgtccgggtcgtcgtgcgcgcggatcgcgtggacgacggaaacgccgggcacggtgctcagcagccgcgccaccgtcgtcttgccggtgccgtcgatgcccaccagggcggcccgcatctcactcaccgtcccgggcggcttccaggttccacaggtggaacgtggtgtccagcacggccgggaggaacggcagttcccggtggctgtgcgcggcggtgtacagctggaggcggctcaggagcatctcacgcagcgcgagcccgtagccgcgccgccactgttccggctgcggcacggtcgcgtccggtccggccgcggcggcgaccgcggcgcggtgcacctccagatggtggtcgacctcgtcggtggtgctgtgcggggtgagggtgaaggccagcagttcggccaggtcgcgctggggcactgcgacggtggccagctcccagtcgtaggcggtgacgcgttcgctctgtcggctgatgttgcgggggttgaagtcgttgtggacgagtgtgcggggcatcgcgtccatctcctgcacccagaactccgcctcggccgccgccgcgagcgccgtcctggtgcgctgcggcgtcatcagttcgggcagttccgcggcgttgtggcggaccagcgcctcccacagttcgcgtgccttgacgaggtgggcggtcgtgccgtcgcggtacagccagccctcggccaggatctgctggtcgcgtccgagccagtgcccgtgcacgggggcgatggcgcgcagcgcgcggtccacgtcggtgcggctccacggcccggtcgtgatgtccaggcgttccatgaggatcacgtacgcttcgcgcgcctcgtcctcgatgatgccgtagcagaccgggagcaggctggtcagtacgccttcgggccgccggaagacggccagttcgcgccggtgggcggcagggaagtcgctgccgccgccccaggtctcccaggccgaggacacctcggggccgcacagcgaggcgatccggccgatgccggcggcgatctcctcgccccgcggtttggccttcgccaccagttcggcggtggtctgcgggccgtcgtcctcggtccagctcacggtcatggggatgacgccggtcagcttgcgccgttcgccgagggcgccgagttcggtggagatgccgtcgccgaccatcgccggcgcccgtacgacgtcggtgacgcgcagccgggggctgtcgagccgctcggccatggccggctggaggaggccggggccgaggtcgtcggcgcgcagccagtccacccgccgggcccggcccagccgccggtgcgcatcggcgaactggccgctgaccagggccgacgcggtcgacacgtcgagcgccagggcgaagccggcgatgatctcggccagccgggccgtgccgccttcgccgcggcatccgaggacaccgagccagtcgcgctggtcgggcaggccggtgccgccgccgacggtgccgatcacgaggttcggcagcaggagcgtcgcgatcaggtcgtcgccgtcggagtcgaaggagagcacggacacagcggactcgtgcacgcaggcgatgtcctgtccggtggccacgaacagcgcggcgatcacgttggccgcgttgatgccgtacccggtcatgccggcctgctggcctccgatgacggcgacccggtgtccgcgggcgatcgccgccggggtggtcttcaggaccgaggcgaccacgtcgccggggatgacgcactcggcggtgacccgggtgccgcggccggccagcagcgagaccgagctgaccttcttgtcactgctcaggttgccttcgagcagcgtgttgcgcggtcgcagcccgggctcgtcggcgagcacctcgttcagccaggtgcagatctgccaggtggccgcggtggtcatgttctgtccggccgcgtccgcggtctcgaagacgaaccgcacgtgcaggtagcggccgatctggtacgggtcgaccgcgaccagccgggcgtgctgcgagaccagccggacctggtcctccagctgcgggcggcgggtgccgagccaccggctgaagcgggcggcgccggccaggtcgtcgaactcgaaggcgggcgcgcggctcatccgctgggagagcacccgggtggacactccgccggccaggctcagggcgcgtgcgccgcgcgaggcggaggctacgagcgcgccctcggtggtggccatcggcgcgacgacggcttcccggacgccctggccgcggaactgcagcggtccggccagccccaccgggacctcgaccgatccggcgaagttctccaggttcccggtcagcgacgcggcctcgatcgccgtgtgtgcggcggaatcgagggtcgcgccggtccgggcgagcagccatgccagccgcgctgcgcgggcctgttcggtgtaccggccacggcccggtatcgcgtcgtcggtcacgcgtgtgccctttcgtcggcggtgatccgcgcccagctgtcgggcgcgggctggaagatcctgtccttgacgcgggtgcggcgctggccggagcggatcgcgtgctcccactcgcgctggaaggcgtcctggtgcacctggagcacctcgacgggggccagggctccggcgtcgcgcgccgcgcggtatccgtcggcggtctcgcccaggtgcttgtccagcagcgtcgcgaaaccggcgggcagcggtcccggcagtgcggaggcgatcgccgcgcggaaccggtccgttccggtctcgaccgtcgcgttgcgcagctccgccccggtgtcggccagggtgccggccagggcgcggaccaccgcgcgttccgtcaggtccacgccgccggccgtgcgcagcacgtcacgtccggtgtaggcgatgcgcggcgtacgcccgacgtggtcgacgacgtgcacgacgtcgttgacggcgcaccggtacagaccgccgatgtggctgagtacgaggtggtagtcgcggccgggttcgagttcggcggcggtcacggtcgggctgtcctcgcggatcggctcggcggcgtcggcgaactcgaagtagcagccgggcagatagagcggggcggcgttgggatggtcgtcgacggggacggcgaccgggccctccgaggatccgatcggcgcggcgaacaggcgtacgccgggaccgtagcgttcgcgcacgcgtggcaggtagagcgaggcgagggcgctgttccacgcgacggcggcccggaggttgggccacaggtggtacggatccaggacgccgtactcgtcggcgcggcgcgcgatctgctcggcgcgccgcggatcgggcgtggtgtgcggcactccgccgaccgtgccgcgggcgatctcctcgacgatccggggccactgggcggccagttggtggggcagcccggcgatcagggccgggttgacgccgatcaggaccttgatgtcgcgttcggcggcgagccgcagttgaaggtatgcccgctcccacgggtcggcgtcggagagctgctcggggatcgtggcccaggcggcgccgtcctccggcccgccgccctcgccgaagagccggtggtcgatctggctggggcccagatgcggccggccgtcggcggtgcgcgcgtgcggcgaggtcggatcgcgccacaggttcagcacgccgccggggtccgcggcgaggtcggggaacgcgccgagcaggacggcgaaactggcgtggtagaagggcaggaagcagcgtttcatgtaggtgggcgtgaccgggatgcgcttctcctggccggtgctgccgctggaggagaaaaacgcgaccggccgctcggcggtcaggaccccgtcctcgccggcaatcgcccgctcgatccagggaccgaacgcgttctgcgtgcggatcggcagggccttgcggaactcctcggcgccgcttcgctcgttcaggccgtgctcgcgcaggtagctcgtcgcaccgttcgccgcgaggagttccgcgaggaccgtctgctgggtctgctcggggtggtcgagcgtggcgaggaacctgtggtgttcggtcagaatgcgttcggtgtgcaagctttccctccagcgcggtcggagaagagggctccgtacaggcggagccgctcggggtcggcggtgtcgtgcgtgatccggctctcgacgcggtgggcacgggccgggtcgagcgcggcgcgcagccgcggtgcgtcgagcgccgggcggtgcgtcacgtgcacggtgtcgccgggacggaccagaccgcgcagcgcggcctcggcgaagaggtgatgggtgtggacgaggtagtccacgacggtgacgtccgcggtcgccatcaccgcctcggcccggaatccggtgtcggggccgggcagccgcacggtgaacggctccggcgcgggggccggtgccggagccgtgaaggcgcgcaacagccgtgccaggttcttcagttgcgccgtcggcaccgggccgtaccagctcagcgcgttgtcgggggtcgcgtacccgtccgggaaccgggccgccaccgtggcgggccggcccgccaggaagatcgtgcgggtgaacgtccgcagccatgcgtcggccggctgctcggggagcgagagcgcgaagtcgaggacgccgcggacgaacgactgcggggtcagggtgtccaccacgaccgccacggtgtggaagtggtgtcccggttggtcgtcgaggcgggccaggcgggccgccgcggaggcttgcagcaacgtctggagcgaaccatccgggtcttcgacgtccgggacgtcgatgtccgggagcgcggggtcgctcacgcggggtgcggtctgtccggtcatacgagcacctcgcgcaacagggcgggcagcggcgccgggcgcggtccgagcagcgagcaggccaggcggtgcgggtcgacatagccgtgcgggctgagcggcagtaccgggtcctcccgttcgtccgacggccccagggccaggatccgggccgcgcgcccgacgcagcggcgcagcaggtcgatccggaaggactggctgcactgccggtcgagcagaccgacgaagtagaggcgcatgccgagccggtaggcgtccgcgtccgccgacccggagatggccgacaggtccgagagcgtcgacccggtccacccgccgtacttgctggagacgaccttgccgccgatcggcacccgggtgagcgggagccgggtggtgtcggcgccgaactcggccaggatccggtcaaggagcaggtagtcgacggcgagcccttcgtcgaagagcaggaggaagagcctgccgggcgcgacggcgggcagcagcgagcgcaggatcggcagcaggtagttcgcgtggccgtccgtcgagacgatctgccggatcggcacgccccagcgggcgccgtccaggtagacgggcccgccgtgcggcctgtggtcgatgagcagcccgcggtccgcgagggccgccagggcgcgctgctccgaccaggtcagcgggcgggtgtcggtcaggccggggtcgcgcacgtgcagcaggtcgagttcggtgcgccacagctccagcaggcggtgggaggccggatggatccagccgtcccgctcgatccgggcgaagtaggggtcgagtgcgcgcgcgtcggtcgtcggacggccgcggtggaactcgacgtagcgccggccgatcgcggtctcgtcctcgccggaccagtcggtgtccggttccgtgcggtccaggtggttccagaacgccgtggtctgggtcgtcagcgtggacatccgcggattccagaccagggtggtggggccgagggtggccgtcgccttgaacagggcgtcggcccacagcaggcctttgacatgggtgggggtcagcggcttggtgggggtgatcgtcaccggggcgatcacgaactccctcgcggcgcggcgggcgccgggatgcgcgcggtgtgcgcggctcgcgggtgtgggcgggccggtcgtcgcggttcgtacggtgctcggggtgcgcacggtgctcgaggtgcgcagggtggtgctcatggatggctcctgtcgatgtctgccgcgaccgggcggacgaggtcgtgcgcggcgcgcgacatgccggtcgcggcgcaggacgtgccggttgcggcgcgcgccgcgtgggccgtgacgcgcgccgcgttcgaatcgacagcggtcacgacgtggtcggccgggacgggtcggacgccgtcccgtcggaggccccgtattcggcgtcgaggaactgcatgaggtcgctcgcgctcgccgtctccaggtcatcggtgccggagttcctgccggctcccccggctccggtgcccgtgccggagagccggtccaggagcgcggagagacgggtggcgagcgcggcccgggtgtctgccggtgtgaacggcgagagcaccttggcctcaaggcggtcgaaatcctcgaggaccggaccgtcgtcggcgggggcttcgtcgaggtccagttccgtgagcagccgctcggcgagcgactcgggcgtggggtggtcgaacgccagcgtggccggcagccgggcgccgacgagttcgccgagcctgttccgcagctccacggacatcatcgagtcgaagccgagttcccgcagcgcccggtccgcctgcacctgttgcgggtcgccccgcccgatgagctgcgccacgtgggtgcgcacgacgtcgagcagtacgggcagccgctcgtcctcgggcagcgcgcccacccggtccttcagcgcgcccgcggtcgtgcccgcgccggggagggcggcggacgctgcgtcccggccgccgcccgccgtgtccgcgggcgcgaatccgctgagcagcgggctggggcgaaggacggtgaacgcgggcaggaagcgcgaccaggccacgtccaccacgaccgggtcggtgtcgcccggcacggcgcgctcgagcgcttcgacggcgctgggcacggcgagcgggagcagcccgcgcttgcgcagttcccggtcctcgtactcggtgaccatgccgccgcccgaccagggcccccaggcgatggagacggcggacgcgccccgctcgcggcgtcggcgggcgagcgcgtcgaggcaggcgttgcccgcggcgtaggcggcctggtccgcggcgccccagatcccggcgatggaggagaacatgacgaactcgtcggcgtcgggcagcagttcgtcgagcaacagcgcgccgttcaccttcgccgccatgtcggtctcgaaagcctcggggtcgacgtccgcgatgcgcgcgtaccggatcacgccggcggcgtggacgaccgtacgcaccgggtcgtcctcggcatcaagacgggagagcagttgcgtgacggcggtgcggtcggtgatgtcgatcgccgcggcttcggcccggacgcccgattcgccgagttctgccaggagttcggcggcaccgggtgactcggcgccgcggcgggagagcagcaccaggcggtcggccgtgccgagagcggccaggcggcgggccacgtgcgcgcccaggccgccggttccgccggtgatcaggacggtgcccttgggctgccaggttcccgcgtcggccgcgggtgcggtgccgagccggcgggcccacaggcgcccgtcgcgcaccgcgagctggtcctcggcgccatcgccggtgagcacgccggcgagcagacgtgcggtggtccgcccggcgttgtcgtcagccgtcccgaggtcggccgtctccggaacgtcgacgagtccggcccagcgcgaagggagttcgagggcggcgacccggccgagcccccaggcgcccgccgcggccacgtccgtcgccgggtcgtcgacaccgacgccgaccgcgccgctggtcacgcaccagaccggcgcgtcgagccgctcggtctcccgcagcgcggcgagcagttcggaggcgtcggcggggcagacgagcacgccttcgaccggtgtcgaggccgatgcatcccattcggtggacgtgaggacgtgctcgaacagctcggcgagtccggggcgtgcggtgccgaagagcagccaggtgccgggcacgcgctcggcgatccgcgcctcgctcagcggctcccaccgcacggcgaaggtcgcggcctgtccgacggccccttcggccgcctggagctgtgtgcgctccacggcgcgcaggatcagcgattccatggcgaggaccggggttccggcggcgtccgtgatccacaccgaggtcgcctccggccgcgggcgcagccgtacgcggacgcgccgcacgtccgtggcgaagaggctgacgccgccgaaggagaagggcaggcgcacctcgtcgtcggtctcgtagaagctgcgggtgatgggcagggcgtgcaacgaggcgtccagcagagccgggtgggcgccgaagccgtagggctggtcctcgggcagcacgacctccgcgaacagatcgtcgccgcgccgccacagcgccttgaccgaccggaaggcggggccgtactcgtagccgcgctcggcgaggtccgggtagaacgtgtcaccggggatctgttccgcgcccgcgggcggccacaccgcgccggtccagtcgggcgtgaagccgtcggtgtccacgcgggaggcggtcacgacgccggtggcgtgcagcgtccagtcctcgcccggcgtacgcgtgcggatcagcagttcgcgctcgccgccctggtcgggtgcgacccacacctggaggtcgcgggcccggccgcccgggaacaccatcggggcgcgcaggacgagttcttcgacgcggcccgcgccgaccgcgcgcgcggcctccagcgccagttccacgaatccggtgccgggcagcagcagggtgcccatgacggcgtggtcgggcagccacgggtccgtgccgggcgcgaggcggccggagaacaggacgccgcccccgccggggaggtccgtgcgctgcgacagcatcgggtgcggcagcgcgtcggccccggcgccggggccggtacccgaggacggggccgtcagccagtagttctcgtgctggaaggggtaggtgggcaggtcgaggtgcgggaccgtggccctgccgcgctcgtgtccgcggccgtcggcggtatcggcgttgtcggcgtccgcctcggcatggtcggcgaaccaggtgaccggcgtcccggtcacgtgcagggtcgcaagggccctgaggaacgtgtcgggctcgggctgcccggcacgcagcgtcggcacgagcgccgcgggagcggcggacgcgtcctccagtgtctcggcggcgagcggcgcgagtgtggggtgcgcggtgagttcgaggtagcgggtggtgccgagtccgtgcagggtggtgacggcgtcggcgtggcggacggtgtggcgcagctgttcggtccagtggtcggcggtcgtgatccggtcctgctcggcgagcagtccggtgagcgtcgagacgatgggaatgcgcggcgcccggtacgtgagccccgccgcgatccggcggaactcgtccaggatctggtcctggtgcggactgtggaaggcgtggctgacggtcagccgcctgctcctgatcccccgctccgccagctgtgcggcgatgtccgcgaggacctcgggatcaccggacaggacggtcgactccggcgcgttgaccgccgcgagcgagaccacgtcctcacgccccgccacgagaccccgcgcggtggcctcgccggcctggagggcgagcatggtgccgggcgtggtgatctgctgcatcagacgggcccggtggaagaccagcgtggccgcatcagccaaagagagcatcccggcggcatgcgccgcggacagctcaccgatcgaatgccccaccagatggtccggccgcacaccgaacgactccagcagccggaacaacgcggtgtgcagaacgaacaacgccggctgcgtgaacgcggtccggttcagcagttccgccccctccgaccccggccccgcgaacatgacctcacgcagcgagcggcccagcagcggatcgaagaccgcacacgcctcatcgacggcagcggcgaacacgggatacgacgcatacaactcccgccccgcacccgggcgttgactgccctgaccggagaacaggaacgcagtcctgcccgtggtgacctgcccgcggacgaggctcgggtgcccggagcccgacgcgagcgccgacaacgcctcggtcagtccggcacggtcggcgccgatgagggcggcgcgctgctcgaactgcgaccgggtcgtcgcgagcgcgcgggcgaggtgaccggtgccggtctgcgggcgggcggcgaggaactccgtcaggcggtcggcctgcgcgcgcagcgcgtcggggcttttcgcggagaccagccatacggcggggtctgcggagtcggcttcggcctcgtacgcggtccgctccttgaccggaggctcttcgaggatcaggtgcgcgttggtaccactgatcccgaacgacgacacggcggcacgacgcggccgctcccccgcctcccagacgaccggcccggtcaacaacctgacctcacccgcctcccagtccacatgcggcgaaggctcgtccacatgcaaggtcctgggaagcaccccgccccgcatcgccatgaccatcttgatcacaccacccacacccgccgcggcctgcgtgtgcccgatgttcgacttcagcgaaccgagccacaacggacgaccctcgaaccggccctgtccataggtggccagcaacgcctgcgcctcgatcgggtcaccgagcgtggtgccggtgccgtgcgcctcgacggcgtcgatgtcggcggcctcgagccccgcgtcggccagggcctgacggatcacccgctgctgcgacgggccgttcggcgcggtcagaccattgctcgcaccgtcctgattgaccgccgagccgcgaatgaccgcgagcacacggtgcccgttgcgccgcgcgtcaccgagacgttcgagcaccagcatgcccacaccctcgccccacgccgtaccgtccgccgccgccgcgaacgacttgcaccgtccgtccggcgccaggccccgctgccggctgaactccacgaaggtgccggggttcgccatcaccgtcacgccgccggccagcgccacctcgcactcgcccgagcgcagcgcctgcgtcgccagatgcatggcgaccagcgaggaggagcacgcggtgtcgagcgtgaccgccgggccctccaggcccagcgtgtaggcgatccggccggacaggacgctggtcgtggtgccggtcagcaggtgaccgctgactccctgccccgtcgcctcgtacaggcgcggcccgtactcctgcggcatcacgccgacgaacacgcccgcccggctcccggcgaggctcagcggatcgatgccggcccgctccaccgcctcccacgaggtctccagcacaaggcgctgctgggggtcgatcgccaaggcctcacggcggctgatgccgaagaacggcgcgtcgaactgtccggcgtcgtgcaggaagcctccctcccggacgtacaccttgccctcggcgtccgggtccgggtcgtagagcccctccaggtcccagccacgtcccgaggggaatccgctcaccgcgtcgattcccccggcggccagctcccacaactgctccgggtccgtcacgccgcccggataacggcatgccatgccgacgatcgcgatcggttcccgcttgcgtgcctcgatgtccctgatgcgcaggcgcgtctggtgcaggtcggcggtgacccgcttgaggtagtcgcggagcttgtcgtcgttcgctggcatggaggaacctgcgccttctgcgtgttctgcatcgcgcggccggtccggtgggaccgaccgcgcggcgtggcggagtcggactgggcggaccgtcaggtcagtccgagttcgttgtcgatgaagtcgaagatctcgtcgtcgctggccgactcgaatcggtcggtggccccggcggcgtcctcggcgtccggccggcccgcttcctgccggccgtgcagccacacgagcaggtcctcgaggcgggcggtgaaccgggtgagtgtcgcctcgtccgcgtcggtggcgatcgccgtgtccaccagccggtccagttcggcgaggaccgccaggctgtcggacgtcgcagtccgcggggtctgcgaggccagtcgcgtgtagaggaagtcggccacggccaagggcgtcgggtggtcgaagatcagtgtgccggggagccgcagaccgctgacggcaccgagccggttgcgcagctccaacgcggtgagcgagtcgaagctcagctccttgaatctccgctccgggtccaccgtcgccgcggtcgcgtgtcccaggaccgccgcggcgctgtcctgaaccagctccagcagcatccctcgtcgcgcggcctcggtcggcagttcggccacgcgccgggccaggtccggtcccgcgtccaccgcggtctgccgcggtgccgggaggcgggcgccgtcggctgttgcggcggcctgtggcagacgccgtacgggccgggccggtgcgacgtgccggaacagcagcggcacctcggcgacgtcccccgctctcccggcaccaccggcgctttcggccagggccgcgaggtcgagccgtaccggcaccacggccggtaccgccgcgtcgcgcgtggcgtcgaagagggcgaggccctcgtcggtggacaggggcagtacgccggtgcgtgccatccggttcagatcggtcgcgctcaacgcctgcgtcataccgccggcggcgtgctcccaggggccccacgccgcggcgaccgcgggcagcccgcaccggtgtcggcggtatgcggccagaccgtccaggaaggcgttcgcggcggcgtagttgccctgtccggcgtggccgatcactccggcgacggacgagaacagtacgaaagccgacaggtcggcgtgggcggtgagttcgtgcagattccacgccgcgtccgccttgggccgcagcacggtgtcgagccgctccggcgtcagtgcttcgaccggggcgtcgtcgagcacgcccgcggcgtgcaccacggcggtcagtggatgcgcctccgggaccgcagcgacgacccgcgccagcgccgtgcggtccgccgcgtcgcaggcctcgacggtcacctcgacggcgcccgccgcgcgcagttccgcgacgatctcggtcacgccctcggcggccgggccgcgccgcccggtgagcaccagtcgcctgacgccgtgccgggtgacgaggtggcgggccacgttgccgcccagcgcgcccgtgccgccggtgatcaggacggtcccctccgggtccagcggcgccgggacggtgaggacgaccttgccgacgtgccggggccggctcaggtgccggaacgcgtcctgcgcgcgccgcacgtcccacgcggcgaccgggagcggacgcagcgcgccgcgctcgaacagctccgtcagctcgcccagcatggcgccgacgtgcgcgggttccagcttcgtcagatcgaacgagcggtggtcgacgcccgggtgccgggcggcgaccgcctcggagtcgggggtgtcggtcctgcccgtctcgacgaaccggccgccggacgacagcagccgcagcgaggcgtccacggcgtctccggccgggcagtcgagtaccacgtccacgccggcgccgccggtcgcggcccggaaccgggccgcgaactcctgggtgtgcgacggcgcgaggtgcgtgtcgtcgagcccatgtgcacgcaggtccgcccattcgccggggcccgctgtgccgaacacctcggcgccccggtgccgggcgagctgcgccgccgccaggccgacgccgtcggccaccgagtggatcagcagccgctcgcccggtacgacggcggccagttcgacgagtgcgtgataggccgtcaggaacgcgacgggcaccgacgcggcctgcgtgaacgtccagcccgccgggatacgagcgagggtgtcccgctccgccacggcctgggggccgaagttgccggtgagcaggccgaacacccggtcgccgacggccagatcggacacgccggggccggtctcggtcaccacacccgcgccttcgagcccgaggatgtcgtggtccggcggtgtgtcgcgggcgaggaccggggcgcggagatcgactccggccgcgcgcaccgcgatcctcacctgcccggtgccgagcggggcgcgctgggccgccccgacctgctcggccacgtcggccggtgcgggccacgggacgtcggcgtcctgcgcggcgcgagcgagtcgcggtacgagcacctcgccgtcgcgcaccgcgagttgcggttcgccggtgcgcagggcggcgggcagggcggcgtatccgtcggacgggtcgccgtcgatgtcgacgagggtgaagcagcccgggttctcggtctgggcgctgcgcaccaggccccacgcggtcgcctgcggcaggccgagtgcgtccggcgccgtcgccgcggccgtcgcatcgtgcgtgacgaggaccaggcggccgaccgtgagggcgggcgcgtcgagccaggacgtgaggagggcgagcgtgcggcgggcgacggcgtgcgcgccggccgcacctccttcgtgctctcccgtccgctctccttcgtgggccggaagttcggctccgtgcgcagcgagatcggcgagcacgacggacgggtgcgggtcaccgttctcgatgccgtggaccagggcgtcgaggttcgggtacgtgcggatgtcgaccgcctcggccgccagcgtggagaccggatcgggcacggggctccgaccgatgagcgcccactgctccggcgcgtccggagccgcggcgccgaccggtgccgctgccggtgtggcggaccactcgacgtggtacagcgaccggggtccgcccgcctccgtgccggagccgtcgctcaaccggtccaacaccacgggacgcagggacagttcccctgcggtcagtatgagggcgccggacggatcgctcgcgacgacgcggacggtggtcggcccggtccgggtgaaccggacccgcagcgcggtggcgcccagcgcgtgcagggcgacatcgccccaggagaagggcagcagcgtgcccgaggcatcggttccggacaccccgtccgcgagcagtccgctgcgcagcagggcgtgcagcgaggcgtcgagcagcgccgggtgcaccgagaaccggtccacgtcgtccgaggcggcggcatcgtcacccagctcgacctgtgcgaacacgtcatcgtccgtgcgccacgcggcggtcagcaaccggaagtcggggccgtactcgtagccggtcagggccagagcggggtagaggtccgtcaggtcgaccggcgccgcgtcggcgggcggccactgcggtgcacggtccgcggcgtcgggcgcctcggtgggccccagggctccgctcgcgtgccgggtccaggaggacgagccggaggcgtcgtcgccggcgggcgccgggcgcgaatggacggcgaaggcgcgcaggccggactcgtccgcctcctggacggtcacccggatgtcgacgccgcgctcgccgggcagcaccagcggtgcctgaagcgcgagttccgcgacagccgggtgctcccccgcgccgtccgacgcggcatgcagcaccagatcgagcagggcggtgccgggcagcagcgtcgtgccgtggatcgcatgatcggcgagccaggggtgcgtgagcgtgccgatacgcccggtgtggacgaagccgccgccctcgggcagttcgacggccgccgcgagcagcggatgcggcgtgcccgtcagcccggcctgcgtgacatcggcgcgcggcgcgggcggcgtgagccagtaacgctcgcgctggaaggcgtacgtgggcagttcgggcagagcagcggagcgagggccggggagtgccggccaggcgacgtcggcaccgctcgtgtgcagggtcgcgagggcgcgcagcagggcgtcgtgctccggccgtccgtggcgcagcaccgggaccagagccgcggggctctcctccagggtctcggcgaccagcgtggccagcgtcggagtgggagtgagttcgaggtagcgggtggtgccgagcccgtgcagggtggtgacggcatcggcgtggcggacggtgcggcgcagctgttcggtccagtagtcggcggtcgtgatccggtcctgctcggcgagcagtccggtgagcgtcgagacgatgggaatgcgcggcgcccggtacgtgagccccgccgcgatccggcggaactcgtccaggatctggtcctggtgcggactgtggaaggcgtgactgacggtcagccgcctgctcctgatcccccgctccgccagctgtgcggcgatgtccgcgaggacctcgggatcaccggacaggacggtcgactccggcgcgttgaccgccgcgagcgagaccacgtcctcacgccccgccacgagaccccgcgcggtggcctcgccggcctggagggcgagcatggtgccgggcgtggtgatctgctgcatcagacgggcccggtggaagaccagcgtggccgcatccgccaaagagagcatcccggcggcatgcgccgcggacagctcaccgatcgaatgccccaccagatggtccggccgcacaccgaacgactccagcagccggaacaacgcggtgtgcagaacgaacaacaccggctgcgtgaacgcggtccggttcagcagttccgccccctccgaccccggccccgcgaacatgacctcacgcagcgagcggcccagcagcggatcgaagaccgcacacgcctcatcgacggcagcggcgaacacgggatacgacgcatacaactcccgccccgcacccgggcgctgactgccctgaccggagaacaggaacgcagtcctgcccaccgtggcccgaccacgtaccaccatgggatgcccggcacccgaggcaagcgcggacagcgcctcggcgagtgcgtcccggtcctgggcgacgaccgccgcccggtggtcgaagtgcgtacggccggtggccagggcccgagcggcccggcggatgccgacctccgtccgggtcctggcgaactcggccagccggccggcctgttccccgagcgcgtcggctttcttcgcggagaccagccatacggcggggtctgcggagtcggcttcggcctcgtacgcggtccgctccttgaccggaggctcttcgaggatcaggtgcgcgttggtaccactgatcccgaacgacgacacggcggcacgacgcggccgctcccccgcctcccagacgaccggcccggtcaacagcctgacctcacccgcctcccagtccacatgcggcgaaggctcgtccacatgcaaggtcctgggaagcaccccgccccgcatcgccatgaccatcttgatcacaccacccacacccgccgcggcctgcgtgtgcccgatgttcgacttcagcgaaccgagccacaacggacgaccctccgaccgaccctgtccataggtggccagcaacgcccgtgcctcgatcgggtcaccgagcgccgttcccgtcccatgagcctcgacggcgtcgacatcggcggcctccagccccgcgtcggccagggcctgacggatcacccgctgctgcgacgggccgttcggcgcggtcagaccattgctcgcaccgtcctgattgaccgccgagccgcgaatgaccgcgagcacacggtgcccgttgcgccgcgcgtcaccgagacgttcgagcaccagcatgcccacaccctcggcccatgaggtgccgtcggcggcggcggagaacgacttgcaccgtccgtcgcccgcgaggccgcgctgcctcgcgaattcgacgaacatgccggggctcgccatgacggcggcgccgcctgcgagcgccagttcgcattcgccgttacgcagcgaccgggccgcgaggtgcgcggcgacgagcgacgacgagcaggcggtgtccaccgtcatcgcggggccctcgaacccgaaggtgtaggcgatgcgtccggaggccacgctgacggtgctgccggtcagcagatagccgccgacgcttcccgccgtctcggggaccgtctcgtgcagccgggggccgtattccatggccgtcgcgccgacgaacacgccggtgcggcttccggccaggccggtcgggtcgatgccggcccgctccaccgcctcccaggaggtctccagcaggagacgctgctgcgggtcgacggccagggcctcgcgcggcgagatgccgaagaactgcgcgtcgaaccggtcggcctcgtagaggaaaccgccctcgcgcgcgtaggtccggcccggtgcgtcggggtccgggtcgtagagcccctccaggtcccagccacggttctccgggaacacgtcgatcgcgtcggcgccctcggcgacaagctgccacagcgcttcgggggaatcggcggcgccggggtaacggcaggccatgccgacgatcgcgatcggctcgtcggagaccgaggccgccgacgtggcgtcgctctgcgtggtaccggccagctccgctcccagcactcgtgccagggctcgtggcgtcggactgtcgtacaacagggttgccggcacgctcagttggagcacagcggccagccgctcgcacaggtcctccgcggactgcgactccaggccgagatcgttgaaggagcgggccaggtccacttcgcgcggatcggagtgaccgagcaccgccgccgcctcgtcacggatgaggtccagcaactgctcgtcacgcccggcaggagcggcctcgaccagccctcgcagccagtcggagtcccgcacggacgcgggtgcggagacagcggacgtcttctccgggcgggccgtcacggccgatgcggccgacgcggtggcacgccgcgcggcacccttcaactccagacccaacacctgcgcgaggaccttcggcgtcgggctctcgtacaacagcgttgcgggcagacgcagttgcagcacggaacccaaccgctcgaccaactccacacccgacgccgactccaggccgaggtccttgaacgagcgggcgagatccacttcgcgcggatcggagtgaccgagcaccgccgccgcctcgtcacggatgaggtccagcaactgctcgtcacgcccggcaggagcggcctcgaccagccctcgcagccagtcggagtcccgcacggacgcagacgcggagacagggggcccggaggcgggcacagcggcgccagcgggagcagcagggttcggcgtcggaacggcggcagcgccctggcgtgccacgggcgcggacgtcggcgtgggctcgggccaataccggcgccggtcgaaggcgtagccgggcagttcgacgcggcgcgccgccggaaggccgtagagcgccggccagtcgacggcagcgccccgcacgtgcgcggcggcgagcgaggacagcagccgcggccggccgccgtcgccgcggcccagggcgggaatgccgaccgcgcccgcggcgtcgaggagttccaggatctcgggcggcagcacggcgtgcgggccgacctcgatgaagacggtgtgcccgtcgtccatcagttcctcgacggccggatggaagggcgccggctgccggaagttgcggtaccagtggtccgcgtccagggcggcggtgtccaccggaccgccgagggtcgtcgactggaagcgcgtcccgctcggcctgggctcgatgccgctcagctcgtcgaggagcgcgtcgcgcaccgcctcggcctgctcgccgtgcgcacggccgcgtgcgacgacgagagccgcggcgtcctggagggtgagcgcgccgatgctgtacgcggcggcgatttcgcccgcggcgtggccgagcacggcatggggctggacgccgagcgtgcgccaggtgtgcgcgagggcggtcgtgacggcgaacagcacgggctggacgtggtcgggggtgtccggcagggtctccggaccggtgaggtggtcgacgagggaccagccggtgagcgggtcgagggccgcggcggcggcctccacgtgctcgcggaagacgggcagggtcgccatcaggtcgcggaccgtgccggcccactgcacgccctggccgggaaacacgaacacggtcttcggcccggtgccgggcgtggtgccggtcccggggtccggcgcggtgccgcgcagcaggccgtccgaggggcggccctgcgcgagggtgcgcagctcggagagcagggcggaccggtcgccgccgaacgcggcggcgcggtgctcgtgatgcgtgcgaatggtggccaggccgcgcgccacggtggcggcgtccagctcggggtgctgctccaggtgcgcggcgagggccgcggcctgaccgcgcagcgccgcctcgctgcgcgccgagatcagccacggcgacgcgacgtcctgcgggacggcaacgggaacaggaacggtcgcgggcggggccgccgatgacgtcgcgggtgacggcgccgacgactcggccggggcgtcgcagagcacgacgtggcagttggtgccgcccatgccgaacgagctgacccctgcgacgatcggaccgtccgggcgcggccacggtgtcagcccgacctggacgcgcaggctgagctcgtcgaaggcgatcttcgggttcggcgtcacgaagttcaggctcggcggcagcttccggtgccggatcgccagagccgtcttgaccaggcccaccacgcccgaagcaccctcgagatgcccgatgttggtcttcgcggagcccaccagcagcgcgttgtcggccacccggcccacgcccgcgccgaaggcggtgccgagcgccgccgcctctatcgggtcgcccacggcggtgcccgtgccgtgcagttccacgtactgcacgtcgccgggggccacgtccgcctgccggcaggcggcgcgcagcagctccgtctgcgcgggcgcgctgggcaccgtcagtccgtcggtggcgccgtcgttgttgaccgcgctgccccggatgacgcagtacacgaagtcgccgtcggcacgggcctgttccagcggcttgagtacgaccagtgcgccgccctcgccgcgaacgtacccgttggcccgcgcgtcgaaggtgtggcagcggccgtcgggcgagagggcgccgaagctcatcgacgcggccatgccctccggcgcggcgatcaggttcacgccgcccgccagcgccacccgcgactcgccgcggcgcaggctctcgcaggcgaggtgcacggcgaccagcgacgaggcctgcgcggcgtccacggtcatgctggggccgcgcaggcccagcgtgtacgacacacggttcgcgatgagcccacggcccatgccggtcatcgtgtgctggttgaaggaggcggttccggcccgggcgacgacgctgcggtagtcgtcccagatcgctccgacgaacactccggtgccgctgccgccgagcgaggcggggacgatcgcggcgtcctccagcgcctcccagctcagttccagcatcagccgctgctgcgggtccatcgcccgtgcctcgtgcggcgagataccgaagaacccggggtcgaaggtgtcgatccggtccaggtaggcgccgtaccgggccgcaccggtcggcgtgccggccgcgtcgggccagcggtcggcgggcgtctcgcccaccgcgtccacgccctcgctcagcagccgccagaaagtcgcagggtccggagccgccggcagccgacaggccataccgacgacggcgatgggcatgaacccttcagacatgaagcgcaccctcgacggatatggaggagtcgccgcggtccgcggcgggccggccgagcgtctccaacgacgcaaggaagtcgagctcttccagcggacgatcggtgaagaagcgcgcgatcgtctcggcccactcggcggcacgcccgaggaacacgagatggtcggcctccgggatgacggcgaacagcgcaccctcgatctcggcggccagggcgcgcgcgccctccatggtggcgaaggtgtcgtgctccccgaccaggcacaggctgggtacgccgctgatgccgccgggcaggacggcatcatcctggaggaggaggtccgagacgttgaggtagccgggaagatcggcctcctcgatcgtgctgaagcgaccgttgagggccctgcggacgatttcacggttgcgcaccgtgaccgcggggtccaggcacatgagcaggtccagcagaccttcggcgaactcggcgaagcggcccgccgtgaggatcggatacatctgcgtcatccgctcccggttgcgcggcggccagtcggcggccccggccaggacgagacgggagacccgcgacgggctctgctgggcgtagcggtaggccgccgggaatccgttgctgatccccagcaggttcacacgcggcagccccagctcgtcgatgaggtgggcgagcgcttcggtctggacgtcatagccgccttcggcgggcacggggtccgccgtgcgcgagcccggcagatccacacagacgatggtggccgtgtcctgccagtacttgtcgaaccgccggtagctgaacttgtcctggtacgcaccggacagcacaacaaaaggctcggtgaccggtgcttcgcattcgaccatccggtagctgaacgcaagacctttgtaatcaagctcttcgacctgctcgcgcggatttccggcgcccacggatcagctcctcgaatttcgggcggatgtgcacggacggacaacggatacacgtccgtgcatgagcccgatctttgtcgccggccagggcaccgacaacccctatttccccccttagccgaaccggcttgccggatcggagctggtcggagctgcgagatgagtcccgatacgaatcctctccagattcaccccctggcacacgacccatcgacatgtattctcggcgtatgcccatcatcgaacttgccgaatacgggccagactttctcgcagatccttacccgtattacgcgaaactccgcgaggagggacccgtgcacgaggtacgggccccggacggctatcgattctggctgatcgtcggatatgccgaggggcgcgccgccctgaccgattcgcggctggtcaaggcacgcgacacgatggcgacgtccgaggcgtcgccactgggcaagcatgtgctgatcgccgacccgccggaccacacccggctgcgcaagctgatctcccgggagttcaccgtgcggcgggtggacaacctgcgcccgcgcatccaggaactcaccgacgacttgctggacgtcatgctgcccgcggggcgggccgacctggtggaggcgctggcccggccgctgccgatcgccgtgctgtgcgaactgctcggagtgccgaacgccgaccgggacgagttccactcctgggccaagggcatcctcgcgccgcagaacccgaccgagacgcacacggccgtcaaggccttgatgagttatctcgacgacctgatcgaggacaagcggcacggagagcccaccggtgacctgctgtcgggtctcatacgcaccagcatcgagaacggcgaccgcctctcctcggaggaagtgcgctccacggccttcctcctgatgatcgccggacacgagacgacggcgaacctcatctccaacggaacgcgggcgctgctcacgcaccgggaccaactggacctgctgcgctccgacatggacctcctcgacggcgccgtcgaggagatgctccgctacgacggctcgctggagagcacgaccaagcggttcaccggtgtgccggtccagatcggcgacacggtcatcccgccgggcgagacggtgctggtcagcctcgcgtcggcggaccgcgacccggcgaacttcgacgaccccgaccgcttcgacatccgtcgcggcaccccggccggcgtcggccacctcgcgttcgggcacgggatccactactgcctgggagcctcactcgcccgcgcagagggccggatcgcgttccgcgcgctgctggagcgctgccccgacctcgaactcgaccccgaggcaccgccgttcgagtggatgccgggcgttctcgtccgcggcgtgcagcggttgtcgctgcgctggtaggccgaacacaggcacgtatacggatgcaacggcgaagcggnnnccgcttcgcgatgacggtgtgaccgccgacggtgtggctgtcggcggcatgaccactcgtcaccaggtgacgggcaggctcttcgctccctggacgtcaagtcccctggagaacgggatctcgttcggcgcgacggccaggcgcagctgcgggatgcggctgaacagccgtccgtagaccacctccagctccatccgtgccacgctgtgcccgacgcacaggtggatgccggagccgaaggcgaggtggtcgcgggccgaccgctggatgtccagctcgtcggggcgctcgtacaccgattcgtcccggtcggctgagctgatcaggcagatgattccgtcgccgggccggatggtctgcccgccgatctcgatctcctccaccgccacgcgcttgggcgcgaactcggcgacggtgaggtagcgcagcagttcctcggtggcccccgcggcccgttcgggctccttggccagcagggcggcctgctcggggtggccgagcagcgccagcacaccggtggtgatcatcttgacggtggtgtcgtatccggcgccgagcagcagcccgatctgcatgagcaggttcgtctccgtgagcctgccttcgtcggcgaagccgaccaggcggctgacgatgttgccgtccggttccgcacggcgcagttcgaccagttccttgagctgctcgaacagttcaccgcgcgcggcgacggcggtttcggccgagccgtcctcgtcgaacagccgggcggtggtctcgccgaaccgggccatcgcctcggcaggcaccccgagcagccagccgacgaccgtcgacgggacaggacgggccagcgccggcacgaggtcggcggggctcgggccctccagcatccggtcgatgagcccgtcgacgagctcctcggtcgccggacgcagcgcccgcacccgcttgatcaggaagtgcggcgcgagcatccgccgcagttcggtgtggtcgggcgggtccatccggcccagcggccgtacgccgccctccgcggtgttcatggccttcgcgacctcactcagccatgggaaaccggggttgagcgcgtccacgctggcccgcgagtcggagaggatcttgcgcacgtcctcgtgccggctgatcagccaggcctcccgcccgttgtagagccgcgccttggacaccggctgctcggcgcgcaacgtccggtagacgggcgggggatccatcgggcagccgggcgccttcggcatcggatatgccggcggcgtctcggaggagctgtccgtcgtcaggggcggtgtaccggtgggcacggcgtcggtcgaggtcatcgtggcgaccctcctgggaagacttctggtggggtggttctgtcgcggcgggcgcgtgtcacgtttcgacaggcctgggcgcgcgtcacatctcggtgggtgggtgtcatgccctggccgacacaagcccgtcgttccgttcggcccgcttcgagccgatgtacgagaggaccgtcgcgcacagcgcgagggcggcgccgaccgcgaccgacgtggcaggacccgaggcctccagcagcacgccggtcaccggcatgctgagcgcggcgccgccggtgcccgcggtggccagccagccgaaggcttccgcccggcgctcctgcggcaccgctccggacacctgcgcgaagttgccggccatgctcggggcgatggcggtgccaccgaggaagagcacgaccagcacgagccagaccggagcggtttcggtcaccggcggcagcagcaccgcgagcagcgccatgccgactgccatcgccgcgaaccgggcgctgtgcggcacgtccttgcgcagggcgcccatgccgaagccgccgaccacggatccgatcgtccagcatgcgatgagcacgccggacagtccggactcgtggctgtcgcgggcccaggcgaccagggagaggttcaccgagaagagggcggccatcatcaccagggtcacgacgatggcgagggtgaacttcggcagggcgaagagcgtacgtcgctcctcgggctccggtgcggatgctctcgtgaccgcggacgccgacgtggccgtgtctccttccgtgccggcgtccgctcccgtttccgctgctgtccccgtctctgcttgtggtttcgcggcggtccgctccgccccgccgcggatcccggcgctgcccagcgcggcggcgaaggcgagcgcgccgacgaacgccacgacgccgcacgcgatgacggcgtatccggggtcgagggcggtcacgagcagtgaggtgagcagcggcccggtggtctgcaccacctccgagccggtggcctccagtgtgaacagcgtgcggccgagttcgccgggcacgatcttgggccacaccgcgcggctgacctgcgagatgggcacggtgctcattccggtgagcagggcgaccagcaccgcgatcggccagccgccgcccggcacggtgcgggtcatcgtcaccagtgcaccgaggccgacgaggtagccgatgccggtcaggaccagcagcttgcggaccgcgccccggtccgcggcgcggccccgggcgggtccgacgagcgcctgccccgcggtgagcgccccgccgagcacgccggcggcgacgtaggaatcgctccagccgaccagcaccagcgtgaggccgatcggcagcatggagacgttgaggcgggccagcatggaccagaggaacaggcgcggcaggtgcggaatgcctgccaatgccttgtaggacttcaccgttcacaccctgtttcgcaagtcatgacgcatacgcgcccgccgtgccgttcggcgccgccggacgctgcgtcttgatcaggtctcttcggacgttcggggccgctcagaaaacctggaaacgggcggcgatgcggtcggcgttctcggcgttctgccgggcccgctcgatcagcttctcgttgctccaggtgctggcggtggtgcctgtctcgtcgcccgcgttctgccgtgcggcgtagatccagccctcggcggcgcagcggaaccggaggatgtcggcgaggaaaccgagttccgcccggtcggcgggcccctcctcgaagtagccgcgcaccagcggctcgccgtccgtctcgtcctccaagtagctgagtgtggtggccagttccagcatgcagggtgcgtacatggcctccgaccagtccagcagacccgcggtgtcgccgaggacccggaactccttggccgcggcgtcgcagttgatcaggccgatggtcaggtcgtcgggcgacagcgcgccgcccgcctgctccagcgtccgccggatccagtcgtgtcccttgaggaagtcctgttcgagcaggaagagttcgagcacctcgttccagcgcggcacgccctccgggacgggggcgtgcagcaggacggagtcgatccgtccgagggtccggccgacggcgcgcaggtgggccgggtcggtctcgtcgacacgctcaccgtcgaggtaggtcagcaggctgtagcagaagtcgccctgataggcggtgacttctccgccggtggtcggcagcgggccgccggcggcgatgccgtgccgctccacctcctgggccaccaggagtcctgcggtgagcttcggccccatgtccttgcggacggccttgacgacgtggcgtacgccgtcgtgccggacgaggtaggtgtgcgagctgtacccctcgtctccgggcagccgctcccacggctcgacggtccagtcctgccagccccagagggtcagcggcaggtcgtgcgcttcgggtaccgcggctgcggcgtcggtcgattccggcattgaacgggttttccttgtctggtcgtgatgttccgagttccatgtccgacggccgcgcggtcgcgcggtcgcgcggtcgcgcggtcgcgcggtcgcgcggtcgcgcggtcgcggcatcatgtggtcatgtggtcatgcggtcacggggcagggtcatggtccgcgggcgcgtcccaggcgacggggagcgaattgacgccgtacaccacggagttcgcgcggaactcgatgtcctcgaagggcacggcgaggcgcagcgtggggaagcgctcgaacaggcgcaggtaggtcaccttcatctcgaccctggcgagctggtggccgatgcacaggtggatgccgtggccgaacgccaggttgcgctcctccgcccggtcgaggcgcagccggtgcgggtcggtgtagacctccgggtcgtggttggccgcggccatcgaaccgaccacggtctcgcccgccttgatgagctgcccgccgatctccacgtcctcggtcgccaggcgggcgaagttgaactgcacgatggacaggtagcgcagcagttcctcgacggcggtgtcgatgagcgacggatcggagcgcagcagggcgagctggtccgggttgcgcagcgcggccagggtgccgaggctgagcatgttcgaggtcgactcgtgcccggccatcagcatcaggccgcacattccggcgatctcgaactcggatatccggtccttctcctccgctggatcgagcagatcgctgatcaggtcgtccgacgggttcttcttcttgtcctcgatcaggtccagcatgaacttggtgccttcgaggacggtgcgctgctgctcctcctcggacagttcggtgtcgaggatgctcagggaccagcgctggaagtcgtcgcgcgcgtcgtagggcacgccgagcaggtcgcagatgatgagcgacgggatgggcagggcgtaggcggtgacgaggtcgaccgggccgccccccgtctcctccatccggtccaggtgctcacgggtgtactgctcgatcttcggctcaagagccttgatcttccggaccgcgaaacggctcgcggccagtcgccggtaacgggtgtggtccggcgggtccatggtgaagaaggcgccgggcagcggcgtggggcggccctcggtacgacgagtgcgggcggccttctcccggaccgagctgaaggacgggttcgccagcatcgccttgacgtccgcatggcgggtgagcagccagccctggttgccgtccgggaaggcgatgggactcacgggcgcctcggcgcgcaccttcgcgtagccgtccggcgggctgaaggggtcgctgcggtgcaggggcaggccctgcaggagagaaagagtgttcatggtcgtccttcctcagagggcgtcggtgtttcgagtcggtgcgtactgaggggtgcggagtggtacggggcgggcgcggacgcgcggcggccccgtacctgctccatcagggccgggtgaggcccggcgtacggctcagccactcgtccaccgccgcggccgtggtctcggagaattcgccgatcatggtgcagtggtcgcccggcacctgcgtctcctcgtggtcgaggggccacgccgcctgccagtcggggccggccatgggttcctcgggggaacccgggatgcagctgtccgggcggacgaagagagtcggcacggcgagttgccggggctgccagccgcggaacataccgcggtacgtaccgagggcggtgaggccgtcgtagtgcatcgacgtgaaccgcatccggcgctcgacgacctcgtaggtcatggccttgcgcatctccagcgtcatgctgtcgggcgggtaggtgtccagcaggacgacgccgaccgggcctgttccgcgctcctccagccaagtggcggcggcctgggcgagccagccgctggacgagtagccgagcagcgcgtacggccggccgtccgccgcacgcagcaccgcctccgccagtgtctcgatcagcacctccagggaagcggcgagaggctcgccggccatgaagcccgggacggtgaccacggagacccggcggcggccacggaagtggttggcgagccgggcgaactggagcgagccgtccacgggcgcgaacggcgggaagcagacgagctgcggctccgcctcgccgtggccgagggtggtgacgtgcgcgccgcggcccaggtcctcggcgccgtggaatctggtgcgcagggcggaggcgctgctgaggaacgcctcgacctcctgcatccggccttgcaacgagagcttgcggtagatgccgacgacggagtcgttggagtcctgcggcggcgcggcggccacggaggcggacggcgccgccgacacgccgggcgccggtgacgcgccggcgtcgccgcccggcgcggcattgtccagcagatgggtcaccagggcgccgagcgtcggatggtcgaacaccagggagctgggcagcgccaggccggtcaacacggtcagccggccgcgcagttcgaccgcggccagcgagtcgaagccgagcgccaggaactcctggtcctcggtgatcgtgccggcgtccgcgtgcccgaggacgcgcgcggcctcgcgacggacgatatcgaggacgtaagggcgctgatcggcgagcggaaggtccggcagccgctgccattcgccgggctgcgcggcaccggtgcgtccggcatcggggtgtccggcaccggactccgtatcgtcgaagtcggcgaacaggtggttcggccggcccgcggtgaagacgccgatgaaccgtggccagtccaggtccgcgacgacgatcgcggtgtcctcctgcctgaccgcccggtcgagggcggtggtggccaagcgcggggtgagcggccgaaggccccggcgctgcatctcctgccggaactcttctccggccatgccgccgccgctccagggcccccaggccagcgtggtggcggcggcgcggcgggcgcggcggcgctcgaccagcgcgtccaggaaggcgttcccggccgcgtacgcaccgccgcgcgtgctgccccaggtccccgcgatggacgagtagacgacgaacgcggcgagtccgtcgcccagcacctcgtccaggatgacggcaccggtgaccttcgcgtcgacgacggcggcgaattcggtcgcgtcgagatcagccagcggatgttcggccgccacgcccgccgtgtgcacgaccgcaccgacgggggcaccgcgcccggccaggtcggcggcgagcgccgcgacctcgtcgcggctggtcacgtcgcaggacaccaggtccaccgtggcaccgtgggcggccagttcggcccgcaggtccgcggcgccgggggcgtcgggcccctggcggctggcgaggacgaggtgcggggcaccctgctcggcgagccgacgcgccgtgtgcgcgccgagggcgccggtgcctcccgtgatgaggaccgagccgtgcgaccaccagggttcgcgcgcggcgggcggctgcggatcaccggtccgcccggtggcgtcggccccctccggggcgacgagggattccggggcgacggggcgtacgggctcgggtgcgccgtccgggcctgcgggccgcaggcggcgcacccgtgctccgtcggcacgcagcgcgacctggtcctcaccactggatccggccagcagcgccgccaggccgaaggaggcctcggccgtcgcggcgagggcgtgcccgtctgccgcggacaggtcgggcgcgggcaggtcgaccaggccgccccacagggtggggtgttcgagggccgcgacccgtccgaggccccagacctgggcctgccacgggtcgggagcgtcgtcggacgccgtcgcgcggaccgctccgcgggtgagcgtccacaaccgggtcgcgctccagcccgtgtcgagcagcgcttggagaaggcacacggaggcccaggcgccggagccgacgccgcgcggcccggtgtgctcgcggccggacagggcgagcagcgagaccactccggcgggagtgtcgtcgagcccgttcagcagcttggcgatggtctggcggtcgatgtcctcgggcgcgagggacagcgacttcacctcggcgccggcatcggtcagcacccgacgcacctcgccgtgcagcccgtcgtgcagcccgtcgttgtcgagcaggtggcccgcgcgcaggtcgccttcgggtacgacgatcagccaggtgccgtgcagggtggcgggcccctcgggggcgtgctgcgcggtcggccgctcccaggcgacgcgatagcgccatccgtccgtctcggaggcctcgatgtgcgtctggtgccagtcgccgagcgcgggcaggacggtgtgcagcggagcgtcggggtccacgccgaggtcgctcgccagccgctggaggtcctgttcctggacgaccttccagaacgcgccgtcgctcccggccgtccgggatgccgccgatccgggccggacggaggcgcccttgagccagtggtgttcgtgctggaaggcgtaggtggggagttcacgggcgaggtcgtcggcccggccgagagcggtccaatcgacctggtgaccacgcgcgtgcacccgggccagcatgccgaggaacgcccgggtgtccgtggaacgccggctcagcgtgggcacgaacgccacgtcccgcgccgccgagtcccgctcggcggacgcggcgcgcacgcgctcgcccagcgccgtcaggacggggtcgggcccgagttcgacgacggtcgcgacgccctgggccaggaccgcgccgactccgtccccgaaccgcaccgcttcgcgcacgtgccgcacccagtactccggcgagcacagctcctcggcgtccgcgatcgtgccggtcacgttggacacgaccggaatcgacggggcacggaactccacctgcgccagcacgtccgcgaactcggcgagcatcggctccatcaacggcgaatggaacgcgtggctgaccgccaacgcccgtgtgcgccgcccccgtccggcaaagatgtccgcgatctggtccaccgcggcgtcctgaccggacacgaccacagcccctggagcgttcacggctgccagcgacaccatgcccccggcagccgccacatccgcgacgagcggcgcgacctcctcctcggtggcctccaccgccaccatccgcccacccgacggcaacgaacccatcaaccgggcccgggccaccaccacccgcaccgcatccgccaacgaccacacacccgccacatacgcggcggacaactcccccagcgaatgcccgatcaacacatccgcacgcacaccgaaagactccgccagccgatacaacgccacctcgaccgcgaacaacgcaggctgagcaacccccgtatcctccaaaacccccgcatcatcaccgaagaccaccccaagcagctctgctcccgtctgcgcctcgacctccgcacacacctcgtccaacgcagccgcgaagaccgggaaccgcccatacaactcacgccccatccccggacgctgcgagccctgacccgagaacgccacacccacaccaccagcgacacgacgctcaaacaccacaccaccggcagcggaaccgtcaccccgcgcaaccccacccacaccggccaacaactcgtccaacgacccaccactgaccacagcactgtgatcgaacaccgaacgcgacgacaccaacgccagacccacaccccccacatccagcgcaccacccccgcgtcccgccacgaacgccgcaagccgcgccgcctgagcccgcaccgcaccctcagtacgacccgacacaacccacggcaactccccagcaaccaccagcgcttgagtggactccaccggaacctgagcggaccccaccggagcttcagtggattccacgggctcgtgctccaggatcacgtgcgcgttcgtcccgctgataccgaacgacgacacacccgcccgccgcgcacgacccgtctgcggccactgccgagcccgcgtcaacaactccaccgcacccgcagaccaatccacatgcggcgacggctgcgacacatgcaacgtccgcggcaacaccccgtgccgcatcgccatcaccatcttgatcacgccaccgacaccggcagccgcctgcgtatgaccgatgttcgacttcaacgaccccagccacaacggacggccctccgcccggccctgcccgtacgtcgcgatcaacgcctgcgcctcgatcggatcacccagcctcgtccccgtcccgtgcgcctccatcacatccacgtccgacgtcgacaaccccgcacccgccaacgcccgcacgatcacccgctgctgcgacggaccgttcggcgccgtcaacccgttcgacgcaccgtcctggttcaccgcactgccccgcaccaccgccaacacctcgtgcccgttgcgccgcgcgtccgacaaacgctccagcaccaccacacccacaccctcggaccagcccgtcccctccgcatccgcggaaaaagaacgacaccggccgtccgccgacagaccaccctgacgaccgaactccacgaacgcgtacggcgtcgccatcaccgtcacaccacccgcgagcgccaacgaacactcccccgcacgcaacgactgcaccgccagatgcaacgccaccaacgacgacgaacacgccgtgtccaccgtcaccgcaggaccctcgaacccgaacgaatacgagacccggcccgagatgacggagctggccgatcccgttccgccgacgccttcgggcgcgtcgacgatctccgtgccgaccaggccgtagccctggacggcgccgcccatgaagacgccaaccggcttgccgcgcaacgagtcggcgctgatgccggaccgctccaccgcctcccagcaggtctccagggcgatgcgctgctgggggtccatggcggcggcatcgcgcggcgagatgccgaagaagcccgcgtcgaactccgcggcgtcgtgcaggaacccgccgcccgcggcaggcagtcgtccgaggtcccagccgcggtcggccgggaacggcgagatcgcgtcccgcccctcggccaccagtcgccacaggtcctcgggcgaggcaaccccgcccggatacttgcaggccatgccgacgagcgcgatcggctcgttcccggctgcctccagttcccgcagccggccgcgggtacgcagcagatcgccggtcagttccttgacgtagtggcgaagcttgtcttcgttggtggacacggtgcgccagctccttgttcgtgctgaggtttgcgaacgccggcgtcaggagatgccgaattccttctcgatcagatcgaagagctgatcgtcggttgccgagtcgagttgctcggcagctgtttccgccgcgccggacgcggcggtgggctcgtcgtcggcgttctggaacctggtcaacaggttggaaagccgcagggtgatacggccgcgcgcggccgggtcggacccggcgtcgagcgcggcgagggccgcttcgagccggtccagctcaccgaggaccgccgacgcgcccgacgcgccgccgacccgctccgcgagggcgctcgcgacgacctgtgccagggccgcgggcgtggggtggtcgaagacgagcgtggcgggcagcttgaggccggtgagcttttcgagccgctgccgcagaccgactgcagccagcgagtcgaacccgagttcctggaaagggcgttccggctcgatcgtgccgcccgaggcgtgcccgagttccgcggcggcctgcgtgcagacggtctccaccagcacccgtcggcgttccccgccggacagcgcggtccagcgggcgacgaagggggtggcccctgcggtaccggcgtcgctgtcgcccgggccggacacgtcgtccgcaccggccgtgccggccgccgtcccgtccgcgccgacgccgccgcgttccgtttccacggtgcggagcgggtcgaacagggggctcggacggttgacggtgaagatgtcggccaggcgtgaccagtcgatgtcggcgaggacgacggtgccgtagtccgccctcacgacgcggccgaacgcggcgacggcctcctccgggtcgagcgcgctgacgccgcgggcctgcatctcccgtgtgaggcgttcgtcggccatgccgcccccgccccaggggccccaggcgagggcggtgccgggacgtccctgggcgcgccgccgctcgatcagcgcgtcgagatgcgcgtttcctgcggcgtaggcaccggcacgagcgctgccccagaccccggcgatggaggagtagacgacgaacgcggcgagtccgtcgcccaagacctcatcgaggacctgcgcgccgacgaccttcgcgcgtacgacggcggcgtagccgtcctcgtcgagctccgcgagcggcagttccgaggcgacgcccgcggtgtggaccacggtgctgaccggcgttcccgcgtcggcaagcctgtcccggagggcggcgagggccacggcgtcggtgacgtcgcaggactcgacgacgacgtccgcaccccgctcctcgagttccgtgcgcagcgcggccaccgcgggtgcggcagggccctgacggctggtgaggacgagggtccgcgcgccgttccgggcgagccagcgcgccgtctgcgcgccgagggcgccggtgccgccggtgatcaggacggagccgtcggtccagggcgccgtggagccggtctccggctccggcacgctcgtgggtaccgtgccgcggatcagccgacgaccgagcaacagctcgcctcgcagggcgatttggtcgtcaccggtggtgttggcaagggcagcggcaaggccggtgagctgcgctgccggactcgtaccggagacgtcgatcagaccgccccagagggtggggtgttcgagggccgcgacccgtccgaggccccagacctgggcctgccacgggtccggcgccgcgtcgtcggcctgcgcacacaccgcgtcgcacgtgagggcccatacgcgggtgtcgactcccgcgtcctgcacggtgtgcaggagatcgagcacggccagggcgccggtcgcgatgccgcgctcccggtcgcggtcgcgctgggcgccgaccgcgggcaggcagagcacgccgcggggcgccactgcggccagccgtgcgcgcagctcggccgtctcgcaccgctccacgcgcgcccccgcgtcgacgagcgccttttcgaccgcaattacgaggtccggttcgacgggctcgccgggcaccgcgaccagccacgtgccgtcgagcggcaccgcgtccgtcggcgacggaagctccgtccaggagacgcggtagcgcagggcgtcggccgctgcgagccgggcctgttccctggaccaggtctggagcgcgggcagcaccgcggtgagcggcgcgtccggcgcgaggccgagggtatgggcgaggccgtcgacgtcctgctgcgcgaccgcgttcaggagtacggcctgctccggcacgtgctcgacgacgccgggctcgggcgcgggggcgtcgagccagtaacgctgatgctggaaggcgtaggtggggagttcacgggcgaggtcgttcgcccggccgagagcggtccagtcgacctggtgaccacgcgcgtgcacccgggccagagccgtcaggaaaccgttcacatcacccgtccgccgccccagggtgggcaggaacacggcaccgttctcgacgactcccggatgcgaggcacccatcgccgtcaacaccgcctcgggccccagctcgacaacggtcgcgacgccctgcgccaggaccgcaccgaccccgtccccgaaccgcaccgcttcccgcacgtgccgcacccagtactccggcgagcacaactccgcagcggatgcgacctcacccgtcacgttcgacacgaccggaatcgacggggcacggaactccacccgcgccagcacctgcgcgaacccggcgagcatcggctccatcaacggcgaatggaacgcgtgcgagacacgaagacgagtcgcacgacgccctcccccgcgcgcccgctccaccaccgcctcaacggcaccctccacacccgaaacaaccaccgccgccggcccgttgacagcagcgatcaccgcaccgtccaccagccagcccgacacctcctcctcggtggcctccaccgccaccatccgcccacccgacggcaacgaacccatcaaccggccccgggccaccaccacccgcaccgcatccgccaacgaccacacacccgccacatacgcggcggacaactcccccagcgaatgcccgatcaacacatccgcacgcacaccgaaagactccgccagccgatacaacgccacctcgaccgcgaacaacgcaggctgagcaacccccgtatcctccaaaacccccgcatcatcaccgaagaccaccgaaagcagctctgctcccgtctgcgcctcgacctccgcacacacctcgtccaacgcagccgcgaagaccgggaaccgcccatacaactcacgccccatccccggacgctgcgagccctgacccgagaacgccacacccacaccacccgcgacacgacgctcaagcaccacaccaccggcagcggaaccgtcaccccgcgcaaccccacccacaccggccaacaactcgtccaacgacccaccactgaccacagcactgtgatcgaacaccgaccgcgacgacaccagcgccagacccacaccccccacatccagcgcccccgcaccgcccccgcgtcccgccacgaacgccgcaagccgcgccgcctgagcccgcaccgcaccctcagtacgacccgacacaacccacggcaactccccagcaaccaacggagcttcagtggactccacccgagcctgcacagaccccaccggaacctgagcggaccccaccggagcttcagtggattccacgggctcgtgctccaggatcacgtgcgcgttcgtcccgctgataccgaacgacgacacccccgcccgccgcgcacgacccgtctccggccactgccgagcccgcgtcaacaactccaccgcacccgcagaccaatccacatgcggcgacggctgcgacacatgcaacgtccgcggcaacaccccgtgccgcatcgccatcaccatcttgatcacaccacccacaccggcagccgcctgcgtatgaccgatgttcgacttcaacgatcccagccacaacggacggccctccgcccggccctgcccgtacgtcgcgatcaacgcctgcgcctcgatcggatcacccagcctcgtccccgtcccgtgcgcctcgaccgcgtccacatccgccacggaaagtcccgcgcccgccagtgcctggcgaatcacgcgctgctgggacgggccgttcggcgccgtgagcccgttcgacgcaccgtcctggttcaccgcactaccccgcaccaccgccaacacctcgtgcccgttgcgccgcgcgtccgacaaacgctccagcaccaccacacccacaccctcggaccaaccggtgcccgatgcgtcggcggagaacgaacggcaccggccgtcgacggccagtccgccgtggcgtccgaactccacgaacgcgtacggcgtcgccatcaccgtcacgccaccggcgagcgccatcgagcactcccccgcacgcaacgactgtgccgccagatgcatcgcgaccagcgacgacgaacacgccgtgtccaccgtcaccgcaggaccctcgaacccgaacgagtacgagacccgtcccgacgcgatgctgccggagcttccgttgctgatgtagccctcgtacccctgcggcgagcggttcaggtggcgggcgccgtagtcgttgtacatgacgcccatgaacacgccggtgcggctgccggtgagcgtctccggccgggtgccggccgactccagggcctcccaggaggtctccaggagcagtcgctgctgcgggtcggtcgcggtcgcctcgcgtggcgagatgccgaagaactccgcgtcgaactgggccgcgtcgtgcaggaacccgccctcacgggtgtacgtcttgccgggctgctgcgggtccgggtcgtagatgccgtcgaggtcccagccgcggtcggccgggaacggcgagatggcgtcccgcccctcggccaccagccgccacaggtcctcgggcgaggcaaccccgcccggatacttgcaggccatgccgacgatgacgatcgggtcgtcgcccgcatcctgcggatggctcgccgacgcccgcgccgcgggctcggcgaccgcgagcgcggtggacccggccggctccccgaggactccgcgagcgagttcgtcgtacaggaattccgcgaccgcgagcggagtcgggtggtcgaagaccagcgtcgccggaaggcgcacgccggtggcggcgccgagctggttgcgcagttcgacggcggtgagggagtcgaggccgagccggttgaacggctgggcgcggtcgacggcctcgcggtcggcgtgtccgagcacgtacgcgaccttctccgcgacgaggccgccgaggatctgaaggcgctcctcgcggtcggccacgcgaagctcggcgagcagcggcgcgctcgcgctgccggcggtcgccgcggtgccgccggccacacgggaagagggtcggggcctggtgctgacgaccgcttggaagacggcgggcagcgatccggccgcggcctgctcgtcgaggacgggggcgttcagccgggcgggcacgagcaggccgtcggcgtgcgttccgacggtctccgggccggcctccgaagcgccgaaggctgcgccggctgctgcgagagcggcgtcgaacagcgtgacgccctgttcgcggctgatctccaggaggccggtgcgcttcagccgggccacgttggcccggtcgagctccgcggtcatcccgccctcggtgctccacaggccccacgcgagcgagacgcccggcagcccgagcgcccggcgccgacgcgccagtgcgtcgaggaaggcgttggctgcggcgtagttggcctgtccggccccgccgaagacaccggcgaccgaggagaacaggacgaacgcggagagcggggcccgcgacgtcagctcgtggaggtgcagcgcggcgtcggccttcgcgcgcagcaccttcgtcagctgcgcgggagtgagggattcgaggagcccgtcgtcgagtacgcccgcggtgtggaccacgccggtgagcggatggtcgcccggtacgccggcgagcagttcggcgacggcggagcggtcggacatgtcgcaggcggcgagcgtcacgtcggccccgagtgcttccagttccgcgatgagctcggcggcgcccggggcgtccggaccacggcggctggtcagcagcaggtgccgcactccgtgcacggtgacgaggtggcgggcgaagagcgagccgaggtcgccggtgccaccggtgatcaggaccgttccctcgccggagaaggcgggggcgtcggcggagccatcggcgtcggtcgtctcgggggccgcgatcgcgcggagtcgcgggacggaggggaccccggcacggagcgcgatctgcggctccgcgacgacgccggtgcccgtgccgatgtctgtgccgttgccggtgccgagcagcgtcggcagggcgcgcagcgaggcctcctccccgtcggtgtcgatcaggcggaaccggcccgggtgctcaagctgggcgctgcgtatcaggccgcccacggcggcggacgccaggtcgacggctgcggcctcggccgcgtcgaccgcgagcgcaccgcgggtgagcagcgtcgcggtgacggatgcgaagcgcggctgcttcagccagtcctggaggagatggaggaccgtctgcgtccgctgatgcgtggccgcggcgatgtcgccgtccactccgcccagggcgggcagcgctatcaggacgtcacgaggcgcctcggccggatcggcgtcgatggcggcggcgagggctgcgaggtcggcgtggcggcgcgccggaacggattcgacggaccagtcggcgccgaggtcgaggagcgccaatccggtcccggacgcggacacggccgcagacacagcggaaccggcgctttccagcggcgaccacgcgatgccgtacagcgattcgacgctcgacacgcgggccgagcgaagctgctccagcgtcacgggacgcagcgccagcgaccgcacggtggcgagtgccgcccccgcctcgtccagcagctccacggaggtcgcgccctcaccgaggcggcgtatgcggacccgggccgaactcacggcggtggcgtgcagcgtgacgccgctccaggagaacggcagatggcactcgtcctgcccggacagcaggtcgggcagcgcgagcgagtgcagggcggtgtccaggagcgccggatggatgctgaaacgcccggcgtcgtccgtctgccgggtgggcagacggacttcggcgtagacggtgtcgccgtcccgccacgccgtgtgcagcccgcggaacgcgggaccgtagtcgaatccgaggccgatcaaccgctcgtagacggcatcgaggtcgaccggtgtggccccgcgcggaggccatgcctgcacctgcgacggctccggggcgagcaccggggccggctccgccgcgtcggaacggagggtgccggtggcgtgccgggtccagggggcgtcctcggcggcgtggacaggccgggagtgcacggtgatcgcacggccgccgccggccgcctcggcctcgccgaccaggacctgcacgacgtgggccgaccccccgtcgaggatgagcggcgcctccagcgtgagttcctcgacgcccgcgcagcccacgcgggccgcggcggtgaaggcgagttcgaggaacgcggtcccgggcagcagcgtcgagccgagcacgacgtggtcgccgagccacgggtgggttcccggggagatcgtgccggtcagcactgtgccaccggtgccgggcaaggtgaccgcggcgccgaggaacgggtggtcgacggcgcgcataccgaggtgcgcggcgtcgacggaggcggcattgccggcaagccagtggtgttcgtgctggaaggcgtaggtggggagttcacgggcgaggtcgttcgcccggccgagcgcggtccagtcgacctggtgaccacgcgcgtgcacccgggccagagccgtcaggaaaccgttcacatcacccgtccgccgccccagggtgggcaggaacacggcaccgttctcgacgactcccggatgcgaggcacccatcgccgtcaacaccgcctcgggccccagctcgacaacggtcgcgacgccctgcgccaggaccgcaccgaccccgtccccgaaccgcaccgcttcccgcacgtgccgcacccagtactccggcgagcacaactccgcagcggatgcgacctcacccgtcacgttcgacacgaccggaatcgacggggcacggaactccacccgcgccagcacctgcgcgaacccggcgagcatcggctccatcaacggcgaatggaacgcgtgcgagacacgaagacgagtcgcacgacgccctcccccgcgcgcccgctccaccaccgcctcaacggcaccctccacacccgaaacaaccaccgccgccggcccgttgacagcagcgatcaccgcaccgtccaccagccagcccgacacctcctcctcggtggcctccaccgccaccatccgcccacccgacggcaacgaacccatcaaccggccccgggccaccaccacccgcaccgcatccgccaacgaccacacacccgccacatacgcggcggacaactcccccagcgaatgcccgatcaacacatccgcacgcacaccgaaagactccgccagccgatacaacgccacctcgaccgcgaacaacgcaggctgagcaacccccgtatcctccaaaacccccgcatcatcaccgaagaccaccgaaagcagctctgctcccgtctgcgcctcgacctccgcacacacctcgtccaacgcagccgcgaagaccgggaaccgcccatacaactcacgccccatccccggacgctgcgagccctgacccgagaacgccacacccacaccacccgcgacacgacgctcaagcaccacaccaccggcagcggaaccgtcaccccgcgcaaccccacccacaccggccaacaactcgtccaacgacccaccactgaccacagcactgtgatcgaacaccgaccgcgacgacaccagcgccagacccacaccccccacatccagcgcccccgcaccgcccccgcgtcccgccacgaacgccgcaagccgcgccgcctgagcccgcaccgcaccctcagtacgacccgacacaacccacggcaactccccagcaaccaacggagcttcagtggactccacccgagcctgcacagaccccaccggaacctgagcggaccccaccggagcttcagtggattccacgggctcgtgctccaggatcacgtgcgcgttcgtcccgctgataccgaacgacgacacacccgcccgccgcgcacgacccgtctccggccactgccgagcccgcgtcaacaactccaccgcacccgcagaccaatccacatgcggcgacggctgcgacacatgcaacgtccgcggcaacaccccgtgccgcatcgccatcaccatcttgatcacaccacccacaccggcagccgcctgcgtatgaccgatgttcgacttcaacgaccccagccacaacggacggccctccgcccggccctgcccatacgtcgcgatcaacgcctgcgcctcgatcggatcacccagcctcgtccccgtcccgtgcgcctccatcacatccacgtccgacgtcgacaaccccgcacccgccaacgcccgcacgatcacccgctgctgcgacggaccgttcggcgccgtcaacccgttcgacgcaccgtcctggttcaccgcactgccccgcaccaccgccaacacctcgtgcccgttgcgccgcgcgtccgacaaacgctccagcaccaccacacccacaccctcggaccagcccgtcccctccgcatccgcggaaaaagaacgacaccggccgtccgccgacagaccaccctgacgaccgaactccacgaacgcgtacggcgtcgccatcaccgtcacaccacccgcgagcgccaacgaacactcccccgcacgcaacgactgcaccgccagatgcaacgccaccaacgacgacgaacacgccgtgtccaccgtcaccgcaggaccctcgaacccgaacgaatacgaaaggcgtccggagagcacggagctggcgttgccggtgatgaggaatccggaggcctcggtcgcacgggactcgcgcaggtgtccgaggtagtcctgcatgccggcgccgatgaacacgccggtgtcgccgccgcgcagcgactcggggacgatgccggtgcgctcgatcgcctcccacgaggtctccagcagcagccgctgctgggggtccatggcggcggcctcgcgcggcgagatgccgaagaagcccgcgtcgaactccgcggcgtcgtgcaggaacccaccgcccgcggtgagcagcccgtcgggagcggacgtcgagccgtcaccggccaccccgccactggccggcccggcgccgacgccgtcgaggtcccagccgcggtcggccgggaacggcgagatcgcgtcccgcccctcggccaccaactcccacaggtcctcgggcgaggccaccccgcccggatacttgcaggccatgccgacgacggcgatgggttcccgggccgcgtcttcgacgtcctggagccgaccacgcgtctcgtgcagctcggcgctgacgcgcttcaggtaatccaacagctggttctcggatgccatttcccgctctccccatcaattcccggagggttctccacttgccgccgacgactcaggactcgtctatcccgggccctccagcggggagatgccgagctgccgggtgacgaagtcgaggacttcgtcctcggtggccgactccagcagcgcccccgtgtccggtgtgcccctctcggacgaggacccggagggcgaggagaacatgaccgtctcggaagcggaccgtttgccttcccagccgtccgcgagcgccctgagccgggacgccgcggccttccgcagggcggcgtccagccggagttcctcgaggctctgcgccactcggtacagctcggcgagggcggattccgcggtgacggcccgctcctccggctggatcaggccgtgcagttgcgcggcgagtgccttgggcgtcgggtggtcgaagaccatcgtggcgggcaggtccacgcccgcggcccgctgcagtctgttccgcagcgccatcgcggtcatcgagtcgatccccagctccttgaagggctggtcgacggcgagggcggcggggtcggcgtagccgagcaccgccgccgcgtgatcacggacggtgtccaggagcaggcggtccgcgtcggtccgcgccatgccgggcagtcgccgggcgagcgtgggaccacccgcgccgcccgcgccgtcggaacgcccggcgtcggagccgtcgtccgcggcttccccggatcgtgcgaatgcggcgagcagcgggctgggccgggccgcggtgaagccgtcggcgaacagcggccattcgatctcggccagcacctggctcgcggggccgtcctgggccagcgcgaggtcgagggcccgcacgccgagttcgggctcgatgggcggcagtccgttgcgccgcatccgctgttcggtggccgcgtcgacgagaccgccgccgccccagggcccccaggcgatcgagagggccgggaggcccgcggcgcggcggtgctcggcgagggcgtcgagcaccgcgttggccgcggcgtagttgccctggccgatgccgccgacggtaccgacgaagccggagtagagcacgaacgacgacaggttcaggccggcggtcagttcgtgcaggtgccaggcgccgagcgccttggggccgagaaccgcgtcgagccggtcggcgtccaggttctccagtgcggcgtcgtcgaggaccgcggcggcgtgcaccacggagacgagcggcgcgtcggccggtaccgactccagcagggcgcgtacggcctcgcggtcggcgagatcgcaggcggcgatcgtcacgcgcgcgcccatgctctcgaggtcggcgccaagtccgtccgcgtccagcgcctcaggaccgcggcggctggtgagcagcagatgttgcgcgccctgcgccgccagccggcgggccaggcggctgccgagggcgccggtgccgccggtgatcaggaccgtgccctcgcgcggccacaccgcatccgcgcccacgttcccgtccgtgtccgtgtccgtgtccgtgtccgacagggaaacctgaccgccgtcgcgtccgccggtcgtggggtccggcagccggaacggggtgcggacgaggcgtcgggcgagcacgcccgtcgggcgcagcgcgagttggtcctcgtcgcccgggtcggccagcaccgcgcagagctgagcggcggtggcggcgtccggcgcggtccgtccgtcctgcccgtcggcctcacggggcgcacggggcaggtcgatcagaccgccccacagcgtggggtgttcgagggcggcgacgcggccgaggccccagaccgcggcggcgtgcggtgccgccggagggtcggtcgggtcggttcccacggcgccgtgcgtgaggcaccacagccggcccggcaggtttgtctgcgcgagcgcccgcaccaggagcgtcgtcccggccaggccgcggccgaccgcggggcgtccaggtagcggccggtcgtcgagcgcgagcagggagaggatcccggccggccgggcatcggccgccgcggcgtgcagcgacgcggcccatgcggccgggtcggtgtcctcgcgcgggtcgagttccacgagcagcgcccggccgcccgcggcctcgacggcgtcgagcacccacccgacgagccggtggtcggggccgaatccgtgctcggcgccgggtatcgcgagcagccaggtgccggcgagggcggggacggcgcccggcgcgtgcaggaccggctcccagcggacgccgtagcgccaactgcccagccgtgcctgctccaggtggtcgcgccgccatgccgcgagggcgggcagcagggcgtccgccgactcgccgtccttcagcccgagggcgtccaggagcgcggcggggtcggcctccagcgcctcccacagacccgagtcggccgcctgcgcggcatgcggcgcggacgctccggccagggccgaggggcgcccgccccggccggcctcggatccggccccggagccattcgtggtgcgcggcgtcggccagaagcggctgcgctggaagggataggtgggcaggtcggtcacccggccgtcgctgtcgcgcagcagggcggcccagtccacgtcgagaccgtgcacccagccctcggcagccgaggtgaggaagcggtcggctccgcccgcgtcgcggcgcagcgtcggcacggtcgccggtcggacgcccgcctcctccgcggtgcgttccatcgccccggtcagcagcgggtgcgggctgatttcgacgaatacggcgtccccggactcggcgaggcggcggaccgcggcctcgaactccaccggggcccgcaggttgcgcacccagtactccgcggtcagttcgccgtcgcggaccggctcgccggtgacggtggacagcatctcgacctcggggcggcccggtgcgacgtccgcgagctccgtcaggatccgcgggccgatccggtcgacgtgcggcgagtgcgacgcgtagtcgaccgcgatccgccggacgcgcacgttgtcggcctcgcagacggtgaagaactcgtccagcgcgtccgcgtcgccggacaccacggtggcctcggggccgttggcggcggcgaggaagacgcggccttcgaagggcgcgaggcgggcggcggtctcggctcggccgagcgcgacggacagcatgccgccggcgcccgcgatcccggtcagcgtgcggctgcgcaacgccacgatcttcgcggcgtcttcgaggctgagggcgcccgcgacgcaggccgccgcgacctcgccctgcgagtggccgaccaccgtgtgggggcggacgccgacggagcgccacagctcggccagggacaccatcatcgcgaacagcacgggctggacgacgtcgacccggtccagggacggttcgccgggctcgccgttcagcacgtcgagcagcgaccagtcgacgtgcggggcgagcgcgtcggcgcactcccgcacccgcgcggcgaacaccggcgagctgtgcagcagttcgcgagccatgccggcccactgggcgccctgtccggggaagacgaacaccacccggcggcgaggggctgcggcgccgtcaccgtccacccggttcgcgctcggcacgcccgcggccagcgactccagttcggcgacgaccgtcgcacggtcgccgccgagcacgacggcccgctgctcgaaggccgtcctggtcgtcgcgagcgacagcgccacatcggccaggggcgactcgtcggcggtcaggcgcctgccgatcagccgggcctgggccgacagggcgtcgggggtgcgtgcggagaccaggaagggcagaactcccgatccctgagccggagttccggtgcccttggccgtcggcgcgggtacgtccggttccgggtcgggggcctgttcgaggatcacgtgtgcgttggtgccgctgatgccgaaggacgagatgccggcgcggcggggccggtcggtccgcggccacggctgtgcctcggtgagcaggcggacggcgcccgccgaccagtcgacgtgcgtggccggccggtcgacgtgcagcgtgcgcggcagcacgccgtcgcgcatggccatcaccatcttgatgatgccgccgaccccggccgcgccctgcgtgtgacccaggttcgacttcaacgaaccaagccagagcgggcgttcggccgggtgctcgcgcccgtaggtggcgagcagcgcccgtgcctcgatcgggtcgccgagcgtcgtgccggtgccgtgcgcctccacgacgtcgacgtctgcggcctggacctgcgcgtcctggagggcgcgccagatcacgcgctcctgcgcggggccgctcggcgcggccagaccgttggacgcgccgtcctggttgaccgccgaaccgcgcacgacggccagcacgcggtgcccgttgcgctgcgcgtccgagaggcgttcgaggacaagcatgccggcgccctcaccccaggaggtgccgtcggcggcgtccgcgaaggacttgctgcggccgtccgcggcgaggccacgctgccgggagaactcggtgaaggtgatgggcgtcggcatgactgtcacgccgcctgcgagcgccagcgagcattcgccgcggcgcagcgactgggcggccagatgcagcgccaccagcgacgacgagcaggcggtgtcgatcgtgaccgcgggcccggacagcccgagggcgtaggagacacggcccgagacgacggcgctgttgttgcccgtcacgaggtagccctcgtactcctcggacgaggcctgcaagggcacgtcgtagaacgagtagtaggcgccggtgaacacgccggtctcgctctcgcgcagcgtgtccggggcgatgccggcgcgctccagggactcccaggccacttcgagcatgaggcgctgctgcgggtccatggcgacggcctcgcgcggcgagacgccgaagaaacctgcgtcgaaccaggcggcgccgtgcaggaacccgccctcgcggacgtacgaggtgccctcctcggtgccgtcgccggcgaagagcgcgtcgaggtcccagtcccggtccttggggaaggcgccgatcgcgtcgcggcccgtgcgcaccaggtcccacagcgcctccggggagtcggagccgccggggaagcggcagcccatgcccacgatcgcgatgggctcggtggcgcggcgctcggtgtccttcaggcgccggcgggagtcctggaggtcgccggtgaccttccgcagcgcctgaagaagctgttcctggcttacgtcgtcagacatccacgccaccgttttccactctcgggcaaagcactgtcactcgaagcccttctcgatcagcgccagcaggtcggaggcgctggcggtcgcgatggccgcgccgccgtccgtgtcgtcgtccgcggcttgtgcggccggttcggtgtcgcggggtgtctcgcgcagccgggcggccagcgcgccgagccgggtggcgagccggtcccggtcggcggcggagccttcgaggtccgcgagcgaccgctccagcgaggcgaggtcggccagcgcgcgggcggcctgtccgccgccgtccgggacggcttggcggcagaggaacgcggcgacgtcggtcggcgtcggatgctcgtagacgagcgtggcgggcaggtcgagaccggtggactcgcgcagccgggtgcgcagttcgacggccatcagggagtcgaagccgaggtccttgaaggcgtggtgcggcgcgacgtcccgggccgcggcgtgaccgagcacggcggcgatctgggcgcgcactaggtcgagcgcgacgcgctgctgttcgtcgggactgtgcccggccagcactttggcgaagtccggcgtggtgccgcccgtctcgtcggtgcccggacccgcgccggtgacgccggcgaggtcggaggcggcaccgggcaccggaacgagtccggcgagcagcgggctggggcgggcggcggtgaagacggcggtgaagcgtccccagtcgacgtccgcgacggtgaccgtggtgtcgccgtgccgcagcgcccggtccagggcggtgacggcgagttccggcgccatcggggcgatgccgagtcggcgcaggtcgtcaccgaagctcccggcagccatgccgccgccgtcccaggggccccaggcgatggccttgcctgcggcgccccgggcgcggcgccgctccatcagcgcgtcgacatgcgcgttcgaggcggcgtaggcgccgccggacgcgttgccccagacgcctgcgacggaggtgaaggcgacgaacgccgacaggccgtcgccgaagacctcgtcgaggttgcgcgcgccggtcaccttcgccgccatcacggtcgcgaaggtgtcggcgtcgagcgcggcgacggggttctggtccccggtgcccgcggcgtgcaccaccgcgccgacgggcgtgtccgcggcggcgaaccgggcggccagttcctcgacgtcggcccggcgggagacgtcgcagtccgcgaggacgagttcggtgccgtgccgcgccagttcggcgcggagccggtccgcgtcggcgaagccactggcgcggcggctcgcgagcacgacgatgggcgcgccggactcggcgagccggcgggcggtgtggacggcgagggcgccggtgccggtgatcagcacgggtcccgaggaccacaggtccgcgtcctcggtgtcctcggcgggccgttcgtgggggctcacggtcggctcggcgcgggtcaggcgccgggcgtggacgctgtcgtcccgcagcgcgagctggtcgtcgccgccggatcgcccggcgaggacggcggccagccgggcgagcccgccgcagccgaggggatcgtcgtccgtgctcgccgcgaggtcgatcaacccgccccagagtcccgggtgttcgagtccgacgacccggccgaggccccagacctgggcctgccacgggtccggcgccgcgtcactctccagcgcccgcaccgcgccacgggtcagggtccacagcggcgggtccgtgcggcccgtgtcgagcagagcctgtacgagcagcagggtggcgtccgcggcggccgacacgccgtcgcggcgcgcggtctcaccgctcggccagggcagggcgagcacgcccgcgaaccgctcgtccgacgccggggtccggtccagttcccgggcgagttccttcgccagggtcgcgcgatcggcggtcgccacgtcgaccgtcaggaggcgggtcgtcgcgcccgcgtcggccagtgcggccgtgacggcgtcggcgagcgcggccgacgcgccgtcttcggcggtgtcggcgtcggattcgggggcgacgatgagccaggtgccgtcaagtgccgccgccgggggcgcggcgtccggaagacgttcccaggtgacgtggtagcgccacctgtcggttctggcgatcatgtcctgtccgcgccgccagttgccgagcgcgggcagcacggcgctcagcggcgcgtcggcgccgaggccgagcgtgcgggccaggccgtcgaggttctgctcctcgacgacctgccagaacgccgtgtcctccgcgctgtgcgtgccgtccggcgcggccggaacgtccgccgtgtccggtgcggcgtccagccagtagcgccggtgccggaaggcgtacgtcggcagtgcgacacgccgggcgccgggcgtgagcgcggtccagtcgaccggcacgccgttgacgtacgtccggctcagcgcggtcagcacggtcgccgcctccggccggccgcgccgggaggaggccgcgacgggccgagtcccctcgaacagcggcgtcagggcggcgtccgggccgatctcgatcagcacgtccgcgtccggcaggccgcgcaccgcgtcggcgaagcgcaccgcgcgccgcgcgtgctcgatccagtactcgggggtgtcgaaggcgtgcgtggtgtccgcggccgggctgacctggatctcggccgggtggaaggtgaggccgtcgaccacggcggcgaactgcgcaagcatcggttccatcagcggggagtggaacgcatggctcacccgcagccgggtcgtcctgcggccctcgccgcgccagtgctccgcgatgcgcgtgaccgcgtcctcggctcccgagacgacgaccgcggaaggcccgttcaccgccgcgatctcggcttccgaagtgtccacgaggtaggcgaggctcgcggcgacctcgtcgtcggtcgcctggatcgcgaccatcgcgccgccctcgggcagcgcctgcatcagccgtccgcgggcgaccacgagccgggcggcgtcctcgacggacagcaccccggcgaccacggccgccgcgatctccccgacggagtgcccggcgagggccgagaactcgacgccccacgacatccacagtcgcgccatggcgagttcgaaggcgaacagggcgggctgcgcgaactcggttcgctccagcacctccgcgggctcctgccacagtacgtcccgcagcggacgtccgagcagcgggtcgatcacgtcgcagatctcgtccagggcggcggcgaacaccgggaagtgctcggcgagttcacgccccatcccgggccgctgtgctccctggccggaaaacagcgccgcggcccgtgtgccgcccgtggtgccggcctcgggaccggtgaccgcctcggccgggtgcaccgcgtccggggtaccgagcgccgcgagcgcaccgagcaggccgtcgcggtcctccgcgacgaccaccgcgcggtggtcgaagtcgctgcgcgtggcggccagcgaatacgccacgtcccgcagaccgagtccggggttggcgaccaggtggccgtgcagggctcgggcctgtgcgtgcagaccgtcccgcgtacgggcgttgaccgggatcgcgacggcgacgggggcgtcgtcgtccgtaccggcctcggggctctgcggcacgtgccgcgcgtcgccctcttccaggaccacgtgcgcgttcgtgccgctgatgccgaacgacgacaccgccgccagccggggacgctccgtgcgcggccagggccgttcctccgtcagcagctccagcgcgcccgccgaccagtccacgtcgctggagggcttctcggcgtgcagggtcctgggcagcagttcgtggcgcagcgcctgcaccatcttgatcacgccgccgacacccgccgccgcctgggcatggccgatgttcgacttgagcgagccgaggtacaccgggcggcctgccggacgccgcgcgccgtactcggcgagcaccgcctgcgcctcgatcgggtcgccgagccgggtgccggtgccgtgcgcctcgaccgcatcgacgtcggcggcggcgacgcgcgcctgctccagcgcggcctggatgacgcgctgctgggccggcccgttcggcgcggtcagaccgttggacgcgccgtcctggttcaccgcggagccgcgcaccacggccagcacgtcgtgcccgaggcgccgcgcgtcggagagccgttccaccgcgagcacgcccacgccctcggcccagccggtgccgtccgcgtcgtcggagaacgacttgcagcggccgtccgcggccaggccgccctggcgggcgaactcgacgaagaggtgcggcgaggccatcacggtcgccccgcccgcgagggcgagatcgcattcgcccgcgcgcagtgcctggcaggccaggtgcagggcgacgagggaggacgagcaggcggtgtccagggtgaccgcggggccttcgagaccgagcagataggagacccggccggacatcacgctgttgctgttggcgatcccgatgatgccttcgaggctctccggggtgccgttcagcctgctgaagtagtcgttgtacatgacgcccgcgaacacaccggtgcggctgccgcgcagcgtcttcgggtcgatcccggcgtgctcgacgctctcccaggctgcctccagcaggagccgctgctgcgggtccatcgccagcgcctcgcggggagggatgccgaagaaggcggcgtcgaacccggcggggtcgtcgaggaatccgccgccacgcgcgtacgtggtgccggtcgcggcggggtcggggctgtagatgcgcgtcaggtcccagccgcggtcggcggggaagggcgcgatggcgtcacggccctcggccagcagccgccacaggtcgtcgggcgcgctgacgccgccggggtagcggcaggccatgccgacgatcgcgatgggctcgtcccgctccgtcctgccggtggtgaagtcgggcttcctgtcgggcattacggtgtcgccggtccccgtcggcggtgcgagccgtatttccaggtggcgggcgagcgcggccgacgtcgggtggtcgaagacgagcgtcgcgggcagctggacaccggtctcggcggagagccggttgcgcagttgcaccgacatgagcgagtcgaagcccagctcgccgagcgggcgatcggcgtcgatccggcccgcgtcggcgtggccgaccgccgcggcgatctcggcgcgcacaaggtcgaggagatccgtgccccggtaggcggaccgcgcggggcccgcccctgcggcggcgcggggcgcggcgccggacgatgccgcgccgcgggcccgtacgaggtcggtgaacagcggggccgggcggacggcggtgaacagcggcaggaaccgcgaccaggcgacgtccgcgaggacgcagtcggtgccgagcgcgagcgagcgggccagcgcggtcacggcgtcggccgcgggcagcgggatcaggccgctgcgacggaaccgctgttcccgggtcgcgtccaccatgccgccgcccgcccacggtccgaaggcgagcgacgtcgcggcgcggccctgggcacggcggcgcgcggcgagcgcgtccagtgcggcgttgccggcgccgtacccggcctgcccggcggcgccccagattccggatacggaggagaagaggacgaaggcatcgaggccggcgtcggcgagcacgtcgtcgaggacgagggcgccgcgcgtcttcccgtcgagcacggcgcggaagtcgtccgcatccgtctccagcagcggcgcctcgtgcgcgatgccggcggcgtggaagacggcgcggatcggcgcgcccgacgccgcggcctcctggacgacagcggccatcgcggcgcggtcggcggtgtcggcggcgacgacgcgcaccgggacgccggtggcggacagctcctccagcagctccgcggcgccgggcgcttcggggccgcggcgggagaccagcagcaacgagcatccgtccgcgacctgttgcgttgcggacccggccgccagtcccgcgacccagcgggcgacgtgcccgccgagcgcccccgtgccgccggtgatcagcaccgtgccggagggctgccagtgcgtcaggtcctggttcggcgccgggtcgttgtccggtgcggacctgacgacgcgtcgtacctcgagcccgtcgcccgccacgcgtacctggtcctcccgggtgtccccggcgagcagggcggccaactgtccgaggggcggttcggcgccgtcggcaggcaggtcgatcaagccgccccaggtatcgggcagttcgagggcggcggcgcggccggctccccagaccgccgcggcgtccgggtcggtgccgctgtcccgggtcaggcaccacaccttggcggtcacgcgggcgtcggtgagcgcctggagcgcggtcagcagggtctcggggcggtccgcgaagcacagtacgcccgcgaagccgtcggcgatcgcgtcggccggctgagcggagagggcgcagagggcggacgcgagcgtggcgcggtccgcggcctcgtgcggcgggacggtcacggtgtgctcgacgagttcgcccacccacgcggggatgtccgacgcgccgtccgccgccgcgggcgggacgaccagccaggtgcccggcacggggacggtacgcggcgcgaggtcggcgggctgccatgtcacctcgtaccgcagccggtcgagggcggcgtcggcctgcgccgacaaccgggccgggtccgcctcgcggagcaccagctcgcggacggtgacgaccggctctccgtcgctcgtcaccgcgtccagccgcgcgctgtcccccgcagtgcgcagccggacccgtatcggggcgccgtccgcggtggccgagccccgcgacggccacacggtggcaccgttgaaggcgaacggcagccggatccgccggctctcgtccccggtcagggcgagcgggtgcagcgcggcgtcgagcagcgccgggtggatgccgaagcgcccgtcgtaccggccgtcgggcagcgcgacctcggagaacaggtcgtcgcccgcccgccatacggcacgcaggccgcggaaggcgggtccgtagccgtagccgcggtcggccagatccgcgtacaggtcctggaccggtacgggttccgccccggcgggcggccacgcgcccacggcccagtcgacgtcgctgtgttcgggctcgtgaggttcatgtcgttccggcaggggctccgtgagtgtcccggtgacgtgcaccgtccactgccctggcacctggcccgtcgcctgccgcgcgcgaatcgtcagctcacgaccggccgtcccttccgacgggccgacccagacctgaacgtccgcgccgccgtcgcccggcaccagcggggcctggatgacgagttcggagatctcacggcagcccgcctgccgggcggcctgtgcggccagttcgacgaagcccgcgccggggaacaggacgctgccggctatcgcgtggtccgcgagccacgggtcggcggacggcgagatgtgcccggtgagcaccaggccgccggggccgggcagttccaccgcgccggacagcagcgggtgcgcgagcgcgtcggtcccggcaccggcggtcgcccggggcggcgcgagccagtagctctggcgttggaacgcgtacggaggcaggtcgacgaggggcgcggcgggcagcagcgccgcccagtcgacgcgcacgccgtgtgcgtaggcctgggcgagggtggtgaggacggtgtgcgtctcggaccggtcgcgccgggtgctggcgaggacggtacgcccgtcgtccccatgggcctgcgcagcacccaccatcggtgcgaggacggcgtccgggccgatctccacgaggacgtcggcggcggcgaggccgcggaccgcgtcgtggaaacggaccgcgttgcgggcgtggtcgatccagtagtccgcgctgtggaaggggtggtcgctcgccgccgaggccgcgatagcagtcttcggaatgctgaaggtcaggcccctgacgacctcggcgaagtcgtcgagcatgggctccatcagagcggagtggaacgcgtggctgacacgcaggcgcgtcgtacggcgtccgcgaccgcgccagatctcgcccacgcgctccacggcgtcgagcgcacccgagacgacgacggcatcgggcccgttgacggcggcgatgtccacggcgccgtcggccgcgtgcggaccgtccgtgaccgtggccagcgtttcggccgcctccgtctcgctcgccgcgatcgcgagcatcgccccgccctcgggcagcgcctgcatcaaccggcctcgggccacgacgagtcgggcggcgtcctgaagggtgagcacctcggcgacgaccgcggcggcgatctccccgacggagtggccggcgagggcggtgaaggtcacgccccaggactgccacaggcgtgccagagcgaattcgaaggcgaacagggcgggttgcgcgaactcggtgcgggcgacggtctgtgcgtcggactcccacatcacctctcgcaggccgcgcccgagcagcgggtcgacgcaggcgcacacctcgtcgagcgcggcggcgaaggccgggaagtgcgcgtcgagttcgcggcccatgccgggccgctgcgcaccctgcccggtgaacagcgcggcgacccgtccggcgcccgcctcgaccggggcggtgtcggcgagcccggcgagcaggcccgcgcggtccccggcgaccgcgacgcggtgggcgaacagcgacctgtggtccacgaggttgcgggcggcgtccacgatcggcagcccggggtggaccgccaagtggtcacgcagcgcggtcgcctgggcccgggccgcttcgggcgtcttgccggagaccaccaccgggacggcgacgcgcgcccagtcctggccgttcgcggcggagtcctgatccacggaccgccgggtgttcgcggcggaaccctggcccgcgagccgctgcgcgtccgcggtcgcacccgcgtcctcggacacgaactcctcgaggatcacgtgcgcgttggtgccgctcatgccgaacgacgagatgcccgcccggcgcgggcccgccggggcgtcccattcccgggcctgcgcgagcagccgcaccgttcccgccgaccagtccacctcggtcgtggggttctccgcgtacaggctgctcggcatgacgccgtggcgcatcgcctgcaccatcttgatgacgcccgcgacaccggccgccgcctgtgtgtgcccgaggttcgacttgagcgagccgaggtagagcgggcggcccgcccggcggctgcgcccgtaggtcgccatcagcgcctgtgcctcgatggggtcgccgagccgggtgccggtgccgtgcgcctcgacgacgtcgacctcgtcggccgagagccctgcgctgctcagggccgtctcgatgaggcgctgctgagccgggccgctcggtgcggtcaggccgttggacgcgccgtcctggttcaccgcggacccacgcaccacggccagcacacggtggcccagccgccgggcgtccgacagccgctccagagccaggacgcccacgccctccgaccatccggtgccgtcgccgtcgtcggcgaaggagcggcagcgcccgtcggtggagacgacccgctggcggctgtactcgatgaagaacagggggctggacatcaccgcgacgccgccggccagcgcgagatcgcactcgccgctgcgcagcgcctgcgcggccaggtgcagcgagaccagcgaggaggagcaggcggtgtcgaccgtgaccgcggggccttcgaacccgtacaggtacgagacccggccgactgccatgctgcccgcgctgcccgagtggatatagccctcgtatccgtcgggcgcggccgtggcgaaccggccgccgtagtcgtggtgcatgacgccggtgaacacgcccgtccggctgccgcgcagggtcgccgggggaatgcccgcgtcctccaaggcccgccaggtcgtctccagcagcagccgctgttgggggtccatggagaaggcctcgcgggggctgagcccgaagaagtcggcgtcgaacaggtcgatgtcgccgaggaatccgccctcgcgcgtatacgaacggccggtcgcgtcggggtcggggtcgtagaggcctgcgacgtcccagccccggtccgtcgggaagccggtgatcgcgtcgcgtccctcggccaccagctcccacagctcctcgggcgaggtcaccccgcccgggtagcggcagctcatgccgacgatcgcgatcggctcgcgggccgcggcctccgcatcgcgcagccgggatcgcgtacgttgcaggtcggcggtcaccttccgcaggtactcgacgagctgctgttcgtcagtcatgttcctcgcccatcggcgtacggcgggttcgctggcttcgcgaacccggcatcgaatgaactgcacgagcccgccgaccggatcgaatccggcggtcttcgtctcggctctcaatgcggcgcggactgcggcggccgtgccgaatcggatttcttcgatccaagcacggaaacagcggcgccccctactcaggcacccccctaaaacacccggcatggccttcggttggggttcgaccaggggtgatgcggcagcgccggatgggcgggcacggcatgcaagagccgggcgcgggcagcggcgatcgcggcccgcgcccggcgcggttcagtcgtccccagggaaccgtggatcaatggctccacgaaccacggatctacggatctggagggaactggaggggatacggatccggaggggacaccagaattcaggattcaggaagccggtgagtccgcacgcttctcggcggctccctcggcacgctcttcggcacaccaccatgccggctgtacggtcgcctgcgggaccgagccgagagcggcccggatgggctgcgcgcccgccgagacggccagcacgccgcgcaccggctccggcgcgaacagcgcctcgcagatcgtctcggcgagatccgtcctggagaccgtgccctcgggctgcgggtcggcgccgccgcgtaccgtgaccaggccggtggccggggagttgtcgagcatccccggacgcaccacgcaccagtccaggtcgtgtccggccagcttccgctccacgtcccgcttctccgcgaggtacgactccagctcgtcaccgagcctggtgtgcagctcgtcgtccggcagatatgcgctgaccaggacgaagcgccggatgccgaccaactgcgcgacccccatcaactcccctacgagcgaggtggacgaggtgtccgtggcgtccgggccccagccggtcccggtggccacggcgatcccgccgcactcgcccatcgcccgtagcgccgccgggctcgtcctgtccgcctcggacgcgatcaccagcggctctgtccctgcggcccgcaacgactcgctgtgcccggcctccccgatgagccccaccggggtcaggccgcgggcgaggatcgactcggcgagccgccggcccagcgcgctcgtgacgccgaggacgacgacgttcttcttccccgccatcggccccgcctgcgccgtccgtgcggcttccgccctcagcgcggcatcggctgtccgcggggcgtccgccgtctcatcggctttcgatatcggcatgttcgagatgtcttctttcatcggctcggtcgccatatcagtccgctcacgccacgtcctggatttccgcgggcgtgtggtccggagcaccgcgcgattcgacgatggcgccgatctcggcgcggcgcctgatatcaaattttcggtaaatacgggtcaagtgctgttcaatggtgctcgcggtgacgtagagcgactccgcgatttcgcggttcgtgtatccctgagccgccattctggcgacattccactcggccggggtgaggctgcgcccgtcgcgtcgtgccgcgggcggcggcaccttgcgcctcgcctgcgatccgccggacatctccgcaagcgcccactgcgccccgcagccgcgcgccgtcttctcggccagccgcatcgtcttacgaccgccggccaggtctccggtgtgcttgtagatctgcgcgagctggccgagcgcgcgggcgtattcgagttcgtcgccgcaggactggagaacggcgatggatttccggcacgccagcgcccgttcccccggcgggaccgtcgccacccgcaggcgcagcccgatgccgcgcgcccgggtgttcgagtccggcgagaacgccagctgctcgtcgatgagctccgccgcctcggcgtactcctccagcttcagcagcgcctccgcgaggtccaggcgccacggcaccaggcccggcatctccatgccccaggccgtgaggatggcgccgcagctgcggaaatccgcgacggcggcgcgcagggcgccggtcgccagccggaaacgcccgcgggcgcgcaggtacaccaggccgtacgggctggccaggtccgcctcggtcatcgggcgggcgagttgcgcggctgcctcctcgaagcgtccttgctcggtcagcatcagcgcggacaggccgcgcggcagaccggcgagcacgccccaccggtccgcgtcccagcgctcgaagccgcggcgcacggccgtctgggccgcgacgaggtcacccttccaccaggcagcgaccgcctcggccacgctcagcatggaggccagcggcagcggcatacgttcgttgtcggcgagggcggcgttgtcggcgagggcggcgtaccagggggcgcgcgggtcgagtcgtcccgtcgccatcaggcagaacagcgccacgagcatcggctccaggctcgaatagtcgaagttggccgactggaggatctcctccgcgcaggtcaccgcggcttccgcgtctttcacggccttcgcggaacctgcggaatccgcggaacccgtgggccgtggaccggagttcggcgcatcggagcactggacgcccggcgcgagcagccacgagagccgttgcgcgctggacagccacgagaatccctcggcgatcagctgggggtgcggctggtcgtcgggcgcgggcgcgtccgggcaggcgtcggggaacagatgacgcaaccacagcccgctgagcgttctctggacctcggcacggacatccgcggcgccgagggcgtcgccgggactgacagcaccgacagcgttgccgcgtccaggcgcgggttcggccaggcgcagcatgtcggacgcctgcgccatctcgccgtgtctggccagatgccgcgagagacgggctaacgaacccggcttgaaagtgccgtcggacgcggcagccgccagccgccgcagccgcggtgcggtgagcgccgggtccatccaccacaccatgtcggtgatccggacgccggcctcctcccgcaggtcgggcccggagccccagaacgaggcggactccaggagttggacagccagccggtgccggccgagacgcgaggcgtgctcggcggcctcatagaggaccccacccgcccagggctgcggcgcgatcccggcctcgtgcagatacggagcgatctcccaggcgggcacgccctgttcgtgcaggacctgggcggcacgcagccgtaaggaccgcagtcccgcggggtgcgtggaccagaggacggcgtcccgcagatacggatgcagggtcatgtcggggcgcacgatcccgctcgcgatggcctggcccgccacgtgggagacgtactcggcatccttctccagcacgttccacagccgctcgggtgtgcactgcgcgtcgaggaccgcgaccgcctccaccaggttcatcacctcacgggaggtgtgctcgcgcagccgccgggccaggccggccgacggaggtccgtccggcgcgtccggatcccggccgatcgcggccgcggcctcgtccagcatcgcgagggtcaggcccgggttgccgccggtgatcggatggatcgcctctgccagagcgtcggcgcgggggccggggacgcgggcgcgcgccagttcagcgaccgcggcccggcccagcggccgtacgcgaacgcgctccgcccaacccgcggcgagctcacgcagttcatcgacgatcgagggcaccgtagcggcgctgacgccggtcagcaggaccctgatccttccggtgtgcgtgaggcggaccacctcgcggacgaacacctgggattcgggggccagcaggtccgtctcgtccaccgccagcagcagcgagcgggtggcggcggccacgagccgccgcgcctgctcgccgggctctcgcgcgccgaactcgcccagttcctcggcgtatcccacgccgcacacactgccgcgcacggtcaggacgccgatccccatccgtcgcgcttcggcgcacgccgcttccagcaaggccgtcttgccgcacagggcagggccctcgatcacgcagagcccggcgctgtcggccggacggcgtcgcagccatcgtcctagttcggcgagctctccatcgcggtcgacgagagacaccgcacaattcgactcggacccggaactcggcttgggcccgcaactcgacctgaacaccgggttaccaccgcccggtcgagtcatggttcacacatcgacgtccccgcgcggccgccgcatataacacgcggtcgacgtctcgcgggccctgacaacgcgacaggtggtcagcccgcccgttcagggtgtaagcgacagctttcatggcttcgtctttcgaaagagtggatcggcgggcacgaaggtgaccgctgcttttgccgtgggcgacagagcgactgcacagcaaagccgtcccccagtccctgcgcccatcgccggcgaatgaaagggtgggccggcgcgatggtcaagtcatgccggcgcgccgacagacggagtgcgggccgcgctgaccacgtccggtccggtccggttcatcgtcccggtgacgcgacgacgcagaggcgggcgtcccgtgggacgcttcggagcgaattgcatgatgagacgttcccccgttgcgtagtgcggcccaccgaccccggtaccggctgcccttttctgaattcttccccaacagacatgtgcgatgggggatcaggttggtcaacaatgagttaaccctatgtgaggtgaggacagcatgctgcaagggggtggtcgagttggcgacatcccgacgccgaccggcctcggccgcggtgaacgtcagcatcccgcggcctaccggcgcagggcagcacagccgaccgggctcacacggtgatcgcctccccctccgggacgatggtcagaggcacctcggtcagcgccgccgggctcaggaaccgggccatcgactcctgaccggtccggctgagcatgatctcgtggatctggacgagacggttgggggcgacctcgcgtacgtagtcggccgcccggccgagctgggtccacggcccgctggtgggcagcagcagcgtgtcgacgggcgcgggcggtacgtggtacgcgtcgccggggtggtggacccggccgtcgacgaggtagccgacgttggtgacgcgggggatgtcgcggtggatcgcggcatgcaggtcgccggacaccgccacgtcaaaaccggcgacgtcgaggcggtcgccgtccgcgaccgcgacgacctggccgcgccgggccgcccagcgtccgaccacacggaccggtccgtagacccgcaggccggggcgggcgtccagggcccgggcgatcaggtcctcgtcgaagtggtcgaagtggtcgtgcgtgatcaggacggcgtcggcggcggcgaccacctccttcgcgtcaggggtgaacgtgcctgggtcgatggcgatccgcccaccgtccttgaccagcgacacacatgcgtgggcgtgcttgatcagctgcactgccggctcctatcggtgtgatgtacagggtgcactgtaatacagtcccccctgtactcttttgctagactggggggtgtgaacgacgcgccttcgaacgaaccggcccccctccccgacgagttggccgtccgcctccgggcggttgtcggcaccctggtccgtagcgcccgtaccgtcgatcggctcgcatccgttccggcggcggtgctcggcctcctcgacacgcgggggccgatgaccacggccgacctggcggcgacccgcggagtgcgccaccagacgatggccgcgaccgtcagggaactgaccgaggccggcttcctggcctcacgcaccgatccgggcgatgcccgcaggaaggtcctcgctctgacgaaagcggggaagaaggcgctcgacacggaccgccgtcagcgcgtcggcgtgcttgccgacgcgctggaggaaacgctggacgatgaggaccggcgcgccttggcgcacgcccttgacctcatcgatcggatcagcggcagcatccggggcggccactccttctccggcgagcgcgagttcaacaccggagcatggtgacgaggcccgagtagcctgctccggctggtttacgcgcgttttccacgcgcgcgcgaacgcgtcggggttgtcgaacatgacgttgtggccggtgtccgggacgatgcggagcttcacccccgcgctctccgcagcccgtccctgccgggcagttcgccgcgcggctcgccctgaaggcggatgcgttccatgcgtgcccgctcgggcatcacccccatcgcggggccggtgccgcgatagaggtcaaatggctgagaagtccctggtgcgagcggatccgtcgccgctacccgcccgagggcggcatggaccagctccacctcgagctccccggacgtgcctccgcgccgcccaacaccaccaacacgacgtctcgttgaccggctgcaccacaagaaaggacataatcccgatctcgctggttcctcggcggccccgggtcccccttgaacggtcgtgaaccgggctgaacgagacggaaaccgggacggcccgtacggggcgttgtcagtggccttacctcgtgccgcatcagacagcgttcaccctgtagcaccccgggaaaactcgtcgaaacagtctccctgcagcaaccgaggatggcccgcctccggcccttgacggctggctgggcggacgcggactcgggggctagagtgggttgcttgccggtggcctccggggctcgcaacggcccgtcccagccgtcgcc

SEQ ID NO: 2 - protein, 3542 aa, Streptomyces pulveraceus
MPGVLGGCLSRGRRCFRAWIEEIRFGTAAAVRAALRAETKTAGFDPVGGLVQFIRCRVREASEPAVRRWARNMTDEQQLVEYLRKVTADLQRTRSRLRDAEAAAREPIAIVGMSCRYPGGVTSPEELWELVAEGRDAITGFPTDRGWDVAGLYDPDPDATGRSYTREGGFLGDIDLFDADFFGLSPREAFSMDPQQRLLLETTWRALEDAGIPPATLRGSRTGVFTGVMHHDYGGRFATAAPDGYEGYIHSGSAGSMAVGRVSYLYGFEGPAVTVDTACSSSLVSLHLAAQALRSGECDLALAGGVAVMSSPLFFIEYSRQRVVSTDGRCRSFADDGDGTGWSEGVGVLALERLSDARRLGHRVLAVVRGSAVNQDGASNGLTAPSGPAQQRLIETALSSAGLSADEVDVVEAHGTGTRLGDPIEAQALMATYGRSRRAGRPLYLGSLKSNLGHTQAAAGVAGVIKMVQAMRHGVMPSSLYAENPTTEVDWSAGTVRLLAQAREWDAPAGPRRAGISSFGMSGTNAHVILEEFVSEDAGATADAQRLAGQGSAANTRRSVDQDSAANGQDWARVAVPVVVSGKTPEAARAQATALRDHLAVHPGLPIVDAARNLVDHRSLFAHRVAVAGDRAGLLAGLADTAPVEAGAGRVAALFTGQGAQRPGMGRELDAHFPAFAAALDEVCACVDPLLGRGLREVMWESDAQTVARTEFAQPALFAFEFALARLWQSWGVTFTALAGHSVGEIAAAVVAEVLTLQDAARLVVARGRLMQALPEGGAMLAIAASETEAAETLATVTDGPHAADGAVDIAAVNGPDAVVVSGALDAVERVGEIWRGRGRRTTRLRVSHAFHSALMEPMLDDFAEVVRGLTFSIPKTAIAASAASDHPFHSADYWIDHARNAVRFHDAVRGLAAADVLVEIGPDAVLAPMVGAAQAHGDDGRTVLASTRRDRSETHTVLTTLAQAYAHGVRVDWAALLPAAPLVDLPPYAFQRQSYWLAPPRATAGAGTDALAHPLLSGAVELPGPGGLVLTGHISPSADPWLADHAIAGSVLFPGAGFVELAAQAARQAGCREISELVIQAPLVPGDGGADVQVWVGPSEGTAGRELTIRARQATGQVPGQWTVHVTGTLTEPLPERHEPHEPEHSDVDWAVGAWPPAGAEPVPVQDLYADLADRGYGYGPAFRGLRAVWRAGDDLFSEVALPDGRYDGRFGIHPALLDAALHPLALTGDESRRIRLPFAFNGATVWPSRGSATADGAPIRVRLRTAGDSARLDAVTSDGEPVVTVRELVLREADPARLSAQADAALDRLRYEVTWQPADLAPRTVPVPGTWLVVPPAAADGASDIPAWVGELVEHTVTVPPHEAADRATLASALCALSAQPADAIADGFAGVLCFADRPETLLTALQALTDARVTAKVWCLTRDSGTDPDAAAVWGAGRAAALELPDTWGGLIDLPADGAEPPLGQLAALLAGDTREDQVRVAGDGLEVRRVVRSAPDNDPAPNQDLTHWQPSGTVLITGGTGALGGHVARWVAGLAAGSATQQVADGCSLLLVSRRGPEAPGAAELLEELSATGVPVRVVAADTADRAAMAAVVQEAAASGAPIRAVFHAAGIAHEAPLLETDADDFRAVLDGKTRGALVLDDVLADAGLDAFVLFSSVSGIWGAAGQAGYGAGNAALDALAARRRAQGRAATSLAFGPWAGGGMVDATREQRFRRSGLIPLPAADAVTALARSLALGTDCVLADVAWSRFLPLFTAVRPAPLFTDLVRARGAASSGAAPRAAAGAGPARSAYRGTDLLDLVRAEIAAAVGHADAGRIDADRPLGELGFDSLMSVQLRNRLSAETGVQLPATLVFDHPTSAALARHLEIRLAPPTGTGDTVMPDRKPDFTTGRTERDEPIAIVGMACRYPGGVSAPDDLWRLLAEGRDAIAPFPADRGWDLTRIYSPDPAATGTTYARGGGFLDDPAGFDAAFFGIPPREALAMDPQQRLLLEAAWESVEHAGIDPKTLRGSRTGVFAGVMYNDYFSRLNGTPESLEGIIGIANSNSVMSGRVSYLLGLEGPAVTLDTACSSSLVALHLACQALRAGECDLALAGGATVMASPHLFVEFARQGGLAADGRCKSFSDDADGTGWAEGVGVLAVERLSDARRLGHDVLAVVRGSAVNQDGASNGLTAPNGPAQQRVIQAALEQARVAAADVDAVEAHGTGTRLGDPIEAQAVLAEYGARRPAGRPVYLGSLKSNIGHAQAAAGVGGVIKMVQALRHELLPRTLHAEKPSSDVDWSAGALELLTEERPWPRTERPRLAAVSSFGISGTNAHVVLEEGDARHVPQSPEAGTDDDAPVAVAIPVNARTRDGLHAQARALHGHLVANPGLGLRDVAYSLAATRSDFDHRAVVVAEDRDGLLGALAALGTPDAVHPAEAVTGPEAGTTGGTRAAALFSGQGAQRPGMGRELAEHFPVFAAALDEICDVIDPLLGRPLRDVLWQEPAEVLERTEFAQPALFAFELAMARLWMSWGVEFSALAGHSVGEIAAAVVAGVLSVEDAARLVVARGRLMQALPEGGAMVAIQATDDEVAASLAYLVDTSEAEIAAVNGPSAVVVSGAEDAVTRIAEHWRGEGRRTTRLRVSHAFHSPLMEPMLAQFAAVVDGLTFHPAEIQVSPAADTTHAFDTPEYWIEHARRAVRFADAVRGLPDADVLIEIGPDAALTPLFEGTRPVAASSRRGRPEAATVLTALSRTYVNGVPVDWTALTPGARRVALPTYAFRHRRYWLDAAPDTADVPAAPDGTHSAEDTAFWQVVEEQNLDGLARTLGLGADAPLSAVLPALGNWRRGQDMIARTDRWRYHVTWERLPDAAPPAAALDGTWLIVAPESDADTAEDGASAALADAVTAALADAGATTRLLTVDVATADRATLAKELARELDRTPASDERFAGVLALPWPSGETARRDGVSAAADATLLLVQALLDTGRTDPPLWTLTRGAVRALESDAAPDPWQAQVWGLGRVVGLEHPGLWGGLIDLAASTDDDPLGCGGLARLAAVLAGRSGGDDQLALRDDSVHARRLTRAEPTVSPHERPAEDTEDADLWSSGPVLITGTGALAVHTARRLAESGAPIVVLASRRASGFADADRLRAELARHGTELVLADCDVSRRADVEELAARFAAADTPVGAVVHAAGTGDQNPVAALDADTFATVMAAKVTGARNLDEVFGDGLSAFVAFTSVAGVWGNASGGAYAASNAHVDALMERRRARGAAGKAIAWGPWDGGGMAAGSFGDDLRRLGIAPMAPELAVTALDRALRHGDTTVTVADVDWGRFTAVFTAARPSPLLAGLVPVPGAASDLAGVTGAGPGTDETGGTTPDFAKVLAGHSPDEQQRVALDLVRAQIAAVLGHAAARDVAPHHAFKDLGFDSLMAVELRTRLRESTGLDLPATLVYEHPTPTDVAAFLCRQAVPDGGGQAARALADLASLERSLADLEGSAADRDRLATRLGALAARLRETPRDTEPAAQAADDDTDGGAAIATASASDLLALIEKGFE

SEQ ID NO: 3 - protein, 1738 aa, Streptomyces pulveraceus
MSDDVSQEQLLQALRKVTGDLQDSRRRLKDTERRATEPIAIVGMGCRFPGGSDSPEALWDLVRTGRDAIGAFPKDRDWDLDALFAGDGTEEGTSYVREGGFLHGAAWFDAGFFGVSPREAVAMDPQQRLMLEVAWESLERAGIAPDTLRESETGVFTGAYYSFYDVPLQASSEEYEGYLVTGNNSAVVSGRVSYALGLSGPAVTIDTACSSSLVALHLAAQSLRRGECSLALAGGVTVMPTPITFTEFSRQRGLAADGRSKSFADAADGTSWGEGAGMLVLERLSDAQRNGHRVLAVVRGSAVNQDGASNGLAAPSGPAQERVIWRALQDAQVQAADVDVVEAHGTGTTLGDPIEARALLATYGREHPAERPLWLGSLKSNLGHTQGAAGVGGIIKMVMAMRDGVLPRTLHVDRPATHVDWSAGAVRLLTEAQPWPRTDRPRRAGISSFGISGTNAHVILEQAPDPEPDVPAPTAKGTGTPAQGSGVLPFLVSARTPDALSAQARLIGRRLTADESPLADVALSLATTRTAFEQRAVVLGGDRATVVAELESLAAGVPSANRVDGDGAAAPRRRVVFVFPGQGAQWAGMARELLHSSPVFAARVRECADALAPHVDWSLLDVLNGEPGEPSLDRVDVVQPVLFAMMVSLAELWRSVGVRPHTVVGHSQGEVAAACVAGALSLEDAAKIVALRSRTLTGIAGAGGMLSVALGRAETAARLAPFEGRVFLAAANGPEATVVSGDADALDEFFTVCEADNVRVRRIAVDYASHSPHVDRIGPRILTELADVAPGRPEVEMLSTVTGEPVRDGELTAEYWVRNLRAPVEFEAAVRRLAESGDAVFVEISPHPLLTGAMERTAEEAGVRPATVPTLRRDAGGADRFLTSAAEGWVHGLDVDWAALLRDSDGRVTDLPTYPFQRSRFWPTPRTTNGSGAGSEAGRGGRPSALAGASAPHAAQAADSGLWEALEADPAALLDALGLKDGESADALLPALAAWRRDHLEQARLGSWRYGVRWEPVLHAPGAVPALAGTWLLAIPGAEHGFGPDHRLVGWVLDAVEAAGGRALLVELDPREDTDPAAWAASLHAAAADARPAGILSLLALDDRPLPGRPAVGRGLAGTTLLVRALAQTNLPGRLWCLTHGAVGTDPTDPPAAPHAAAVWGLGRVAALEHPTLWGGLIDLPRAPREADGQDGRTAPDAATAAQLCAVLADPGDEDQLALRPTGVLARRLVRTPFRLPDPTTGGRDGGQVSLSDTDTDTDTDGNVGADAVWPREGTVLITGGTGALGSRLARRLAAQGAQHLLLTSRRGPEALDADGLGADLESMGARVTIAACDLADREAVRALLESVPADAPLVSVVHAAAVLDDAALENLDADRLDAVLGPKALGAWHLHELTAGLNLSSFVLYSGFVGTVGGIGQGNYAAANAVLDALAEHRRAAGLPALSIAWGPWGGGGLVDAATEQRMRRNGLPPIEPELGVRALDLALAQDGPASQVLAEIEWPLFADGFTAARPSPLLAAFARSGEAADDGSDAGRSDGAGGAGGPTLARRLPGMARTDADRLLLDTVRDHAAAVLGYADPAALAVDQPFKELGIDSMTAMALRNRLQRAAGVDLPATMVFDHPTPKALAAQLHGLIQPEERAVTAESALAELYRVAQSLEELRLDAALRKAAASRLRALADGWEGKRSASETVMFSSPSGSSSERGTPDTGALLESATEDEVLDFVTRQLGISPLEGPG

SEQ ID NO: 4 - protein, 3537 aa, Streptomyces pulveraceus
MASENQLLDYLKRVSAELHETRGRLQDVEDAAREPIAVVGMACKYPGGVASPEDLWELVAEGRDAISPFPADRGWDLDGVGAGPASGGVAGDGSTSAPDGLLTAGGGFLHDAAEFDAGFFGISPREAAAMDPQQRLLLETSWEAIERTGIVPESLRGGDTGVFIGAGMQDYLGHLRESRATEASGFLITGNASSVLSGRLSYSFGFEGPAVTVDTACSSSLVALHLAVQSLRAGECSLALAGGVTVMATPYAFVEFGRQGGLSADGRCRSFSADAEGTGWSEGVGVVVLERLSDARRNGHEVLAVVRGSAVNQDGASNGLTAPNGPSQQRVIVRALAGAGLSTSDVDVMEAHGTGTRLGDPIEAQALIATYGQGRAEGRPLWLGSLKSNIGHTQAAAGVGGVIKMVMAMRHGVLPRTLHVSQPSPHVDWSAGAVELLTRARQWPETGRARRAGVSSFGISGTNAHVILEHEPVESTEAPVGSAQVPVGSVQARVESTEAPLVAGELPWVVSGRTEGAVRAQAARLAAFVAGRGGGAGALDVGGVGLALVSSRSVFDHSAVVSGGSLDELLAGVGGVARGDGSAAGGVVLERRVAGGVGVAFSGQGSQRPGMGRELYGRFPVFAAALDEVCAEVEAQTGAELLSVVFGDDAGVLEDTGVAQPALFAVEVALYRLAESFGVRADVLIGHSLGELSAAYVAGVWSLADAVRVVVARGRLMGSLPSGGRMVAVEATEEEVSGWLVDGAVIAAVNGPAAVVVSGVEGAVEAVVERARGGGRRATRLRVSHAFHSPLMEPMLAGFAQVLARVEFRAPSIPVVSNVTGEVASAAELCSPEYWVRHVREAVRFGDGVGAVLAQGVATVVELGPEAVLTAMGASHPGVVENGAVFLPTLGRRTGDVNGFLTALARVHARGHQVDWTALGRANDLARELPTYAFQHEHHWLAGNAASVDAAHLGMRAVDHPFLGAAVTLPGTGGTVLTGTISPGTHPWLGDHVVLGSTLLPGTAFLELAFTAAARVGCAGVEELTLEAPLILDGGSAHVVQVLVGEAEAAGGGRAITVHSRPVHAAEDAPWTRHATGTLRSDAAEPAPVLAPEPSQVQAWPPRGATPVDLDAVYERLIGLGFDYGPAFRGLHTAWRDGDTVYAEVRLPTRQTDDAGRFSIHPALLDTALHSLALPDLLSGQDECHLPFSWSGVTLHATAVSSARVRIRRLGEGATSVELLDEAGAALATVRSLALRPVTLEQLRSARVSSVESLYGIAWSPLESAGSAVSAAVSASGTGLALLDLGADWSVESVPARRHADLAALAAAIDADPAEAPRDVLIALPALGGVDGDIAAATHQRTQTVLHLLQDWLKQPRFASVTATLLTRGALAVDAAEAAAVDLASAAVGGLIRSAQLEHPGRFRLIDTDGEEASLRALPTLLGTGNGTDIGTGTGVVAEPQIALRAGVPSVPRLRAIAAPETTDADGSADAPAFSGEGTVLITGGTGDLGSLFARHLVTVHGVRHLLLTSRRGPDAPGAAELIAELEALGADVTLAACDMSDRSAVAELLAGVPGDHPLTGVVHTAGVLDDGLLESLTPAQLTKVLRAKADAALHLHELTSRAPLSAFVLFSSVAGVFGGAGQANYAAANAFLDALARRRRALGLPGVSLAWGLWSTEGGMTAELDRANVARLKRTGLLEISREQGVTLFDAALAAAGAAFGASEAGPETVGTHADGLLVPARLNAPVLDEQAAAGSLPAVFQAVVSTRPRPSSRVAGGTAATAGSASAPLLAELRVADREERLQILGGLVAEKVAYVLGHADREAVDRAQPFNRLGLDSLTAVELRNQLGAATGVRLPATLVFDHPTPLAVAEFLYDELARGVLGEPAGSTALAVAEPAARASASHPQDAGDDPIVIVGMACKYPGGVASPEDLWRLVAEGRDAISPFPADRGWDLDGIYDPDPQQPGKTYTREGGFLHDAAQFDAEFFGISPREATATDPQQRLLLETSWEALESAGTRPETLTGSRTGVFMGVMYNDYGARHLNRSPQGYEGYISNGSSGSIASGRVSYSFGFEGPAVTVDTACSSSLVAMHLAAQSLRAGECSMALAGGVTVMATPYAFVEFGRHGGLAVDGRCRSFSADASGTGWSEGVGVVVLERLSDARRNGHEVLAVVRGSAVNQDGASNGLTAPNGPSQQRVIRQALAGAGLSVADVDAVEAHGTGTRLGDPIEAQALIATYGQGRAEGRPLWLGSLKSNIGHTQAAAGVGGVIKMVMAMRHGVLPRTLHVSQPSPHVDWSAGAVELLTRARQWPETGRARRAGVSSFGISGTNAHVILEHEPVESTEAPVGSAQVPVGSVQARVESTEAPLVAGELPWVVSGRTEGAVRAQAARLAAFVAGRGGGAGALDVGGVGLALVSSRSVFDHSAVVSGGSLDELLAGVGGVARGDGSAAGGVVLERRVAGGVGVAFSGQGSQRPGMGRELYGRFPVFAAALDEVCAEVEAQTGAELLSVVFGDDAGVLEDTGVAQPALFAVEVALYRLAESFGVRADVLIGHSLGELSAAYVAGVWSLADAVRVVVARGRLMGSLPSGGRMVAVEATEEEVSGWLVDGAVIAAVNGPAAVVVSGVEGAVEAVVERARGGGRRATRLRVSHAFHSPLMEPMLAGFAQVLARVEFRAPSIPVVSNVTGEVASAAELCSPEYWVRHVREAVRFGDGVGAVLAQGVATVVELGPEAVLTAMGASHPGVVENGAVFLPTLGRRTGDVNGFLTALARVHARGHQVDWTALGRANDLARELPTYAFQHQRYWLDAPAPEPGVVEHVPEQAVLLNAVAQQDVDGLAHTLGLAPDAPLTAVLPALQTWSREQARLAAADALRYRVSWTELPSPTDAVPLDGTWLVAVPGEPVEPDLVIAVEKALVDAGARVERCETAELRARLAAVAPRGVLCLPAVGAQRDRDRERGIATGALAVLDLLHTVQDAGVDTRVWALTCDAVCAQADDAAPDPWQAQVWGLGRVAALEHPTLWGGLIDVSGTSPAAQLTGLAAALANTTGDDQIALRGELLLGRRLIRGTVPTSVPEPETGSTAPWTDGSVLITGGTGALGAQTARWLARNGARTLVLTSRQGPAAPAVAALRTELEERGADVVVESCDVTDAVALAALRDRLADAGTPVSTVVHTAGVASELPLAELDEDGYAAVVRAKVVGAQVLDEVLGDGLAAFVVYSSIAGVWGSARAGAYAAGNAHLDALIERRRAQGRPGTALAWGPWGGGGMADERLTREMQARGVSALDPEEAVAAFGRVVRADYGTVVLADIDWSRLADIFTVNRPSPLFDPLRTVETERGGVGADGTAAGTAGADDVSGPGDSDAGTAGATPFVARWTALSGGERRRVLVETVCTQAAAELGHASGGTIEPERPFQELGFDSLAAVGLRQRLEKLTGLKLPATLVFDHPTPAALAQVVASALAERVGGASGASAVLGELDRLEAALAALDAGSDPAARGRITLRLSNLLTRFQNADDEPTAASGAAETAAEQLDSATDDQLFDLIEKEFGIS

SEQ ID NO: 5 - protein, 1931 aa, Streptomyces pulveraceus
MSTNEDKLRHYVKELTGDLLRTRGRLRELEAAGNEPIALVGMACKYPGGVASPEDLWRLVAEGRDAISPFPADRGWDLGRLPAAGGGFLHDAAEFDAGFFGISPRDAAAMDPQQRIALETCWEAVERSGISADSLRGKPVGVFMGGAVQGYGLVGTEIVDAPEGVGGTGSASSVISGRVSYSFGFEGPAVTVDTACSSSLVALHLAVQSLRAGECSLALAGGVTVMATPYAFVEFGRQGGLSADGRCRSFSADAEGTGWSEGVGVVVLERLSDARRNGHEVLAVVRGSAVNQDGASNGLTAPNGPSQQRVIVRALAGAGLSTSDVDVMEAHGTGTRLGDPIEAQALIATYGQGRAEGRPLWLGSLKSNIGHTQAAAGVGGVIKMVMAMRHGVLPRTLHVSQPSPHVDWSAGAVELLTRARQWPQTGRARRAGVSSFGISGTNAHVILEHEPVESTEAPVGSAQVPVESTQALVVAGELPWVVSGRTEGAVRAQAARLAAFVAGRGGGALDVGGVGLALVSSRSVFDHSAVVSGGSLDELLAGVGGVARGDGSAAGGVVFERRVAGGVGVAFSGQGSQRPGMGRELYGRFPVFAAALDEVCAEVEAQTGAELLGVVFGDDAGVLEDTGVAQPALFAVEVALYRLAESFGVRADVLIGHSLGELSAAYVAGVWSLADAVRVVVARARLMGSLPSGGRMVAVEATEEEVAPLVADVAAAGGMVSLAAVNAPGAVVVSGQDAAVDQIADIFAGRGRRTRALAVSHAFHSPLMEPMLAEFADVLAQVEFRAPSIPVVSNVTGTIADAEELCSPEYWVRHVREAVRFGDGVGAVLAQGVATVVELGPDPVLTALGERVRAASAERDSAARDVAFVPTLSRRSTDTRAFLGMLARVHARGHQVDWTALGRADDLARELPTYAFQHEHHWLKGASVRPGSAASRTAGSDGAFWKVVQEQDLQRLASDLGVDPDAPLHTVLPALGDWHQTHIEASETDGWRYRVAWERPTAQHAPEGPATLHGTWLIVVPEGDLRAGHLLDNDGLHDGLHGEVRRVLTDAGAEVKSLSLAPEDIDRQTIAKLLNGLDDTPAGVVSLLALSGREHTGPRGVGSGAWASVCLLQALLDTGWSATRLWTLTRGAVRATASDDAPDPWQAQVWGLGRVAALEHPTLWGGLVDLPAPDLSAADGHALAATAEASFGLAALLAGSSGEDQVALRADGARVRRLRPAGPDGAPEPVRPVAPESLVAPEGADATGRTGDPQPPAAREPWWSHGSVLITGGTGALGAHTARRLAEQGAPHLVLASRQGPDAPGAADLRAELAAHGATVDLVSCDVTSRDEVAALAADLAGRGAPVGAVVHTAGVAAEHPLADLDATEFAAVVDAKVTGAVILDEVLGDGLAAFVVYSSIAGTWGSTRGGAYAAGNAFLDALVERRRARRAAATTLAWGPWSGGGMAGEEFRQEMQRRGLRPLTPRLATTALDRAVRQEDTAIVVADLDWPRFIGVFTAGRPNHLFADFDDTESGAGHPDAGRTGAAQPGEWQRLPDLPLADQRPYVLDIVRREAARVLGHADAGTITEDQEFLALGFDSLAAVELRGRLTVLTGLALPSSLVFDHPTLGALVTHLLDNAAPGGDAGASPAPGVSAAPSASVAAAPPQDSNDSVVGIYRKLSLQGRMQEVEAFLSSASALRTRFHGAEDLGRGAHVTTLGHGEAEPQLVCFPPFAPVDGSLQFARLANHFRGRRRVSVVTVPGFMAGEPLAASLEVLIETLAEAVLRAADGRPYALLGYSSSGWLAQAAATWLEERGTGPVGVVLLDTYPPDSMTLEMRKAMTYEVVERRMRFTSMHYDGLTALGTYRGMFRGWQPRQLAVPTLFVRPDSCIPGSPEEPMAGPDWQAAWPLDHEETQVPGDHCTMIGEFSETTAAAVDEWLSRTPGLTRP

SEQ ID NO: 6 - protein, 3414 aa, Streptomyces pulveraceus
MMPSCPAASAAYPACAWSGSTTPSPPWRARAPWPPRSRVRCSPSSRRPTISCSSGVPPSGPRRSRASSPIVRWKSSTSLRRWRRSAGPPRTAATPPYPSRVRFMSEGFMPIAVVGMACRLPAAPDPATFWRLLSEGVDAVGETPADRWPDAAGTPTGAARYGAYLDRIDTFDPGFFGISPHEARAMDPQQRLMLELSWEALEDAAIVPASLGGSGTGVFVGAIWDDYRSVVARAGTASFNQHTMTGMGRGLIANRVSYTLGLRGPSMTVDAAQASSLVAVHLACESLRRGESRVALAGGVNLIAAPEGMAASMSFGALSPDGRCHTFDARANGYVRGEGGALVVLKPLEQARADGDFVYCVIRGSAVNNDGATDGLTVPSAPAQTELLRAACRQADVAPGDVQYVELHGTGTAVGDPIEAAALGTAFGAGVGRVADNALLVGSAKTNIGHLEGASGVVGLVKTALAIRHRKLPPSLNFVTPNPKIAFDELSLRVQVGLTPWPRPDGPIVAGVSSFGMGGTNCHVVLCDAPAESSAPSPATSSAAPPATVPVPVAVPQDVASPWLISARSEAALRGQAAALAAHLEQHPELDAATVARGLATIRTHHEHRAAAFGGDRSALLSELRTLAQGRPSDGLLRGTAPDPGTGTTPGTGPKTVFVFPGQGVQWAGTVRDLMATLPVFREHVEAAAAALDPLTGWSLVDHLTGPETLPDTPDHVQPVLFAVTTALAHTWRTLGVQPHAVLGHAAGEIAAAYSIGALTLQDAAALVVARGRAHGEQAEAVRDALLDELSGIEPRPSGTRFQSTTLGGPVDTAALDADHWYRNFRQPAPFHPAVEELMDDGHTVFIEVGPHAVLPPEILELLDAAGAVGIPALGRGDGGRPRLLSSLAAAHVRGAAVDWPALYGLPAARRVELPGYAFDRRRYWPEPTPTSAPVARQGAAAVPTPNPAAPAGAAVPASGPPVSASASVRDSDWLRGLVEAAPAGRDEQLLDLIRDEAAAVLGHSDPREVDLARSFKDLGLESASGVELVERLGSVLQLRLPATLLYESPTPKVLAQVLGLELKGAARRATASAASAVTARPEKTSAVSAPASVRDSDWLRGLVEAAPAGRDEQLLDLIRDEAAAVLGHSDPREVDLARSFNDLGLESQSAEDLCERLAAVLQLSVPATLLYDSPTPRALARVLGAELAGTTQSDATSAASVSDEPIAIVGMACRYPGAADSPEALWQLVAEGADAIDVFPENRGWDLEGLYDPDPDAPGRTYAREGGFLYEADRFDAQFFGISPREALAVDPQQRLLLETSWEAVERAGIDPTGLAGSRTGVFVGATAMEYGPRLHETVPETAGSVGGYLLTGSTVSVASGRIAYTFGFEGPAMTVDTACSSSLVAAHLAARSLRNGECELALAGGAAVMASPGMFVEFARQRGLAGDGRCKSFSAAADGTSWAEGVGMLVLERLGDARRNGHRVLAVIRGSAVNQDGASNGLTAPNGPSQQRVIRQALADAGLEAADVDAVEAHGTGTALGDPIEARALLATYGQGRSEGRPLWLGSLKSNIGHTQAAAGVGGVIKMVMAMRGGVLPRTLHVDEPSPHVDWEAGEVRLLTGPVVWEAGERPRRAAVSSFGISGTNAHLILEEPPVKERTAYEAEADSADPAVWLVSAKKADALGEQAGRLAEFARTRTEVGIRRAARALATGRTHFDHRAAVVAQDRDALAEALSALASGAGHPMVVRGRATVGRTAFLFSGQGSQRPGAGRELYASYPVFAAAVDEACAVFDPLLGRSLREVMFAGPGSEGAELLNRTAFTQPVLFVLHTALFRLLESFGVRPDHLVGHSIGELSAAHAAGMLSLADAATLVFHRARLMQQITTPGTMLALQAGEATARGLVAGREDVVSLAAVNAPESTVLSGDPEVLADIAAQLAERGIRSRRLTVSHAFHSPHQDQILDEFRRIAAGLTYRAPRIPIVSTLTGLLAEQDRITTADYWTEQLRRTVRHADAVTTLHGLGTTRYLELTPTPTLATLVAETLEESPAALVPVLRHGRPEHDALLRALATLHTSGADVAWPALPGPRSAALPELPTYAFQRERYWLTPPAPRADVTQAGLTGTPHPLLAAAVELPEGGGFVHTGRIGTLTHPWLADHAIHGTTLLPGTALLDLVLHAASDGAGEHPAVAELALQAPLVLPGERGVDIRVTVQEADESGLRAFAVHSRPAPAGDDASGSSSWTRHASGALGPTEAPDAADRAPQWPPADAAPVDLTDLYPALALTGYEYGPDFRLLTAAWRTDDDVFAQVELGDDAAASDDVDRFSVHPALLDASLHALLRSGLLADGVSGTDASGTLLPFSWGDVALHALGATALRVRFTRTGPTTVRVVASDPSGALILTAGELSLRPVVLDRLSDGSGTEAGGPRSLYHVEWSATPAAAPVGAAAPDAPEQWALIGRSPVPDPVSTLAAEAVDIRTYPNLDALVHGIENGDPHPSVVLADLAAHGAELPAHEGERTGEHEGGAAGAHAVARRTLALLTSWLDAPALTVGRLVLVTHDATAAATAPDALGLPQATAWGLVRSAQTENPGCFTLVDIDGDPSDGYAALPAALRTGEPQLAVRDGEVLVPRLARAAQDADVPWPAPADVAEQVGAAQRAPLGTGQVRIAVRAAGVDLRAPVLARDTPPDHDILGLEGAGVVTETGPGVSDLAVGDRVFGLLTGNFGPQAVAERDTLARIPAGWTFTQAASVPVAFLTAYHALVELAAVVPGERLLIHSVADGVGLAAAQLARHRGAEVFGTAGPGEWADLRAHGLDDTHLAPSHTQEFAARFRAATGGAGVDVVLDCPAGDAVDASLRLLSSGGRFVETGRTDTPDSEAVAARHPGVDHRSFDLTKLEPAHVGAMLGELTELFERGALRPLPVAAWDVRRAQDAFRHLSRPRHVGKVVLTVPAPLDPEGTVLITGGTGALGGNVARHLVTRHGVRRLVLTGRRGPAAEGVTEIVAELRAAGAVEVTVEACDAADRTALARVVAAVPEAHPLTAVVHAAGVLDDAPVEALTPERLDTVLRPKADAAWNLHELTAHADLSAFVLFSSVAGVIGHAGQGNYAAANAFLDGLAAYRRHRCGLPAVAAAWGPWEHAAGGMTQALSATDLNRMARTGVLPLSTDEGLALFDATRDAAVPAVVPVRLDLAALAESAGGAGRAGDVAEVPLLFRHVAPARPVRRLPQAAATADGARLPAPRQTAVDAGPDLARRVAELPTEAARRGMLLELVQDSAAAVLGHATAATVDPERRFKELSFDSLTALELRNRLGAVSGLRLPGTLIFDHPTPLAVADFLYTRLASQTPRTATSDSLAVLAELDRLVDTAIATDADEATLTRFTARLEDLLVWLHGRQEAGRPDAEDAAGATDRFESASDDEIFDFIDNELGLT

SEQ ID NO: 7 - protein, 1880 aa, Streptomyces pulveraceus
MPANDDKLRDYLKRVTADLHQTRLRIRDIEARKREPIAIVGMACRYPGGVTDPEQLWELAAGGIDAVSGFPSGRGWDLEGLYDPDPDAEGKVYVREGGFLHDAGQFDAPFFGISRREALAIDPQQRLVLETSWEAVERAGIDPLSLAGSRAGVFVGVMPQEYGPRLYEATGQGVSGHLLTGTTTSVLSGRIAYTLGLEGPAVTLDTACSSSLVAMHLATQALRSGECEVALAGGVTVMANPGTFVEFSRQRGLAPDGRCKSFAAAADGTAWGEGVGMLVLERLGDARRNGHRVLAVIRGSAVNQDGASNGLTAPNGPSQQRVIRQALADAGLEAADIDAVEAHGTGTTLGDPIEAQALLATYGQGRFEGRPLWLGSLKSNIGHTQAAAGVGGVIKMVMAMRGGVLPRTLHVDEPSPHVDWEAGEVRLLTGPVVWEAGERPRRAAVSSFGISGTNAHLILEEPPVKERTAYEAEADSADPAVWLVSAKSPDALRAQADRLTEFLAARPQTGTGHLARALATTRSQFEQRAALIGADRAGLTEALSALASGSGHPSLVRGQVTTGRTAFLFSGQGSQRPGAGRELYASYPVFAAAVDEACAVFDPLLGRSLREVMFAGPGSEGAELLNRTAFTQPALFVLHTALFRLLESFGVRPDHLVGHSIGELSAAHAAGMLSLADAATLVFHRARLMQQITTPGTMLALQAGEATARGLVAGREDVVSLAAVNAPESTVLSGDPEVLADIAAQLAERGIRSRRLTVSHAFHSPHQDQILDEFRRIAAGLTYRAPRIPIVSTLTGLLAEQDRITTADHWTEQLRHTVRHADAVTTLHGLGTTRYLELTAHPTLAPLAAETLEDASAAPAALVPTLRAGQPEPDTFLRALATLHVTGTPVTWFADHAEADADNADTADGRGHERGRATVPHLDLPTYPFQHENYWLTAPSSGTGPGAGADALPHPMLSQRTDLPGGGGVLFSGRLAPGTDPWLPDHAVMGTLLLPGTGFVELALEAARAVGAGRVEELVLRAPMVFPGGRARDLQVWVAPDQGGERELLIRTRTPGEDWTLHATGVVTASRVDTDGFTPDWTGAVWPPAGAEQIPGDTFYPDLAERGYEYGPAFRSVKALWRRGDDLFAEVVLPEDQPYGFGAHPALLDASLHALPITRSFYETDDEVRLPFSFGGVSLFATDVRRVRVRLRPRPEATSVWITDAAGTPVLAMESLILRAVERTQLQAAEGAVGQAATFAVRWEPLSEARIAERVPGTWLLFGTARPGLAELFEHVLTSTEWDASASTPVEGVLVCPADASELLAALRETERLDAPVWCVTSGAVGVGVDDPATDVAAAGAWGLGRVAALELPSRWAGLVDVPETADLGTADDNAGRTTARLLAGVLTGDGAEDQLAVRDGRLWARRLGTAPAADAGTWQPKGTVLITGGTGGLGAHVARRLAALGTADRLVLLSRRGAESPGAAELLAELGESGVRAEAAAIDITDRTAVTQLLSRLDAEDDPVRTVVHAAGVIRYARIADVDPEAFETDMAAKVNGALLLDELLPDADEFVMFSSIAGIWGAADQAAYAAGNACLDALARRRRERGASAVSIAWGPWSGGGMVTEYEDRELRKRGLLPLAVPSAVEALERAVPGDTDPVVVDVAWSRFLPAFTVLRPSPLLSGFAPADTAGGGRDAASAALPGAGTTAGALKDRVGALPEDERLPVLLDVVRTHVAQLIGRGDPQQVQADRALRELGFDSMMSVELRNRLGELVGARLPATLAFDHPTPESLAERLLTELDLDEAPADDGPVLEDFDRLEAKVLSPFTPADTRAALATRLSALLDRLSGTGTGAGGAGRNSGTDDLETASASDLMQFLDAEYGASDGTASDPSRPTTS

SEQ ID NO: 8 - protein, 213 aa, Streptomyces pulveraceus
MQLIKHAHACVSLVKDGGRIAIDPGTFTPDAKEVVAAADAVLITHDHFDHFDEDLIARALDARPGLRVYGPVRVVGRWAARRGQVVAVADGDRLDVAGFDVAVSGDLHAAIHRDIPRVTNVGYLVDGRVHHPGDAYHVPPAPVDTLLLPTSGPWTQLGRAADYVREVAPNRLVQIHEIMLSRTGQESMARFLSPAALTEVPLTIVPEGEAITV

SEQ ID NO: 9 - protein, 967 aa, Streptomyces pulveraceus
MTRPGGGNPVFRSSCGPKPSSGSESNCAVSLVDRDGELAELGRWLRRRPADSAGLCVIEGPALCGKTALLEAACAEARRMGIGVLTVRGSVCGVGYAEELGEFGAREPGEQARRLVAAATRSLLLAVDETDLLAPESQVFVREVVRLTHTGRIRVLLTGVSAATVPSIVDELRELAAGWAERVRVRPLGRAAVAELARARVPGPRADALAEAIHPITGGNPGLTLAMLDEAAAAIGRDPDAPDGPPSAGLARRLREHTSREVMNLVEAVAVLDAQCTPERLWNVLEKDAEYVSHVAGQAIASGIVRPDMTLHPYLRDAVLWSTHPAGLRSLRLRAAQVLHEQGVPAWEIAPYLHEAGIAPQPWAGGVLYEAAEHASRLGRHRLAVQLLESASFWGSGPDLREEAGVRITDMVWWMDPALTAPRLRRLAAAASDGTFKPGSLARLSRHLARHGEMAQASDMLRLAEPAPGRGNAVGAVSPGDALGAADVRAEVQRTLSGLWLRHLFPDACPDAPAPDDQPHPQLIAEGFSWLSSAQRLSWLLAPGVQCSDAPNSGPRPTGSADSAGSAKAVKDAEAAVTCAEEILQSANFDYSSLEPMLVALFCLMATGRLDPRAPWYAALADNAALADNERMPLPLASMLSVAEAVAAWWKGDLVAAQTAVRRGFERWDADRWGVLAGLPRGLSALMLTEQGRFEEAAAQLARPMTEADLASPYGLVYLRARGRFRLATGALRAAVADFRSCGAILTAWGMEMPGLVPWRLDLAEALLKLEEYAEAAELIDEQLAFSPDSNTRARGIGLRLRVATVPPGERALACRKSIAVLQSCGDELEYARALGQLAQIYKHTGDLAGGRKTMRLAEKTARGCGAQWALAEMSGGSQARRKVPPPAARRDGRSLTPAEWNVARMAAQGYTNREIAESLYVTASTIEQHLTRIYRKFDIRRRAEIGAIVESRGAPDHTPAEIQDVA

SEQ ID NO: 10 - protein, 295 aa, Streptomyces pulveraceus
MATEPMKEDISNMPISKADETADAPRTADAALRAEAARTAQAGPMAGKKNVVVLGVTSALGRRLAESILARGLTPVGLIGEAGHSESLRAAGTEPLVIASEADRTSPAALRAMGECGGIAVATGTGWGPDATDTSSTSLVGELMGVAQLVGIRRFVLVSAYLPDDELHTRLGDELESYLAEKRDVERKLAGHDLDWCVVRPGMLDNSPATGLVTVRGGADPQPEGTVSRTDLAETICEALFAPEPVRGVLAVSAGAQPIRAALGSVPQATVQPAWWCAEERAEGAAEKRADSPAS

SEQ ID NO: 11 - protein, 409 aa, Streptomyces pulveraceus
MNTLSLLQGLPLHRSDPFSPPDGYAKVRAEAPVSPIAFPDGNQGWLLTRHADVKAMLANPSFSSVREKAARTRRTEGRPTPLPGAFFTMDPPDHTRYRRLAASRFAVRKIKALEPKIEQYTREHLDRMEETGGGPVDLVTAYALPIPSLIICDLLGVPYDARDDFQRWSLSILDTELSEEEQQRTVLEGTKFMLDLIEDKKKNPSDDLISDLLDPAEEKDRISEFEIAGMCGLMLMAGHESTSNMLSLGTLAALRNPDQLALLRSDPSLIDTAVEELLRYLSIVQFNFARLATEDVEIGGQLIKAGETVVGSMAAANHDPEVYTDPHRLRLDRAEERNLAFGHGIHLCIGHQLARVEMKVTYLRLFERFPTLRLAVPFEDIEFRANSVVYGVNSLPVAWDAPADHDPAP

SEQ ID NO: 12 - protein, 316 aa, Streptomyces pulveraceus
MPESTDAAAAVPEAHDLPLTLWGWQDWTVEPWERLPGDEGYSSHTYLVRHDGVRHVVKAVRKDMGPKLTAGLLVAQEVERHGIAAGGPLPTTGGEVTAYQGDFCYSLLTYLDGERVDETDPAHLRAVGRTLGRIDSVLLHAPVPEGVPRWNEVLELFLLEQDFLKGHDWIRRTLEQAGGALSPDDLTIGLINCDAAAKEFRVLGDTAGLLDWSEAMYAPCMLELATTLSYLEDETDGEPLVRGYFEEGPADRAELGFLADILRFRCAAEGWIYAARQNAGDETGTTASTWSNEKLIERARQNAENADRIAARFQVF

SEQ ID NO: 13 - protein, 444 aa, Streptomyces pulveraceus
MKSYKALAGIPHLPRLFLWSMLARLNVSMLPIGLTLVLVGWSDSYVAAGVLGGALTAGQALVGPARGRAADRGAVRKLLVLTGIGYLVGLGALVTMTRTVPGGGWPIAVLVALLTGMSTVPISQVSRAVWPKIVPGELGRTLFTLEATGSEVVQTTGPLLTSLLVTALDPGYAVIACGVVAFVGALAFAAALGSAGIRGGAERTAAKPQAETGTAAETGADAGTEGDTATSASAVTRASAPEPEERRTLFALPKFTLAIVVTLVMMAALFSVNLSLVAWARDSHESGLSGVLIACWTIGSVVGGFGMGALRKDVPHSARFAAMAVGMALLAVLLPPVTETAPVWLVLVVLFLGGTAIAPSMAGNFAQVSGAVPQERRAEAFGWLATAGTGGAALSMPVTGVLLEASGPATSVAVGAALALCATVLSYIGSKRAERNDGLVSARA

SEQ ID NO: 14 - protein, 420 aa, Streptomyces pulveraceus
MTSTDAVPTGTPPLTTDSSSETPPAYPMPKAPGCPMDPPPVYRTLRAEQPVSKARLYNGREAWLISRHEDVRKILSDSRASVDALNPGFPWLSEVAKAMNTAEGGVRPLGRMDPPDHTELRRMLAPHFLIKRVRALRPATEELVDGLIDRMLEGPSPADLVPALARPVPSTVVGWLLGVPAEAMARFGETTARLFDEDGSAETAVAARGELFEQLKELVELRRAEPDGNIVSRLVGFADEGRLTETNLLMQIGLLLGAGYDTTVKMITTGVLALLGHPEQAALLAKEPERAAGATEELLRYLTVAEFAPKRVAVEEIEIGGQTIRPGDGIICLISSADRDESVYERPDELDIQRSARDHLAFGSGIHLCVGHSVARMELEVVYGRLFSRIPQLRLAVAPNEIPFSRGLDVQGAKSLPVTW

SEQ ID NO: 15 - protein, 398 aa, Streptomyces pulveraceus
MYSRRMPIIELAEYGPDFLADPYPYYAKLREEGPVHEVRAPDGYRFWLIVGYAEGRAALTDSRLVKARDTMATSEASPLGKHVLIADPPDHTRLRKLISREFTVRRVDNLRPRIQELTDDLLDVMLPAGRADLVEALARPLPIAVLCELLGVPNADRDEFHSWAKGILAPQNPTETHTAVKALMSYLDDLIEDKRHGEPTGDLLSGLIRTSIENGDRLSSEEVRSTAFLLMIAGHETTANLISNGTRALLTHRDQLDLLRSDMDLLDGAVEEMLRYDGSLESTTKRFTGVPVQIGDTVIPPGETVLVSLASADRDPANFDDPDRFDIRRGTPAGVGHLAFGHGIHYCLGASLARAEGRIAFRALLERCPDLELDPEAPPFEWMPGVLVRGVQRLSLRW

SEQ ID NO: 16 - protein, 538 aa, Streptomyces pulveraceus
MHTERILTEHHRFLATLDHPEQTQQTVLAELLAANGATSYLREHGLNERSGAEEFRKALPIRTQNAFGPWIERAIAGEDGVLTAERPVAFFSSSGSTGQEKRIPVTPTYMKRCFLPFYHASFAVLLGAFPDLAADPGGVLNLWRDPTSPHARTADGRPHLGPSQIDHRLFGEGGGPEDGAAWATIPEQLSDADPWERAYLQLRLAAERDIKVLIGVNPALIAGLPHQLAAQWPRIVEEIARGTVGGVPHTTPDPRRAEQIARRADEYGVLDPYHLWPNLRAAVAWNSALASLYLPRVRERYGPGVRLFAAPIGSSEGPVAVPVDDHPNAAPLYLPGCYFEFADAAEPIREDSPTVTAAELEPGRDYHLVLSHIGGLYRCAVNDVVHVVDHVGRTPRIAYTGRDVLRTAGGVDLTERAVVRALAGTLADTGAELRNATVETGTDRFRAAIASALPGPLPAGFATLLDKHLGETADGYRAARDAGALAPVEVLQVHQDAFQREWEHAIRSGQRRTRVKDRIFQPAPDSWARITADERAHA

SEQ ID NO: 17 - protein, 781 aa, Streptomyces pulveraceus
MTDDAIPGRGRYTEQARAARLAWLLARTGATLDSAAHTAIEAASLTGNLENFAGSVEVPVGLAGPLQFRGQGVREAVVAPMATTEGALVASASRGARALSLAGGVSTRVLSQRMSRAPAFEFDDLAGAARFSRWLGTRRPQLEDQVRLVSQHARLVAVDPYQIGRYLHVRFVFETADAAGQNMTTAATWQICTWLNEVLADEPGLRPRNTLLEGNLSSDKKVSSVSLLAGRGTRVTAECVIPGDVVASVLKTTPAAIARGHRVAVIGGQQAGMTGYGINAANVIAALFVATGQDIACVHESAVSVLSFDSDGDDLIATLLLPNLVIGTVGGGTGLPDQRDWLGVLGCRGEGGTARLAEIIAGFALALDVSTASALVSGQFADAHRRLGRARRVDWLRADDLGPGLLQPAMAERLDSPRLRVTDVVRAPAMVGDGISTELGALGERRKLTGVIPMTVSWTEDDGPQTTAELVAKAKPRGEEIAAGIGRIASLCGPEVSSAWETWGGGSDFPAAHRRELAVFRRPEGVLTSLLPVCYGIIEDEAREAYVILMERLDITTGPWSRTDVDRALRAIAPVHGHWLGRDQQILAEGWLYRDGTTAHLVKARELWEALVRHNAAELPELMTPQRTRTALAAAAEAEFWVQEMDAMPRTLVHNDFNPRNISRQSERVTAYDWELATVAVPQRDLAELLAFTLTPHSTTDEVDHHLEVHRAAVAAAAGPDATVPQPEQWRRGYGLALREMLLSRLQLYTAAHSHRELPFLPAVLDTTFHLWNLEAARDGE

SEQ ID NO: 18 - protein, 258 aa, Streptomyces pulveraceus
MYAPTPKPSTDRQAWLRRYTNAPDARHRLVCLPHAGGSASFYMPLARALAPEIDIVAVQYPGRQDRRADPFPATLQDLAAHVAEALCGEPAVPTAFFGHSMGAAVAFEVIRLLEDSTTPVTALFASGRGAPSVNRGERVHAMSQEDVLAELRGLEGTDSRMFDDPEIIEMIMPPLRNDYRLIETYRYVPGPPVACPIRGFLGAQDPKVDEGEMKLWADHTAGSFDLTLLPGGHFYLVQHQPEIVEAIRNTLLVAPPYV

SEQ ID NO: 19 - protein, 633 aa, Streptomyces pulveraceus
MATETFEFQVEARQLLQLMIHSVYSNKDVFLRELVSNASDALDKLRLAALRDDGLDADTSDPHIEIELDQKARTLTVRDNGIGMSYDEVGKLIGTIANSGTAAFLQELKEAQDAAGAEGLIGQFGVGFYSGFMVADEMTLVTRRAGERSGTRWSSRGEGTYTLETVDDVPQGSAVTLHLKPADADDQLHDYTSAWKIKEIVKRYSDFITWPVRLLPQATDGEETPEPETLNSMKALWARSRDEVSDDEYHELYKHVSHDWRDPLETIRLQAEGTFEYQALLFLPAHAPHDLFTRDFRRGLQLYVKRVLIMDDCEALLPPHLRFVKGVVDAQDLSLNVSREILQQDRHIRMIQRRLTKKVLSSVKEMKANDADKYAAFWREFGAVLKEGLLGDTDDRDALLAVASFASTHAEETPTTLQQYVERMKEGQDDIYYMTGASRQTIENSPHMEAFRDRGLEVLLLTDPVDEVWVDVVGEFEGKRLRSVAKGEIDLDVQGGEQADGGREKQAETYAALLGWMKEHLGEEMKDVRLSTRLTVSPACVVSDAHDLTPALESMYRAMGQEIPSARRILELNPAHPLVQGLNQAYQEGEDRSGLAETADLLYGLAVLAEGGRPTHPGRFVKLVAERLERTLR

SEQ ID NO: 20 - protein, 165 aa, Streptomyces pulveraceus
MNDAPSNEPAPLPDELAVRLRAVVGTLVRSARTVDRLASVPAAVLGLLDTRGPMTTADLAATRGVRHQTMAATVRELTEAGFLASRTDPGDARRKVLALTKAGKKALDTDRRQRVGVLADALEETLDDEDRRALAHALDLIDRISGSIRGGHSFSGEREFNTGAW

SEQ ID NO: 21 - DNA, 22 bp, Artificial
  T7cos primer
cataatacgactcactataggg

SEQ ID NO: 22 - DNA, 18 bp, Artificial
  T3 cos primer
ttccccgaaaagtgccac

SEQ ID NO: 23 - DNA, 19595 bp, Streptomyces pulveraceus
  misc_feature (1)..(7316): Contained in cosmids 3F45 and 2F15
tcgatccggaaggactggctgcactgccggtcgagcagaccgacgaagtagaggcgcatgccgagccggtaggcgtccgcgtccgccgacccggagatggccgacaggtccgagagcgtcgacccggtccacccgccgtacttgctggagacgaccttgccgccgatcggcacccgggtgagcgggagccgggtggtgtcggcgccgaactcggccaggatccggtcaaggagcaggtagtcgacggcgagcccttcgtcgaagagcaggaggaagagcctgccgggcgcgacggcgggcagcagcgagcgcaggatcggcagcaggtagttcgcgtggccgtccgtcgagacgatctgccggatcggcacgccccagcgggcgccgtccaggtagacgggcccgccgtgcggcctgtggtcgatgagcagcccgcggtccgcgagggccgccagggcgcgctgctccgaccaggtcagcgggcgggtgtcggtcaggccggggtcgcgcacgtgcagcaggtcgagttcggtgcgccacagctccagcaggcggtgggaggccggatggatccagccgtcccgctcgatccgggcgaagtaggggtcgagtgcgcgcgcgtcggtcgtcggacggccgcggtggaactcgacgtagcgccggccgatcgcggtctcgtcctcgccggaccagtcggtgtccggttccgtgcggtccaggtggttccagaacgccgtggtctgggtcgtcagcgtggacatccgcggattccagaccagggtggtggggccgagggtggccgtcgccttgaacagggcgtcggcccacagcaggcctttgacatgggtgggggtcagcggcttggtgggggtgatcgtcaccggggcgatcacgaactccctcgcggcgcggcgggcgccgggatgcgcgcggtgtgcgcggctcgcgggtgtgggcgggccggtcgtcgcggttcgtacggtgctcggggtgcgcacggtgctcgaggtgcgcagggtggtgctcatggatggctcctgtcgatgtctgccgcgaccgggcggacgaggtcgtgcgcggcgcgcgacatgccggtcgcggcgcaggacgtgccggttgcggcgcgcgccgcgtgggccgtgacgcgcgccgcgttcgaatcgacagcggtcacgacgtggtcggccgggacgggtcggacgccgtcccgtcggaggccccgtattcggcgtcgaggaactgcatgaggtcgctcgcgctcgccgtctccaggtcatcggtgccggagttcctgccggctcccccggctccggtgcccgtgccggagagccggtccaggagcgcggagagacgggtggcgagcgcggcccgggtgtctgccggtgtgaacggcgagagcaccttggcctcaaggcggtcgaaatcctcgaggaccggaccgtcgtcggcgggggcttcgtcgaggtccagttccgtgagcagccgctcggcgagcgactcgggcgtggggtggtcgaacgccagcgtggccggcagccgggcgccgacgagttcgccgagcctgttccgcagctccacggacatcatcgagtcgaagccgagttcccgcagcgcccggtccgcctgcacctgttgcgggtcgccccgcccgatgagctgcgccacgtgggtgcgcacgacgtcgagcagtacgggcagccgctcgtcctcgggcagcgcgcccacccggtctttagcgcgcccgcggtcnnnnntgcgttcccggccgccgccccgccgtgtcgcgggcgcgaatccgctgagcagcgggctggggcgaaggacggtgaacgcgggcaggaagcgcgaccaggccacgtccaccacgaccgggtcggtgtcgcccggcacggcgcgctcgagcgcttcgacggcgctgggcacggcgagcgggagcagcccgcgcttgcgcagttcccggtcctcgtactcggtgaccatgccgccgcccgaccagggcccccaggcgatggagacggcggacgcgccccgctcgcggcgtcggcgggcgagcgcgtcgaggcaggcgttgcccgcggcgtaggcggcctggtccgcggcgccccagatcccggcgatggaggagaacatgacgaactcgtcggcgtcgggcagcagttcgtcgagcaacagcgcgccgttcaccttcgccgccatgtcggtctcgaaagcctcggggtcgacgtccgcgatgcgcgcgtaccggatcacgccggcggcgtggacgaccgtacgcaccgggtcgtcctcggcatcaagacgggagagcagttgcgtgacggcggtgcggtcggtgatgtcgatcgccgcggcttcggcccggacgcccgattcgccgagttctgccaggagttcggcggcaccgggtgactcggcgccgcggcgggagagcagcaccaggcggtcggccgtgccgagagcggccaggcggcgggccacgtgcgcgcccaggccgccggttccgccggtgatcaggacggtgcccttgggctgccaggttcccgcgtcggccgcgggtgcggtgccgagccggcgggcccacaggcgcccgtcgcgcaccgcgagctggtcctcggcgccatcgccggtgagcacgccggcgagcagacgtgcggtggtccgcccggcgttgtcgtcagccgtcccgaggtcggccgtctccggaacgtcgacgagtccggcccagcgcgaagggagttcgagggcggcgacccggccgagcccccaggcgcccgccgcggccacgtccgtcgccgggtcgtcgacaccgacgccgaccgcgccgctggtcacgcaccagaccggcgcgtcgagccgctcggtctcccgcagcgcggcgagcagttcggaggcgtcggcggggcagacgagcacgccttcgaccggtgtcgaggccgatgcatcccattcggtggacgtgaggacgtgctcgaacagctcggcgagtccggggcgtgcggtgccgaagagcagccaggtgccgggcacgcgctcggcgatccgcgcctcgctcagcggctcccaccgcacggcgaaggtcgcggcctgtccgacggccccttcggccgcctggagctgtgtgcgctccacggcgcgcaggatcagcgattccatggcgaggaccggggttccggcggcgtccgtgatccacaccgaggtcgcctccggccgcgggcgcagccgtacgcggacgcgccgcacgtccgtggcgaagaggctgacgccgccgaaggagaagggcaggcgcacctcgtcgtcggtctcgtagaagctgcgggtgatgggcagggcgtgcaacgaggcgtccagcagagccgggtgggcgccgaagccgtagggctggtcctcgggcagcacgacctccgcgaacagatcgtcgccgcgccgccacagcgccttgaccgaccggaaggcggggccgtactcgtagccgcgctcggcgaggtccgggtagaacgtgtcaccggggatctgttccgcgcccgcgggcggccacaccgcgccggtccagtcgggcgtgaagccgtcggtgtccacgcgggaggcggtcacgacgccggtggcgtgcagcgtccagtcctcgcccggcgtacgcgtgcggatcagcagttcgcgctcgccgccctggtcgggtgcgacccacacctggaggtcgcgggcccggccgcccgggaacaccatcggggcgcgcaggacgagttcttcgacgcggcccgcgccgaccgcgcgcgcggcctccagcgccagttccacgaatccggtgccgggcagcagcagggtgcccatgacggcgtggtcgggcagccacgggtccgtgccgggcgcgaggcggccggagaacaggacgccgcccccgccggggaggtccgtgcgctgcgacagcatcgggtgcggcagcgcgtcggccccggcgccggggccggtacccgaggacggggccgtcagccagtagttctcgtgctggaaggggtaggtgggcaggtcgaggtgcgggaccgtggccctgccgcgctcgtgtccgcggccgtcggcggtatcggcgttgtcggcgtccgcctcggcatggtcggcgaaccaggtgaccggcgtcccggtcacgtgcagggtcgcaagggccctgaggaacgtgtcgggctcgggctgcccggcacgcagcgtcggcacgagcgccgcgggagcggcggacgcgtcctccagtgtctcggcggcgagcggcgcgagtgtggggtgcgcggtgagttcgaggtagcgggtggtgccgagtccgtgcagggtggtgacggcgtcggcgtggcggacggtgtggcgcagctgttcggtccagtggtcggcggtcgtgatccggtcctgctcggcgagcagtccggtgagcgtcgagacgatgggaatgcgcggcgcccggtacgtgagccccgccgcgatccggcggaactcgtccaggatctggtcctggtgcggactgtggaaggcgtggctgacggtcagccgcctgctcctgatcccccgctccgccagctgtgcggcgatgtccgcgaggacctcgggatcaccggacaggacggtcgactccggcgcgttgaccgccgcgagcgagaccacgtcctcacgccccgccacgagaccccgcgcggtggcctcgccggcctggagggcgagcatggtgccgggcgtggtgatctgctgcatcagacgggcccggtggaagaccagcgtggccgcatcagccaaagagagcatcccggcggcatgcgccgcggacagctcaccgatcgaatgccccaccagatggtccggccgcacaccgaacgactccagcagccggaacaacgcggtgtgcagaacgaacaacgccggctgcgtgaacgcggtccggttcagcagttccgccccctccgaccccggccccgcgaacatgacctcacgcagcgagcggcccagcagcggatcgaagaccgcacacgcctcatcgacggcagcggcgaacacgggatacgacgcatacaactcccgccccgcacccgggcgttgactgccctgaccggagaacaggaacgcagtcctgcccgtggtgacctgcccgcggacgaggctcgggtgcccggagcccgacgcgagcgccgacaacgcctcggtcagtccggcacggtcggcgccgatgagggcggcgcgctgctcgaactgcgaccgggtcgtcgcgagcgcgcgggcgaggtgaccggtgccggtctgcgggcgggcggcgaggaactccgtcaggcggtcggcctgcgcgcgcagcgcgtcggggcttttcgcggagaccagccatacggcggggtctgcggagtcggcttcggcctcgtacgcggtccgctccttgaccggaggctcttcgaggatcaggtgcgcgttggtaccactgatcccgaacgacgacacggcggcacgacgcggccgctcccccgcctcccagacgaccggcccggtcaacaacctgacctcacccgcctcccagtccacatgcggcgaaggctcgtccacatgcaaggtcctgggaagcaccccgccccgcatcgccatgaccatcttgatcacaccacccacacccgccgcggcctgcgtgtgcccgatgttcgacttcagcgaaccgagccacaacggacgaccctcgaaccggccctgtccataggtggccagcaacgcctgcgcctcgatcgggtcaccgagcgtggtgccggtgccgtgcgcctcgacggcgtcgatgtcggcggcctcgagccccgcgtcggccagggcctgacggatcacccgctgctgcgacgggccgttcggcgcggtcagaccattgctcgcaccgtcctgattgaccgccgagccgcgaatgaccgcgagcacacggtgcccgttgcgccgcgcgtcaccgagacgttcgagcaccagcatgcccacaccctcgccccacgccgtaccgtccgccgccgccgcgaacgacttgcaccgtccgtccggcgccaggccccgctgccggctgaactccacgaaggtgccggggttcgccatcaccgtcacgccgccggccagcgccacctcgcactcgcccgagcgcagcgcctgcgtcgccagatgcatggcgaccagcgaggaggagcacgcggtgtcgagcgtgaccgccgggccctccaggcccagcgtgtaggcgatccggccggacaggacgctggtcgtggtgccggtcagcaggtgaccgctgactccctgccccgtcgcctcgtacaggcgcggcccgtactcctgcggcatcacgccgacgaacacgcccgcccggctcccggcgaggctcagcggatcgatgccggcccgctccaccgcctcccacgaggtctccagcacaaggcgctgctgggggtcgatcgccaaggcctcacggcggctgatgccgaagaacggcgcgtcgaactgtccggcgtcgtgcaggaagcctccctcccggacgtacaccttgccctcggcgtccgggtccgggtcgtagagcccctccaggtcccagccacgtcccgaggggaatccgctcaccgcgtcgattcccccggcggccagctcccacaactgctccgggtccgtcacgccgcccggataacggcatgccatgccgacgatcgcgatcggttcccgcttgcgtgcctcgatgtccctgatgcgcaggcgcgtctggtgcaggtcggcggtgacccgcttgaggtagtcgcggagcttgtcgtcgttcgctggcatggaggaacctgcgccttctgcgtgttctgcatcgcgcggccggtccggtgggaccgaccgcgcggcgtggcggagtcggactgggcggaccgtcaggtcagtccgagttcgttgtcgatgaagtcgaagatctcgtcgtcgctggccgactcgaatcggtcggtggccccggcggcgtcctcggcgtccggccggcccgcttcctgccggccgtgcagccacacgagcaggtcctcgaggcgggcggtgaaccgggtgagtgtcgcctcgtccgcgtcggtggcgatcgccgtgtccaccagccggtccagttcggcgaggaccgccaggctgtcggacgtcgcagtccgcggggtctgcgaggccagtcgcgtgtagaggaagtcggccacggccaagggcgtcgggtggtcgaagatcagtgtgccggggagccgcagaccgctgacggcaccgagccggttgcgcagctccaacgcggtgagcgagtcgaagctcagctccttgaatctccgctccgggtccaccgtcgccgcggtcgcgtgtcccaggaccgccgcggcgctgtcctgaaccagctccagcagcatccctcgtcgcgcggcctcggtcggcagttcggccacgcgccgggccaggtccggtcccgcgtccaccgcggtctgccgcggtgccgggaggcgggcgccgtcggctgttgcggcggcctgtggcagacgccgtacgggccgggccggtgcgacgtgccggaacagcagcggcacctcggcgacgtcccccgctctcccggcaccaccggcgctttcggccagggccgcgaggtcgagccgtaccggcaccacggccggtaccgccgcgtcgcgcgtggcgtcgaagagggcgaggccctcgtcggtggacaggggcagtacgccggtgcgtgccatccggttcagatcggtcgcgctcaacgcctgcgtcataccgccggcggcgtgctcccaggggccccacgccgcggcgaccgcgggcagcccgcaccggtgtcggcggtatgcggccagaccgtccaggaaggcgttcgcggcggcgtagttgccctgtccggcgtggccgatcactccggcgacggacgagaacagtacgaaagccgacaggtcggcgtgggcggtgagttcgtgcagattccacgccgcgtccgccttgggccgcagcacggtgtcgagccgctccggcgtcagtgcttcgaccggggcgtcgtcgagcacgcccgcggcgtgcaccacggcggtcagtggatgcgcctccgggaccgcagcgacgacccgcgccagcgccgtgcggtccgccgcgtcgcaggcctcgacggtcacctcgacggcgcccgccgcgcgcagttccgcgacgatctcggtcacgccctcggcggccgggccgcgccgcccggtgagcaccagtcgcctgacgccgtgccgggtgacgaggtggcgggccacgttgccgcccagcgcgcccgtgccgccggtgatcaggacggtcccctccgggtccagcggcgccgggacggtgaggacgaccttgccgacgtgccggggccggctcaggtgccggaacgcgtcctgcgcgcgccgcacgtcccacgcggcgaccgggagcggacgcagcgcgccgcgctcgaacagctccgtcagctcgcccagcatggcgccgacgtgcgcgggttccagcttcgtcagatcgaacgagcggtggtcgacgcccgggtgccgggcggcgaccgcctcggagtcgggggtgtcggtcctgcccgtctcgacgaaccggccgccggacgacagcagccgcagcgaggcgtccacggcgtctccggccgggcagtcgagtaccacgtccacgccggcgccgccggtcgcggcccggaaccgggccgcgaactcctgggtgtgcgacggcgcgaggtgcgtgtcgtcgagcccatgtgcacgcaggtccgcccattcgccggggcccgctgtgccgaacacctcggcgccccggtgccgggcgagctgcgccgccgccaggccgacgccgtcggccaccgagtggatcagcagccgctcgcccggtacgacggcggccagttcgacgagtgcgtgataggccgtcaggaacgcgacgggcaccgacgcggcctgcgtgaacgtccagcccgccgggatacgagcgagggtgtcccgctccgccacggcctgggggccgaagttgccggtgagcaggccgaacacccggtcgccgacggccagatcggacacgccggggccggtctcggtcaccacacccgcgccttcgagcccgaggatgtcgtggtccggcggtgtgtcgcgggcgaggaccggggcgcggagatcgactccggccgcgcgcaccgcgatcctcacctgcccggtgccgagcggggcgcgctgggccgccccgacctgctcggccacgtcggccggtgcgggccacgggacgtcggcgtcctgcgcggcgcgagcgagtcgcggtacgagcacctcgccgtcgcgcaccgcgagttgcggttcgccggtgcgcagggcggcgggcagggcggcgtatccgtcggacgggtcgccgtcgatgtcgacgagggtgaagcagcccgggttctcggtctgggcgctgcgcaccaggccccacgcggtcgcctgcggcaggccgagtgcgtccggcgccgtcgccgcggccgtcgcatcgtgcgtgacgaggaccaggcggccgaccgtgagggcgggcgcgtcgagccaggacgtgaggagggcgagcgtgcggcgggcgacggcgtgcgcgccggccgcacctccttcgtgctctcccgtccgctctccttcgtgggccggaagttcggctccgtgcgcagcgagatcggcgagcacgacggacgggtgcgggtcaccgttctcgatgccgtggaccagggcgtcgaggttcgggtacgtgcggatgtcgaccgcctcggccgccagcgtggagaccggatcgggcacggggctccgaccgatgagcgcccactgctccggcgcgtccggagccgcggcgccgaccggtgccgctgccggtgtggcggaccactcgacgtggtacagcgaccggggtccgcccgcctccgtgccggagccgtcgctcaaccggtccaacaccacgggacgcagggacagttcccctgcggtcagtatgagggcgccggacggatcgctcgcgacgacgcggacggtggtcggcccggtccgggtgaaccggacccgcagcgcggtggcgcccagcgcgtgcagggcgacatcgccccaggagaagggcagcagcgtgcccgaggcatcggttccggacaccccgtccgcgagcagtccgctgcgcagcagggcgtgcagcgaggcgtcgagcagcgccgggtgcaccgagaaccggtccacgtcgtccgaggcggcggcatcgtcacccagctcgacctgtgcgaacacgtcatcgtccgtgcgccacgcggcggtcagcaaccggaagtcggggccgtactcgtagccggtcagggccagagcggggtagaggtccgtcaggtcgaccggcgccgcgtcggcgggcggccactgcggtgcacggtccgcggcgtcgggcgcctcggtgggccccagggctccgctcgcgtgccgggtccaggaggacgagccggaggcgtcgtcgccggcgggcgccgggcgcgaatggacggcgaaggcgcgcaggccggactcgtccgcctcctggacggtcacccggatgtcgacgccgcgctcgccgggcagcaccagcggtgcctgaagcgcgagttccgcgacagccgggtgctcccccgcgccgtccgacgcggcatgcagcaccagatcgagcagggcggtgccgggcagcagcgtcgtgccgtggatcgcatgatcggcgagccaggggtgcgtgagcgtgccgatacgcccggtgtggacgaagccgccgccctcgggcagttcgacggccgccgcgagcagcggatgcggcgtgcccgtcagcccggcctgcgtgacatcggcgcgcggcgcgggcggcgtgagccagtaacgctcgcgctggaaggcgtacgtgggcagttcgggcagagcagcggagcgagggccggggagtgccggccaggcgacgtcggcaccgctcgtgtgcagggtcgcgagggcgcgcagcagggcgtcgtgctccggccgtccgtggcgcagcaccgggaccagagccgcggggctctcctccagggtctcggcgaccagcgtggccagcgtcggagtgggagtgagttcgaggtagcgggtggtgccgagcccgtgcagggtggtgacggcatcggcgtggcggacggtgcggcgcagctgttcggtccagtagtcggcggtcgtgatccggtcctgctcggcgagcagtccggtgagcgtcgagacgatgggaatgcgcggcgcccggtacgtgagccccgccgcgatccggcggaactcgtccaggatctggtcctggtgcggactgtggaaggcgtgactgacggtcagccgcctgctcctgatcccccgctccgccagctgtgcggcgatgtccgcgaggacctcgggatcaccggacaggacggtcgactccggcgcgttgaccgccgcgagcgagaccacgtcctcacgccccgccacgagaccccgcgcggtggcctcgccggcctggagggcgagcatggtgccgggcgtggtgatctgctgcatcagacgggcccggtggaagaccagcgtggccgcatccgccaaagagagcatcccggcggcatgcgccgcggacagctcaccgatcgaatgccccaccagatggtccggccgcacaccgaacgactccagcagccggaacaacgcggtgtgcagaacgaacaacaccggctgcgtgaacgcggtccggttcagcagttccgccccctccgaccccggccccgcgaacatgacctcacgcagcgagcggcccagcagcggatcgaagaccgcacacgcctcatcgacggcagcggcgaacacgggatacgacgcatacaactcccgccccgcacccgggcgctgactgccctgaccggagaacaggaacgcagtcctgcccaccgtggcccgaccacgtaccaccatgggatgcccggcacccgaggcaagcgcggacagcgcctcggcgagtgcgtcccggtcctgggcgacgaccgccgcccggtggtcgaagtgcgtacggccggtggccagggcccgagcggcccggcggatgccgacctccgtccgggtcctggcgaactcggccagccggccggcctgttccccgagcgcgtcggctttcttcgcggagaccagccatacggcggggtctgcggagtcggcttcggcctcgtacgcggtccgctccttgaccggaggctcttcgaggatcaggtgcgcgttggtaccactgatcccgaacgacgacacggcggcacgacgcggccgctcccccgcctcccagacgaccggcccggtcaacagcctgacctcacccgcctcccagtccacatgcggcgaaggctcgtccacatgcaaggtcctgggaagcaccccgccccgcatcgccatgaccatcttgatctccgctgccactttgtttaccccttaagtcttcaatatcgagggtggccactcctggttgaggggtgggcgggcaccgcgcaggccctgccgctgtggttgcgtacgggccgctgcgcgacgggcgcgccgcacacgcgccntgagccccggatgaggggtgtaaagaaccttcttaccggacggacggcgtcgaggacgagtccatagaaccaagatcnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngatccggcaccggaagctgccgccgagcctgaacttcgtgacgccgaacccgaagatcacggacggtgtccaggagcaggcggtccgcgtcggtccgcgccatgccgggcagtcgccgggcgagcgtgggaccacccgcgccgcccgcgccgtcggaacgcccggcgtcggagccgtcgtccgcggcttccccggatcgggtcaccgagcgccgttcccgtcccatgagcctcgacggcgtcgacatcggcggcctccagccccgcgtcggccagggcctgacggatcacccgctgctgcgacgggccgttcggcgcggtcagaccattgctcgcaccgtcctgattgaccgccgagccgcgaatgaccgcgagcacacggtgcccgttgcgccgcgcgtcaccgagacgttcgagcaccagcatgcccacaccctcggcccatgaggtgccgtcggcggcggcggagaacgacttgcaccgtccgtcgcccgcgaggccgcgctgcctcgcgaattcgacgaacatgccggggctcgccatgacggcggcgccgcctgcgagcgccagttcgcattcgccgttacgcagcgaccgggccgcgaggtgcgcggcgacgagcgacgacgagcaggcggtgtccaccgtcatcgcggggccctcgaacccgaaggtgtaggcgatgcgtccggaggccacgctgacggtgctgccggtcagcagatagccgccgacgcttcccgccgtctcggggaccgtctcgtgcagccgggggccgtattccatggccgtcgcgccgacgaacacgccggtgcggcttccggccaggccggtcgggtcgatgccggcccgctccaccgcctcccaggaggtctccagcaggagacgctgctgcgggtcgacggccagggcctcgcgcggcgagatgccgaagaactgcgcgtcgaaccggtcggcctcgtagaggaaaccgccctcgcgcgcgtaggtccggcccggtgcgtcggggtccgggtcgtagagcccctccaggtcccagccacggttctccgggaacacgtcgatcgcgtcggcgccctcggcgacaagctgccacagcgcttcgggggaatcggcggcgccggggtaacggcaggccatgccgacgatcgcgatcggctcgtcggagaccgaggccgccgacgtggcgtcgctctgcgtggtaccggccagctccgctcccagcactcgtgccagggctcgtggcgtcggactgtcgtacaacagggttgccggcacgctcagttggagcacagcggccagccgctcgcacaggtcctccgcggactgcgactccaggccgagatcgttgaaggagcgggccaggtccacttcgcgcggatcggagtgaccgagcaccgccgccgcctcgtcacggatgaggtccagcaactgctcgtcacgcccggcaggagcggcctcgaccagccctcgcagccagtcggagtcccgcacggacgcgggtgcggagacagcggacgtcttctccgggcgggccgtcacggccgatgcggccgacgcggtggcacgccgcgcggcacccttcaactccagacccaacacctgcgcgaggaccttcggcgtcgggctctcgtacaacagcgttgcgggcagacgcagttgcagcacggaacccaaccgctcgaccaactccacacccgacgccgactccaggccgaggtccttgaacgagcgggcgagatccacttcgcgcggatcggagtgaccgagcaccgccgccgcctcgtcacggatgaggtccagcaactgctcgtcacgcccggcaggagcggcctcgaccagccctcgcagccagtcggagtcccgcacggacgcagacgcggagacagggggcccggaggcgggcacagcggcgccagcgggagcagcagggttcggcgtcggaacggcggcagcgccctggcgtgccacgggcgcggacgtcggcgtgggctcgggccaataccggcgccggtcgaaggcgtagccgggcagttcgacgcggcgcgccgccggaaggccgtagagcgccggccagtcgacggcagcgccccgcacgtgcgcggcggcgagcgaggacagcagccgcggccggccgccgtcgccgcggcccagggcgggaatgccgaccgcgcccgcggcgtcgaggagttccaggatctcgggcggcagcacggcgtgcgggccgacctcgatgaagacggtgtgcccgtcgtccatcagttcctcgacggccggatggaagggcgccggctgccggaagttgcggtaccagtggtccgcgtccagggcggcggtgtccaccggaccgccgagggtcgtcgactggaagcgcgtcccgctcggcctgggctcgatgccgctcagctcgtcgaggagcgcgtcgcgcaccgcctcggcctgctcgccgtgcgcacggccgcgtgcgacgacgagagccgcggcgtcctggagggtgagcgcgccgatgctgtacgcggcggcgatttcgcccgcggcgtggccgagcacggcatggggctggacgccgagcgtgcgccaggtgtgcgcgagggcggtcgtgacggcgaacagcacgggctggacgtggtcgggggtgtccggcagggtctccggaccggtgaggtggtcgacgagggaccagccggtgagcgggtcgagggccgcggcggcggcctccacgtgctcgcggaagacgggcagggtcgccatcaggtcgcggaccgtgccggcccactgcacgccctggccgggaaacacgaacacggtcttcggcccggtgccgggcgtggtgccggtcccggggtccggcgcggtgccgcgcagcaggccgtccgaggggcggccctgcgcgagggtgcgcagctcggagagcagggcggaccggtcgccgccgaacgcggcggcgcggtgctcgtgatgcgtgcgaatggtggccaggccgcgcgccacggtggcggcgtccagctcggggtgctgctccaggtgcgcggcgagggccgcggcctgaccgcgcagcgccgcctcgctgcgcgccgagatcagccacggcgacgcgacgtcctgcgggacggcaacgggaacaggaacggtcgcgggcggggccgccgatgacgtcgcgggtgacggcgccgacgactcggccggggcgtcgcagagcacgacgtggcagttggtgccgcccatgccgaacgagctgacccctgcgacgatcggaccgtccgggcgcggccacggtgtcagcccgacctggacgcgcaggctgagctcgtcgaaggcgatcttcgggttcggcgtcacgaagttcaggctcggcggcagcttccggtgccggatcgccagagccgtcttgaccaggcccaccacgcccgaagcaccctcgagatgcccgatgttggtcttcgcggagcccaccagcagcgcgttgtcggccacccggcccacgcccgcgccgaaggcggtgccgagcgccgccgcctctatcgggtcgcccacggcggtgcccgtgccgtgcagttccacgtactgcacgtcgccgggggccacgtccgcctgccggcaggcggcgcgcagcagctccgtctgcgcgggcgcgctgggcaccgtcagtccgtcggtggcgccgtcgttgttgaccgcgctgccccggatgacgcagtacacgaagtcgccgtcggcacgggcctgttccagcggcttgagtacgaccagtgcgccgccctcgccgcgaacgtacccgttggcccgcgcgtcgaaggtgtggcagcggccgtcgggcgagagggcgccgaagctcatcgacgcggccatgccctccggcgcggcgatcaggttcacgccgcccgccagcgccacccgcgactcgccgcggcgcaggctctcgcaggcgaggtgcacggcgaccagcgacgaggcctgcgcggcgtccacggtcatgctggggccgcgcaggcccagcgtgtacgacacacggttcgcgatgagcccacggcccatgccggtcatcgtgtgctggttgaaggaggcggttccggcccgggcgacgacgctgcggtagtcgtcccagatcgctccgacgaacactccggtgccgctgccgccgagcgaggcggggacgatcgcggcgtcctccagcgcctcccagctcagttccagcatcagccgctgctgcgggtccatcgcccgtgcctcgtgcggcgagataccgaagaacccggggtcgaaggtgtcgatccggtccaggtaggcgccgtaccgggccgcaccggtcggcgtgccggccgcgtcgggccagcggtcggcgggcgtctcgcccaccgcgtccacgccctcgctcagcagccgccagaaagtcgcagggtccggagccgccggcagccgacaggccataccgacgacggcgatgggcatgaacccttcagacatgaagcgcaccctcgacggatatggaggagtcgccgcggtccgcggcgggccggccgagcgtctccaacgacgcaaggaagtcgagctcttccagcggacgatcggtgaagaagcgcgcgatcgtctcggcccactcggcggcacgcccgaggaacacgagatggtcggcctccgggatgacggcgaacagcgcaccctcgatctcggcggccagggcgcgcgcgccctccatggtggcgaaggtgtcgtgctccccgaccaggcacaggctgggtacgccgctgatgccgccgggcaggacggcatcatcctggaggaggaggtccgagacgttgaggtagccgggaagatcggcctcctcgatcgtgctgaagcgaccgttgagggccctgcggacgatttcacggttgcgcaccgtgaccgcggggtccaggcacatgagcaggtccagcagaccttcggcgaactcggcgaagcggcccgccgtgaggatcggatacatctgcgtcatccgctcccggttgcgcggcggccagtcggcggccccggccaggacgagacgggagacccgcgacgggctctgctgggcgtagcggtaggccgccgggaatccgttgctgatccccagcaggttcacacgcggcagccccagctcgtcgatgaggtgggcgagcgcttcggtctggacgtcatagccgccttcggcgggcacggggtccgccgtgcgcgagcccggcagatccacacagacgatggtggccgtgtcctgccagtacttgtcgaaccgccggtagctgaacttgtcctggtacgcaccggacagcacaacaaaaggctcggtgaccggtgcttcgcattcgaccatccggtagctgaacgcaagacctttgtaatcaagctcttcgacctgctcgcgcggatttccggcgcccacggatcagctcctcgaatttcgggcggatgtgcacggacggacaacggatacacgtccgtgcatgagcccgatctttgtcgccggccagggcaccgacaacccctatttccccccttagccgaaccggcttgccggatcggagctggtcggagctgcgagatgagtcccgatacgaatcctctccagattcaccccctggcacacgacccatcgacatgtattctcggcgtatgcccatcatcgaacttgccgaatacgggccagactttctcgcagatccttacccgtattacgcgaaactccgcgaggagggacccgtgcacgaggtacgggccccggacggctatcgattctggctgatcgtcggatatgccgaggggcgcgccgccctgaccgattcgcggctggtcaaggcacgcgacacgatggcgacgtccgaggcgtcgccactgggcaagcatgtgctgatcgccgacccgccggaccacacccggctgcgcaagctgatctcccgggagttcaccgtgcggcgggtggacaacctgcgcccgcgcatccaggaactcaccgacgacttgctggacgtcatgctgcccgcggggcgggccgacctggtggaggcgctggcccggccgctgccgatcgccgtgctgtgcgaactgctcggagtgccgaacgccgaccgggacgagttccactcctgggccaagggcatcctcgcgccgcagaacccgaccgagacgcacacggccgtcaaggccttgatgagttatctcgacgacctgatcgaggacaagcggcacggagagcccaccggtgacctgctgtcgggtctcatacgcaccagcatcgagaacggcgaccgcctctcctcggaggaagtgcgctccacggccttcctcctgatgatcgccggacacgagacgacggcgaacctcatctccaacggaacgcgggcgctgctcacgcaccgggaccaactggacctgctgcgctccgacatggacctcctcgacggcgccgtcgaggagatgctccgctacgacggctcgctggagagcacgaccaagcggttcaccggtgtgccggtccagatcggcgacacggtcatcccgccgggcgagacggtgctggtcagcctcgcgtcggcggaccgcgacccggcgaacttcgacgaccccgaccgcttcgacatccgtcgcggcaccccggccggcgtcggccacctcgcgttcgggcacgggatccactactgcctgggagcctcactcgcccgcgcagagggccggatcgcgttccgcgcgctgctggagcgctgccccgacctcgaactcgaccccgaggcaccgccgttcgagtggatgccgggcgttctcgtccgcggcgtgcagcggttgtcgctgcgctggtag

SEQ ID NO: 24 - protein, 1872 aa, Streptomyces pulveraceus
  MISC_FEATURE: CDS  complement (>1755..6789) FosB (nter); PKS (module 2)
MPANDDKLRDYLKRVTADLHQTRLRIRDIEARKREPIAIVGMACRYPGGVTDPEQLWELAAGGIDAVSGFPSGRGWDLEGLYDPDPDAEGKVYVREGGFLHDAGQFDAPFFGISRREALAIDPQQRLVLETSWEAVERAGIDPLSLAGSRAGVFVGVMPQEYGPRLYEATGQGVSGHLLTGTTTSVLSGRIAYTLGLEGPAVTLDTACSSSLVAMHLATQALRSGECEVALAGGVTVMANPGTFVEFSRQRGLAPDGRCKSFAAAADGTAWGEGVGMLVLERLGDARRNGHRVLAVIRGSAVNQDGASNGLTAPNGPSQQRVIRQALADAGLEAADIDAVEAHGTGTTLGDPIEAQALLATYGQGRFEGRPLWLGSLKSNIGHTQAAAGVGGVIKMVMAMRGGVLPRTLHVDEPSPHVDWEAGEVRLLTGPVVWEAGERPRRAAVSSFGISGTNAHLILEEPPVKERTAYEAEADSADPAVWLVSAKSPDALRAQADRLTEFLAARPQTGTGHLARALATTRSQFEQRAALIGADRAGLTEALSALASGSGHPSLVRGQVTTGRTAFLFSGQGSQRPGAGRELYASYPVFAAAVDEACAVFDPLLGRSLREVMFAGPGSEGAELLNRTAFTQPALFVLHTALFRLLESFGVRPDHLVGHSIGELSAAHAAGMLSLADAATLVFHRARLMQQITTPGTMLALQAGEATARGLVAGREDVVSLAAVNAPESTVLSGDPEVLADIAAQLAERGIRSRRLTVSHAFHSPHQDQILDEFRRIAAGLTYRAPRIPIVSTLTGLLAEQDRITTADHWTEQLRHTVRHADAVTTLHGLGTTRYLELTAHPTLAPLAAETLEDASAAPAALVPTLRAGQPEPDTFLRALATLHVTGTPVTWFADHAEADADNADTADGRGHERGRATVPHLDLPTYPFQHENYWLTAPSSGTGPGAGADALPHPMLSQRTDLPGGGGVLFSGRLAPGTDPWLPDHAVMGTLLLPGTGFVELALEAARAVGAGRVEELVLRAPMVFPGGRARDLQVWVAPDQGGERELLIRTRTPGEDWTLHATGVVTASRVDTDGFTPDWTGAVWPPAGAEQIPGDTFYPDLAERGYEYGPAFRSVKALWRRGDDLFAEVVLPEDQPYGFGAHPALLDASLHALPITRSFYETDDEVRLPFSFGGVSLFATDVRRVRVRLRPRPEATSVWITDAAGTPVLAMESLILRAVERTQLQAAEGAVGQAATFAVRWEPLSEARIAERVPGTWLLFGTARPGLAELFEHVLTSTEWDASASTPVEGVLVCPADASELLAALRETERLDAPVWCVTSGAVGVGVDDPATDVAAAGAWGLGRVAALELPSRWAGLVDVPETADLGTADDNAGRTTARLLAGVLTGDGAEDQLAVRDGRLWARRLGTAPAADAGTWQPKGTVLITGGTGGLGAHVARRLAALGTADRLVLLSRRGAESPGAAELLAELGESGVRAEAAAIDITDRTAVTQLLSRLDAEDDPVRTVVHAAGVIRYARIADVDPEAFETDMAAKVNGALLLDELLPDADEFVMFSSIAGIWGAADQAAYAAGNACLDALARRRRERGASAVSIAWGPWSGGGMVTEYEDRELRKRGLLPLAVPSAVEALERAVPGDTDPVVVDVAWSRFLPAFTVLRPSPLLSGFAPATRRGGGRERXXDRGRAKDRVGALPEDERLPVLLDVVRTHVAQLIGRGDPQQVQADRALRELGFDSMMSVELRNRLGELVGARLPATLAFDHPTPESLAERLLTELDLDEAPADDGPVLEDFDRLEAKVLSPFTPADTRAALATRLSALLDRLSGTGTGAGGAGRNSGTDDLETASASDLMQFLDAEYGASDGTASDPSRPTTS

SEQ ID NO: 25 - protein, 1571 aa, Streptomyces pulveraceus
  MISC_FEATURE: CDS  complement (6882 . . <12440)
MMPSCPAASAAYPACAWSGSTTPSPPWRARAPWPPRSRVRCSPSSRRPTISCSSGVPPSGPRRSRASSPIVRWKSSTSLRRWRRSAGPPRTAATPPYPSRVRFMSEGFMPIAVVGMACRLPAAPDPATFWRLLSEGVDAVGETPADRWPDAAGTPTGAARYGAYLDRIDTFDPGFFGISPHEARAMDPQQRLMLELSWEALEDAAIVPASLGGSGTGVFVGAIWDDYRSVVARAGTASFNQHTMTGMGRGLIANRVSYTLGLRGPSMTVDAAQASSLVAVHLACESLRRGESRVALAGGVNLIAAPEGMAASMSFGALSPDGRCHTFDARANGYVRGEGGALVVLKPLEQARADGDFVYCVIRGSAVNNDGATDGLTVPSAPAQTELLRAACRQADVAPGDVQYVELHGTGTAVGDPIEAAALGTAFGAGVGRVADNALLVGSAKTNIGHLEGASGVVGLVKTALAIRHRKLPPSLNFVTPNPKIAFDELSLRVQVGLTPWPRPDGPIVAGVSSFGMGGTNCHVVLCDAPAESSAPSPATSSAAPPATVPVPVAVPQDVASPWLISARSEAALRGQAAALAAHLEQHPELDAATVARGLATIRTHHEHRAAAFGGDRSALLSELRTLAQGRPSDGLLRGTAPDPGTGTTPGTGPKTVFVFPGQGVQWAGTVRDLMATLPVFREHVEAAAAALDPLTGWSLVDHLTGPETLPDTPDHVQPVLFAVTTALAHTWRTLGVQPHAVLGHAAGEIAAAYSIGALTLQDAAALVVARGRAHGEQAEAVRDALLDELSGIEPRPSGTRFQSTTLGGPVDTAALDADHWYRNFRQPAPFHPAVEELMDDGHTVFIEVGPHAVLPPEILELLDAAGAVGIPALGRGDGGRPRLLSSLAAAHVRGAAVDWPALYGLPAARRVELPGYAFDRRRYWPEPTPTSAPVARQGAAAVPTPNPAAPAGAAVPASGPPVSASASVRDSDWLRGLVEAAPAGRDEQLLDLIRDEAAAVLGHSDPREVDLARSFKDLGLESASGVELVERLGSVLQLRLPATLLYESPTPKVLAQVLGLELKGAARRATASAASAVTARPEKTSAVSAPASVRDSDWLRGLVEAAPAGRDEQLLDLIRDEAAAVLGHSDPREVDLARSFNDLGLESQSAEDLCERLAAVLQLSVPATLLYDSPTPRALARVLGAELAGTTQSDATSAASVSDEPIAIVGMACRYPGAADSPEALWQLVAEGADAIDVFPENRGWDLEGLYDPDPDAPGRTYAREGGFLYEADRFDAQFFGISPREALAVDPQQRLLLETSWEAVERAGIDPTGLAGSRTGVFVGATAMEYGPRLHETVPETAGSVGGYLLTGSTVSVASGRIAYTFGFEGPAMTVDTACSSSLVAAHLAARSLRNGECELALAGGAAVMASPGMFVEFARQRGLAGDGRCKSFSAAADGTSWAEGVGMLVLERLGDARRNGHRVLAVIRGSAVNQDGASNGLTAPNGPSQQRVIRQALADAGLEAADVDAVEAHGTGTALGDPIRGSRGRRLRRRAFRRRGRRGWSHARPATARHGADRRGPPAPGHRP

SEQ ID NO: 26 - protein, 398 aa, Streptomyces pulveraceus; identical to SEQ ID NO: 15
  misc_feature (1)..(398): Complement (18399 . . 19595) P450

SEQ ID NO: 27 - DNA, 7316 bp, Streptomyces pulveraceus
  misc_feature (1)..(7316): source   <1 . . 8019
agacggtgtgaccgccgacggtgtggctgtcggcggcatgaccactcgtcaccaggtgacgggcaggctcttcgctccctggacgtcaagtcccctggagaacgggatctcgttcggcgcgacggccaggcgcagctgcgggatgcggctgaacagccgtccgtagaccacctccagctccatccgtgccacgctgtgcccgacgcacaggtggatgccggagccgaaggcgaggtggtcgcgggccgaccgctggatgtccagctcgtcggggcgctcgtacaccgattcgtcccggtcggctgagctgatcaggcagatgattccgtcgccgggccggatggtctgcccgccgatctcgatctcctccaccgccacgcgcttgggcgcgaactcggcgacggtgaggtagcgcagcagttcctcggtggcccccgcggcccgttcgggctccttggccagcagggcggcctgctcggggtggccgagcagcgccagcacaccggtggtgatcatcttgacggtggtgtcgtatccggcgccgagcagcagcccgatctgcatgagcaggttcgtctccgtgagcctgccttcgtcggcgaagccgaccaggcggctgacgatgttgccgtccggttccgcacggcgcagttcgaccagttccttgagctgctcgaacagttcaccgcgcgcggcgacggcggtttcggccgagccgtcctcgtcgaacagccgggcggtggtctcgccgaaccgggccatcgcctcggcaggcaccccgagcagccagccgacgaccgtcgacgggacaggacgggccagcgccggcacgaggtcggcggggctcgggccctccagcatccggtcgatgagcccgtcgacgagctcctcggtcgccggacgcagcgcccgcacccgcttgatcaggaagtgcggcgcgagcatccgccgcagttcggtgtggtcgggcgggtccatccggcccagcggccgtacgccgccctccgcggtgttcatggccttcgcgacctcactcagccatgggaaaccggggttgagcgcgtccacgctggcccgcgagtcggagaggatcttgcgcacgtcctcgtgccggctgatcagccaggcctcccgcccgttgtagagccgcgccttggacaccggctgctcggcgcgcaacgtccggtagacgggcgggggatccatcgggcagccgggcgccttcggcatcggatatgccggcggcgtctcggaggagctgtccgtcgtcaggggcggtgtaccggtgggcacggcgtcggtcgaggtcatcgtggcgaccctcctgggaagacttctggtggggtggttctgtcgcggcgggcgcgtgtcacgtttcgacaggcctgggcgcgcgtcacatctcggtgggtgggtgtcatgccctggccgacacaagcccgtcgttccgttcggcccgcttcgagccgatgtacgagaggaccgtcgcgcacagcgcgagggcggcgccgaccgcgaccgacgtggcaggacccgaggcctccagcagcacgccggtcaccggcatgctgagcgcggcgccgccggtgcccgcggtggccagccagccgaaggcttccgcccggcgctcctgcggcaccgctccggacacctgcgcgaagttgccggccatgctcggggcgatggcggtgccaccgaggaagagcacgaccagcacgagccagaccggagcggtttcggtcaccggcggcagcagcaccgcgagcagcgccatgccgactgccatcgccgcgaaccgggcgctgtgcggcacgtccttgcgcagggcgcccatgccgaagccgccgaccacggatccgatcgtccagcatgcgatgagcacgccggacagtccggactcgtggctgtcgcgggcccaggcgaccagggagaggttcaccgagaagagggcggccatcatcaccagggtcacgacgatggcgagggtgaacttcggcagggcgaagagcgtacgtcgctcctcgggctccggtgcggatgctctcgtgaccgcggacgccgacgtggccgtgtctccttccgtgccggcgtccgctcccgtttccgctgctgtccccgtctctgcttgtggtttcgcggcggtccgctccgccccgccgcggatcccggcgctgcccagcgcggcggcgaaggcgagcgcgccgacgaacgccacgacgccgcacgcgatgacggcgtatccggggtcgagggcggtcacgagcagtgaggtgagcagcggcccggtggtctgcaccacctccgagccggtggcctccagtgtgaacagcgtgcggccgagttcgccgggcacgatcttgggccacaccgcgcggctgacctgcgagatgggcacggtgctcattccggtgagcagggcgaccagcaccgcgatcggccagccgccgcccggcacggtgcgggtcatcgtcaccagtgcaccgaggccgacgaggtagccgatgccggtcaggaccagcagcttgcggaccgcgccccggtccgcggcgcggccccgggcgggtccgacgagcgcctgccccgcggtgagcgccccgccgagcacgccggcggcgacgtaggaatcgctccagccgaccagcaccagcgtgaggccgatcggcagcatggagacgttgaggcgggccagcatggaccagaggaacaggcgcggcaggtgcggaatgcctgccaatgccttgtaggacttcaccgttcacaccctgtttcgcaagtcatgacgcatacgcgcccgccgtgccgttcggcgccgccggacgctgcgtcttgatcaggtctcttcggacgttcggggccgctcagaaaacctggaaacgggcggcgatgcggtcggcgttctcggcgttctgccgggcccgctcgatcagcttctcgttgctccaggtgctggcggtggtgcctgtctcgtcgcccgcgttctgccgtgcggcgtagatccagccctcggcggcgcagcggaaccggaggatgtcggcgaggaaaccgagttccgcccggtcggcgggcccctcctcgaagtagccgcgcaccagcggctcgccgtccgtctcgtcctccaagtagctgagtgtggtggccagttccagcatgcagggtgcgtacatggcctccgaccagtccagcagacccgcggtgtcgccgaggacccggaactccttggccgcggcgtcgcagttgatcaggccgatggtcaggtcgtcgggcgacagcgcgccgcccgcctgctccagcgtccgccggatccagtcgtgtcccttgaggaagtcctgttcgagcaggaagagttcgagcacctcgttccagcgcggcacgccctccgggacgggggcgtgcagcaggacggagtcgatccgtccgagggtccggccgacggcgcgcaggtgggccgggtcggtctcgtcgacacgctcaccgtcgaggtaggtcagcaggctgtagcagaagtcgccctgataggcggtgacttctccgccggtggtcggcagcgggccgccggcggcgatgccgtgccgctccacctcctgggccaccaggagtcctgcggtgagcttcggccccatgtccttgcggacggccttgacgacgtggcgtacgccgtcgtgccggacgaggtaggtgtgcgagctgtacccctcgtctccgggcagccgctcccacggctcgacggtccagtcctgccagccccagagggtcagcggcaggtcgtgcgcttcgggtaccgcggctgcggcgtcggtcgattccggcattgaacgggttttccttgtctggtcgtgatgttccgagttccatgtccgacggccgcgcggtcgcgcggtcgcgcggtcgcgcggtcgcgcggtcgcgcggtcgcgcggtcgcggcatcatgtggtcatgtggtcatgcggtcacggggcagggtcatggtccgcgggcgcgtcccaggcgacggggagcgaattgacgccgtacaccacggagttcgcgcggaactcgatgtcctcgaagggcacggcgaggcgcagcgtggggaagcgctcgaacaggcgcaggtaggtcaccttcatctcgaccctggcgagctggtggccgatgcacaggtggatgccgtggccgaacgccaggttgcgctcctccgcccggtcgaggcgcagccggtgcgggtcggtgtagacctccgggtcgtggttggccgcggccatcgaaccgaccacggtctcgcccgccttgatgagctgcccgccgatctccacgtcctcggtcgccaggcgggcgaagttgaactgcacgatggacaggtagcgcagcagttcctcgacggcggtgtcgatgagcgacggatcggagcgcagcagggcgagctggtccgggttgcgcagcgcggccagggtgccgaggctgagcatgttcgaggtcgactcgtgcccggccatcagcatcaggccgcacattccggcgatctcgaactcggatatccggtccttctcctccgctggatcgagcagatcgctgatcaggtcgtccgacgggttcttcttcttgtcctcgatcaggtccagcatgaacttggtgccttcgaggacggtgcgctgctgctcctcctcggacagttcggtgtcgaggatgctcagggaccagcgctggaagtcgtcgcgcgcgtcgtagggcacgccgagcaggtcgcagatgatgagcgacgggatgggcagggcgtaggcggtgacgaggtcgaccgggccgccccccgtctcctccatccggtccaggtgctcacgggtgtactgctcgatcttcggctcaagagccttgatcttccggaccgcgaaacggctcgcggccagtcgccggtaacgggtgtggtccggcgggtccatggtgaagaaggcgccgggcagcggcgtggggcggccctcggtacgacgagtgcgggcggccttctcccggaccgagctgaaggacgggttcgccagcatcgccttgacgtccgcatggcgggtgagcagccagccctggttgccgtccgggaaggcgatgggactcacgggcgcctcggcgcgcaccttcgcgtagccgtccggcgggctgaaggggtcgctgcggtgcaggggcaggccctgcaggagagaaagagtgttcatggtcgtccttcctcagagggcgtcggtgtttcgagtcggtgcgtactgaggggtgcggagtggtacggggcgggcgcggacgcgcggcggccccgtacctgctccatcagggccgggtgaggcccggcgtacggctcagccactcgtccaccgccgcggccgtggtctcggagaattcgccgatcatggtgcagtggtcgcccggcacctgcgtctcctcgtggtcgaggggccacgccgcctgccagtcggggccggccatgggttcctcgggggaacccgggatgcagctgtccgggcggacgaagagagtcggcacggcgagttgccggggctgccagccgcggaacataccgcggtacgtaccgagggcggtgaggccgtcgtagtgcatcgacgtgaaccgcatccggcgctcgacgacctcgtaggtcatggccttgcgcatctccagcgtcatgctgtcgggcgggtaggtgtccagcaggacgacgccgaccgggcctgttccgcgctcctccagccaagtggcggcggcctgggcgagccagccgctggacgagtagccgagcagcgcgtacggccggccgtccgccgcacgcagcaccgcctccgccagtgtctcgatcagcacctccagggaagcggcgagaggctcgccggccatgaagcccgggacggtgaccacggagacccggcggcggccacggaagtggttggcgagccgggcgaactggagcgagccgtccacgggcgcgaacggcgggaagcagacgagctgcggctccgcctcgccgtggccgagggtggtgacgtgcgcgccgcggcccaggtcctcggcgccgtggaatctggtgcgcagggcggaggcgctgctgaggaacgcctcgacctcctgcatccggccttgcaacgagagcttgcggtagatgccgacgacggagtcgttggagtcctgcggcggcgcggcggccacggaggcggacggcgccgccgacacgccgggcgccggtgacgcgccggcgtcgccgcccggcgcggcattgtccagcagatgggtcaccagggcgccgagcgtcggatggtcgaacaccagggagctgggcagcgccaggccggtcaacacggtcagccggccgcgcagttcgaccgcggccagcgagtcgaagccgagcgccaggaactcctggtcctcggtgatcgtgccggcgtccgcgtgcccgaggacgcgcgcggcctcgcgacggacgatatcgaggacgtaagggcgctgatcggcgagcggaaggtccggcagccgctgccattcgccgggctgcgcggcaccggtgcgtccggcatcggggtgtccggcaccggactccgtatcgtcgaagtcggcgaacaggtggttcggccggcccgcggtgaagacgccgatgaaccgtggccagtccaggtccgcgacgacgatcgcggtgtcctcctgcctgaccgcccggtcgagggcggtggtggccaagcgcggggtgagcggccgaaggccccggcgctgcatctcctgccggaactcttctccggccatgccgccgccgctccagggcccccaggccagcgtggtggcggcggcgcggcgggcgcggcggcgctcgaccagcgcgtccaggaaggcgttcccggccgcgtacgcaccgccgcgcgtgctgccccaggtccccgcgatggacgagtagacgacgaacgcggcgagtccgtcgcccagcacctcgtccaggatgacggcaccggtgaccttcgcgtcgacgacggcggcgaattcggtcgcgtcgagatcagccagcggatgttcggccgccacgcccgccgtgtgcacgaccgcaccgacgggggcaccgcgcccggccaggtcggcggcgagcgccgcgacctcgtcgcggctggtcacgtcgcaggacaccaggtccaccgtggcaccgtgggcggccagttcggcccgcaggtccgcggcgccgggggcgtcgggcccctggcggctggcgaggacgaggtgcggggcaccctgctcggcgagccgacgcgccgtgtgcgcgccgagggcgccggtgcctcccgtgatgaggaccgagccgtgcgaccaccagggttcgcgcgcggcggg

SEQ ID NO: 28 - protein, 420 aa, Streptomyces pulveraceus; identical to SEQ ID NO: 14
  MISC_FEATURE: Complement (1418 . . 2752) P450

SEQ ID NO: 29 - protein, 444 aa, Streptomyces pulveraceus
  misc_feature (1)..(444): Complement (1418 . .2752)
VKSYKALAGIPHLPRLFLWSMLARLNVSMLPIGLTLVLVGWSDSYVAAGVLGGALTAGQALVGPARGRAADRGAVRKLLVLTGIGYLVGLGALVTMTRTVPGGGWPIAVLVALLTGMSTVPISQVSRAVWPKIVPGELGRTLFTLEATGSEVVQTTGPLLTSLLVTALDPGYAVIACGVVAFVGALAFAAALGSAGIRGGAERTAAKPQAETGTAAETGADAGTEGDTATSASAVTRASAPEPEERRTLFALPKFTLAIVVTLVMMAALFSVNLSLVAWARDSHESGLSGVLIACWTIGSVVGGFGMGALRKDVPHSARFAAMAVGMALLAVLLPPVTETAPVWLVLVVLFLGGTAIAPSMAGNFAQVSGAVPQERRAEAFGWLATAGTGGAALSMPVTGVLLEASGPATSVAVGAALALCATVLSYIGSKRAERNDGLVSARA

SEQ ID NO: 30 - protein, 356 aa, Streptomyces pulveraceus
  MISC_FEATURE (1)..(356): Complement (2859 . . 3929)
MMPRPRDRATARPRDRATARPRGRRTWNSEHHDQTRKTRSMPESTDAAAAVPEAHDLPLTLWGWQDWTVEPWERLPGDEGYSSHTYLVRHDGVRHVVKAVRKDMGPKLTAGLLVAQEVERHGIAAGGPLPTTGGEVTAYQGDFCYSLLTYLDGERVDETDPAHLRAVGRTLGRIDSVLLHAPVPEGVPRWNEVLELFLLEQDFLKGHDWIRRTLEQAGGALSPDDLTIGLINCDAAAKEFRVLGDTAGLLDWSEAMYAPCMLELATTLSYLEDETDGEPLVRGYFEEGPADRAELGFLADILRFRCAAEGWIYAARQNAGDETGTTASTWSNEKLIERARQNAENADRIAARFQVF

SEQ ID NO: 31 - protein, 409 aa, Streptomyces pulveraceus; identical to SEQ ID NO: 11
  MISC_FEATURE (1)..(409): Complement (3950 . . 5179)

SEQ ID NO: 32 - protein, 676 aa, Streptomyces pulveraceus
  MISC_FEATURE (1)..(676): Complement (5286 . . <7316)
PAAREPWWSHGSVLITGGTGALGAHTARRLAEQGAPHLVLASRQGPDAPGAADLRAELAAHGATVDLVSCDVTSRDEVAALAADLAGRGAPVGAVVHTAGVAAEHPLADLDATEFAAVVDAKVTGAVILDEVLGDGLAAFVVYSSIAGTWGSTRGGAYAAGNAFLDALVERRRARRAAATTLAWGPWSGGGMAGEEFRQEMQRRGLRPLTPRLATTALDRAVRQEDTAIVVADLDWPRFIGVFTAGRPNHLFADFDDTESGAGHPDAGRTGAAQPGEWQRLPDLPLADQRPYVLDIVRREAARVLGHADAGTITEDQEFLALGFDSLAAVELRGRLTVLTGLALPSSLVFDHPTLGALVTHLLDNAAPGGDAGASPAPGVSAAPSASVAAAPPQDSNDSVVGIYRKLSLQGRMQEVEAFLSSASALRTRFHGAEDLGRGAHVTTLGHGEAEPQLVCFPPFAPVDGSLQFARLANHFRGRRRVSVVTVPGFMAGEPLAASLEVLIETLAEAVLRAADGRPYALLGYSSSGWLAQAAATWLEERGTGPVGVVLLDTYPPDSMTLEMRKAMTYEVVERRMRFTSMHYDGLTALGTYRGMFRGWQPRQLAVPTLFVRPDSCIPGSPEEPMAGPDWQAAWPLDHEETQVPGDHCTMIGEFSETTAAAVDEWLSRTPGLTRP

SEQ ID NO: 33 - DNA, 8019 bp, Streptomyces pulveraceus
cccgggccgcgtcttcgacgtcctggagccgaccacgcgtctcgtgcagctcggcgctgacgcgcttcaggtaatccaacagctggttctcggatgccatttcccgctctccccatcaattcccggagggttctccacttgccgccgacgactcaggactcgtctatcccgggccctccagcggggagatgccgagctgccgggtgacgaagtcgaggacttcgtcctcggtggccgactccagcagcgcccccgtgtccggtgtgcccctctcggacgaggacccggagggcgaggagaacatgaccgtctcggaagcggaccgtttgccttcccagccgtccgcgagcgccctgagccgggacgccgcggccttccgcagggcggcgtccagccggagttcctcgaggctctgcgccactcggtacagctcggcgagggcggattccgcggtgacggcccgctcctccggctggatcaggccgtgcagttgcgcggcgagtgccttgggcgtcgggtggtcgaagaccatcgtggcgggcaggtccacgcccgcggcccgctgcagtctgttccgcagcgccatcgcggtcatcgagtcgatccccagctccttgaagggctggtcgacggcgagggcggcggggtcggcgtagccgagcaccgccgccgcgtgatcacggacggtgtccaggagcaggcggtccgcgtcggtccgcgccatgccgggcagtcgccgggcgagcgtgggaccacccgcgccgcccgcgccgtcggaacgcccggcgtcggagccgtcgtccgcggcttccccggatcgtgcgaatgcggcgagcagcgggctgggccgggccgcggtgaagccgtcggcgaacagcggccattcgatctcggccagcacctggctcgcggggccgtcctgggccagcgcgaggtcgagggcccgcacgccgagttcgggctcgatgggcggcagtccgttgcgccgcatccgctgttcggtggccgcgtcgacgagaccgccgccgccccagggcccccaggcgatcgagagggccgggaggcccgcggcgcggcggtgctcggcgagggcgtcgagcaccgcgttggccgcggcgtagttgccctggccgatgccgccgacggtaccgacgaagccggagtagagcacgaacgacgacaggttcaggccggcggtcagttcgtgcaggtgccaggcgccgagcgccttggggccgagaaccgcgtcgagccggtcggcgtccaggttctccagtgcggcgtcgtcgaggaccgcggcggcgtgcaccacggagacgagcggcgcgtcggccggtaccgactccagcagggcgcgtacggcctcgcggtcggcgagatcgcaggcggcgatcgtcacgcgcgcgcccatgctctcgaggtcggcgccaagtccgtccgcgtccagcgcctcaggaccgcggcggctggtgagcagcagatgttgcgcgccctgcgccgccagccggcgggccaggcggctgccgagggcgccggtgccgccggtgatcaggaccgtgccctcgcgcggccacaccgcatccgcgcccacgttcccgtccgtgtccgtgtccgtgtccgtgtccgacagggaaacctgaccgccgtcgcgtccgccggtcgtggggtccggcagccggaacggggtgcggacgaggcgtcgggcgagcacgcccgtcgggcgcagcgcgagttggtcctcgtcgcccgggtcggccagcaccgcgcagagctgagcggcggtggcggcgtccggcgcggtccgtccgtcctgcccgtcggcctcacggggcgcacggggcaggtcgatcagaccgccccacagcgtggggtgttcgagggcggcgacgcggccgaggccccagaccgcggcggcgtgcggtgccgccggagggtcggtcgggtcggttcccacggcgccgtgcgtgaggcaccacagccggcccggcaggtttgtctgcgcgagcgcccgcaccaggagcgtcgtcccggccaggccgcggccgaccgcggggcgtccaggtagcggccggtcgtcgagcgcgagcagggagaggatcccggccggccgggcatcggccgccgcggcgtgcagcgacgcggcccatgcggccgggtcggtgtcctcgcgcgggtcgagttccacgagcagcgcccggccgcccgcggcctcgacggcgtcgagcacccacccgacgagccggtggtcggggccgaatccgtgctcggcgccgggtatcgcgagcagccaggtgccggcgagggcggggacggcgcccggcgcgtgcaggaccggctcccagcggacgccgtagcgccaactgcccagccgtgcctgctccaggtggtcgcgccgccatgccgcgagggcgggcagcagggcgtccgccgactcgccgtccttcagcccgagggcgtccaggagcgcggcggggtcggcctccagcgcctcccacagacccgagtcggccgcctgcgcggcatgcggcgcggacgctccggccagggccgaggggcgcccgccccggccggcctcggatccggccccggagccattcgtggtgcgcggcgtcggccagaagcggctgcgctggaagggataggtgggcaggtcggtcacccggccgtcgctgtcgcgcagcagggcggcccagtccacgtcgagaccgtgcacccagccctcggcagccgaggtgaggaagcggtcggctccgcccgcgtcgcggcgcagcgtcggcacggtcgccggtcggacgcccgcctcctccgcggtgcgttccatcgccccggtcagcagcgggtgcgggctgatttcgacgaatacggcgtccccggactcggcgaggcggcggaccgcggcctcgaactccaccggggcccgcaggttgcgcacccagtactccgcggtcagttcgccgtcgcggaccggctcgccggtgacggtggacagcatctcgacctcggggcggcccggtgcgacgtccgcgagctccgtcaggatccgcgggccgatccggtcgacgtgcggcgagtgcgacgcgtagtcgaccgcgatccgccggacgcgcacgttgtcggcctcgcagacggtgaagaactcgtccagcgcgtccgcgtcgccggacaccacggtggcctcggggccgttggcggcggcgaggaagacgcggccttcgaagggcgcgaggcgggcggcggtctcggctcggccgagcgcgacggacagcatgccgccggcgcccgcgatcccggtcagcgtgcggctgcgcaacgccacgatcttcgcggcgtcttcgaggctgagggcgcccgcgacgcaggccgccgcgacctcgccctgcgagtggccgaccaccgtgtgggggcggacgccgacggagcgccacagctcggccagggacaccatcatcgcgaacagcacgggctggacgacgtcgacccggtccagggacggttcgccgggctcgccgttcagcacgtcgagcagcgaccagtcgacgtgcggggcgagcgcgtcggcgcactcccgcacccgcgcggcgaacaccggcgagctgtgcagcagttcgcgagccatgccggcccactgggcgccctgtccggggaagacgaacaccacccggcggcgaggggctgcggcgccgtcaccgtccacccggttcgcgctcggcacgcccgcggccagcgactccagttcggcgacgaccgtcgcacggtcgccgccgagcacgacggcccgctgctcgaaggccgtcctggtcgtcgcgagcgacagcgccacatcggccaggggcgactcgtcggcggtcaggcgcctgccgatcagccgggcctgggccgacagggcgtcgggggtgcgtgcggagaccaggaagggcagaactcccgatccctgagccggagttccggtgcccttggccgtcggcgcgggtacgtccggttccgggtcgggggcctgttcgaggatcacgtgtgcgttggtgccgctgatgccgaaggacgagatgccggcgcggcggggccggtcggtccgcggccacggctgtgcctcggtgagcaggcggacggcgcccgccgaccagtcgacgtgcgtggccggccggtcgacgtgcagcgtgcgcggcagcacgccgtcgcgcatggccatcaccatcttgatgatgccgccgaccccggccgcgccctgcgtgtgacccaggttcgacttcaacgaaccaagccagagcgggcgttcggccgggtgctcgcgcccgtaggtggcgagcagcgcccgtgcctcgatcgggtcgccgagcgtcgtgccggtgccgtgcgcctccacgacgtcgacgtctgcggcctggacctgcgcgtcctggagggcgcgccagatcacgcgctcctgcgcggggccgctcggcgcggccagaccgttggacgcgccgtcctggttgaccgccgaaccgcgcacgacggccagcacgcggtgcccgttgcgctgcgcgtccgagaggcgttcgaggacaagcatgccggcgccctcaccccaggaggtgccgtcggcggcgtccgcgaaggacttgctgcggccgtccgcggcgaggccacgctgccgggagaactcggtgaaggtgatgggcgtcggcatgactgtcacgccgcctgcgagcgccagcgagcattcgccgcggcgcagcgactgggcggccagatgcagcgccaccagcgacgacgagcaggcggtgtcgatcgtgaccgcgggcccggacagcccgagggcgtaggagacacggcccgagacgacggcgctgttgttgcccgtcacgaggtagccctcgtactcctcggacgaggcctgcaagggcacgtcgtagaacgagtagtaggcgccggtgaacacgccggtctcgctctcgcgcagcgtgtccggggcgatgccggcgcgctccagggactcccaggccacttcgagcatgaggcgctgctgcgggtccatggcgacggcctcgcgcggcgagacgccgaagaaacctgcgtcgaaccaggcggcgccgtgcaggaacccgccctcgcggacgtacgaggtgccctcctcggtgccgtcgccggcgaagagcgcgtcgaggtcccagtcccggtccttggggaaggcgccgatcgcgtcgcggcccgtgcgcaccaggtcccacagcgcctccggggagtcggagccgccggggaagcggcagcccatgcccacgatcgcgatgggctcggtggcgcggcgctcggtgtccttcaggcgccggcgggagtcctggaggtcgccggtgaccttccgcagcgcctgaagaagctgttcctggcttacgtcgtcagacatccacgccaccgttttccactctcgggcaaagcactgtcactcgaagcccttctcgatcagcgccagcaggtcggaggcgctggcggtcgcgatggccgcgccgccgtccgtgtcgtcgtccgcggcttgtgcggccggttcggtgtcgcggggtgtctcgcgcagccgggcggccagcgcgccgagccgggtggcgagccggtcccggtcggcggcggagccttcgaggtccgcgagcgaccgctccagcgaggcgaggtcggccagcgcgcgggcggcctgtccgccgccgtccgggacggcttggcggcagaggaacgcggcgacgtcggtcggcgtcggatgctcgtagacgagcgtggcgggcaggtcgagaccggtggactcgcgcagccgggtgcgcagttcgacggccatcagggagtcgaagccgaggtccttgaaggcgtggtgcggcgcgacgtcccgggccgcggcgtgaccgagcacggcggcgatctgggcgcgcactaggtcgagcgcgacgcgctgctgttcgtcgggactgtgcccggccagcactttggcgaagtccggcgtggtgccgcccgtctcgtcggtgcccggacccgcgccggtgacgccggcgaggtcggaggcggcaccgggcaccggaacgagtccggcgagcagcgggctggggcgggcggcggtgaagacggcggtgaagcgtccccagtcgacgtccgcgacggtgaccgtggtgtcgccgtgccgcagcgcccggtccagggcggtgacggcgagttccggcgccatcggggcgatgccgagtcggcgcaggtcgtcaccgaagctcccggcagccatgccgccgccgtcccaggggccccaggcgatggccttgcctgcggcgccccgggcgcggcgccgctccatcagcgcgtcgacatgcgcgttcgaggcggcgtaggcgccgccggacgcgttgccccagacgcctgcgacggaggtgaaggcgacgaacgccgacaggccgtcgccgaagacctcgtcgaggttgcgcgcgccggtcaccttcgccgccatcacggtcgcgaaggtgtcggcgtcgagcgcggcgacggggttctggtccccggtgcccgcggcgtgcaccaccgcgccgacgggcgtgtccgcggcggcgaaccgggcggccagttcctcgacgtcggcccggcgggagacgtcgcagtccgcgaggacgagttcggtgccgtgccgcgccagttcggcgcggagccggtccgcgtcggcgaagccactggcgcggcggctcgcgagcacgacgatgggcgcgccggactcggcgagccggcgggcggtgtggacggcgagggcgccggtgccggtgatcagcacgggtcccgaggaccacaggtccgcgtcctcggtgtcctcggcgggccgttcgtgggggctcacggtcggctcggcgcgggtcaggcgccgggcgtggacgctgtcgtcccgcagcgcgagctggtcgtcgccgccggatcgcccggcgaggacggcggccagccgggcgagcccgccgcagccgaggggatcgtcgtccgtgctcgccgcgaggtcgatcaacccgccccagagtcccgggtgttcgagtccgacgacccggccgaggccccagacctgggcctgccacgggtccggcgccgcgtcactctccagcgcccgcaccgcgcccaagggtcagggtccacagcggcgggtccgtgcggcccgtttcgagcagagcctgtacgagcagcaggggtggcgtccgcggcggccgacacgccgtcgcggcgcgcggtctcaccgctcggccagggcagggcgagcacgcccgcgaaccgctcgtccgacgccggggtccggtccagttcccgggcgagttccttcgccagggtcgcgcgatcggcggtcgccacgtcgaccgtcaggaggcgggtcgtcgcgcccgcgtcggccagtgcggccgtgacggcgtcggcgagcgcggccgacgcgccgtcttcggcggtgtcggcgtcggattcgggggcgacgatgagccaggtgccgtcaagtgccgccgccgggggcgcggcgtccggaagacgttcccaggtgacgtggtagcgccacctgtcggttctggcgatcatgtcctgtccgcgccgccagttgccgagcgcgggcagcacggcgctcagcggcgcgtcggcgccgaggccgagcgtgcgggccaggccgtcgaggttctgctcctcgacgacctgccagaacgccgtgtcctccgcgctgtgcgtgccgtccggcgcggccggaacgtccgccgtgtccggtgcggcgtccagccagtagcgccggtgccggaaggcgtacgtcggcagtgcgacacgccgggcgccgggcgtgagcgcggtccagtcgaccggcacgccgttgacgtacgtccggctcagcgcggtcagcacggtcgccgcctccggccggccgcgccgggaggaggccgcgacgggccgagtcccctcgaacagcggcgtcagggcggcgtccgggccgatctcgatcagcacgtccgcgtccggcaggccgcgcaccgcgtcggcgaagcgcaccgcgcgccgcgcgtgctcgatccagta

SEQ ID NO: 34 - protein, 1738 aa, Streptomyces pulveraceus; identical to SEQ ID NO: 3
  MISC_FEATURE (1)..(1738): CDS complement (164 . .5380) FosPKS subunit, module 5

SEQ ID NO: 35 - protein, 129 aa, Streptomyces pulveraceus
  MISC_FEATURE (1)..(129): FosA fragment
SSGSASRSSGSAAASGAGSXXXXXXXXXXXXXXXXXXXXXXXXXXXXXXXXXXXXXXXXXXXXXXILVLWTRPRRRPSGKKVLYTPHPGLXARVRRARRAAARTQPQRQGLRGARPPLNQEWPPSILKT

SEQ ID NO: 36 - protein, 1856 aa, Streptomyces pulveraceus
  MISC_FEATURE (1)..(1856): FosA fragment
GVNKVAAEIKMVMAMRGGVLPRTLHVDEPSPHVDWEAGEVRLLTGPVVWEAGERPRRAAVSSFGISGTNAHLILEEPPVKERTAYEAEADSADPAVWLVSAKKADALGEQAGRLAEFARTRTEVGIRRAARALATGRTHFDHRAAVVAQDRDALAEALSALASGAGHPMVVRGRATVGRTAFLFSGQGSQRPGAGRELYASYPVFAAAVDEACAVFDPLLGRSLREVMFAGPGSEGAELLNRTAFTQPVLFVLHTALFRLLESFGVRPDHLVGHSIGELSAAHAAGMLSLADAATLVFHRARLMQQITTPGTMLALQAGEATARGLVAGREDVVSLAAVNAPESTVLSGDPEVLADIAAQLAERGIRSRRLTVSHAFHSPHQDQILDEFRRIAAGLTYRAPRIPIVSTLTGLLAEQDRITTADYWTEQLRRTVRHADAVTTLHGLGTTRYLELTPTPTLATLVAETLEESPAALVPVLRHGRPEHDALLRALATLHTSGADVAWPALPGPRSAALPELPTYAFQRERYWLTPPAPRADVTQAGLTGTPHPLLAAAVELPEGGGFVHTGRIGTLTHPWLADHAIHGTTLLPGTALLDLVLHAASDGAGEHPAVAELALQAPLVLPGERGVDIRVTVQEADESGLRAFAVHSRPAPAGDDASGSSSWTRHASGALGPTEAPDAADRAPQWPPADAAPVDLTDLYPALALTGYEYGPDFRLLTAAWRTDDDVFAQVELGDDAAASDDVDRFSVHPALLDASLHALLRSGLLADGVSGTDASGTLLPFSWGDVALHALGATALRVRFTRTGPTTVRVVASDPSGALILTAGELSLRPVVLDRLSDGSGTEAGGPRSLYHVEWSATPAAAPVGAAAPDAPEQWALIGRSPVPDPVSTLAAEAVDIRTYPNLDALVHGIENGDPHPSVVLADLAAHGAELPAHEGERTGEHEGGAAGAHAVARRTLALLTSWLDAPALTVGRLVLVTHDATAAATAPDALGLPQATAWGLVRSAQTENPGCFTLVDIDGDPSDGYAALPAALRTGEPQLAVRDGEVLVPRLARAAQDADVPWPAPADVAEQVGAAQRAPLGTGQVRIAVRAAGVDLRAPVLARDTPPDHDILGLEGAGVVTETGPGVSDLAVGDRVFGLLTGNFGPQAVAERDTLARIPAGWTFTQAASVPVAFLTAYHALVELAAVVPGERLLIHSVADGVGLAAAQLARHRGAEVFGTAGPGEWADLRAHGLDDTHLAPSHTQEFAARFRAATGGAGVDVVLDCPAGDAVDASLRLLSSGGRFVETGRTDTPDSEAVAARHPGVDHRSFDLTKLEPAHVGAMLGELTELFERGALRPLPVAAWDVRRAQDAFRHLSRPRHVGKVVLTVPAPLDPEGTVLITGGTGALGGNVARHLVTRHGVRRLVLTGRRGPAAEGVTEIVAELRAAGAVEVTVEACDAADRTALARVVAAVPEAHPLTAVVHAAGVLDDAPVEALTPERLDTVLRPKADAAWNLHELTAHADLSAFVLFSSVAGVIGHAGQGNYAAANAFLDGLAAYRRHRCGLPAVAAAWGPWEHAAGGMTQALSATDLNRMARTGVLPLSTDEGLALFDATRDAAVPAVVPVRLDLAALAESAGGAGRAGDVAEVPLLFRHVAPARPVRRLPQAAATADGARLPAPRQTAVDAGPDLARRVAELPTEAARRGMLLELVQDSAAAVLGHATAATVDPERRFKELSFDSLTALELRNRLGAVSGLRLPGTLIFDHPTPLAVADFLYTRLASQTPRTATSDSLAVLAELDRLVDTAIATDADEATLTRFTARLEDLLVWLHGRQEAGRPDAEDAAGATDRFESASDDEIFDFIDNELGLT